(19) United States
(12) Patent Application Publication
(10) Pub. No.: US 2022/0323626 A1
(43) Pub. Date: Oct. 13, 2022

(54) Compositions, Kits, Methods and Uses for Preventing Microbial Growth

(71) Applicant: Collidion, Inc., Petaluma, CA (US)

(72) Inventors: Hojabr Alimi, Petaluma, CA (US); Sridhar Govinda Prasad, San Diego, CA (US); Santosh C. Sinha, San Diego, CA (US)

(73) Assignee: Collidion, Inc., Petaluma, CA (US)

(21) Appl. No.: 17/596,570

(22) PCT Filed: Jun. 14, 2020
(86) PCT No.: PCT/US20/37665

§ 371 Date: Dec. 13, 2021

Related U.S. Application Data
(60) Provisional application No. 62/977,090, filed on Feb. 14, 2020.
(60) Provisional application No. 62/861,987, filed on Jun. 14, 2019.

(51) Int. Cl. A61L 2/18; A01N 29/02; A01N 59/16

(52) U.S. Cl. CPC A61L 2/18; A01N 29/02; A01N 59/16; A61L 2101/06

(57) ABSTRACT

The present specification discloses a composition comprising, consisting essentially of, or consisting of hypochlorous acid or free available chlorine alone or in combination with one or more quaternary compounds or silicon quaternary compounds, one or more guanide-containing compounds, one or more alcohols, one or more metallic particles, one or more metal salts, or any combination thereof. The present specification further discloses a kit comprising, consisting essentially of, or consisting of a one or more containers including a composition disclosed herein or components which make up such compositions as well as methods and uses for such compositions and kits.

This application is a 35 U.S.C. § 371 U.S. national stage patent application which claims the benefit of priority and is entitled to the filing date pursuant to 35 U.S.C. § 119(e) of U.S. Provisional Patent Application 62/861,987, filed Jun. 14, 2019, and U.S. Provisional Patent Application 62/977,090, filed Feb. 14, 2020, the content of each of which is hereby incorporated by reference in its entirety.

Water plays an important role in human health and also plays an important role in the health care field as well as in a variety of residential, commercial and industrial settings. Water or other liquid sources may become contaminated with organic materials and various other compounds that support microbial growth and residual microbes, including bacteria, algae, fungi (including mold, mildew, and yeast), and viruses. Control and prevention of microbial growth within such water or other liquid sources itself is a critical aspect in a wide range of industries and uses. Non-sterile water is most often used within medical devices operated at home or hospital (I.s. CPAP). As such, it is of vital importance for many individuals, especially those with weakened immune systems or disease, that non-sterile water when used does not promote an environment for further bacterial propagation leading to higher chances of infection for patient.

For example, in the healthcare industry the use of sterile and non-sterile water or another liquid free of pathogenic levels of microbes is needed in the manufacture and/or delivery of pharmaceutical drugs and other regulated and non-regulated products for internal or external use by an individual as well as in the operation and/or performance of medical devices, equipment, machinery or other apparatus. Although medical devices, equipment, and machinery are themselves sterilized or disinfected prior to use, the water or other liquid added to these devices, equipment, or machinery after such cleaning treatment may either introduce microbes or subsequently become contaminated by many factors including but not limited to patient use during device operation or separately during storage of the device. For instance, the use of water within heart-lung bypass machines used in conducting open heart surgery is currently under investigation for probable cause of primary infection in patients undergone such surgical procedure where symptoms of such infection may take up to several months to reveal itself with mortality rate of greater than 40% in this patient population. Other medical devices which also rely on water as part of their operation have similar contamination issues, such as, e.g., Continuous Positive Airway Pressure (CPAP). Further, microbes can contaminate adjacent medical equipment and the patient environment in general including surgical rooms. Hospital acquired infections (HAI) are a major cause of mortality and morbidity and represent a significant cost to the healthcare system in the U.S. and globally. As such, the type and certification of water or other liquid used in these medical applications are of high importance to FDA and as such are highly controlled by this regulatory agency.

Similarly, considering the apparatus, microbial growth can readily spread from contaminated water or other liquid contained source into associated residential, commercial or industrial equipment, appliances or machinery as well as a house, building or facility. Such apparatus include, without limitation, refrigerators, freezers, dishwashers, clothes washers, water heaters, air conditioners, cooling towers, or any other apparatus using water in the course of its operation. Thus, it is critically important to have water or other liquid free of pathogenic levels of microbes which can also prevent subsequent microbial growth that could arise from a contamination event during the storage or use of a device, equipment, machinery or other apparatus as well as a substance like a drug or food.

Simply diluting a sterilizing agent or disinfectant to a concentration that restrains or prevents microbial growth is not a viable solution. For example, chemical agents used to sterilize or disinfect a device, such as a medical device tend to be either an irritant or toxic to an individual. As such, even in lower concentrations such chemical agents are not useful in applications where an individual will be exposed to water or another liquid containing these chemical agents. As another example, microbiocidal agents, including antibiotics, used at lower concentrations act as a mutagen facilitating the development of microbes that are resistant to these microbiocidal agents. Furthermore, microbiocidal agents in various industries, like the agricultural industry or the paper manufacturing industry inevitably enter the waterways caused adverse environmental impact as well as increasing the likelihood of creating resistant microbes.

Microbiostatic water products are currently marketed as injectable additives for mixing with pharmaceutical preparations that are then administered by intravenous, intramuscular, or subcutaneous injection. Such preparations use lower levels of microbiostatic agents to safely prevent adverse levels of bacterial contamination within the injectable prior to patient exposure. One example of these products may include 0.9% benzyl alcohol in combination with sterile water. However, benzyl alcohol has been associated with toxicity and hypersensitivity in some individuals and may result in local reactions and histamine release. In cases where patients are unable to metabolize benzyl alcohols properly, results can be fatal with prolonged exposure.

Microbiostatic agents are also added to water to prevent microbial contamination in home humidifiers. The agents used in these solutions can include alkyl dimethyl and diethyl ammonium chlorides. These agents are known to damage mucosal and other human tissues. The use in home humidifiers typically prevents the vaporization of the microbiostatic agent and is also typically used in general (room) proximity to an individual. Medical applications using humidity, however, require the use be directly on or in a patient, thus, microbiostatic compositions comprising alkyl dimethyl and diethyl ammonium chloride are not suitable for such medical applications.

Other microbiostatic agents can also be created using lower-than-effective, pre-existing anti-microbial agents that are capable of killing bacteria and microbes, respectively, at therapeutic concentrations. However, such formulation will partially expose microbes to low concentrations of the anti-microbial agents. Historically, antimicrobial resistance (AMR) is created, in part, through overuse, misuse, prolonged use of prophylactic anti-microbial products. As microbes are exposed to therapeutic agents, they build resistance to the anti-microbial agents. One mechanism of such adaptation would be survival of strains of microbes that naturally develop resistant mechanisms through their own rapid multiplication and genetic diversity. Through a natural selection process, survivability is optimized with greater exposure.

As such, there is a need to develop safe, non-toxic compositions that can either substitute for or be added to water or another liquid that restrain, prohibit, or otherwise prevents microbial population growth. The present specification discloses compositions, methods and uses that provide superior prevention of microbial population growth in water or other liquid. The water or other liquid can be manufactured as sterile or non-sterile. A disclosed composition can be added to water or other liquid or substitute for these fluids and can be used prior to or during an application. In addition to being effective, the discloses compositions, methods and uses are safe and non-toxic to an individual and the environment and can be used in any application where the disclosed compositions are used directly on or in an individual or will be exposed to the environment. Furthermore, the likelihood of development of antibiotic resistance bacteria and other microbes is greatly reduced as the discloses compositions, methods and uses are not mutagenic to the microbes and have not been shown to induce antimicrobial resistance. In addition, in preventing microbial population growth in water or other liquid, the disclosed compositions, methods and uses reduce and prevent subsequent microbial contamination to other individuals or devices exposed to the treated water or other liquid. A disclosed composition provides a unique product that is shelf-stable and safe for individuals who using microbiostatic water-containing solutions.

### SUMMARY

Aspects of the present specification disclose a composition comprising one or more microbiostatic agents. The amount of one or more microbiostatic agents can be an amount that restrains or inhibits microbial population growth. A microbiostatic agent comprises hypochlorous acid or free available chlorine, one or more guanide-containing compounds, one or more surfactants, one or more quaternary compound or silicon quaternary compounds, one or more metallic particles, one or more metal salts and/or a phosphate buffer like calcium phosphate or sodium phosphate. A composition disclosed herein is stable and is effective under environmental conditions that promote microbial growth.

Aspects of the present specification disclose a composition comprising hypochlorous acid or free available chlorine. The amount of hypochlorous acid or free available chlorine can be an amount that restrains, prohibits, or otherwise prevents microbial population growth. A composition disclosed herein can further comprise one or more guanide-containing compounds, one or more surfactants, one or more quaternary compound or silicon quaternary compounds, one or more metallic particles, one or more metal salts and/or a phosphate buffer like calcium phosphate or sodium phosphate.

Aspects of the present specification disclose a composition comprising hypochlorous acid or free available chlorine and one or more quaternary compounds or silicon quaternary compounds. The amount of hypochlorous acid or free available chlorine and/or one or more quaternary compound or silicon quaternary compounds can be an amount that restrains, prohibits, or otherwise prevents microbial population growth. A composition disclosed herein can further comprise one or more guanide-containing compounds, one or more surfactants, one or more metallic particles, one or more metal salts and/or a phosphate buffer like calcium phosphate or sodium phosphate.

Other aspects of the present specification disclose a composition comprising hypochlorous acid or free available chlorine and one or more guanide-containing compounds. The amount of hypochlorous acid or free available chlorine and/or one or more guanide-containing compounds can be an amount that, prohibits, or otherwise prevents microbial population growth. A disclosed guanide-containing compound includes an organic compound containing a biguanide functional group, a biguanidine functional group, a triguanide functional group, or any combination thereof. A composition disclosed herein can further comprise one or more quaternary compound or silicon quaternary compounds, one or more surfactants, one or more metallic particles, one or more metal salts and/or a phosphate buffer like calcium phosphate or sodium phosphate.

Aspects of the present specification disclose a composition comprising hypochlorous acid or free available chlorine and one or more guanide-containing compounds, and one or more surfactants. The amount of hypochlorous acid or free available chlorine and/or one or more guanide-containing compounds and/or one or more surfactants can be an amount that restrains, prohibits, or otherwise prevents microbial population growth. A disclosed guanide-containing compound includes an organic compound containing a biguanide functional group, a biguanidine functional group, a triguanide functional group, or any combination thereof. A composition disclosed herein can further comprise one or more quaternary compound or silicon quaternary compounds, one or more metallic particles, one or more metal salts and/or a phosphate buffer like calcium phosphate or sodium phosphate.

Other aspects of the present specification disclose a composition comprising one or more alcohols and one or more quaternary compound or silicon quaternary compounds. The amount of one or more alcohols and/or one or more quaternary compound or silicon quaternary compounds can be an amount that restrains, prohibits, or otherwise prevents microbial population growth. A disclosed alcohol includes ethanol, methanol, isopropyl alcohol, or any combination thereof. A composition disclosed herein can further comprise one or more guanide-containing compounds, one or more surfactants, one or more metallic particles, one or more metal salts and/or a phosphate buffer like calcium phosphate or sodium phosphate.

Other aspects of the present specification disclose a composition comprising one or more alcohols compound and one or more guanide-containing compounds. The amount of one or more alcohols and/or one or more guanide-containing compounds can be an amount that restrains, prohibits, or otherwise prevents microbial population growth. A disclosed alcohol includes ethanol, methanol, isopropyl alcohol, or any combination thereof. A disclosed guanide-containing compound includes an organic compound containing a biguanide functional group, a biguanidine functional group, a triguanide functional group, or any combination thereof. A composition disclosed herein can further comprise one or more quaternary compound or silicon quaternary compounds, one or more surfactants, one or more metallic particles, one or more metal salts and/or a phosphate buffer like calcium phosphate or sodium phosphate.

Other aspects of the present specification disclose a composition comprising hypochlorous acid or free available chlorine and one or more metallic particles. The amount of hypochlorous acid or free available chlorine and/or one or more metallic particles can be an amount that restrains, prohibits, or otherwise prevents microbial population growth. A composition disclosed herein can further comprise one or more metal salts and/or a phosphate buffer like calcium phosphate or sodium phosphate.

Other aspects of the present specification disclose a composition comprising hypochlorous acid or free available chlorine and one or more metal salts. The amount of hypochlorous acid or free available chlorine and/or one or more metallic salts can be an amount that restrains, prohibits, or otherwise prevents microbial population growth. A composition disclosed herein can further comprise one or more metallic particles and/or a phosphate buffer like calcium phosphate or sodium phosphate.

Other aspects of the present specification disclose a kit comprising one or more containers including a composition disclosed herein and/or one or more containers including components of a composition disclosed herein. A disclosed kit further comprises one or more delivery or application systems, and/or instructions, and/or a container.

Other aspects of the present specification disclose a making a microbiostatic water or other liquid. In aspects, a disclosed method comprises applying a microbiostatic agent disclosed herein to water or other liquid in a microbiostatic effective amount, wherein application of the composition reduces or inhibits microbial growth. In other aspects, a disclosed method comprises applying a microbiostatic agent disclosed herein to water or other liquid in an amount that restrains, prohibits, or otherwise prevents microbial population growth without free ting the microbes, wherein application of the composition reduces or inhibits microbial growth. In yet other aspects, a disclosed method comprises applying a microbiostatic agent disclosed herein to water or other liquid in an amount that restrains, prohibits, or otherwise prevents microbial population growth without eradicating the microbes to a degree that achieves disinfection or sterilization, wherein application of the composition reduces or inhibits microbial growth.

Other aspects of the present specification disclose a method of reducing or inhibiting microbial growth in water or other liquid. In aspects, a disclosed method comprises applying a composition disclosed herein to water or other liquid in an amount that restrains, prohibits, or otherwise prevents microbial population growth without eradicating the microbes, wherein application of the composition reduces or inhibits microbial growth. The water or other liquid can be manufactured as sterile or non-sterile. In other aspects, a disclosed method comprises applying a composition disclosed herein to water or other liquid in an amount that restrains, prohibits, or otherwise prevents microbial population growth without eradicating the microbes to a degree that achieves disinfection or sterilization, wherein application of the composition reduces or inhibits microbial growth. In yet other aspects of the present specification disclose a composition disclosed herein for use in reducing or inhibiting microbial growth in water or other liquid. In still other aspects of the present specification disclose a use of a disclosed composition to reduce or inhibit microbial growth in water or other liquid.

Other aspects of the present specification disclose a method of reducing or inhibiting microbial growth in a medical device. In aspects, a disclosed method comprises applying a composition disclosed herein to water or other liquid present in a medical device in an amount that restrains microbial growth, wherein application of the composition reduces or inhibits microbial growth in the water or other liquid. In other aspects, a disclosed method comprises applying a composition disclosed herein to water or other liquid present in a medical device in an amount that restrains, prohibits, or otherwise prevents microbial population growth without eradicating the microbes to a degree that achieves disinfection or sterilization, wherein application of the composition reduces or inhibits microbial growth in the water or other liquid. In yet other aspects, a disclosed method comprises applying a composition disclosed herein to a medical device, wherein application of the composition reduces or inhibits microbial growth prior and during operation of the device. In still other aspects of the present specification disclose a composition disclosed herein for use in reducing or inhibiting microbial growth in water or other liquid present in a medical device. In other aspects of the present specification disclose a use of a disclosed composition to reduce or inhibit microbial growth in a medical device.

Other aspects of the present specification disclose a method to wash or irrigate a surface area. In aspects a disclosed method comprises applying a composition disclosed herein to a surface area, wherein application of the composition washes or irrigates the surface area. In other aspects of the present specification disclose a composition disclosed herein for use in washing or irrigating a surface area. In yet other aspects of the present specification disclose a use of a disclosed composition to wash or irrigate a surface area.

Other aspects of the present specification disclose a method to wash or irrigate a biological surface, a tissue, a skin, or a body cavity. In aspects a disclosed method comprises applying a composition disclosed herein to a surface area, wherein application of the composition washes or irrigates a biological surface, a tissue, a skin, or a body cavity. In other aspects of the present specification disclose a composition disclosed herein for use in washing or irrigating a biological surface, a tissue, a skin, or a body cavity. In yet other aspects of the present specification disclose a use of a disclosed composition to wash or irrigate a biological surface, a tissue, a skin, or a body cavity.

Other aspects of the present specification disclose a method to wash or irrigate a wound in an individual. In aspects, a disclosed method comprises applying a composition disclosed herein to the wound of an individual, wherein application of the composition washes or irrigates the wound. In other aspects of the present specification disclose a composition disclosed herein for use in washing or irrigating a wound in an individual. In yet other aspects of the present specification disclose a use of a disclosed composition to wash or irrigate a wound in an individual. In still other aspects of the present specification disclose a use of a disclosed composition in the manufacture of a medicament to wash or irrigate a wound in an individual.

Other aspects of the present specification disclose a method to treat and/or provide relief from a wound in an individual. In aspects a disclosed method comprising applying a composition disclosed herein to an individual, wherein application of the composition promotes healing of a wound. In other aspects of the present specification disclose a composition disclosed herein for use in treating and/or providing relief of a wound in an individual. In yet other aspects of the present specification disclose a use of a disclosed composition to treat and/or provide relief from a wound in an individual. In still other aspects of the present specification disclose a use of a disclosed composition in the manufacture of a medicament to treat and/or provide relief from a wound in an individual.

Other aspects of the present specification disclose a method to wash or irrigate an infection in an individual. In aspects, a disclosed method comprising applying a composition disclosed herein to an individual, wherein application of the composition washes or irrigates the infection. In other aspects of the present specification disclose a composition disclosed herein for use in washing or irrigating an infection in an individual. In yet other aspects of the present specification disclose a use of a disclosed composition to wash or irrigate an infection in an individual. In still other of the present specification disclose a use of a disclosed composition in the manufacture of a medicament to wash or irrigate an infection in an individual. An infection can be a microbial infection.

Other aspects of the present specification disclose a method to treat and/or provide relief from an infection in an individual. In aspects a disclosed method comprising applying a composition disclosed herein to an individual, wherein application of the composition promotes healing of an infection. In other aspects of the present specification disclose a composition disclosed herein for use in treating and/or providing relief of an infection in an individual. In yet other aspects of the present specification disclose a use of a disclosed composition to treat and/or provide relief from an infection in an individual. In still other aspects of the present specification disclose a use of a disclosed composition in the manufacture of a medicament to treat and/or provide relief from an infection in an individual.

Other aspects of the present specification disclose a method to wash or irrigate an inflammation in an individual. In aspects, a disclosed method comprising applying a composition disclosed herein to an individual, wherein application of the composition washes or irrigates the inflammation. In other aspects of the present specification disclose a composition disclosed herein for use in washing or irrigating an inflammation in an individual. In yet other aspects of the present specification disclose a use of a disclosed composition to wash or irrigate an inflammation in an individual. In still other aspects of the present specification disclose a use of a disclosed composition in the manufacture of a medicament to wash or irrigate an inflammation in an individual.

Other aspects of the present specification disclose a method to treat and/or provide relief from an inflammation in an individual. In aspects a disclosed method comprising applying a composition disclosed herein to an individual, wherein application of the composition promotes healing of an inflammation. In other aspects of the present specification disclose a composition disclosed herein for use in treating and/or providing relief of an inflammation in an individual. In yet other aspects of the present specification disclose a use of a disclosed composition to treat and/or provide relief from an inflammation in an individual. In still other aspects of the present specification disclose a use of a disclosed composition in the manufacture of a medicament to treat and/or provide relief from an inflammation in an individual.

### DETAILED DESCRIPTION

A composition disclosed herein comprises one or more microbiostatic agents that reduces or prevents growth of microbes. Microbiostatic agents are biological or chemical agents that directly or indirectly restrain, prohibit or otherwise prevent growth of microbes for given time period. Exposure to a microbiostatic agent disclosed herein can be reversed such that microbial growth could resume once the microbiostatic agent is removed. In addition, when added to water or other fluids, a microbiostatic agent disclosed herein is not designed to disinfect, sterilize or otherwise eliminate microbial contamination. Furthermore, microbiostatic agents classified by regulatory agencies including EPA and FDA and they require distinct testing protocols that are vastly different from disinfection and sterilization test assays adopted by the same regulatory agencies. As such, a microbiostatic agent disclosed herein is not a microbiocidal agent because it does not necessarily irreversibly eradicate microbes. A microbiostatic agent disclosed herein includes, without limitation, hypochlorous acid, free available chlorine, one or more guanide-containing compounds, one or more surfactants, one or more quaternary compound or silicon quaternary compounds, one or more metallic particles and/or one or more metal salts. A microbe, includes, without limitation, a bacterium, an alga, a fungus (including a mold, a mildew, and a yeast), and a virus.

A composition disclosed herein restrains, prohibits or otherwise prevents growth of populations of microbes for given time period. Exposure to a composition disclosed herein can be reversed such that microbial growth could resume once the composition is removed. When used as a substitute for water or other liquid, a composition disclosed herein is not designed to disinfect, sterilize or otherwise eliminate microbial contamination on a surface, or in a device, equipment, machinery or other apparatus. When added to water or other liquid, a composition disclosed herein is not designed to disinfect, sterilize or otherwise eliminate microbial contamination either in the water or water-based solution, or a surface, or a device, equipment, machinery or other apparatus. As such, a composition disclosed herein is a microbiostatic composition and not a microbiocidal composition.

Without wishing to be limited by theory, a composition disclosed herein directly or indirectly restrains, prohibits or otherwise prevents microbial growth by, e.g., preventing cell division or an aspect thereof, such as karyokinesis or cytokinesis. For example, a composition disclosed herein can directly restrain, inhibit or otherwise prevent an aspect of cell division by, e.g., inactivating an enzyme or other component needed for an aspect of cell division. As another example, a composition disclosed herein can indirectly restrain, inhibit or otherwise prevent an aspect of cell division by, e.g., sequestering a metabolite or nutrient needed for microbial health (unhealthy microbes will not reproduce) or an aspect of cell division. As such, a composition disclosed herein is a broad-spectrum microbiostatic composition that restrains, prohibits, or otherwise prevents microbial population growth without eradicating the microbes. In an aspect of this embodiment, a composition disclosed herein is a broad-spectrum microbiostatic composition that restrains, prohibits, or otherwise prevents microbial population growth without eradicating the microbes to a degree that achieves disinfection or sterilization.

A disclosed composition can be formulated to be used directly as an undiluted composition, e.g., in a method disclosed herein. Such a composition is formulated with a microbiostatic effective amount of one or more microbiostatic agents. In addition, a disclosed composition can be formulated as a concentrate and used in a diluted form or as an additive to a water or another fluid, e.g., in a method disclosed herein. Such a composition is formulated with one or more microbiostatic agents in a manner that the final concentration of one or more microbiostatic agents used in an application will be a microbiostatic effective amount. A composition disclosed herein can be manufactured under sterile conditions and/or subject to subsequent sterilization procedure, such as, e.g., filtration.

A composition disclosed herein contains a microbiostatic effective amount of a microbiostatic agent. A microbiostatic effective amount is an amount that restrains, prohibits or otherwise prevents growth of populations of microbes for given time period. Microbial growth or microbial population growth refers to a process whereby microbes multiply or reproduce themselves to create new microbes by, e.g., cell division, sporation, or replication. As such, a microbiostatic effective amount directly or indirectly prevents microbes from undergoing a process that multiplies or reproduces themselves by, e.g., preventing cell division or an aspect thereof, such as karyokinesis or cytokinesis, but does not eradicate these microbes. In an aspect of this embodiment, a microbiostatic effective amount is an amount that restrains, prohibits or otherwise prevents growth of populations of microbes for given time period without entirely eradicating the microbes. In another aspect of this embodiment, a microbiostatic effective amount is an amount that restrains, prohibits or otherwise prevents growth of populations of microbes for given time period without eradicating the microbes to a degree that achieves disinfection or sterilization. In yet another aspect of this embodiment, a microbiostatic effective amount includes a microbial sub-lethal amount (also referred to as a microbial non-lethal amount), or a minimum inhibitory concentration (MIC).

In aspects of this embodiment, a microbiostatic effective amount can be a bacteriostatic effective amount, an algaestatic effective amount, a fungistatic effective amount and/or a virustatic effective amount. A bacteriostatic effective amount is an amount that restrains bacterial growth for given time period without eradicating the bacteria. In an aspect of this embodiment, a bacteriostatic effective amount is an amount that restrains bacterial growth for given time period without eradicating the bacteria to a degree that achieves disinfection or sterilization. An algeastatic effective amount is an amount that restrains algal growth for given time period without eradicating the algae to a degree that achieves disinfection or sterilization. A fungistatic effective amount is an amount that restrains fungal growth for given time period without eradicating the fungi. In an aspect of this embodiment, a fungistatic effective amount is an amount that restrains fungal growth for given time period without eradicating the fungi to a degree that achieves disinfection or sterilization. A virustatic effective amount is an amount that restrains viral replication for given time period without eradicating the virus to a degree that achieves disinfection or sterilization. In an aspect of this embodiment, a virustatic effective amount is an amount that restrains viral replication for given time period without eradicating the virus to a degree that achieves disinfection or sterilization.

In aspects of this embodiment, a microbiostatic effective amount of a microbiostatic agent disclosed herein is an amount that 1) restrains, prohibits or otherwise prevents growth of a microbial population; 2) results in no more than a 1×10² reduction in the microbial population within the first seven days of continuous exposure to the microbiostatic agent; and 3) results in a minimum of a 1×10² reduction in the microbial population after at least 14 days of continuous exposure to the microbiostatic agent without achieving full kill of the microbial population. In other aspects of this embodiment, a microbiostatic effective amount of a microbiostatic agent disclosed herein is an amount that 1) restrains, prohibits or otherwise prevents growth of a microbial population; 2) results in no more than a 1×10² reduction in the microbial population within the first seven days of continuous exposure to the microbiostatic agent; and 3) results in a minimum of a 1×10² reduction in the microbial population after at least 14 days of continuous exposure to the microbiostatic agent but no more than 1×10⁴ reduction of the microbial population, no more than 1×10⁵ reduction of the microbial population, no more than 1×10⁶ reduction of the microbial population, or no more than 1×10⁷ reduction of the microbial population.

In aspects of this embodiment, a microbiostatic effective amount of a microbiostatic agent disclosed herein is an amount that 1) restrains, prohibits or otherwise prevents growth of a microbial population; 2) results in about 1×10⁰ to about 1×10² reduction in the microbial population within the first seven days of continuous exposure to the microbiostatic agent; and 3) results in a minimum of a 1×10² reduction in the microbial population after at least 14 days of continuous exposure to the microbiostatic agent without achieving full kill of the microbial population. In other aspects of this embodiment, a microbiostatic effective amount of a microbiostatic agent disclosed herein is an amount that 1) restrains, prohibits or otherwise prevents growth of a microbial population; 2) results in about 1×10⁰ to about 1×10² reduction in the microbial population within the first seven days of continuous exposure to the microbiostatic agent; and 3) results in a minimum of a 1×10² reduction in the microbial population after at least 14 days of continuous exposure to the microbiostatic agent but no more than 1×10⁴ reduction of the microbial population, no more than 1×10⁵ reduction of the microbial population, no more than 1×10⁶ reduction of the microbial population, or no more than 1×10⁷ reduction of the microbial population.

In aspects of this embodiment, a sub-lethal amount of a microbiostatic agent disclosed herein is an amount that 1) restrains, prohibits or otherwise prevents growth of a microbial population; 2) results in no more than a 1×10² reduction in the microbial population within the first seven days of continuous exposure to the microbiostatic agent; and 3) results in a minimum of a 1×10² reduction in the microbial population after at least 14 days of continuous exposure to the microbiostatic agent without achieving full kill of the microbial population. In other aspects of this embodiment, a sub-lethal amount of a microbiostatic agent disclosed herein is an amount that 1) restrains, prohibits or otherwise prevents growth of a microbial population; 2) results in no more than a 1×10² reduction in the microbial population within the first seven days of continuous exposure to the microbiostatic agent; and 3) results in a minimum of a 1×10² reduction in the microbial population after at least 14 days of continuous exposure to the microbiostatic agent but no more than 1×10⁴ reduction of the microbial population, no more than 1×10⁵ reduction of the microbial population, no more than 1×10⁶ reduction of the microbial population, or no more than 1×10⁷ reduction of the microbial population.

In aspects of this embodiment, a sub-lethal amount of a microbiostatic agent disclosed herein is an amount that 1) restrains, prohibits or otherwise prevents growth of a microbial population; 2) results in about 1×10⁰ to about 1×10² reduction in the microbial population within the first seven days of continuous exposure to the microbiostatic agent; and 3) results in a minimum of a 1×10² reduction in the microbial population after at least 14 days of continuous exposure to the microbiostatic agent without achieving full kill of the microbial population. In other aspects of this embodiment, a sub-lethal amount of microbiostatic agent disclosed herein is an amount that 1) restrains, prohibits or otherwise prevents growth of a microbial population; 2) results in about 1×10⁰ to about 1×10² reduction in the microbial population within the first seven days of continuous exposure to the microbiostatic agent; and 3) results in a minimum of a 1×10² reduction in the microbial population after at least 14 days of continuous exposure to the microbiostatic agent but no more than 1×10⁴ reduction of the microbial population, no more than 1×10⁵ reduction of the microbial population, no more than 1×10⁶ reduction of the microbial population, or no more than 1×10⁷ reduction of the microbial population.

A minimum microbiocidal concentration is the lowest amount of a microbiostatic agent disclosed herein or a composition disclosed herein that will eradicate the microbes comprising a microbial population within one day. In aspects of this embodiment, a minimum microbiocidal concentration of a microbiostatic agent disclosed herein or a composition disclosed herein achieves in one day, e.g., more than a 1×10⁴ reduction of the microbial population, more than a 1×10⁵ reduction of the microbial population, more than a 1×10⁶ reduction of the microbial population, or more than a 1×10⁷ reduction of the microbial population. In an embodiment, a composition disclosed herein is formulated with one or more microbiostatic agents disclosed herein in an amount that is not a minimum microbiocidal concentration. In an embodiment, a composition disclosed herein is formulated with one or more microbiostatic agents disclosed herein in an amount that is below the minimum microbiocidal concentration of the one or more microbiostatic agents. In another embodiment, the amount of one or more microbiostatic agents disclosed herein or a composition disclosed herein used in a method disclosed herein is an amount that is not a minimum microbiocidal concentration of the one or more microbiostatic agents or the composition. In another embodiment, the amount of one or more microbiostatic agents disclosed herein or a composition disclosed herein used in a method disclosed herein is an amount below the minimum microbiocidal concentration of the one or more microbiostatic agents or the composition.

In an embodiment, a microbiostatic effective amount of a microbiostatic agent disclosed herein is an amount that restrains, prohibits or otherwise prevents growth of populations of microbes for a given period of time. In aspects of this embodiment, a microbiostatic effective amount of a microbiostatic agent disclosed herein is an amount that restrains, prohibits or otherwise prevents growth of populations of microbes for, e.g., about 4 hours, about 8 hours, about 12 hours, about 16 hours, about 20 hours or about 24 hours. In other aspects of this embodiment, a microbiostatic effective amount of a microbiostatic agent disclosed herein is an amount that restrains, prohibits or otherwise prevents growth of populations of microbes for, e.g., at least 4 hours, at least 8 hours, at least 12 hours, at least 16 hours, at least 20 hours or at least 24 hours. In yet other aspects of this embodiment, a microbiostatic effective amount of a microbiostatic agent disclosed herein is an amount that restrains, prohibits or otherwise prevents growth of populations of microbes for, e.g., at most 4 hours, at most 8 hours, at most 12 hours, at most 16 hours, at most 20 hours or at most 24 hours. In still other aspects of this embodiment, a microbiostatic effective amount of a microbiostatic agent disclosed herein is an amount that restrains, prohibits or otherwise prevents growth of populations of microbes for, e.g., about 4 hours to about 8 hours, about 4 hours to about 12 hours, about 4 hours to about 16 hours, about 4 hours to about 20 hours, about 4 hours to about 24 hours, about 8 hours to about 12 hours, about 8 hours to about 16 hours, about 8 hours to about 20 hours, about 8 hours to about 24 hours, about 12 hours to about 16 hours, about 12 hours to about 20 hours, about 12 hours to about 24 hours, about 16 hours to about 20 hours, about 16 hours to about 24 hours, or about 20 hours to about 24 hours.

In other aspects of this embodiment, a microbiostatic effective amount of a microbiostatic agent disclosed herein is an amount that restrains, prohibits or otherwise prevents growth of populations of microbes for e.g., about 1 day, about 2 days, 3 days, 4 days, 5 days, 6, days, 7 days, 8 days, 9 days, 10 days, 11 days, 12 days, 13 days, 14 days, 15 days, about 20 days, about 25 days, or about 30 days. In yet other aspects of this embodiment, a microbiostatic effective amount of a microbiostatic agent disclosed herein is an amount that restrains, prohibits or otherwise prevents growth of populations of microbes for, e.g., at least 1 day, at least 2 days, at least 3 days, at least 4 days, at least 5 days, at least 6, days, at least 7 days, at least 8 days, at least 9 days, at least 10 days, at least 11 days, at least 12 days, at least 13 days, at least 14 days, at least 15 days, at least 20 days, at least 25 days, or at least 30 days. In still other aspects of this embodiment, a microbiostatic effective amount of a microbiostatic agent disclosed herein is an amount that restrains, prohibits or otherwise prevents growth of populations of microbes for, e.g., at most 1 day, at most 2 days, at most 3 days, at most 4 days, at most 5 days, at most 6, days, at most 7 days, at most 8 days, at most 9 days, at most 10 days, at most 11 days, at most 12 days, at most 13 days, at most 14 days, at most 15 days, at most 20 days, at most 25 days, or at most 30 days.

In other aspects of this embodiment, a microbiostatic effective amount of a microbiostatic agent disclosed herein is an amount that restrains, prohibits or otherwise prevents growth of populations of microbes for, e.g., about 1 day to about 2 days, about 1 day to about 3 days, about 1 day to about 4 days, about 1 day to about 5 days, about 1 day to about 6 days, about 1 day to about 7 days, about 1 day to about 8 days, about 1 day to about 9 days, about 1 day to about 10 days, about 1 day to about 11 days, about 1 day to about 12 days, about 1 day to about 13 days, about 1 day to about 14 days, about 1 day to about 15 days, about 1 day to about 20 days, about 1 day to about 25 days, about 1 day to about 30 days, about 2 days to about 3 days, about 2 days to about 4 days, about 2 days to about 5 days, about 2 days to about 6 days, about 2 days to about 7 days, about 2 days to about 8 days, about 2 days to about 9 days, about 2 days to about 10 days, about 2 days to about 11 days, about 2 days to about 12 days, about 2 days to about 13 days, about 2 days to about 14 days, about 2 days to about 15 days, about 2 days to about 20 days, about 2 days to about 25 days, about 2 days to about 30 days, about 3 days to about 4 days, about 3 days to about 5 days, about 3 days to about 6 days, about 3 days to about 7 days, about 3 days to about 8 days, about 3 days to about 9 days, about 3 days to about 10 days, about 3 days to about 11 days, about 3 days to about 12 days, about 3 days to about 13 days, about 3 days to about 14 days, about 3 days to about 15 days, about 3 days to about 20 days, about 3 days to about 25 days, about 3 days to about 30 days, about 4 days to about 5 days, about 4 days to about 6 days, about 4 days to about 7 days, about 4 days to about 8 days, about 4 days to about 9 days, about 4 days to about 10 days, about 4 days to about 11 days, about 4 days to about 12 days, about 4 days to about 13 days, about 4 days to about 14 days, about 4 days to about 15 days, about 4 days to about 20 days, about 4 days to about 25 days, about 4 days to about 30 days, about 5 days to about 6 days, about 5 days to about 7 days, about 5 days to about 8 days, about 5 days to about 9 days, about 5 days to about 10 days, about 5 days to about 11 days, about 5 days to about 12 days, about 5 days to about 13 days, about 5 days to about 14 days, about 5 days to about 15 days, about 5 days to about 20 days, about 5 days to about 25 days, about 5 days to about 30 days, about 6 days to about 7 days, about 6 days to about 8 days, about 6 days to about 9 days, about 6 days to about 10 days, about 6 days to about 11 days, about 6 days to about 12 days, about 6 days to about 13 days, about 6 days to about 14 days, about 6 days to about 15 days, about 6 days to about 20 days, about 6 days to about 25 days, about 6 days to about 30 days, about 7 days to about 8 days, about 7 days to about 9 days, about 7 days to about 10 days, about 7 days to about 11 days, about 7 days to about 12 days, about 7 days to about 13 days, about 7 days to about 14 days, about 7 days to about 15 days, about 7 days to about 20 days, about 7 days to about 25 days, about 7 days to about 30 days, about 8 days to about 9 days, about 8 days to about 10 days, about 8 days to about 11 days, about 8 days to about 12 days, about 8 days to about 13 days, about 8 days to about 14 days, about 8 days to about 15 days, about 8 days to about 20 days, about 8 days to about 25 days, about 8 days to about 30 days, about 9 days to about 10 days, about 9 days to about 11 days, about 9 days to about 12 days, about 9 days to about 13 days, about 9 days to about 14 days, about 9 days to about 15 days, about 9 days to about 20 days, about 9 days to about 25 days, about 9 days to about 30 days, about 10 days to about 11 days, about 10 days to about 12 days, about 10 days to about 13 days, about 10 days to about 14 days, about 10 days to about 15 days, about 10 days to about 20 days, about 10 days to about 25 days, about 10 days to about 30 days, about 11 days to about 12 days, about 11 days to about 13 days, about 11 days to about 14 days, about 11 days to about 15 days, about 11 days to about 20 days, about 11 days to about 25 days, about 11 days to about 30 days, about 12 days to about 13 days, about 12 days to about 14 days, about 12 days to about 15 days, about 12 days to about 20 days, about 12 days to about 25 days, about 12 days to about 30 days, about 13 days to about 14 days, about 13 days to about 15 days, about 13 days to about 20 days, about 13 days to about 25 days, about 13 days to about 30 days, about 14 days to about 15 days, about 14 days to about 20 days, about 14 days to about 25 days, about 14 days to about 30 days, about 15 days to about 20 days, about 15 days to about 25 days, about 15 days to about 30 days, about 20 days to about 25 days, about 20 days to about 30 days, or about 25 days to about 30 days.

In other aspects of this embodiment, a microbiostatic effective amount of a microbiostatic agent disclosed herein is an amount that restrains, prohibits or otherwise prevents growth of populations of microbes for, e.g., about 1 week, about 2 weeks, 3 weeks, or 4 weeks. In yet other aspects of this embodiment, a microbiostatic effective amount of a microbiostatic agent disclosed herein is an amount that restrains, prohibits or otherwise prevents growth of populations of microbes for, e.g., at least 1 week, at least 2 weeks, at least 3 weeks, or at least 4 weeks. In still other aspects of this embodiment, a microbiostatic effective amount of a microbiostatic agent disclosed herein is an amount that restrains, prohibits or otherwise prevents growth of populations of microbes for, e.g., at most 1 week, at most 2 weeks, at most 3 weeks, or at most 4 weeks. In aspects of this embodiment, a microbiostatic effective amount of a microbiostatic agent disclosed herein is an amount that restrains, prohibits or otherwise prevents growth of populations of microbes for, e.g., about 1 week to about 2 weeks, about 1 week to about 3 weeks, about 1 week to about 4 weeks, about 2 weeks to about 3 weeks, about 2 weeks to about 4 weeks, or about 3 weeks to about 4 weeks.

A composition disclosed herein has increased stability and efficacy. In one embodiment, this increased stability and efficacy is due to the protection of hypochlorous acid. In part, the composition may protect hypochlorous acid from exposure to harmful agents, including, e.g., organic compounds, reactive compounds, positively charged molecules, or other compounds that promote or facilitate degradation of hypochlorous acid. For example, the disclosed compositions are stable in blood serum and in plastic packaging material. In addition, a synergistic effect in efficacy is observed when hypochlorous acid is protected against this exposure. This significantly improved efficacy allows for lower amounts of hypochlorous acid to be used in the disclosed compositions. As concentrations of hypochlorous acid above 350 ppm, particularly above 500 ppm produces instability, the lower amounts of hypochlorous acid used in the disclosed compositions increases stability of the composition even further. As such, a composition disclosed herein is seen as a replacement to any and all prior hypochlorous acid compositions currently in the market or being developed.

The stability of a composition disclosed herein may be due to the positively charged N atom of a quaternary compound or silicon quaternary compound, a metallic particle, a metal salt, a phosphate buffer like calcium phosphate or sodium phosphate, and/or a guanide-containing compound disclosed herein. Such compounds appear to form a strong ionic interaction with the negatively charged (OCl⁻), and the complex thus formed retains its stability over period of time compared to hypochlorous acid alone, which is a weaker complex in solution relative to a composition disclosed herein. For example, in the presence of copper chloride hypochlorous acid forms copper hypochlorite and HCl and in the presence of zinc chloride hypochlorous acid forms zinc hypochlorite and HCl. By replacing sodium hydroxide with calcium oxide, a less basic solution is formed because in the presence of hypochlorous acid calcium oxide forms calcium hypochlorite and water. The reduced levels of base enhances the stability of copper hypochlorite and zinc hypochlorite because there is less base to react with zinc and copper ions, thereby maintaining the stability of copper hypochlorite and zinc hypochlorite. Further, the use of 500 ppm or lower, an in particular 350 ppm or lower of hypochlorous acid further increases the stability of a composition disclosed herein.

The improved efficacy of a composition disclosed herein may in part be a result of chemical bond formation of a quaternary compound or silicon quaternary compound, a metallic particle, a metal salt, aphosphate buffer like calcium phosphate or sodium phosphate, and/or a guanide-containing compound disclosed herein with the substrates thereby providing a long-lasting activity combined with the free OCl⁻, which has its own antimicrobial activity.

A composition disclosed herein may comprise hypochlorous acid. A weak acid, the chemical formula of hypochlorous acid is HOCl, while its molecular formula is written as HClO. As shown in formula I, hypochlorous acid is a simple molecule with the central oxygen connected to chlorine and hydrogen atoms through single bonds and has molar mass is 52.46 g/mol.

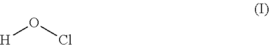

Hypochlorous acid is a colorless solution, and its exact physical properties are variable, depending on the concentration of hypochlorous in solution. Hypochlorous acid reacts with bases to form salts called hypochlorites. For example, sodium hypochlorite (NaOCl), the active ingredient in bleach, is formed by reacting hypochlorous acid with sodium hydroxide. Hypochlorous acid also readily reacts with a variety of organic molecules and biomolecules.

The hypochlorous acid solution can be produced, e.g., by dissolving chlorine in water, hydrolysis of chlorine gas, electrolysis of a salt solution or acidification of hypochloride or sodium hypochloride. For example, stable hypochlorous salts, such as, e.g., alkali metal hypochlorites like sodium hypochlorite, calcium hypochlorite, potassium hypochlorite, lithium hypochlorite and magnesium hypochlorite, can be obtained by dissolving chlorine gas into an aqueous alkali metal hydroxide solution, like a sodium hydroxide solution, a calcium hydroxide solution, a potassium hydroxide solution, a lithium hydroxide solution, or a magnesium hydroxide solution. Hypochlorous acid can also be prepared by dissolving dichlorine monoxide in water. As another example, hypochlorous acid can also be produced by electrolytically treating a saline solution. In one method, an electrical current is applied to a one-, two-, or three-compartment cell comprising a cathode chamber, an anode chamber, and a central saline solution chamber interposed between the other two chambers where each chamber is separated by a semi-permeable membrane. During electrolysis, sodium chloride (NaCl) dissociates into negatively charged chloride (Cl⁻) and positively charged sodium (Na⁺). At the same time, water dissociates into hydroxide (OH⁻) and hydrogen (H⁺) ions are formed. The negatively charged chloride (Cl⁻) and hydroxide (OH⁻) ions move to the anode to lose electrons and form hypochlorous acid (HOCl) as well as hypochlorite ions (OCl⁻) and oxygen (O₂) and chlorine (Cl₂) gases. The reductive water comprising the hypochlorous acid is then dispensed into a collection chamber for subsequent use. Methods to produce hypochlorous acid are described in, e.g., U.S. Pat. Nos. 3,914,397, 4,190,638, 4,908,215, 5,322,677, 6,426,066, 7,323,118, 8,062,500, 8,945,630, 9,168,318, and 9,486,479, each of which is hereby incorporated by reference in its entirety.

In an embodiment, a composition disclosed herein comprises an amount of hypochlorous acid that provides a desired beneficial effect to a composition disclosed herein. For example, when a composition comprising hypochlorous acid is formulated to be used in an undiluted state, the amount of hypochlorous acid used is a microbiostatic effective amount. Alternatively, when a composition comprising hypochlorous acid is formulated to be used in methods where the composition will be diluted, the amount of hypochlorous acid used in such concentrated formulations can be any amount that does not cause precipitation of components in the composition disclosed herein or otherwise create unwanted effects that make the composition inoperable.

In aspects of this embodiment, a composition disclosed herein comprises hypochlorous acid in an amount of, e.g., 0.01 ppm, 0.05 ppm, 0.10 ppm, 0.15 ppm, 0.20 ppm, 0.25 ppm, 0.30 ppm, 0.35 ppm, 0.40 ppm, 0.45 ppm, 0.50 ppm, 0.55 ppm, 0.60 ppm, 0.65 ppm, 0.70 ppm, 0.75 ppm, 0.80 ppm, 0.85 ppm, 0.90 ppm, 0.95 ppm, 1 ppm, 5 ppm, 10 ppm, 15 ppm, 20 ppm, 25 ppm, 30 ppm, 35 ppm, 40 ppm, 45 ppm, 50 ppm, 55 ppm, 60 ppm, 65 ppm, 70 ppm, 75 ppm, 80 ppm, 85 ppm, 90 ppm, 95 ppm, 100 ppm, 125 ppm, 150 ppm, 175 ppm, 200 ppm, 225 ppm, 250 ppm, 275 ppm, 300 ppm, 325 ppm, 350 ppm, 375 ppm, 400 ppm, 425 ppm, 450 ppm, 475 ppm, 500 ppm, 525 ppm, 550 ppm, 575 ppm, 600 ppm, 625 ppm, 650 ppm, 675 ppm, 700 ppm, 725 ppm, 750 ppm, 775 ppm, 800 ppm, 825 ppm, 850 ppm, 875 ppm, 900 ppm, 925 ppm, 950 ppm, 975 ppm, 1,000 ppm, 1,025 ppm, 1,050 ppm, 1075 ppm, 1,100 ppm, 1,125 ppm, 1,150 ppm, 1,175 ppm, 1,200 ppm, 1,225 ppm, 1,250 ppm, 1,275 ppm, 1,300 ppm, 1,325 ppm, 1,350 ppm, 1,375 ppm, 1,400 ppm, 1,425 ppm, 1,450 ppm, 1,475 ppm, 1,500 ppm, 1,600 ppm, 1,700 ppm, 1,800 ppm, 1,900 ppm, or 2,000 ppm. In other aspects of this embodiment, a composition disclosed herein comprises hypochlorous acid in an amount of, e.g., at least 0.01 ppm, at least 0.05 ppm, at least 0.10 ppm, at least 0.20 ppm, at least 0.30 ppm, at least 0.40 ppm, at least 0.50 ppm, at least 0.60 ppm, at least 0.70 ppm, at least 0.80 ppm, at least 0.90 ppm, at least 1 ppm, at least 10 ppm, at least 20 ppm, at least 30 ppm, at least 40 ppm, at least 50 ppm, at least 60 ppm, at least 70 ppm, at least 80 ppm, at least 90 ppm, at least 100 ppm, at least 125 ppm, at least 150 ppm, at least 175 ppm, at least 200 ppm, at least 225 ppm, at least 250 ppm, at least 275 ppm, at least 300 ppm, at least 325 ppm, at least 350 ppm, at least 375 ppm, at least 400 ppm, at least 425 ppm, at least 450 ppm, at least 475 ppm, at least 500 ppm, at least 525 ppm, at least 550 ppm, at least 575 ppm, at least 600 ppm, at least 625 ppm, at least 650 ppm, at least 675 ppm, at least 700 ppm, at least 725 ppm, at least 750 ppm, at least 775 ppm, at least 800 ppm, at least 825 ppm, at least 850 ppm, at least 875 ppm, at least 900 ppm, at least 925 ppm, at least 950 ppm, at least 975 ppm, at least 1,000 ppm, at least 1,025 ppm, at least 1,050 ppm, at least 1075 ppm, at least 1,100 ppm, at least 1,125 ppm, at least 1,150 ppm, at least 1,175 ppm, at least 1,200 ppm, at least 1,225 ppm, at least 1,250 ppm, at least 1,275 ppm, at least 1,300 ppm, at least 1,325 ppm, at least 1,350 ppm, at least 1,375 ppm, at least 1,400 ppm, at least 1,425 ppm, at least 1,450 ppm, at least 1,475 ppm, at least 1,500 ppm, at least 1,600 ppm, at least 1,700 ppm, at least 1,800 ppm, at least 1,900 ppm, or at least 2,000 ppm. In yet other aspects of this embodiment, a composition disclosed herein comprises hypochlorous acid in an amount of, e.g., at most 0.01 ppm, at most 0.05 ppm, at most 0.10 ppm, at most 0.20 ppm, at most 0.30 ppm, at most 0.40 ppm, at most 0.50 ppm, at most 0.60 ppm, at most 0.70 ppm, at most 0.80 ppm, at most 0.90 ppm, at most 1 ppm, at most 10 ppm, at most 20 ppm, at most 30 ppm, at most 40 ppm, at most 50 ppm, at most 60 ppm, at most 70 ppm, at most 80 ppm, at most 90 ppm, at most 100 ppm, at most 125 ppm, at most 150 ppm, at most 175 ppm, at most 200 ppm, at most 225 ppm, at most 250 ppm, at most 275 ppm, at most 300 ppm, at most 325 ppm, at most 350 ppm, at most 375 ppm, at most 400 ppm, at most 425 ppm, at most 450 ppm, at most 475 ppm, at most 500 ppm, at most 525 ppm, at most 550 ppm, at most 575 ppm, at most 600 ppm, at most 625 ppm, at most 650 ppm, at most 675 ppm, at most 700 ppm, at most 725 ppm, at most 750 ppm, at most 775 ppm, at most 800 ppm, at most 825 ppm, at most 850 ppm, at most 875 ppm, at most 900 ppm, at most 925 ppm, at most 950 ppm, at most 975 ppm, at most 1,000 ppm, at most 1,025 ppm, at most 1,050 ppm, at most 1075 ppm, at most 1,100 ppm, at most 1,125 ppm, at most 1,150 ppm, at most 1,175 ppm, at most 1,200 ppm, at most 1,225 ppm, at most 1,250 ppm, at most 1,275 ppm, at most 1,300 ppm, at most 1,325 ppm, at most 1,350 ppm, at most 1,375 ppm, at most 1,400 ppm, at most 1,425 ppm, at most 1,450 ppm, at most 1,475 ppm, at most 1,500 ppm, at most 1,600 ppm, at most 1,700 ppm, at most 1,800 ppm, at most 1,900 ppm, or at most 2,000 ppm.

In still other aspects of this embodiment, a composition disclosed herein comprises hypochlorous acid in an amount of from, e.g., about 0.01 ppm to about 0.1 ppm, about 0.01 ppm to about 0.2 ppm, about 0.01 ppm to about 0.25 ppm, about 0.01 ppm to about 0.3 ppm, about 0.01 ppm to about 0.4 ppm, about 0.01 ppm to about 0.5 ppm, about 0.01 ppm to about 1 ppm, about 0.01 ppm to about 5 ppm, about 0.01 ppm to about 10 ppm, about 0.01 ppm to about 15 ppm, about 0.01 ppm to about 20 ppm, about 0.01 ppm to about 25 ppm, about 0.01 ppm to about 30 ppm, about 0.01 ppm to about 35 ppm, about 0.01 ppm to about 40 ppm, about 0.01 ppm to about 45 ppm, about 0.01 ppm to about 50 ppm, about 0.05 ppm to about 0.1 ppm, about 0.05 ppm to about 0.5 ppm, about 0.05 ppm to about 1 ppm, about 0.05 ppm to about 5 ppm, about 0.05 ppm to about 10 ppm, about 0.05 ppm to about 15 ppm, about 0.05 ppm to about 20 ppm, about 0.05 ppm to about 25 ppm, about 0.05 ppm to about 30 ppm, about 0.05 ppm to about 35 ppm, about 0.05 ppm to about 40 ppm, about 0.05 ppm to about 45 ppm, about 0.05 ppm to about 50 ppm, about 0.1 ppm to about 1 ppm, about 0.1 ppm to about 5 ppm, about 0.1 ppm to about 10 ppm, about 0.1 ppm to about 15 ppm, about 0.1 ppm to about 20 ppm, about 0.1 ppm to about 25 ppm, about 0.1 ppm to about 30 ppm, about 0.1 ppm to about 35 ppm, about 0.1 ppm to about 40 ppm, about 0.1 ppm to about 45 ppm, about 0.1 ppm to about 50 ppm, about 0.5 ppm to about 1 ppm, about 0.5 ppm to about 5 ppm, about 0.5 ppm to about 10 ppm, about 0.5 ppm to about 15 ppm, about 0.5 ppm to about 20 ppm, about 0.5 ppm to about 25 ppm, about 0.5 ppm to about 30 ppm, about 0.5 ppm to about 35 ppm, about 0.5 ppm to about 40 ppm, about 0.5 ppm to about 45 ppm, about 0.5 ppm to about 50 ppm, about 0.5 ppm to about 55 ppm, about 0.5 ppm to about 60 ppm, about 0.5 ppm to about 65 ppm, about 0.5 ppm to about 70 ppm, about 0.5 ppm to about 75 ppm, about 0.5 ppm to about 80 ppm, about 0.5 ppm to about 85 ppm, about 0.5 ppm to about 90 ppm, about 0.5 ppm to about 95 ppm, about 0.5 ppm to about 100 ppm, about 0.75 ppm to about 20 ppm, about 0.75 ppm to about 25 ppm, about 0.75 ppm to about 30 ppm, about 0.75 ppm to about 35 ppm, about 0.75 ppm to about 40 ppm, about 0.75 ppm to about 45 ppm, about 0.75 ppm to about 50 ppm, about 0.75 ppm to about 55 ppm, about 0.75 ppm to about 60 ppm, about 0.75 ppm to about 65 ppm, about 0.75 ppm to about 70 ppm, about 0.75 ppm to about 75 ppm, about 0.75 ppm to about 80 ppm, about 0.75 ppm to about 85 ppm, about 0.75 ppm to about 90 ppm, about 0.75 ppm to about 95 ppm, about 0.75 ppm to about 100 ppm, about 1 ppm to about 10 ppm, about 1 ppm to about 15 ppm, about 1 ppm to about 20 ppm, about 1 ppm to about 25 ppm, about 1 ppm to about 30 ppm, about 1 ppm to about 35 ppm, about 1 ppm to about 40 ppm, about 1 ppm to about 45 ppm, about 1 ppm to about 50 ppm, about 1 ppm to about 55 ppm, about 1 ppm to about 60 ppm, about 1 ppm to about 65 ppm, about 1 ppm to about 70 ppm, about 1 ppm to about 75 ppm, about 1 ppm to about 80 ppm, about 1 ppm to about 85 ppm, about 1 ppm to about 90 ppm, about 1 ppm to about 95 ppm, about 1 ppm to about 100 ppm, about 5 ppm to about 10 ppm, about 5 ppm to about 15 ppm, about 5 ppm to about 20 ppm, about 5 ppm to about 25 ppm, about 5 ppm to about 30 ppm, about 5 ppm to about 35 ppm, about 5 ppm to about 40 ppm, about 5 ppm to about 45 ppm, about 5 ppm to about 50 ppm, about 5 ppm to about 55 ppm, about 5 ppm to about 60 ppm, about 5 ppm to about 65 ppm, about 5 ppm to about 70 ppm, about 5 ppm to about 75 ppm, about 5 ppm to about 80 ppm, about 5 ppm to about 85 ppm, about 5 ppm to about 90 ppm, about 5 ppm to about 95 ppm, about 5 ppm to about 100 ppm, about 10 ppm to about 20 ppm, about 10 ppm to about 25 ppm, about 10 ppm to about 30 ppm, about 10 ppm to about 35 ppm, about 10 ppm to about 40 ppm, about 10 ppm to about 45 ppm, about 10 ppm to about 50 ppm, about 10 ppm to about 55 ppm, about 10 ppm to about 60 ppm, about 10 ppm to about 65 ppm, about 10 ppm to about 70 ppm, about 10 ppm to about 75 ppm, about 10 ppm to about 80 ppm, about 10 ppm to about 85 ppm, about 10 ppm to about 90 ppm, about 10 ppm to about 95 ppm, or about 10 ppm to about 100 ppm.

In other aspects of this embodiment, a composition disclosed herein comprises hypochlorous acid in an amount of from, e.g., about 1 ppm to about 25 ppm, about 1 ppm to about 50 ppm, about 1 ppm to about 75 ppm, about 1 ppm to about 100 ppm, about 1 ppm to about 125 ppm, about 1 ppm to about 150 ppm, about 1 ppm to about 175 ppm, about 1 ppm to about 200 ppm, about 1 ppm to about 225 ppm, about 1 ppm to about 250 ppm, about 1 ppm to about 275 ppm, about 1 ppm to about 300 ppm, about 1 ppm to about 325 ppm, about 1 ppm to about 350 ppm, about 1 ppm to about 375 ppm, about 1 ppm to about 400 ppm, about 10 ppm to about 25 ppm, about 10 ppm to about 50 ppm, about 10 ppm to about 75 ppm, about 10 ppm to about 100 ppm, about 10 ppm to about 125 ppm, about 10 ppm to about 150 ppm, about 10 ppm to about 175 ppm, about 10 ppm to about 200 ppm, about 10 ppm to about 225 ppm, about 10 ppm to about 250 ppm, about 10 ppm to about 275 ppm, about 10 ppm to about 300 ppm, about 10 ppm to about 325 ppm, about 10 ppm to about 350 ppm, about 10 ppm to about 375 ppm, about 10 ppm to about 400 ppm, about 25 ppm to about 50 ppm, about 25 ppm to about 75 ppm, about 25 ppm to about 100 ppm, about 25 ppm to about 125 ppm, about 25 ppm to about 150 ppm, about 25 ppm to about 175 ppm, about 25 ppm to about 200 ppm, about 25 ppm to about 225 ppm, about 25 ppm to about 250 ppm, about 25 ppm to about 275 ppm, about 25 ppm to about 300 ppm, about 25 ppm to about 325 ppm, about 25 ppm to about 350 ppm, about 25 ppm to about 375 ppm, about 25 ppm to about 400 ppm, about 50 ppm to about 75 ppm, about 50 ppm to about 100 ppm, about 50 ppm to about 125 ppm, about 50 ppm to about 150 ppm, about 50 ppm to about 175 ppm, about 50 ppm to about 200 ppm, about 50 ppm to about 225 ppm, about 50 ppm to about 250 ppm, about 50 ppm to about 275 ppm, about 50 ppm to about 300 ppm, about 50 ppm to about 325 ppm, about 50 ppm to about 350 ppm, about 50 ppm to about 375 ppm, about 50 ppm to about 400 ppm, about 75 ppm to about 100 ppm, about 75 ppm to about 125 ppm, about 75 ppm to about 150 ppm, about 75 ppm to about 175 ppm, about 75 ppm to about 200 ppm, about 75 ppm to about 225 ppm, about 75 ppm to about 250 ppm, about 75 ppm to about 275 ppm, about 75 ppm to about 300 ppm, about 75 ppm to about 325 ppm, about 75 ppm to about 350 ppm, about 75 ppm to about 375 ppm, about 75 ppm to about 400 ppm, about 100 ppm to about 125 ppm, about 100 ppm to about 150 ppm, about 100 ppm to about 175 ppm, about 100 ppm to about 200 ppm, about 100 ppm to about 225 ppm, about 100 ppm to about 250 ppm, about 100 ppm to about 275 ppm, about 100 ppm to about 300 ppm, about 100 ppm to about 325 ppm, about 100 ppm to about 350 ppm, about 100 ppm to about 375 ppm, about 100 ppm to about 400 ppm, about 150 ppm to about 175 ppm, about 150 ppm to about 200 ppm, about 150 ppm to about 225 ppm, about 150 ppm to about 250 ppm, about 150 ppm to about 275 ppm, about 150 ppm to about 300 ppm, about 150 ppm to about 325 ppm, about 150 ppm to about 350 ppm, about 150 ppm to about 375 ppm, about 150 ppm to about 400 ppm, about 200 ppm to about 225 ppm, about 200 ppm to about 250 ppm, about 200 ppm to about 275 ppm, about 200 ppm to about 300 ppm, about 200 ppm to about 325 ppm, about 200 ppm to about 350 ppm, about 200 ppm to about 375 ppm, about 200 ppm to about 400 ppm, about 250 ppm to about 275 ppm, about 250 ppm to about 300 ppm, about 250 ppm to about 325 ppm, about 250 ppm to about 350 ppm, about 250 ppm to about 375 ppm, about 250 ppm to about 400 ppm, about 300 ppm to about 325 ppm, about 300 ppm to about 350 ppm, about 300 ppm to about 375 ppm, about 300 ppm to about 400 ppm, about 350 ppm to about 375 ppm, about 350 ppm to about 400 ppm, or about 375 ppm to about 400 ppm.

In other aspects of this embodiment, a composition disclosed herein comprises hypochlorous acid in an amount of, e.g., about 400 ppm to about 500 ppm, about 400 ppm to about 600 ppm, about 400 ppm to about 700 ppm, about 400 ppm to about 800 ppm, about 400 ppm to about 900 ppm, about 400 ppm to about 1,000 ppm, about 400 ppm to about 1,100 ppm, about 400 ppm to about 1,200 ppm, about 400 ppm to about 1,300 ppm, about 400 ppm to about 1,400 ppm, about 400 ppm to about 1,500 ppm, about 400 ppm to about 1,600 ppm, about 400 ppm to about 1,700 ppm, about 400 ppm to about 1,800 ppm, about 400 ppm to about 1,900 ppm, about 400 ppm to about 2,000 ppm, about 500 ppm to about 600 ppm, about 500 ppm to about 700 ppm, about 500 ppm to about 800 ppm, about 500 ppm to about 900 ppm, about 500 ppm to about 1,000 ppm, about 500 ppm to about 1,100 ppm, about 500 ppm to about 1,200 ppm, about 500 ppm to about 1,300 ppm, about 500 ppm to about 1,400 ppm, about 500 ppm to about 1,500 ppm, about 500 ppm to about 1,600 ppm, about 500 ppm to about 1,700 ppm, about 500 ppm to about 1,800 ppm, about 500 ppm to about 1,900 ppm, about 500 ppm to about 2,000 ppm, about 600 ppm to about 700 ppm, about 600 ppm to about 800 ppm, about 600 ppm to about 900 ppm, about 600 ppm to about 1,000 ppm, about 600 ppm to about 1,100 ppm, about 600 ppm to about 1,200 ppm, about 600 ppm to about 1,300 ppm, about 600 ppm to about 1,400 ppm, about 600 ppm to about 1,500 ppm, about 600 ppm to about 1,600 ppm, about 600 ppm to about 1,700 ppm, about 600 ppm to about 1,800 ppm, about 600 ppm to about 1,900 ppm, about 600 ppm to about 2,000 ppm, about 700 ppm to about 800 ppm, about 700 ppm to about 900 ppm, about 700 ppm to about 1,000 ppm, about 700 ppm to about 1,100 ppm, about 700 ppm to about 1,200 ppm, about 700 ppm to about 1,300 ppm, about 700 ppm to about 1,400 ppm, about 700 ppm to about 1,500 ppm, about 700 ppm to about 1,600 ppm, about 700 ppm to about 1,700 ppm, about 700 ppm to about 1,800 ppm, about 700 ppm to about 1,900 ppm, about 700 ppm to about 2,000 ppm, about 800 ppm to about 900 ppm, about 800 ppm to about 1,000 ppm, about 800 ppm to about 1,100 ppm, about 800 ppm to about 1,200 ppm, about 800 ppm to about 1,300 ppm, about 800 ppm to about 1,400 ppm, about 800 ppm to about 1,500 ppm, about 800 ppm to about 1,600 ppm, about 800 ppm to about 1,700 ppm, about 800 ppm to about 1,800 ppm, about 800 ppm to about 1,900 ppm, about 800 ppm to about 2,000 ppm, about 900 ppm to about 1,000 ppm, about 900 ppm to about 1,100 ppm, about 900 ppm to about 1,200 ppm, about 900 ppm to about 1,300 ppm, about 900 ppm to about 1,400 ppm, about 900 ppm to about 1,500 ppm, about 900 ppm to about 1,600 ppm, about 900 ppm to about 1,700 ppm, about 900 ppm to about 1,800 ppm, about 900 ppm to about 1,900 ppm, about 900 ppm to about 2,000 ppm, about 1,000 ppm to about 1,100 ppm, about 1,000 ppm to about 1,200 ppm, about 1,000 ppm to about 1,300 ppm, about 1,000 ppm to about 1,400 ppm, about 1,000 ppm to about 1,500 ppm, about 1,000 ppm to about 1,600 ppm, about 1,000 ppm to about 1,700 ppm, about 1,000 ppm to about 1,800 ppm, about 1,000 ppm to about 1,900 ppm, about 1,000 ppm to about 2,000 ppm, about 1,100 ppm to about 1,200 ppm, about 1,100 ppm to about 1,300 ppm, about 1,100 ppm to about 1,400 ppm, about 1,100 ppm to about 1,500 ppm, about 1,100 ppm to about 1,600 ppm, about 1,100 ppm to about 1,700 ppm, about 1,100 ppm to about 1,800 ppm, about 1,100 ppm to about 1,900 ppm, about 1,100 ppm to about 2,000 ppm, about 1,200 ppm to about 1,300 ppm, about 1,200 ppm to about 1,400 ppm, about 1,200 ppm to about 1,500 ppm, about 1,200 ppm to about 1,600 ppm, about 1,200 ppm to about 1,700 ppm, about 1,200 ppm to about 1,800 ppm, about 1,200 ppm to about 1,900 ppm, about 1,200 ppm to about 2,000 ppm, about 1,300 ppm to about 1,400 ppm, about 1,300 ppm to about 1,500 ppm, about 1,300 ppm to about 1,600 ppm, about 1,300 ppm to about 1,700 ppm, about 1,300 ppm to about 1,800 ppm, about 1,300 ppm to about 1,900 ppm, about 1,300 ppm to about 2,000 ppm, about 1,400 ppm to about 1,500 ppm, about 1,400 ppm to about 1,600 ppm, about 1,400 ppm to about 1,700 ppm, about 1,400 ppm to about 1,800 ppm, about 1,400 ppm to about 1,900 ppm, about 1,400 ppm to about 2,000 ppm, about 1,500 ppm to about 1,600 ppm, about 1,500 ppm to about 1,700 ppm, about 1,500 ppm to about 1,800 ppm, about 1,500 ppm to about 1,900 ppm, about 1,500 ppm to about 2,000 ppm, about 1,600 ppm to about 1,700 ppm, about 1,600 ppm to about 1,800 ppm, about 1,600 ppm to about 1,900 ppm, about 1,600 ppm to about 2,000 ppm, about 1,700 ppm to about 1,800 ppm, about 1,700 ppm to about 1,900 ppm, about 1,700 ppm to about 2,000 ppm, about 1,800 ppm to about 1,900 ppm, about 1,800 ppm to about 2,000 ppm, or about 1,900 ppm to about 2,000 ppm.

In aspects of this embodiment, a composition disclosed herein comprises hypochlorous acid in an amount of, e.g., about 0.001%, about 0.005%, about 0.01%, about 0.05%, about 0.10%, about 0.11%, about 0.12%, about 0.13%, about 0.14%, about 0.15%, about 0.16%, about 0.17%, about 0.18%, about 0.19%, about 0.2%, about 0.25%, about 0.3%, about 0.35%, about 0.4%, about 0.45%, or about 0.5% by weight of the composition. In other aspects of this embodiment, a composition disclosed herein comprises hypochlorous acid in an amount of, e.g., at least 0.001%, at least 0.005%, at least 0.01%, at least 0.05%, at least 0.10%, at least 0.11%, at least 0.12%, at least 0.13%, at least 0.14%, at least 0.15%, at least 0.16%, at least 0.17%, at least 0.18%, at least 0.19%, at least 0.2%, at least 0.25%, at least 0.3%, at least 0.35%, at least 0.4%, at least 0.45%, or at least 0.5% by weight of the composition. In yet other aspects of this embodiment, a composition disclosed herein comprises hypochlorous acid in an amount of, e.g., at most 0.001%, at most 0.005%, at most 0.01%, at most 0.05%, at most 0.10%, at most 0.11%, at most 0.12%, at most 0.13%, at most 0.14%, at most 0.15%, at most 0.16%, at most 0.17%, at most 0.18%, at most 0.19%, at most 0.2%, at most 0.25%, at most 0.3%, at most 0.35%, at most 0.4%, at most 0.45%, or at most 0.5% by weight of the composition. In still other aspects of this embodiment, a composition disclosed herein comprises hypochlorous acid in an amount of, e.g., about 0.0010% to about 0.005%, about 0.0010% to about 0.010%, about 0.0010% to about 0.050%, about 0.0010% to about 0.10%, about 0.0010% to about 0.15%, about 0.0010% to about 0.20%, about 0.0010% to about 0.25%, about 0.0010% to about 0.30%, about 0.0010% to about 0.35%, about 0.0010% to about 0.40%, about 0.0010% to about 0.45%, about 0.0010% to about 0.50%, about 0.010% to about 0.050%, about 0.010% to about 0.10%, about 0.010% to about 0.15%, about 0.010% to about 0.20%, about 0.010% to about 0.25%, about 0.010% to about 0.30%, about 0.010% to about 0.35%, about 0.010% to about 0.40%, about 0.010% to about 0.45%, about 0.010% to about 0.50%, about 0.10% to about 0.15%, about 0.10% to about 0.20%, about 0.10% to about 0.25%, about 0.10% to about 0.30%, about 0.10% to about 0.35%, about 0.10% to about 0.40%, about 0.10% to about 0.45%, about 0.10% to about 0.50%, about 0.20% to about 0.25%, about 0.20% to about 0.30%, about 0.20% to about 0.35%, about 0.20% to about 0.40%, about 0.20% to about 0.45%, about 0.20% to about 0.50%, about 0.30% to about 0.35%, about 0.30% to about 0.40%, about 0.30% to about 0.45%, about 0.30% to about 0.50%, about 0.40% to about 0.45%, about 0.40% to about 0.50%, or about 0.40% to about 0.50% by weight of the composition.

In one embodiment, a composition disclosed herein does not comprise hypochlorous acid.

The concentration of hypochlorous acid in solution may be described as free available chlorine in parts per million. Hypochlorous acid is in equilibrium with hypochlorite ions (OCl⁻) and dissolved chlorine gas (Cl₂). The extent of the equilibrium is determined predominately by the salt concentration and pH of the solution. Temperature also impacts the ratio of the free chlorine component. Therefore, both FAC and pH need to be known to understand the amount of chlorine present as hypochlorous acid. In general, when the pH range is about 4.0 to about 5.6, approximately 100% of the available chlorine is present as HOCl. As the pH is lowered below about 4, there is an increase in dissolved chlorine gas (Cl₂). Thus, at a pH of about 3, about 90% of the available chlorine is present as hypochlorous acid, at a pH of about 2, about 75% of the available chlorine is present as hypochlorous acid, at a pH of about 1.5, about 50% of the available chlorine is present as hypochlorous acid, while at a pH of about 1, about 25% of the available chlorine is present as hypochlorous acid. As the pH is increase above about 5.6, there is an increase in hypochlorite ions (OCl⁻). Thus, at a pH a pH of about 6.5, about 90% of the available chlorine is present as hypochlorous acid, at a pH of about 7, about 75% of the available chlorine is present as hypochlorous acid, at a pH of about 7.5, about 50% of the available chlorine is present as hypochlorous acid, while at a pH of about 8, about 25% of the available chlorine is present as hypochlorous acid.

The chlorine amount may be measured by methods known in the art, such as a DPD colorimeter method (Lamotte Company, Chestertown, Md.), a UV spectrophotometry method, or other known methods established by the Environmental Protection Agency. In the DPD colorimeter method, a yellow color is formed by the reaction of free chlorine With N,N-diethyl-p-phenylenediamine (DPD) and the intensity is measured With a calibrated calorimeter that provides the output in parts per million. Further addition of potassium iodide turns the solution a pink color to provide the total chlorine value.

In an embodiment, a composition disclosed herein comprises an amount of free available chlorine that provides a desired beneficial effect to a composition disclosed herein. For example, when a composition comprising free available chlorine is formulated to be used in an undiluted state, the amount of hypochlorous acid used is a microbiostatic effective amount. A microbiostatic effective amount of free available chlorine is an amount that restrains, prohibits, or otherwise prevents microbial population growth without eradicating the microbes. In an aspect of this embodiment, a microbiostatic effective amount of free available chlorine is an amount that restrains, prohibits, or otherwise prevents microbial population growth without eradicating the microbes to a degree that achieves disinfection or sterilization. Alternatively, when a composition comprising free available chlorine is formulated to be used in methods where the composition will be diluted, the amount of free available chlorine used in such concentrated formulations can be any amount that does not cause precipitation of components in the composition disclosed herein or otherwise create unwanted effects that make the composition inoperable.

In aspects of this embodiment, a composition disclosed herein comprises free available chlorine in an amount of, e.g., 0.01 ppm, 0.05 ppm, 0.10 ppm, 0.15 ppm, 0.20 ppm, 0.25 ppm, 0.30 ppm, 0.35 ppm, 0.40 ppm, 0.45 ppm, 0.50 ppm, 0.55 ppm, 0.60 ppm, 0.65 ppm, 0.70 ppm, 0.75 ppm, 0.80 ppm, 0.85 ppm, 0.90 ppm, 0.95 ppm, 1 ppm, 5 ppm, 10 ppm, 15 ppm, 20 ppm, 25 ppm, 30 ppm, 35 ppm, 40 ppm, 45 ppm, 50 ppm, 55 ppm, 60 ppm, 65 ppm, 70 ppm, 75 ppm, 80 ppm, 85 ppm, 90 ppm, 95 ppm, 100 ppm, 125 ppm, 150 ppm, 175 ppm, 200 ppm, 225 ppm, 250 ppm, 275 ppm, 300 ppm, 325 ppm, 350 ppm, 375 ppm, 400 ppm, 425 ppm, 450 ppm, 475 ppm, 500 ppm, 525 ppm, 550 ppm, 575 ppm, 600 ppm, 625 ppm, 650 ppm, 675 ppm, 700 ppm, 725 ppm, 750 ppm, 775 ppm, 800 ppm, 825 ppm, 850 ppm, 875 ppm, 900 ppm, 925 ppm, 950 ppm, 975 ppm, 1,000 ppm, 1,025 ppm, 1,050 ppm, 1075 ppm, 1,100 ppm, 1,125 ppm, 1,150 ppm, 1,175 ppm, 1,200 ppm, 1,225 ppm, 1,250 ppm, 1,275 ppm, 1,300 ppm, 1,325 ppm, 1,350 ppm, 1,375 ppm, 1,400 ppm, 1,425 ppm, 1,450 ppm, 1,475 ppm, 1,500 ppm, 1,600 ppm, 1,700 ppm, 1,800 ppm, 1,900 ppm, or 2,000 ppm. In other aspects of this embodiment, a composition disclosed herein comprises free available chlorine in an amount of, e.g., at least 0.01 ppm, at least 0.05 ppm, at least 0.10 ppm, at least 0.20 ppm, at least 0.30 ppm, at least 0.40 ppm, at least 0.50 ppm, at least 0.60 ppm, at least 0.70 ppm, at least 0.80 ppm, at least 0.90 ppm, at least 1 ppm, at least 10 ppm, at least 20 ppm, at least 30 ppm, at least 40 ppm, at least 50 ppm, at least 60 ppm, at least 70 ppm, at least 80 ppm, at least 90 ppm, at least 100 ppm, at least 125 ppm, at least 150 ppm, at least 175 ppm, at least 200 ppm, at least 225 ppm, at least 250 ppm, at least 275 ppm, at least 300 ppm, at least 325 ppm, at least 350 ppm, at least 375 ppm, at least 400 ppm, at least 425 ppm, at least 450 ppm, at least 475 ppm, at least 500 ppm, at least 525 ppm, at least 550 ppm, at least 575 ppm, at least 600 ppm, at least 625 ppm, at least 650 ppm, at least 675 ppm, at least 700 ppm, at least 725 ppm, at least 750 ppm, at least 775 ppm, at least 800 ppm, at least 825 ppm, at least 850 ppm, at least 875 ppm, at least 900 ppm, at least 925 ppm, at least 950 ppm, at least 975 ppm, at least 1,000 ppm, at least 1,025 ppm, at least 1,050 ppm, at least 1075 ppm, at least 1,100 ppm, at least 1,125 ppm, at least 1,150 ppm, at least 1,175 ppm, at least 1,200 ppm, at least 1,225 ppm, at least 1,250 ppm, at least 1,275 ppm, at least 1,300 ppm, at least 1,325 ppm, at least 1,350 ppm, at least 1,375 ppm, at least 1,400 ppm, at least 1,425 ppm, at least 1,450 ppm, at least 1,475 ppm, at least 1,500 ppm, at least 1,600 ppm, at least 1,700 ppm, at least 1,800 ppm, at least 1,900 ppm, or at least 2,000 ppm. In yet other aspects of this embodiment, a composition disclosed herein comprises free available chlorine in an amount of, e.g., at most 0.01 ppm, at most 0.05 ppm, at most 0.10 ppm, at most 0.20 ppm, at most 0.30 ppm, at most 0.40 ppm, at most 0.50 ppm, at most 0.60 ppm, at most 0.70 ppm, at most 0.80 ppm, at most 0.90 ppm, at most 1 ppm, at most 10 ppm, at most 20 ppm, at most 30 ppm, at most 40 ppm, at most 50 ppm, at most 60 ppm, at most 70 ppm, at most 80 ppm, at most 90 ppm, at most 100 ppm, at most 125 ppm, at most 150 ppm, at most 175 ppm, at most 200 ppm, at most 225 ppm, at most 250 ppm, at most 275 ppm, at most 300 ppm, at most 325 ppm, at most 350 ppm, at most 375 ppm, at most 400 ppm, at most 425 ppm, at most 450 ppm, at most 475 ppm, at most 500 ppm, at most 525 ppm, at most 550 ppm, at most 575 ppm, at most 600 ppm, at most 625 ppm, at most 650 ppm, at most 675 ppm, at most 700 ppm, at most 725 ppm, at most 750 ppm, at most 775 ppm, at most 800 ppm, at most 825 ppm, at most 850 ppm, at most 875 ppm, at most 900 ppm, at most 925 ppm, at most 950 ppm, at most 975 ppm, at most 1,000 ppm, at most 1,025 ppm, at most 1,050 ppm, at most 1075 ppm, at most 1,100 ppm, at most 1,125 ppm, at most 1,150 ppm, at most 1,175 ppm, at most 1,200 ppm, at most 1,225 ppm, at most 1,250 ppm, at most 1,275 ppm, at most 1,300 ppm, at most 1,325 ppm, at most 1,350 ppm, at most 1,375 ppm, at most 1,400 ppm, at most 1,425 ppm, at most 1,450 ppm, at most 1,475 ppm, at most 1,500 ppm, at most 1,600 ppm, at most 1,700 ppm, at most 1,800 ppm, at most 1,900 ppm, or at most 2,000 ppm.

In still other aspects of this embodiment, a composition disclosed herein comprises free available chlorine in an amount of from, e.g., about 0.01 ppm to about 0.1 ppm, about 0.01 ppm to about 0.2 ppm, about 0.01 ppm to about 0.25 ppm, about 0.01 ppm to about 0.3 ppm, about 0.01 ppm to about 0.4 ppm, about 0.01 ppm to about 0.5 ppm, about 0.01 ppm to about 0.1 ppm, about 0.01 ppm to about 0.5 ppm, about 0.01 ppm to about 1 ppm, about 0.01 ppm to about 5 ppm, about 0.01 ppm to about 10 ppm, about 0.01 ppm to about 15 ppm, about 0.01 ppm to about 20 ppm, about 0.01 ppm to about 25 ppm, about 0.01 ppm to about 30 ppm, about 0.01 ppm to about 35 ppm, about 0.01 ppm to about 40 ppm, about 0.01 ppm to about 45 ppm, about 0.01 ppm to about 50 ppm, about 0.05 ppm to about 0.1 ppm, about 0.05 ppm to about 0.5 ppm, about 0.05 ppm to about 1 ppm, about 0.05 ppm to about 5 ppm, about 0.05 ppm to about 10 ppm, about 0.05 ppm to about 15 ppm, about 0.05 ppm to about 20 ppm, about 0.05 ppm to about 25 ppm, about 0.05 ppm to about 30 ppm, about 0.05 ppm to about 35 ppm, about 0.05 ppm to about 40 ppm, about 0.05 ppm to about 45 ppm, about 0.05 ppm to about 50 ppm, about 0.1 ppm to about 1 ppm, about 0.1 ppm to about 5 ppm, about 0.1 ppm to about 10 ppm, about 0.1 ppm to about 15 ppm, about 0.1 ppm to about 20 ppm, about 0.1 ppm to about 25 ppm, about 0.1 ppm to about 30 ppm, about 0.1 ppm to about 35 ppm, about 0.1 ppm to about 40 ppm, about 0.1 ppm to about 45 ppm, about 0.1 ppm to about 50 ppm, about 0.5 ppm to about 1 ppm, about 0.5 ppm to about 5 ppm, about 0.5 ppm to about 10 ppm, about 0.5 ppm to about 15 ppm, about 0.5 ppm to about 20 ppm, about 0.5 ppm to about 25 ppm, about 0.5 ppm to about 30 ppm, about 0.5 ppm to about 35 ppm, about 0.5 ppm to about 40 ppm, about 0.5 ppm to about 45 ppm, about 0.5 ppm to about 50 ppm, about 0.5 ppm to about 55 ppm, about 0.5 ppm to about 60 ppm, about 0.5 ppm to about 65 ppm, about 0.5 ppm to about 70 ppm, about 0.5 ppm to about 75 ppm, about 0.5 ppm to about 80 ppm, about 0.5 ppm to about 85 ppm, about 0.5 ppm to about 90 ppm, about 0.5 ppm to about 95 ppm, about 0.5 ppm to about 100 ppm, about 0.75 ppm to about 20 ppm, about 0.75 ppm to about 25 ppm, about 0.75 ppm to about 30 ppm, about 0.75 ppm to about 35 ppm, about 0.75 ppm to about 40 ppm, about 0.75 ppm to about 45 ppm, about 0.75 ppm to about 50 ppm, about 0.75 ppm to about 55 ppm, about 0.75 ppm to about 60 ppm, about 0.75 ppm to about 65 ppm, about 0.75 ppm to about 70 ppm, about 0.75 ppm to about 75 ppm, about 0.75 ppm to about 80 ppm, about 0.75 ppm to about 85 ppm, about 0.75 ppm to about 90 ppm, about 0.75 ppm to about 95 ppm, about 0.75 ppm to about 100 ppm, about 1 ppm to about 10 ppm, about 1 ppm to about 15 ppm, about 1 ppm to about 20 ppm, about 1 ppm to about 25 ppm, about 1 ppm to about 30 ppm, about 1 ppm to about 35 ppm, about 1 ppm to about 40 ppm, about 1 ppm to about 45 ppm, about 1 ppm to about 50 ppm, about 1 ppm to about 55 ppm, about 1 ppm to about 60 ppm, about 1 ppm to about 65 ppm, about 1 ppm to about 70 ppm, about 1 ppm to about 75 ppm, about 1 ppm to about 80 ppm, about 1 ppm to about 85 ppm, about 1 ppm to about 90 ppm, about 1 ppm to about 95 ppm, about 1 ppm to about 100 ppm, about 5 ppm to about 10 ppm, about 5 ppm to about 15 ppm, about 5 ppm to about 20 ppm, about 5 ppm to about 25 ppm, about 5 ppm to about 30 ppm, about 5 ppm to about 35 ppm, about 5 ppm to about 40 ppm, about 5 ppm to about 45 ppm, about 5 ppm to about 50 ppm, about 5 ppm to about 55 ppm, about 5 ppm to about 60 ppm, about 5 ppm to about 65 ppm, about 5 ppm to about 70 ppm, about 5 ppm to about 75 ppm, about 5 ppm to about 80 ppm, about 5 ppm to about 85 ppm, about 5 ppm to about 90 ppm, about 5 ppm to about 95 ppm, about 5 ppm to about 100 ppm, about 10 ppm to about 20 ppm, about 10 ppm to about 25 ppm, about 10 ppm to about 30 ppm, about 10 ppm to about 35 ppm, about 10 ppm to about 40 ppm, about 10 ppm to about 45 ppm, about 10 ppm to about 50 ppm, about 10 ppm to about 55 ppm, about 10 ppm to about 60 ppm, about 10 ppm to about 65 ppm, about 10 ppm to about 70 ppm, about 10 ppm to about 75 ppm, about 10 ppm to about 80 ppm, about 10 ppm to about 85 ppm, about 10 ppm to about 90 ppm, about 10 ppm to about 95 ppm, or about 10 ppm to about 100 ppm.

In other aspects of this embodiment, a composition disclosed herein comprises free available chlorine in an amount of from, e.g., about 1 ppm to about 25 ppm, about 1 ppm to about 50 ppm, about 1 ppm to about 75 ppm, about 1 ppm to about 100 ppm, about 1 ppm to about 125 ppm, about 1 ppm to about 150 ppm, about 1 ppm to about 175 ppm, about 1 ppm to about 200 ppm, about 1 ppm to about 225 ppm, about 1 ppm to about 250 ppm, about 1 ppm to about 275 ppm, about 1 ppm to about 300 ppm, about 1 ppm to about 325 ppm, about 1 ppm to about 350 ppm, about 1 ppm to about 375 ppm, about 1 ppm to about 400 ppm, about 10 ppm to about 25 ppm, about 10 ppm to about 50 ppm, about 10 ppm to about 75 ppm, about 10 ppm to about 100 ppm, about 10 ppm to about 125 ppm, about 10 ppm to about 150 ppm, about 10 ppm to about 175 ppm, about 10 ppm to about 200 ppm, about 10 ppm to about 225 ppm, about 10 ppm to about 250 ppm, about 10 ppm to about 275 ppm, about 10 ppm to about 300 ppm, about 10 ppm to about 325 ppm, about 10 ppm to about 350 ppm, about 10 ppm to about 375 ppm, about 10 ppm to about 400 ppm, about 25 ppm to about 50 ppm, about 25 ppm to about 75 ppm, about 25 ppm to about 100 ppm, about 25 ppm to about 125 ppm, about 25 ppm to about 150 ppm, about 25 ppm to about 175 ppm, about 25 ppm to about 200 ppm, about 25 ppm to about 225 ppm, about 25 ppm to about 250 ppm, about 25 ppm to about 275 ppm, about 25 ppm to about 300 ppm, about 25 ppm to about 325 ppm, about 25 ppm to about 350 ppm, about 25 ppm to about 375 ppm, about 25 ppm to about 400 ppm, about 50 ppm to about 75 ppm, about 50 ppm to about 100 ppm, about 50 ppm to about 125 ppm, about 50 ppm to about 150 ppm, about 50 ppm to about 175 ppm, about 50 ppm to about 200 ppm, about 50 ppm to about 225 ppm, about 50 ppm to about 250 ppm, about 50 ppm to about 275 ppm, about 50 ppm to about 300 ppm, about 50 ppm to about 325 ppm, about 50 ppm to about 350 ppm, about 50 ppm to about 375 ppm, about 50 ppm to about 400 ppm, about 75 ppm to about 100 ppm, about 75 ppm to about 125 ppm, about 75 ppm to about 150 ppm, about 75 ppm to about 175 ppm, about 75 ppm to about 200 ppm, about 75 ppm to about 225 ppm, about 75 ppm to about 250 ppm, about 75 ppm to about 275 ppm, about 75 ppm to about 300 ppm, about 75 ppm to about 325 ppm, about 75 ppm to about 350 ppm, about 75 ppm to about 375 ppm, about 75 ppm to about 400 ppm, about 100 ppm to about 125 ppm, about 100 ppm to about 150 ppm, about 100 ppm to about 175 ppm, about 100 ppm to about 200 ppm, about 100 ppm to about 225 ppm, about 100 ppm to about 250 ppm, about 100 ppm to about 275 ppm, about 100 ppm to about 300 ppm, about 100 ppm to about 325 ppm, about 100 ppm to about 350 ppm, about 100 ppm to about 375 ppm, about 100 ppm to about 400 ppm, about 150 ppm to about 175 ppm, about 150 ppm to about 200 ppm, about 150 ppm to about 225 ppm, about 150 ppm to about 250 ppm, about 150 ppm to about 275 ppm, about 150 ppm to about 300 ppm, about 150 ppm to about 325 ppm, about 150 ppm to about 350 ppm, about 150 ppm to about 375 ppm, about 150 ppm to about 400 ppm, about 200 ppm to about 225 ppm, about 200 ppm to about 250 ppm, about 200 ppm to about 275 ppm, about 200 ppm to about 300 ppm, about 200 ppm to about 325 ppm, about 200 ppm to about 350 ppm, about 200 ppm to about 375 ppm, about 200 ppm to about 400 ppm, about 250 ppm to about 275 ppm, about 250 ppm to about 300 ppm, about 250 ppm to about 325 ppm, about 250 ppm to about 350 ppm, about 250 ppm to about 375 ppm, about 250 ppm to about 400 ppm, about 300 ppm to about 325 ppm, about 300 ppm to about 350 ppm, about 300 ppm to about 375 ppm, about 300 ppm to about 400 ppm, about 350 ppm to about 375 ppm, about 350 ppm to about 400 ppm, or about 375 ppm to about 400 ppm.

In other aspects of this embodiment, a composition disclosed herein comprises free available chlorine in an amount of, e.g., about 400 ppm to about 500 ppm, about 400 ppm to about 600 ppm, about 400 ppm to about 700 ppm, about 400 ppm to about 800 ppm, about 400 ppm to about 900 ppm, about 400 ppm to about 1,000 ppm, about 400 ppm to about 1,100 ppm, about 400 ppm to about 1,200 ppm, about 400 ppm to about 1,300 ppm, about 400 ppm to about 1,400 ppm, about 400 ppm to about 1,500 ppm, about 400 ppm to about 1,600 ppm, about 400 ppm to about 1,700 ppm, about 400 ppm to about 1,800 ppm, about 400 ppm to about 1,900 ppm, about 400 ppm to about 2,000 ppm, about 500 ppm to about 600 ppm, about 500 ppm to about 700 ppm, about 500 ppm to about 800 ppm, about 500 ppm to about 900 ppm, about 500 ppm to about 1,000 ppm, about 500 ppm to about 1,100 ppm, about 500 ppm to about 1,200 ppm, about 500 ppm to about 1,300 ppm, about 500 ppm to about 1,400 ppm, about 500 ppm to about 1,500 ppm, about 500 ppm to about 1,600 ppm, about 500 ppm to about 1,700 ppm, about 500 ppm to about 1,800 ppm, about 500 ppm to about 1,900 ppm, about 500 ppm to about 2,000 ppm, about 600 ppm to about 700 ppm, about 600 ppm to about 800 ppm, about 600 ppm to about 900 ppm, about 600 ppm to about 1,000 ppm, about 600 ppm to about 1,100 ppm, about 600 ppm to about 1,200 ppm, about 600 ppm to about 1,300 ppm, about 600 ppm to about 1,400 ppm, about 600 ppm to about 1,500 ppm, about 600 ppm to about 1,600 ppm, about 600 ppm to about 1,700 ppm, about 600 ppm to about 1,800 ppm, about 600 ppm to about 1,900 ppm, about 600 ppm to about 2,000 ppm, about 700 ppm to about 800 ppm, about 700 ppm to about 900 ppm, about 700 ppm to about 1,000 ppm, about 700 ppm to about 1,100 ppm, about 700 ppm to about 1,200 ppm, about 700 ppm to about 1,300 ppm, about 700 ppm to about 1,400 ppm, about 700 ppm to about 1,500 ppm, about 700 ppm to about 1,600 ppm, about 700 ppm to about 1,700 ppm, about 700 ppm to about 1,800 ppm, about 700 ppm to about 1,900 ppm, about 700 ppm to about 2,000 ppm, about 800 ppm to about 900 ppm, about 800 ppm to about 1,000 ppm, about 800 ppm to about 1,100 ppm, about 800 ppm to about 1,200 ppm, about 800 ppm to about 1,300 ppm, about 800 ppm to about 1,400 ppm, about 800 ppm to about 1,500 ppm, about 800 ppm to about 1,600 ppm, about 800 ppm to about 1,700 ppm, about 800 ppm to about 1,800 ppm, about 800 ppm to about 1,900 ppm, about 800 ppm to about 2,000 ppm, about 900 ppm to about 1,000 ppm, about 900 ppm to about 1,100 ppm, about 900 ppm to about 1,200 ppm, about 900 ppm to about 1,300 ppm, about 900 ppm to about 1,400 ppm, about 900 ppm to about 1,500 ppm, about 900 ppm to about 1,600 ppm, about 900 ppm to about 1,700 ppm, about 900 ppm to about 1,800 ppm, about 900 ppm to about 1,900 ppm, about 900 ppm to about 2,000 ppm, about 1,000 ppm to about 1,100 ppm, about 1,000 ppm to about 1,200 ppm, about 1,000 ppm to about 1,300 ppm, about 1,000 ppm to about 1,400 ppm, about 1,000 ppm to about 1,500 ppm, about 1,000 ppm to about 1,600 ppm, about 1,000 ppm to about 1,700 ppm, about 1,000 ppm to about 1,800 ppm, about 1,000 ppm to about 1,900 ppm, about 1,000 ppm to about 2,000 ppm, about 1,100 ppm to about 1,200 ppm, about 1,100 ppm to about 1,300 ppm, about 1,100 ppm to about 1,400 ppm, about 1,100 ppm to about 1,500 ppm, about 1,100 ppm to about 1,600 ppm, about 1,100 ppm to about 1,700 ppm, about 1,100 ppm to about 1,800 ppm, about 1,100 ppm to about 1,900 ppm, about 1,100 ppm to about 2,000 ppm, about 1,200 ppm to about 1,300 ppm, about 1,200 ppm to about 1,400 ppm, about 1,200 ppm to about 1,500 ppm, about 1,200 ppm to about 1,600 ppm, about 1,200 ppm to about 1,700 ppm, about 1,200 ppm to about 1,800 ppm, about 1,200 ppm to about 1,900 ppm, about 1,200 ppm to about 2,000 ppm, about 1,300 ppm to about 1,400 ppm, about 1,300 ppm to about 1,500 ppm, about 1,300 ppm to about 1,600 ppm, about 1,300 ppm to about 1,700 ppm, about 1,300 ppm to about 1,800 ppm, about 1,300 ppm to about 1,900 ppm, about 1,300 ppm to about 2,000 ppm, about 1,400 ppm to about 1,500 ppm, about 1,400 ppm to about 1,600 ppm, about 1,400 ppm to about 1,700 ppm, about 1,400 ppm to about 1,800 ppm, about 1,400 ppm to about 1,900 ppm, about 1,400 ppm to about 2,000 ppm, about 1,500 ppm to about 1,600 ppm, about 1,500 ppm to about 1,700 ppm, about 1,500 ppm to about 1,800 ppm, about 1,500 ppm to about 1,900 ppm, about 1,500 ppm to about 2,000 ppm, about 1,600 ppm to about 1,700 ppm, about 1,600 ppm to about 1,800 ppm, about 1,600 ppm to about 1,900 ppm, about 1,600 ppm to about 2,000 ppm, about 1,700 ppm to about 1,800 ppm, about 1,700 ppm to about 1,900 ppm, about 1,700 ppm to about 2,000 ppm, about 1,800 ppm to about 1,900 ppm, about 1,800 ppm to about 2,000 ppm, or about 1,900 ppm to about 2,000 ppm.

In aspects of this embodiment, a composition disclosed herein comprises free available chlorine in an amount of, e.g., about 0.001%, about 0.005%, about 0.01%, about 0.05%, about 0.001%, about 0.01%, about 0.10%, about 0.11%, about 0.12%, about 0.13%, about 0.14%, about 0.15%, about 0.16%, about 0.17%, about 0.18%, about 0.19%, about 0.2%, about 0.25%, about 0.3%, about 0.35%, about 0.4%, about 0.45%, or about 0.5% by weight of the composition. In other aspects of this embodiment, a composition disclosed herein comprises free available chlorine in an amount of, e.g., at least 0.001%, at least 0.005%, at least 0.01%, at least 0.05%, at least 0.10%, at least 0.11%, at least 0.12%, at least 0.13%, at least 0.14%, at least 0.15%, at least 0.16%, at least 0.17%, at least 0.18%, at least 0.19%, at least 0.2%, at least 0.25%, at least 0.3%, at least 0.35%, at least 0.4%, at least 0.45%, or at least 0.5% by weight of the composition. In yet other aspects of this embodiment, a composition disclosed herein comprises free available chlorine in an amount of, e.g., at most 0.001%, at most 0.005%, at most 0.01%, at most 0.05%, at most 0.10%, at most 0.11%, at most 0.12%, at most 0.13%, at most 0.14%, at most 0.15%, at most 0.16%, at most 0.17%, at most 0.18%, at most 0.19%, at most 0.2%, at most 0.25%, at most 0.3%, at most 0.35%, at most 0.4%, at most 0.45%, or at most 0.5% by weight of the composition. In still other aspects of this embodiment, a composition disclosed herein comprises free available chlorine in an amount of, e.g., about 0.0010% to about 0.005%, about 0.0010% to about 0.010%, about 0.0010% to about 0.050%, about 0.0010% to about 0.10%, about 0.0010% to about 0.15%, about 0.0010% to about 0.20%, about 0.0010% to about 0.25%, about 0.0010% to about 0.30%, about 0.0010% to about 0.35%, about 0.0010% to about 0.40%, about 0.0010% to about 0.45%, about 0.0010% to about 0.50%, about 0.010% to about 0.050%, about 0.010% to about 0.10%, about 0.010% to about 0.15%, about 0.010% to about 0.20%, about 0.010% to about 0.25%, about 0.010% to about 0.30%, about 0.010% to about 0.35%, about 0.010% to about 0.40%, about 0.010% to about 0.45%, about 0.010% to about 0.50%, about 0.10% to about 0.15%, about 0.10% to about 0.20%, about 0.10% to about 0.25%, about 0.10% to about 0.30%, about 0.10% to about 0.35%, about 0.10% to about 0.40%, about 0.10% to about 0.45%, about 0.10% to about 0.50%, about 0.20% to about 0.25%, about 0.20% to about 0.30%, about 0.20% to about 0.35%, about 0.20% to about 0.40%, about 0.20% to about 0.45%, about 0.20% to about 0.50%, about 0.30% to about 0.35%, about 0.30% to about 0.40%, about 0.30% to about 0.45%, about 0.30% to about 0.50%, about 0.40% to about 0.45%, about 0.40% to about 0.50%, or about 0.40% to about 0.50% by weight of the composition.

In one embodiment, a composition disclosed herein does not comprise free available chlorine.

In one embodiment, a composition disclosed herein does not comprise ozone. In another embodiment, a composition disclosed herein can comprise ozone.

In one embodiment, a composition disclosed herein does not comprise hydrogen peroxide. In another embodiment, a composition disclosed herein can comprise hydrogen peroxide.

A composition disclosed herein may comprise a quaternary compound. Non-limiting examples of a quaternary compound includes a dialkyl quaternary compound and a polyether fatty quaternary compound. Examples of a dialkyl quaternary compound includes, didodecyldimethylammonium chloride and di-n-alkyldimethyl ammonium chloride. A quaternary compound includes a silicon quaternary compound. Non-limiting examples of a silicon quaternary compound includes a silicone dialkyl quaternary compound and a silicone polyether fatty quaternary compound. Examples of a silicone dialkyl quaternary compound includes, without limitation, dimethyloctadecyl[3-(trimethoxysilyl)propyl]ammonium chloride (DTSACI) and tetradecyldimethyl-(3-trimethoxysilyl)propyl ammonium chloride (TTSACI). Examples of other quaternary compounds include, without limitation, a quaternary ammonium salt, such as, e.g., Quaternium-15 and Quaternium-18 and a polycationic polymer, such as, e.g., cetylpyridinium salt, like cetylpyridinium chloride or cetylpyridinium bromide, and a polyquaternium like Polyquaterniums 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, and 47. Other none-limiting examples of a quaternary compound include those with the formula NR₄⁺, where R is an alkyl or an aryl group.

In one embodiment, a single quaternary compound or silicon quaternary compound is present in a composition disclosed herein. In another embodiment, a plurality of quaternary compounds or silicon quaternary compounds is present in a composition disclosed herein. In aspects of this embodiment, a composition disclosed herein comprises, e.g., one or more quaternary compounds or silicon quaternary compounds, two or more quaternary compounds or silicon quaternary compounds, three or more quaternary compounds or silicon quaternary compounds, four or more quaternary compounds or silicon quaternary compounds or five or more quaternary compounds or silicon quaternary compounds. In other aspects of this embodiment, a composition disclosed herein comprises, e.g., only one quaternary compound or silicon quaternary compound, at most two quaternary compounds or silicon quaternary compounds, at most three quaternary compounds or silicon quaternary compounds, at most four quaternary compounds or silicon quaternary compounds, or at most five quaternary compounds or silicon quaternary compounds. In yet other aspects of this embodiment, a composition disclosed herein comprises from, e.g., 1 to 2 quaternary compounds or silicon quaternary compounds, 1 to 3 quaternary compounds or silicon quaternary compounds, 1 to 4 quaternary compounds or silicon quaternary compounds, 1 to 5 quaternary compounds or silicon quaternary compounds, 2 to 3 quaternary compounds or silicon quaternary compounds, 2 to 4 quaternary compounds or silicon quaternary compounds, 2 to 5 quaternary compounds or silicon quaternary compounds, 3 to 4 quaternary compounds or silicon quaternary compounds, 3 to 5 quaternary compounds or silicon quaternary compounds or 4 to 5 quaternary compounds or silicon quaternary compounds.

In an embodiment, a composition disclosed herein comprises an amount of a quaternary compound or silicon quaternary compound that provides a desired beneficial effect to a composition disclosed herein. For example, when a composition comprising a quaternary compound or silicon quaternary compound is formulated to be used in an undiluted state, the amount of a quaternary compound or silicon quaternary compound used is a microbiostatic effective amount. A microbiostatic effective amount of a quaternary compound or silicon quaternary compound is an amount that restrains, prohibits, or otherwise prevents microbial population growth without eradicating the microbes. In an aspect of this embodiment, a microbiostatic effective amount of a quaternary compound or silicon quaternary compound is an amount that restrains, prohibits, or otherwise prevents microbial population growth without eradicating the microbes to a degree that achieves disinfection or sterilization. Alternatively, when a composition comprising a quaternary compound or silicon quaternary compound is formulated to be used in methods where the composition will be diluted, the amount of a quaternary compound or silicon quaternary compound used in such concentrated formulations can be any amount that does not cause precipitation of components in the composition disclosed herein or otherwise create unwanted effects that make the composition inoperable.

In aspects of this embodiment, a composition disclosed herein comprises a quaternary compound or silicon quaternary compound in an amount of, e.g., 0.05 ppm, 0.10 ppm, 0.15 ppm, 0.20 ppm, 0.25 ppm, 0.30 ppm, 0.35 ppm, 0.40 ppm, 0.45 ppm, 0.50 ppm, 0.55 ppm, 0.60 ppm, 0.65 ppm, 0.70 ppm, 0.75 ppm, 0.80 ppm, 0.85 ppm, 0.90 ppm, 0.95 ppm, 1 ppm, 5 ppm, 10 ppm, 15 ppm, 20 ppm, 25 ppm, 30 ppm, 35 ppm, 40 ppm, 45 ppm, 50 ppm, 55 ppm, 60 ppm, 65 ppm, 70 ppm, 75 ppm, 80 ppm, 85 ppm, 90 ppm, 95 ppm, 100 ppm, 125 ppm, 150 ppm, 175 ppm, 200 ppm, 225 ppm, 250 ppm, 275 ppm, 300 ppm, 325 ppm, 350 ppm, 375 ppm, 400 ppm, 425 ppm, 450 ppm, 475 ppm, 500 ppm, 525 ppm, 550 ppm, 575 ppm, 600 ppm, 625 ppm, 650 ppm, 675 ppm, 700 ppm, 725 ppm, 750 ppm, 775 ppm, 800 ppm, 825 ppm, 850 ppm, 875 ppm, 900 ppm, 925 ppm, 950 ppm, 975 ppm, 1,000 ppm, 1,025 ppm, 1,050 ppm, 1075 ppm, 1,100 ppm, 1,125 ppm, 1,150 ppm, 1,175 ppm, 1,200 ppm, 1,225 ppm, 1,250 ppm, 1,275 ppm, 1,300 ppm, 1,325 ppm, 1,350 ppm, 1,375 ppm, 1,400 ppm, 1,425 ppm, 1,450 ppm, 1,475 ppm, 1,500 ppm, 1,600 ppm, 1,700 ppm, 1,800 ppm, 1,900 ppm, or 2,000 ppm. In other aspects of this embodiment, a composition disclosed herein comprises a quaternary compound or silicon quaternary compound in an amount of, e.g., at least 0.05 ppm, at least 0.10 ppm, at least 0.20 ppm, at least 0.30 ppm, at least 0.40 ppm, at least 0.50 ppm, at least 0.60 ppm, at least 0.70 ppm, at least 0.80 ppm, at least 0.90 ppm, at least 1 ppm, at least 10 ppm, at least 20 ppm, at least 30 ppm, at least 40 ppm, at least 50 ppm, at least 60 ppm, at least 70 ppm, at least 80 ppm, at least 90 ppm, at least 100 ppm, at least 125 ppm, at least 150 ppm, at least 175 ppm, at least 200 ppm, at least 225 ppm, at least 250 ppm, at least 275 ppm, at least 300 ppm, at least 325 ppm, at least 350 ppm, at least 375 ppm, at least 400 ppm, at least 425 ppm, at least 450 ppm, at least 475 ppm, at least 500 ppm, at least 525 ppm, at least 550 ppm, at least 575 ppm, at least 600 ppm, at least 625 ppm, at least 650 ppm, at least 675 ppm, at least 700 ppm, at least 725 ppm, at least 750 ppm, at least 775 ppm, at least 800 ppm, at least 825 ppm, at least 850 ppm, at least 875 ppm, at least 900 ppm, at least 925 ppm, at least 950 ppm, at least 975 ppm, at least 1,000 ppm, at least 1,025 ppm, at least 1,050 ppm, at least 1075 ppm, at least 1,100 ppm, at least 1,125 ppm, at least 1,150 ppm, at least 1,175 ppm, at least 1,200 ppm, at least 1,225 ppm, at least 1,250 ppm, at least 1,275 ppm, at least 1,300 ppm, at least 1,325 ppm, at least 1,350 ppm, at least 1,375 ppm, at least 1,400 ppm, at least 1,425 ppm, at least 1,450 ppm, at least 1,475 ppm, at least 1,500 ppm, at least 1,600 ppm, at least 1,700 ppm, at least 1,800 ppm, at least 1,900 ppm, or at least 2,000 ppm. In yet other aspects of this embodiment, a composition disclosed herein comprises a quaternary compound or silicon quaternary compound in an amount of, e.g., at most 0.05 ppm, at most 0.10 ppm, at most 0.20 ppm, at most 0.30 ppm, at most 0.40 ppm, at most 0.50 ppm, at most 0.60 ppm, at most 0.70 ppm, at most 0.80 ppm, at most 0.90 ppm, at most 1 ppm, at most 10 ppm, at most 20 ppm, at most 30 ppm, at most 40 ppm, at most 50 ppm, at most 60 ppm, at most 70 ppm, at most 80 ppm, at most 90 ppm, at most 100 ppm, at most 125 ppm, at most 150 ppm, at most 175 ppm, at most 200 ppm, at most 225 ppm, at most 250 ppm, at most 275 ppm, at most 300 ppm, at most 325 ppm, at most 350 ppm, at most 375 ppm, at most 400 ppm, at most 425 ppm, at most 450 ppm, at most 475 ppm, at most 500 ppm, at most 525 ppm, at most 550 ppm, at most 575 ppm, at most 600 ppm, at most 625 ppm, at most 650 ppm, at most 675 ppm, at most 700 ppm, at most 725 ppm, at most 750 ppm, at most 775 ppm, at most 800 ppm, at most 825 ppm, at most 850 ppm, at most 875 ppm, at most 900 ppm, at most 925 ppm, at most 950 ppm, at most 975 ppm, at most 1,000 ppm, at most 1,025 ppm, at most 1,050 ppm, at most 1075 ppm, at most 1,100 ppm, at most 1,125 ppm, at most 1,150 ppm, at most 1,175 ppm, at most 1,200 ppm, at most 1,225 ppm, at most 1,250 ppm, at most 1,275 ppm, at most 1,300 ppm, at most 1,325 ppm, at most 1,350 ppm, at most 1,375 ppm, at most 1,400 ppm, at most 1,425 ppm, at most 1,450 ppm, at most 1,475 ppm, at most 1,500 ppm, at most 1,600 ppm, at most 1,700 ppm, at most 1,800 ppm, at most 1,900 ppm, or at most 2,000 ppm.

In still other aspects of this embodiment, a composition disclosed herein comprises a quaternary compound or silicon quaternary compound in an amount of from, e.g., about 0.5 ppm to about 20 ppm, about 0.5 ppm to about 25 ppm, about 0.5 ppm to about 30 ppm, about 0.5 ppm to about 35 ppm, about 0.5 ppm to about 40 ppm, about 0.5 ppm to about 45 ppm, about 0.5 ppm to about 50 ppm, about 0.5 ppm to about 55 ppm, about 0.5 ppm to about 60 ppm, about 0.5 ppm to about 65 ppm, about 0.5 ppm to about 70 ppm, about 0.5 ppm to about 75 ppm, about 0.5 ppm to about 80 ppm, about 0.5 ppm to about 85 ppm, about 0.5 ppm to about 90 ppm, about 0.5 ppm to about 95 ppm, about 0.5 ppm to about 100 ppm, about 0.75 ppm to about 20 ppm, about 0.75 ppm to about 25 ppm, about 0.75 ppm to about 30 ppm, about 0.75 ppm to about 35 ppm, about 0.75 ppm to about 40 ppm, about 0.75 ppm to about 45 ppm, about 0.75 ppm to about 50 ppm, about 0.75 ppm to about 55 ppm, about 0.75 ppm to about 60 ppm, about 0.75 ppm to about 65 ppm, about 0.75 ppm to about 70 ppm, about 0.75 ppm to about 75 ppm, about 0.75 ppm to about 80 ppm, about 0.75 ppm to about 85 ppm, about 0.75 ppm to about 90 ppm, about 0.75 ppm to about 95 ppm, about 0.75 ppm to about 100 ppm, about 1 ppm to about 20 ppm, about 1 ppm to about 25 ppm, about 1 ppm to about 30 ppm, about 1 ppm to about 35 ppm, about 1 ppm to about 40 ppm, about 1 ppm to about 45 ppm, about 1 ppm to about 50 ppm, about 1 ppm to about 55 ppm, about 1 ppm to about 60 ppm, about 1 ppm to about 65 ppm, about 1 ppm to about 70 ppm, about 1 ppm to about 75 ppm, about 1 ppm to about 80 ppm, about 1 ppm to about 85 ppm, about 1 ppm to about 90 ppm, about 1 ppm to about 95 ppm, about 1 ppm to about 100 ppm, about 5 ppm to about 20 ppm, about 5 ppm to about 25 ppm, about 5 ppm to about 30 ppm, about 5 ppm to about 35 ppm, about 5 ppm to about 40 ppm, about 5 ppm to about 45 ppm, about 5 ppm to about 50 ppm, about 5 ppm to about 55 ppm, about 5 ppm to about 60 ppm, about 5 ppm to about 65 ppm, about 5 ppm to about 70 ppm, about 5 ppm to about 75 ppm, about 5 ppm to about 80 ppm, about 5 ppm to about 85 ppm, about 5 ppm to about 90 ppm, about 5 ppm to about 95 ppm, about 5 ppm to about 100 ppm, about 10 ppm to about 20 ppm, about 10 ppm to about 25 ppm, about 10 ppm to about 30 ppm, about 10 ppm to about 35 ppm, about 10 ppm to about 40 ppm, about 10 ppm to about 45 ppm, about 10 ppm to about 50 ppm, about 10 ppm to about 55 ppm, about 10 ppm to about 60 ppm, about 10 ppm to about 65 ppm, about 10 ppm to about 70 ppm, about 10 ppm to about 75 ppm, about 10 ppm to about 80 ppm, about 10 ppm to about 85 ppm, about 10 ppm to about 90 ppm, about 10 ppm to about 95 ppm, or about 10 ppm to about 100 ppm.

In other aspects of this embodiment, a composition disclosed herein comprises a quaternary compound or silicon quaternary compound in an amount of from, e.g., about 1 ppm to about 25 ppm, about 1 ppm to about 50 ppm, about 1 ppm to about 75 ppm, about 1 ppm to about 100 ppm, about 1 ppm to about 125 ppm, about 1 ppm to about 150 ppm, about 1 ppm to about 175 ppm, about 1 ppm to about 200 ppm, about 1 ppm to about 225 ppm, about 1 ppm to about 250 ppm, about 1 ppm to about 275 ppm, about 1 ppm to about 300 ppm, about 1 ppm to about 325 ppm, about 1 ppm to about 350 ppm, about 1 ppm to about 375 ppm, about 1 ppm to about 400 ppm, about 10 ppm to about 25 ppm, about 10 ppm to about 50 ppm, about 10 ppm to about 75 ppm, about 10 ppm to about 100 ppm, about 10 ppm to about 125 ppm, about 10 ppm to about 150 ppm, about 10 ppm to about 175 ppm, about 10 ppm to about 200 ppm, about 10 ppm to about 225 ppm, about 10 ppm to about 250 ppm, about 10 ppm to about 275 ppm, about 10 ppm to about 300 ppm, about 10 ppm to about 325 ppm, about 10 ppm to about 350 ppm, about 10 ppm to about 375 ppm, about 10 ppm to about 400 ppm, about 25 ppm to about 50 ppm, about 25 ppm to about 75 ppm, about 25 ppm to about 100 ppm, about 25 ppm to about 125 ppm, about 25 ppm to about 150 ppm, about 25 ppm to about 175 ppm, about 25 ppm to about 200 ppm, about 25 ppm to about 225 ppm, about 25 ppm to about 250 ppm, about 25 ppm to about 275 ppm, about 25 ppm to about 300 ppm, about 25 ppm to about 325 ppm, about 25 ppm to about 350 ppm, about 25 ppm to about 375 ppm, about 25 ppm to about 400 ppm, about 50 ppm to about 75 ppm, about 50 ppm to about 100 ppm, about 50 ppm to about 125 ppm, about 50 ppm to about 150 ppm, about 50 ppm to about 175 ppm, about 50 ppm to about 200 ppm, about 50 ppm to about 225 ppm, about 50 ppm to about 250 ppm, about 50 ppm to about 275 ppm, about 50 ppm to about 300 ppm, about 50 ppm to about 325 ppm, about 50 ppm to about 350 ppm, about 50 ppm to about 375 ppm, about 50 ppm to about 400 ppm, about 75 ppm to about 100 ppm, about 75 ppm to about 125 ppm, about 75 ppm to about 150 ppm, about 75 ppm to about 175 ppm, about 75 ppm to about 200 ppm, about 75 ppm to about 225 ppm, about 75 ppm to about 250 ppm, about 75 ppm to about 275 ppm, about 75 ppm to about 300 ppm, about 75 ppm to about 325 ppm, about 75 ppm to about 350 ppm, about 75 ppm to about 375 ppm, about 75 ppm to about 400 ppm, about 100 ppm to about 125 ppm, about 100 ppm to about 150 ppm, about 100 ppm to about 175 ppm, about 100 ppm to about 200 ppm, about 100 ppm to about 225 ppm, about 100 ppm to about 250 ppm, about 100 ppm to about 275 ppm, about 100 ppm to about 300 ppm, about 100 ppm to about 325 ppm, about 100 ppm to about 350 ppm, about 100 ppm to about 375 ppm, about 100 ppm to about 400 ppm, about 150 ppm to about 175 ppm, about 150 ppm to about 200 ppm, about 150 ppm to about 225 ppm, about 150 ppm to about 250 ppm, about 150 ppm to about 275 ppm, about 150 ppm to about 300 ppm, about 150 ppm to about 325 ppm, about 150 ppm to about 350 ppm, about 150 ppm to about 375 ppm, about 150 ppm to about 400 ppm, about 200 ppm to about 225 ppm, about 200 ppm to about 250 ppm, about 200 ppm to about 275 ppm, about 200 ppm to about 300 ppm, about 200 ppm to about 325 ppm, about 200 ppm to about 350 ppm, about 200 ppm to about 375 ppm, about 200 ppm to about 400 ppm, about 250 ppm to about 275 ppm, about 250 ppm to about 300 ppm, about 250 ppm to about 325 ppm, about 250 ppm to about 350 ppm, about 250 ppm to about 375 ppm, about 250 ppm to about 400 ppm, about 300 ppm to about 325 ppm, about 300 ppm to about 350 ppm, about 300 ppm to about 375 ppm, about 300 ppm to about 400 ppm, about 350 ppm to about 375 ppm, about 350 ppm to about 400 ppm, or about 375 ppm to about 400 ppm.

In other aspects of this embodiment, a composition disclosed herein comprises a quaternary compound or silicon quaternary compound in an amount of, e.g., about 400 ppm to about 500 ppm, about 400 ppm to about 600 ppm, about 400 ppm to about 700 ppm, about 400 ppm to about 800 ppm, about 400 ppm to about 900 ppm, about 400 ppm to about 1,000 ppm, about 400 ppm to about 1,100 ppm, about 400 ppm to about 1,200 ppm, about 400 ppm to about 1,300 ppm, about 400 ppm to about 1,400 ppm, about 400 ppm to about 1,500 ppm, about 400 ppm to about 1,600 ppm, about 400 ppm to about 1,700 ppm, about 400 ppm to about 1,800 ppm, about 400 ppm to about 1,900 ppm, about 400 ppm to about 2,000 ppm, about 500 ppm to about 600 ppm, about 500 ppm to about 700 ppm, about 500 ppm to about 800 ppm, about 500 ppm to about 900 ppm, about 500 ppm to about 1,000 ppm, about 500 ppm to about 1,100 ppm, about 500 ppm to about 1,200 ppm, about 500 ppm to about 1,300 ppm, about 500 ppm to about 1,400 ppm, about 500 ppm to about 1,500 ppm, about 500 ppm to about 1,600 ppm, about 500 ppm to about 1,700 ppm, about 500 ppm to about 1,800 ppm, about 500 ppm to about 1,900 ppm, about 500 ppm to about 2,000 ppm, about 600 ppm to about 700 ppm, about 600 ppm to about 800 ppm, about 600 ppm to about 900 ppm, about 600 ppm to about 1,000 ppm, about 600 ppm to about 1,100 ppm, about 600 ppm to about 1,200 ppm, about 600 ppm to about 1,300 ppm, about 600 ppm to about 1,400 ppm, about 600 ppm to about 1,500 ppm, about 600 ppm to about 1,600 ppm, about 600 ppm to about 1,700 ppm, about 600 ppm to about 1,800 ppm, about 600 ppm to about 1,900 ppm, about 600 ppm to about 2,000 ppm, about 700 ppm to about 800 ppm, about 700 ppm to about 900 ppm, about 700 ppm to about 1,000 ppm, about 700 ppm to about 1,100 ppm, about 700 ppm to about 1,200 ppm, about 700 ppm to about 1,300 ppm, about 700 ppm to about 1,400 ppm, about 700 ppm to about 1,500 ppm, about 700 ppm to about 1,600 ppm, about 700 ppm to about 1,700 ppm, about 700 ppm to about 1,800 ppm, about 700 ppm to about 1,900 ppm, about 700 ppm to about 2,000 ppm, about 800 ppm to about 900 ppm, about 800 ppm to about 1,000 ppm, about 800 ppm to about 1,100 ppm, about 800 ppm to about 1,200 ppm, about 800 ppm to about 1,300 ppm, about 800 ppm to about 1,400 ppm, about 800 ppm to about 1,500 ppm, about 800 ppm to about 1,600 ppm, about 800 ppm to about 1,700 ppm, about 800 ppm to about 1,800 ppm, about 800 ppm to about 1,900 ppm, about 800 ppm to about 2,000 ppm, about 900 ppm to about 1,000 ppm, about 900 ppm to about 1,100 ppm, about 900 ppm to about 1,200 ppm, about 900 ppm to about 1,300 ppm, about 900 ppm to about 1,400 ppm, about 900 ppm to about 1,500 ppm, about 900 ppm to about 1,600 ppm, about 900 ppm to about 1,700 ppm, about 900 ppm to about 1,800 ppm, about 900 ppm to about 1,900 ppm, about 900 ppm to about 2,000 ppm, about 1,000 ppm to about 1,100 ppm, about 1,000 ppm to about 1,200 ppm, about 1,000 ppm to about 1,300 ppm, about 1,000 ppm to about 1,400 ppm, about 1,000 ppm to about 1,500 ppm, about 1,000 ppm to about 1,600 ppm, about 1,000 ppm to about 1,700 ppm, about 1,000 ppm to about 1,800 ppm, about 1,000 ppm to about 1,900 ppm, about 1,000 ppm to about 2,000 ppm, about 1,100 ppm to about 1,200 ppm, about 1,100 ppm to about 1,300 ppm, about 1,100 ppm to about 1,400 ppm, about 1,100 ppm to about 1,500 ppm, about 1,100 ppm to about 1,600 ppm, about 1,100 ppm to about 1,700 ppm, about 1,100 ppm to about 1,800 ppm, about 1,100 ppm to about 1,900 ppm, about 1,100 ppm to about 2,000 ppm, about 1,200 ppm to about 1,300 ppm, about 1,200 ppm to about 1,400 ppm, about 1,200 ppm to about 1,500 ppm, about 1,200 ppm to about 1,600 ppm, about 1,200 ppm to about 1,700 ppm, about 1,200 ppm to about 1,800 ppm, about 1,200 ppm to about 1,900 ppm, about 1,200 ppm to about 2,000 ppm, about 1,300 ppm to about 1,400 ppm, about 1,300 ppm to about 1,500 ppm, about 1,300 ppm to about 1,600 ppm, about 1,300 ppm to about 1,700 ppm, about 1,300 ppm to about 1,800 ppm, about 1,300 ppm to about 1,900 ppm, about 1,300 ppm to about 2,000 ppm, about 1,400 ppm to about 1,500 ppm, about 1,400 ppm to about 1,600 ppm, about 1,400 ppm to about 1,700 ppm, about 1,400 ppm to about 1,800 ppm, about 1,400 ppm to about 1,900 ppm, about 1,400 ppm to about 2,000 ppm, about 1,500 ppm to about 1,600 ppm, about 1,500 ppm to about 1,700 ppm, about 1,500 ppm to about 1,800 ppm, about 1,500 ppm to about 1,900 ppm, about 1,500 ppm to about 2,000 ppm, about 1,600 ppm to about 1,700 ppm, about 1,600 ppm to about 1,800 ppm, about 1,600 ppm to about 1,900 ppm, about 1,600 ppm to about 2,000 ppm, about 1,700 ppm to about 1,800 ppm, about 1,700 ppm to about 1,900 ppm, about 1,700 ppm to about 2,000 ppm, about 1,800 ppm to about 1,900 ppm, about 1,800 ppm to about 2,000 ppm, or about 1,900 ppm to about 2,000 ppm.

In aspects of this embodiment, a composition disclosed herein comprises a quaternary compound or silicon quaternary compound in an amount of, e.g., about 0.001%, about 0.005%, about 0.01%, about 0.05%, about 0.10%, about 0.11%, about 0.12%, about 0.13%, about 0.14%, about 0.15%, about 0.16%, about 0.17%, about 0.18%, about 0.19%, about 0.2%, about 0.25%, about 0.3%, about 0.35%, about 0.4%, about 0.45%, or about 0.5% by weight of the composition. In other aspects of this embodiment, a composition disclosed herein comprises a quaternary compound or silicon quaternary compound in an amount of, e.g., at least 0.001%, at least 0.005%, at least 0.01%, at least 0.05%, at least 0.10%, at least 0.11%, at least 0.12%, at least 0.13%, at least 0.14%, at least 0.15%, at least 0.16%, at least 0.17%, at least 0.18%, at least 0.19%, at least 0.2%, at least 0.25%, at least 0.3%, at least 0.35%, at least 0.4%, at least 0.45%, or at least 0.5% by weight of the composition. In yet other aspects of this embodiment, a composition disclosed herein comprises a quaternary compound or silicon quaternary compound in an amount of, e.g., at most 0.001%, at most 0.005%, at most 0.01%, at most 0.05%, at most 0.10%, at most 0.11%, at most 0.12%, at most 0.13%, at most 0.14%, at most 0.15%, at most 0.16%, at most 0.17%, at most 0.18%, at most 0.19%, at most 0.2%, at most 0.25%, at most 0.3%, at most 0.35%, at most 0.4%, at most 0.45%, or at most 0.5% by weight of the composition. In still other aspects of this embodiment, a composition disclosed herein comprises a quaternary compound or silicon quaternary compound in an amount of, e.g., about 0.0010% to about 0.005%, about 0.0010% to about 0.010%, about 0.0010% to about 0.050%, about 0.0010% to about 0.10%, about 0.0010% to about 0.15%, about 0.0010% to about 0.20%, about 0.0010% to about 0.25%, about 0.0010% to about 0.30%, about 0.0010% to about 0.35%, about 0.0010% to about 0.40%, about 0.0010% to about 0.45%, about 0.0010% to about 0.50%, about 0.010% to about 0.050%, about 0.010% to about 0.10%, about 0.010% to about 0.15%, about 0.010% to about 0.20%, about 0.010% to about 0.25%, about 0.010% to about 0.30%, about 0.010% to about 0.35%, about 0.010% to about 0.40%, about 0.010% to about 0.45%, about 0.010% to about 0.50%, about 0.10% to about 0.15%, about 0.10% to about 0.20%, about 0.10% to about 0.25%, about 0.10% to about 0.30%, about 0.10% to about 0.35%, about 0.10% to about 0.40%, about 0.10% to about 0.45%, about 0.10% to about 0.50%, about 0.20% to about 0.25%, about 0.20% to about 0.30%, about 0.20% to about 0.35%, about 0.20% to about 0.40%, about 0.20% to about 0.45%, about 0.20% to about 0.50%, about 0.30% to about 0.35%, about 0.30% to about 0.40%, about 0.30% to about 0.45%, about 0.30% to about 0.50%, about 0.40% to about 0.45%, about 0.40% to about 0.50%, or about 0.40% to about 0.50% by weight of the composition.

In aspects of this embodiment, a composition disclosed herein comprises a quaternary compound or silicon quaternary compound in an amount of, e.g., about 1.0%, about 2.0%, about 3.0%, about 4.0%, about 5.0%, about 6.0%, about 7.0%, about 8.0%, about 9.0%, about 10%, about 11%, about 12%, about 13%, about 14%, or about 15% by weight of the composition. In other aspects of this embodiment, a composition disclosed herein comprises a quaternary compound or silicon quaternary compound in an amount of, e.g., at least 1.0%, at least 2.0%, at least 3.0%, at least 4.0%, at least 5.0%, at least 6.0%, at least 7.0%, at least 8.0%, at least 9.0%, at least 10%, at least 11%, at least 12%, at least 13%, at least 14%, or at least 15% by weight of the composition. In yet other aspects of this embodiment, a composition disclosed herein comprises a quaternary compound or silicon quaternary compound in an amount of, e.g., at most 1.0%, at most 2.0%, at most 3.0%, at most 4.0%, at most 5.0%, at most 6.0%, at most 7.0%, at most 8.0%, at most 9.0%, at most 10%, at most 11%, at most 12%, at most 13%, at most 14%, or at most 15% by weight of the composition. In still other aspects of this embodiment, a composition disclosed herein comprises a quaternary compound or silicon quaternary compound in an amount of, e.g., about 1.0% to about 2.0%, about 1.0% to about 3.0%, about 1.0% to about 4.0%, about 1.0% to about 5.0%, about 1.0% to about 6.0%, about 1.0% to about 7.0%, about 1.0% to about 8.0%, about 1.0% to about 9.0%, about 1.0% to about 10%, about 1.0% to about 11%, about 1.0% to about 12%, about 1.0% to about 13%, about 1.0% to about 14%, about 1.0% to about 15%, about 2.0% to about 3.0%, about 2.0% to about 4.0%, about 2.0% to about 5.0%, about 2.0% to about 6.0%, about 2.0% to about 7.0%, about 2.0% to about 8.0%, about 2.0% to about 9.0%, about 2.0% to about 10%, about 2.0% to about 11%, about 2.0% to about 12%, about 2.0% to about 13%, about 2.0% to about 14%, about 2.0% to about 15%, about 3.0% to about 4.0%, about 3.0% to about 5.0%, about 3.0% to about 6.0%, about 3.0% to about 7.0%, about 3.0% to about 8.0%, about 3.0% to about 9.0%, about 3.0% to about 10%, about 3.0% to about 11%, about 3.0% to about 12%, about 3.0% to about 13%, about 3.0% to about 14%, about 3.0% to about 15%, about 4.0% to about 5.0%, about 4.0% to about 6.0%, about 4.0% to about 7.0%, about 4.0% to about 8.0%, about 4.0% to about 9.0%, about 4.0% to about 10%, about 4.0% to about 11%, about 4.0% to about 12%, about 4.0% to about 13%, about 4.0% to about 14%, about 4.0% to about 15%, about 5.0% to about 6.0%, about 5.0% to about 7.0%, about 5.0% to about 8.0%, about 5.0% to about 9.0%, about 5.0% to about 10%, about 5.0% to about 11%, about 5.0% to about 12%, about 5.0% to about 13%, about 5.0% to about 14%, about 5.0% to about 15%, about 6.0% to about 7.0%, about 6.0% to about 8.0%, about 6.0% to about 9.0%, about 6.0% to about 10%, about 6.0% to about 11%, about 6.0% to about 12%, about 6.0% to about 13%, about 6.0% to about 14%, about 6.0% to about 15%, about 7.0% to about 8.0%, about 7.0% to about 9.0%, about 7.0% to about 10%, about 7.0% to about 11%, about 7.0% to about 12%, about 7.0% to about 13%, about 7.0% to about 14%, about 7.0% to about 15%, about 8.0% to about 9.0%, about 8.0% to about 10%, about 8.0% to about 11%, about 8.0% to about 12%, about 8.0% to about 13%, about 8.0% to about 14%, about 8.0% to about 15%, about 9.0% to about 10%, about 9.0% to about 11%, about 9.0% to about 12%, about 9.0% to about 13%, about 9.0% to about 14%, about 9.0% to about 15%, about 10% to about 11%, about 10% to about 12%, about 10% to about 13%, about 10% to about 14%, about 10% to about 15%, about 11% to about 12%, about 11% to about 13%, about 11% to about 14%, about 11% to about 15%, about 12% to about 13%, about 12% to about 14%, about 12% to about 15%, about 13% to about 14%, about 13% to about 15%, or 14% to about 15% by weight of the composition.

In an embodiment, a composition disclosed herein does not comprise a quaternary compound. In an embodiment, a composition disclosed herein does not comprise a silicon quaternary compound. In an embodiment, a composition disclosed herein does not comprise a quaternary compound or a silicon quaternary compound.

A composition disclosed herein may comprise a compound containing a guanide moiety and/or functional group. Non-limiting examples of a guanide-containing compound include an organic compound containing a biguanide functional group, a biguanidine functional group or a triguanide functional group. In one embodiment, a guanide-containing compound disclosed herein is a biguanide or a biguanide-containing compound. In an aspect of this embodiment, a biguanide-containing compound comprises one, two, three, four or five biguanide functional groups. In an aspect of this embodiment, a biguanide-containing compound comprises formula II,

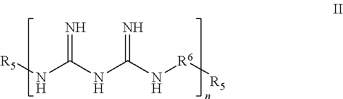

wherein R⁵ and R⁶ can each independently be a bond, H, C, NH, NH², a C₁₋₁₀ alkyl, a C₁₋₁₀ alkane, a C₁₋₁₀ alkyne, a 5 or 6 carbon aromatic ring, optionally substituted with a halogen; and n is 1-5. A halogen is F, Br, Cl, I, and At. Non-limiting examples of a biguanide-containing compound include a polyhexamethylene biguanide (PHMB), a polyaminopropyl biguanide (PAPB), a 1,1′-(1,6-Hexanediyl)bis{2-[N′-(2-ethylhexyl)carbamimidoyl]guanidine} (alexidine), a chlorhexidine and a chlorhexidine gluconate.

In one embodiment, a guanide-containing compound disclosed herein is a biguanidine or a biguanidine-containing compound. In an aspect of this embodiment, a biguanidine-containing compound comprises one, two, three, four or five biguanidine functional groups. In an aspect of this embodiment, a biguanidine-containing compound comprises formula III,

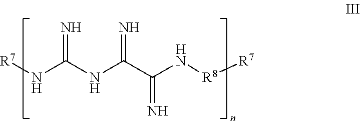

wherein R⁷ and R⁸ can each independently be a bond, H, C, NH, NH², a C₁₋₁₀ alkyl, a C₁₋₁₀ alkane, a C₁₋₁₀ alkyne, a 5 or 6 carbon aromatic ring, optionally substituted with a halogen; and n is 1-5. A halogen is F, Br, Cl, I, and At.

In one embodiment, a guanide-containing compound disclosed herein is a triguanide or a triguanide-containing compound. In an aspect of this embodiment, a triguanide-containing compound comprises one, two, three, four or five triguanide functional groups. In an aspect of this embodiment, a triguanide-containing compound comprises formula IV,

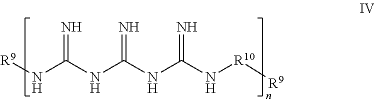

wherein R⁹ and R¹⁰ can each independently be a bond, H, C, NH, NH², a C₁₋₁₀ alkyl, a C₁₋₁₀ alkane, a C₁₋₁₀ alkyne, a 5 or 6 carbon aromatic ring, optionally substituted with a halogen; and n is 1-5. A halogen is F, Br, Cl, I, and At.

In one embodiment, a single guanide-containing compound is present in a composition disclosed herein. In another embodiment, a plurality of guanide-containing compounds is present in a composition disclosed herein. In aspects of this embodiment, a composition disclosed herein comprises, e.g., one or more guanide-containing compounds, two or more guanide-containing compounds, three or more guanide-containing compounds, four or more guanide-containing compounds or five or more guanide-containing compounds. In other aspects of this embodiment, a composition disclosed herein comprises, e.g., only one guanide-containing compound, at most two guanide-containing compounds, at most three guanide-containing compounds, at most four guanide-containing compounds, or at most five guanide-containing compounds. In yet other aspects of this embodiment, a composition disclosed herein comprises from, e.g., 1 to 2 guanide-containing compounds, 1 to 3 guanide-containing compounds, 1 to 4 guanide-containing compounds, 1 to 5 guanide-containing compounds, 2 to 3 guanide-containing compounds, 2 to 4 guanide-containing compounds, 2 to 5 guanide-containing compounds, 3 to 4 guanide-containing compounds, 3 to 5 guanide-containing compounds or 4 to 5 guanide-containing compounds.

Any amount of a guanide-containing compound disclosed herein may be used, with the proviso that the amount is useful to practice the methods disclosed herein. For example, when a composition comprising a guanide-containing compound is formulated to be used in an undiluted state, the amount of a guanide-containing compound used is a microbiostatic effective amount. A microbiostatic effective amount of a guanide-containing compound is an amount that restrains, prohibits, or otherwise prevents microbial population growth without eradicating the microbes. In an aspect of this embodiment, a microbiostatic effective amount of a guanide-containing compound is an amount that restrains, prohibits, or otherwise prevents microbial population growth without eradicating the microbes to a degree that achieves disinfection or sterilization. Alternatively, when a composition comprising a guanide-containing compound is formulated to be used in methods where the composition will be diluted, the amount of a guanide-containing compound used in such concentrated formulations can be any amount that does not cause precipitation of components in the composition disclosed herein or otherwise create unwanted effects that make the composition inoperable.

In aspects of this embodiment, a composition disclosed herein comprises a guanide-containing compound in an amount of, e.g., about 0.01%, about 0.05%, about 0.075%, about 0.1%, about 0.2%, about 0.3%, about 0.4%, about 0.5%, about 0.6%, about 0.7%, about 0.8%, about 0.9%, about 1.0%, about 1.5%, about 2.0%, about 2.5%, about 3.0%, about 4.0%, about 5.0%, about 6.0%, about 7.0%, about 7.5%, about 8.0%, about 9.0%, about 10.0%. about 11%, about 12%, about 13%, about 14%, about 15%, about 20%, about 25%, or about 30% by weight of the composition. In other aspects of this embodiment, a composition disclosed herein comprises a guanide-containing compound in an amount of, e.g., at least 0.01%, at least 0.05%, at least 0.075%, at least 0.1%, at least 0.25%, at least 0.5%, at least 0.75%, at least 1.0%, at least 1.5%, at least 2.0%, at least 2.5%, at least 3.0%, at least 4.0%, at least 5.0%, at least 6.0%, at least 7.0%, at least 7.5%, at least 8.0%, at least 9.0%, at least 10.0%, at least 11%, at least 12%, at least 13%, at least 14%, at least 15%, at least 20%, at least 25%, or at least 30% by weight of the composition. In yet other aspects of this embodiment, a composition disclosed herein comprises a guanide-containing compound in an amount of, e.g., at most 0.01%, at most 0.05%, at most 0.075%, at most 0.1%, at most 0.25%, at most 0.5%, at most 0.75%, at most 1.0%, at most 1.5%, at most 2.0%, at most 2.5%, at most 3.0%, at most 4.0%, at most 5.0%, at most 6.0%, at most 7.5%, at most 8.0%, at most 9.0%, at most 10.0%, at most 11%, at most 12%, at most 13%, at most 14%, at most 15%, at most 20%, at most 25%, or at most 30% by weight of the composition.

In still other aspects of this embodiment, a composition disclosed herein comprises a guanide-containing compound in an amount of, e.g., about 0.1% to about 0.5%, about 0.1% to about 0.75%, about 0.1% to about 1.0%, about 0.1% to about 1.5%, about 0.1% to about 2.0%, about 0.1% to about 2.5%, about 0.1% to about 3.0%, about 0.1% to about 3.5%, about 0.1% to about 4.0%, about 0.1% to about 4.5%, about 0.1% to about 5.0%, about 0.2% to about 0.5%, about 0.2% to about 0.75%, about 0.2% to about 1.0%, about 0.2% to about 1.5%, about 0.2% to about 2.0%, about 0.2% to about 2.5%, about 0.2% to about 3.0%, about 0.2% to about 3.5%, about 0.2% to about 4.0%, about 0.2% to about 4.5%, about 0.2% to about 5.0%, about 0.5% to about 1.0%, about 0.5% to about 1.5%, about 0.5% to about 2.0%, about 0.5% to about 2.5%, about 0.5% to about 3.0%, about 0.5% to about 4.0%, about 0.5% to about 5.0%, about 0.5% to about 6.0%, about 0.5% to about 7.0%, about 0.5% to about 8.0%, about 0.5% to about 9.0%, about 0.5% to about 10.0%, about 1.0% to about 2.5%, about 1.0% to about 3.0%, about 1.0% to about 4.0%, about 1.0% to about 5.0%, about 1.0% to about 6.0%, about 1.0% to about 7.0%, about 1.0% to about 7.5%, about 1.0% to about 8.0%, about 1.0% to about 9.0%, about 1.0% to about 10.0%, about 1.0% to about 15.0%, about 1.0% to about 20.0%, about 1.0% to about 25.0%, about 1.0% to about 30.0%, about 2.0% to about 2.5%, about 2.0% to about 3.0%, about 2.0% to about 4.0%, about 2.0% to about 5.0%, about 2.0% to about 6.0%, about 2.0% to about 7.0%, about 2.0% to about 7.5%, about 2.0% to about 8.0%, about 2.0% to about 9.0%, about 2.0% to about 10.0%, about 2.0% to about 15.0%, about 2.0% to about 20.0%, about 2.0% to about 25.0%, about 2.0% to about 30.0%, about 5.0% to about 6.0%, about 5.0% to about 7.0%, about 5.0% to about 7.5%, about 5.0% to about 8.0%, about 5.0% to about 9.0%, about 5.0% to about 10.0%, about 5.0% to about 11.0%, about 5.0% to about 12.0%, about 5.0% to about 13.0%, about 5.0% to about 14.0%, about 5.0% to about 15.0%, about 5.0% to about 20.0%, about 5.0% to about 25.0%, about 5.0% to about 30.0%, about 10.0% to about 15.0%, about 10.0% to about 20.0%, about 10.0% to about 25.0%, about 10.0% to about 30.0%, about 15.0% to about 20.0%, about 15.0% to about 25.0%, about 15.0% to about 30.0%, about 20.0% to about 25.0%, about 20.0% to about 30.0%, or about 20.0% to about 30.0%, by weight of the composition.

In another embodiment, a composition disclosed herein does not comprise a guanide-containing compound. In another embodiment, a composition disclosed herein does not comprise biguanide. In another embodiment, a composition disclosed herein does not comprise a biguanide-containing compound. In another embodiment, a composition disclosed herein does not comprise biguanidine. In another embodiment, a composition disclosed herein does not comprise a biguanidine-containing compound. In another embodiment, a composition disclosed herein does not comprise triguanide. In another embodiment, a composition disclosed herein does not comprise a triguanide-containing compound.

A composition disclosed herein may comprise an alcohol. An alcohol is an organic molecule comprising a hydroxyl functional group (—OH) bond to a carbon atom, where the carbon atom is saturated. In aspects of this embodiment, the alcohol may be, e.g., a C₂₋₄ alcohol, a C₁₋₄ alcohol, a C₁₋₅ alcohol, a C₁₋₇ alcohol, a C₁₋₁₀ alcohol, a C₁₋₁₅ alcohol, or a C₁₋₂₀ alcohol. In other aspects of this embodiment, an alcohol may be, e.g., a primary alcohol, a secondary alcohol, or a tertiary alcohol. In other aspects of this embodiment, an alcohol may be, e.g., an acyclic alcohol, a monohydric alcohol, a polyhydric alcohol (also known as a polyol or sugar alcohol), an unsaturated aliphatic alcohol, an alicyclic alcohol, or a combination thereof. Examples of a monohydric alcohol include, without limitation, methanol, ethanol, propanol, isopropanol, butanol, pentanol, and 1-hexadecanol. Examples of a polyhydric alcohol include, without limitation, glycol, glycerol, arabitol, erythritol, xylitol, maltitol, sorbitol (gluctiol), mannitol, inositol, lactitol, galactitol (iditol), and isomalt. Examples of an unsaturated aliphatic alcohol include, without limitation, prop-2-ene-1-ol, 3,7-dimethylocta-2,6-dien-1-ol, and prop-2-in-1-ol. Examples of an alicyclic alcohol include, without limitation, cyclohexane-1,2,3,4,5,6-hexol and 2-(2-propyl)-5-methyl-cyclohexane-1-ol.

In one embodiment, a single alcohol is present in a composition disclosed herein. In another embodiment, a plurality of alcohols is present in a composition disclosed herein. In aspects of this embodiment, a composition disclosed herein comprises, e.g., one or more alcohols, two or more alcohols, three or more alcohols, four or more alcohols or five or more alcohols. In other aspects of this embodiment, a composition disclosed herein comprises, e.g., only one alcohol, at most two alcohols, at most three alcohols, at most four alcohols, or at most five alcohols. In yet other aspects of this embodiment, a composition disclosed herein comprises from, e.g., 1 to 2 alcohols, 1 to 3 alcohols, 1 to 4 alcohols, 1 to 5 alcohols, 2 to 3 alcohols, 2 to 4 alcohols, 2 to 5 alcohols, 3 to 4 alcohols, 3 to 5 alcohols or 4 to 5 alcohols.

In aspects of this embodiment, a plurality of alcohols is present in a composition disclosed herein may comprise, e.g., about 99% of a first alcohol and about 1% of a second alcohol, about 98% of a first alcohol and about 2% of a second alcohol, about 97% of a first alcohol and about 3% of a second alcohol, about 96% of a first alcohol and about 4% of a second alcohol, about 95% of a first alcohol and about 5% of a second alcohol, about 90% of a first alcohol and about 10% of a second alcohol, about 80% of a first alcohol and about 20% of a second alcohol, about 70% of a first alcohol and about 30% of a second alcohol, about 60% of a first alcohol and about 40% of a second alcohol, about 50% of a first alcohol and about 50% of a second alcohol, about 40% of a first alcohol and about 60% of a second alcohol, about 30% of a first alcohol and about 70% of a second alcohol, about 20% of a first alcohol and about 80% of a second alcohol, about 10% of a first alcohol and about 90% of a second alcohol, about 5% of a first alcohol and about 95% of a second alcohol. In other aspects of this embodiment, the ratio of a first alcohol to a second alcohol is, e.g., 100:1, 90:1, 80:1, 70:1, 60:1, 50:1, 40:1, 30:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:30, 1:40, 1:50, 1:60, 1:70, 1:80, 1:90, or 1:100. In other aspects, the first alcohol is isopropanol and the second alcohol is ethanol.

Any amount of an alcohol disclosed herein may be used, with the proviso that the amount is useful to practice the methods disclosed herein. For example, when a composition comprising an alcohol is formulated to be used in an undiluted state, the amount of an alcohol used is a microbiostatic effective amount. A microbiostatic effective amount of an alcohol is an amount that restrains, prohibits, or otherwise prevents microbial population growth without eradicating the microbes. In an aspect of this embodiment, a microbiostatic effective amount of an alcohol is an amount that restrains, prohibits, or otherwise prevents microbial population growth without eradicating the microbes to a degree that achieves disinfection or sterilization. Alternatively, when a composition comprising an alcohol is formulated to be used in methods where the composition will be diluted, the amount of an alcohol used in such concentrated formulations can be any amount that does not cause precipitation of components in the composition disclosed herein or otherwise create unwanted effects that make the composition inoperable.

In aspects of this embodiment, a composition disclosed herein comprises an alcohol in an amount of, e.g., about 0.5%, about 1%, about 2%, about 3%, about 4%, about 5%, about 6%, about 7%, about 8%, about 9%, about 10%, about 11%, about 12%, about 13%, about 14%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, or about 99%, by weight of the composition. In other aspects of this embodiment, a composition disclosed herein comprises an alcohol in an amount of, e.g., at least 0.5%, at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99%, by weight of the composition. In yet other aspects of this embodiment, a composition disclosed herein comprises an alcohol in an amount of, e.g., at most 0.5%, at most 1%, at most 2%, at most 3%, at most 4%, at most 5%, at most 6%, at most 7%, at most 8%, at most 9%, at most 10%, at most 11%, at most 12%, at most 13%, at most 14%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95%, at most 96%, at most 97%, at most 98%, or at most 99%, by weight of the composition.

In still other aspects of this embodiment, a composition disclosed herein comprises an alcohol in an amount of, e.g., about 0.05% to about 0.75%, about 0.05% to about 1.0%, about 0.05% to about 1.5%, about 0.05% to about 2.0%, about 0.05% to about 2.5%, about 0.1% to about 0.5%, about 0.1% to about 0.75%, about 0.1% to about 1.0%, about 0.1% to about 1.5%, about 0.1% to about 2.0%, about 0.1% to about 2.5%, about 0.1% to about 3.0%, about 0.1% to about 4.0%, about 0.1% to about 5.0%, about 0.2% to about 0.5%, about 0.2% to about 0.75%, about 0.2% to about 1.0%, about 0.2% to about 1.5%, about 0.2% to about 2.0%, about 0.2% to about 2.5%, about 0.2% to about 3.0%, about 0.2% to about 4.0%, about 0.2% to about 5.0%, about 0.5% to about 1.0%, about 0.5% to about 1.5%, about 0.5% to about 2.0%, about 0.5% to about 2.5%, about 0.5% to about 3.0%, about 0.5% to about 4.0%, about 0.5% to about 5.0%, about 0.5% to about 6.0%, about 0.5% to about 7.0%, about 0.5% to about 8.0%, about 0.5% to about 9.0%, about 0.5% to about 10.0%, about 1.0% to about 2.5%, about 1.0% to about 3.0%, about 1.0% to about 4.0%, about 1.0% to about 5.0%, about 1.0% to about 6.0%, about 1.0% to about 7.0%, about 1.0% to about 7.5%, about 1.0% to about 8.0%, about 1.0% to about 9.0%, about 1.0% to about 10.0%, about 2.0% to about 2.5%, about 2.0% to about 3.0%, about 2.0% to about 4.0%, about 2.0% to about 5.0%, about 2.0% to about 6.0%, about 2.0% to about 7.0%, about 2.0% to about 7.5%, about 2.0% to about 8.0%, about 2.0% to about 9.0%, about 2.0% to about 10.0%, about 5.0% to about 6.0%, about 5.0% to about 7.0%, about 5.0% to about 7.5%, about 5.0% to about 8.0%, about 5.0% to about 9.0%, about 5.0% to about 10.0%, about 5.0% to about 11.0%, about 5.0% to about 12.0%, about 5.0% to about 13.0%, about 5.0% to about 14.0% or about 5.0% to about 15.0% by weight of the composition, or about 10% to about 20%, about 10% to about 30%, about 10% to about 40%, about 10% to about 50%, about 10% to about 60%, about 10% to about 70%, about 10% to about 80%, about 10% to about 90%, about 10% to about 95%, about 10% to about 97%, about 10% to about 99%, about 20% to about 30%, about 20% to about 40%, about 20% to about 50%, about 20% to about 60%, about 20% to about 70%, about 20% to about 80%, about 20% to about 90%, about 20% to about 95%, about 20% to about 97%, about 20% to about 99%, about 30% to about 40%, about 30% to about 50%, about 30% to about 60%, about 30% to about 70%, about 30% to about 80%, about 30% to about 90%, about 30% to about 95%, about 30% to about 97%, about 30% to about 99%, about 40% to about 50%, about 40% to about 60%, about 40% to about 70%, about 40% to about 80%, about 40% to about 90%, about 40% to about 95%, about 40% to about 97%, about 40% to about 99%, about 50% to about 60%, about 50% to about 70%, about 50% to about 80%, about 50% to about 90%, about 50% to about 95%, about 50% to about 97%, about 50% to about 99%, about 60% to about 70%, about 60% to about 80%, about 60% to about 90%, about 60% to about 95%, about 60% to about 97%, about 60% to about 99%, about 70% to about 80%, about 70% to about 90%, about 70% to about 95%, about 70% to about 97%, about 70% to about 99%, about 80% to about 90%, about 80% to about 95%, about 80% to about 97%, about 80% to about 99%, about 90% to about 95%, about 90% to about 97%, about 90% to about 99%, by weight of the composition.

In aspects of this embodiment, a composition disclosed herein comprises a plurality of alcohols in a total amount of, e.g., about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, or about 99%, by weight of the composition. In other aspects of this embodiment, a composition disclosed herein comprises a plurality of alcohols in a total amount of, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99%, by weight of the composition. In yet other aspects of this embodiment, a composition disclosed herein comprises a plurality of alcohols in a total amount of, e.g., at most 10%, at most 15%, at most 20%, at most 25%, at most 30%, at most 35%, at most 40%, at most 40%, at most 45%, at most 50%, at most 55%, at most 60%, at most 65%, at most 70%, at most 75%, at most 80%, at most 85%, at most 90%, at most 95%, at most 96%, at most 97%, at most 98%, or at most 99%, by weight of the composition. In still other aspects of this embodiment, a composition disclosed herein comprises a plurality of alcohols in a total amount of, e.g., about 10% to about 20%, about 10% to about 30%, about 10% to about 40%, about 10% to about 50%, about 10% to about 60%, about 10% to about 70%, about 10% to about 80%, about 10% to about 90%, about 10% to about 95%, about 10% to about 97%, about 10% to about 99%, about 20% to about 30%, about 20% to about 40%, about 20% to about 50%, about 20% to about 60%, about 20% to about 70%, about 20% to about 80%, about 20% to about 90%, about 20% to about 95%, about 20% to about 97%, about 20% to about 99%, about 30% to about 40%, about 30% to about 50%, about 30% to about 60%, about 30% to about 70%, about 30% to about 80%, about 30% to about 90%, about 30% to about 95%, about 30% to about 97%, about 30% to about 99%, about 40% to about 50%, about 40% to about 60%, about 40% to about 70%, about 40% to about 80%, about 40% to about 90%, about 40% to about 95%, about 40% to about 97%, about 40% to about 99%, about 50% to about 60%, about 50% to about 70%, about 50% to about 80%, about 50% to about 90%, about 50% to about 95%, about 50% to about 97%, about 50% to about 99%, about 60% to about 70%, about 60% to about 80%, about 60% to about 90%, about 60% to about 95%, about 60% to about 97%, about 60% to about 99%, about 70% to about 80%, about 70% to about 90%, about 70% to about 95%, about 70% to about 97%, about 70% to about 99%, about 80% to about 90%, about 80% to about 95%, about 80% to about 97%, about 80% to about 99%, about 90% to about 95%, about 90% to about 97%, about 90% to about 99%, by weight of the composition.

In another embodiment, a composition disclosed herein does not comprise an alcohol.

A composition disclosed herein may comprise metallic particles. A metallic particle can be composed of a single element, such as, e.g., copper, iron, silver, titanium or zinc or be a mixed metallic particle composed of various combinations of different elements, such as, e.g., various combinations of two or more of the following: copper, iron, silver, titanium or zinc. Non-limiting examples of a metallic particle includes a copper particle, an iron particle, a potassium particle, a silver particle, a titanium particle, and a zinc particle. Other non-limiting examples of a metallic particle include a metal acetate particle, a metal chloride particle, a metal nitrate particle, or a metal oxide particle. A metal acetate particle includes, without limitation, copper acetate, iron acetate, e.g. iron (II) acetate and iron (III) acetate, silver acetate, titanium acetate, zinc acetate, or any combination thereof. A metal nitrate particle includes, without limitation, copper nitrate, iron nitrate, e.g., iron (II) nitrate, iron (III) nitrate, silver nitrate, titanium nitrate, zinc nitrate, or any combination thereof. A metal chloride particle includes, without limitation, copper chloride, iron chloride, e.g., iron (II) chloride or iron (III) chloride, silver chloride, titanium chloride, zinc chloride, or any combination thereof. A metal oxide particle includes, without limitation, copper oxide particle, iron oxide particle, e.g., iron (II) oxide particle, iron (III) oxide particle, silver oxide particle, titanium dioxide particle, zinc oxide particle, copper zinc iron oxide particle, or any combination thereof.

A metallic particle includes nanoparticles and microparticles. A nanoparticle has an average diameter of about 1 nm to about 1,000 nm. A microparticle has an average diameter of about 1 μm to about 1,000 μm.

In an embodiment, a composition disclosed herein comprises an amount of metallic particles that provides a desired beneficial effect to a composition disclosed herein. For example, when a composition comprising metallic particles is formulated to be used in an undiluted state, the amount of metallic particles used is a microbiostatic effective amount. A microbiostatic effective amount of metallic particles is an amount that restrains, prohibits, or otherwise prevents microbial population growth without eradicating the microbes. In an aspect of this embodiment, a microbiostatic effective amount of metallic particles is an amount that restrains, prohibits, or otherwise prevents microbial population growth without eradicating the microbes to a degree that achieves disinfection or sterilization. Alternatively, when a composition comprising metallic particles is formulated to be used in methods where the composition will be diluted, the amount of metallic particles used in such concentrated formulations can be any amount that does not cause precipitation of components in the composition disclosed herein or otherwise create unwanted effects that make the composition inoperable.

In aspects of this embodiment, a composition disclosed herein comprises metallic particles in an amount of, e.g., 0.05 ppm, 0.10 ppm, 0.15 ppm, 0.20 ppm, 0.25 ppm, 0.30 ppm, 0.35 ppm, 0.40 ppm, 0.45 ppm, 0.50 ppm, 0.55 ppm, 0.60 ppm, 0.65 ppm, 0.70 ppm, 0.75 ppm, 0.80 ppm, 0.85 ppm, 0.90 ppm, 0.95 ppm, 1 ppm, 5 ppm, 10 ppm, 15 ppm, 20 ppm, 25 ppm, 30 ppm, 35 ppm, 40 ppm, 45 ppm, 50 ppm, 55 ppm, 60 ppm, 65 ppm, 70 ppm, 75 ppm, 80 ppm, 85 ppm, 90 ppm, 95 ppm, 100 ppm, 125 ppm, 150 ppm, 175 ppm, 200 ppm, 225 ppm, 250 ppm, 275 ppm, 300 ppm, 325 ppm, 350 ppm, 375 ppm, 400 ppm, 425 ppm, 450 ppm, 475 ppm, 500 ppm, 525 ppm, 550 ppm, 575 ppm, 600 ppm, 625 ppm, 650 ppm, 675 ppm, 700 ppm, 725 ppm, 750 ppm, 775 ppm, 800 ppm, 825 ppm, 850 ppm, 875 ppm, 900 ppm, 925 ppm, 950 ppm, 975 ppm, 1,000 ppm, 1,025 ppm, 1,050 ppm, 1075 ppm, 1,100 ppm, 1,125 ppm, 1,150 ppm, 1,175 ppm, 1,200 ppm, 1,225 ppm, 1,250 ppm, 1,275 ppm, 1,300 ppm, 1,325 ppm, 1,350 ppm, 1,375 ppm, 1,400 ppm, 1,425 ppm, 1,450 ppm, 1,475 ppm, 1,500 ppm, 1,600 ppm, 1,700 ppm, 1,800 ppm, 1,900 ppm, or 2,000 ppm. In other aspects of this embodiment, a composition disclosed herein comprises metallic particles in an amount of, e.g., at least 0.05 ppm, at least 0.10 ppm, at least 0.20 ppm, at least 0.30 ppm, at least 0.40 ppm, at least 0.50 ppm, at least 0.60 ppm, at least 0.70 ppm, at least 0.80 ppm, at least 0.90 ppm, at least 1 ppm, at least 10 ppm, at least 20 ppm, at least 30 ppm, at least 40 ppm, at least 50 ppm, at least 60 ppm, at least 70 ppm, at least 80 ppm, at least 90 ppm, at least 100 ppm, at least 125 ppm, at least 150 ppm, at least 175 ppm, at least 200 ppm, at least 225 ppm, at least 250 ppm, at least 275 ppm, at least 300 ppm, at least 325 ppm, at least 350 ppm, at least 375 ppm, at least 400 ppm, at least 425 ppm, at least 450 ppm, at least 475 ppm, at least 500 ppm, at least 525 ppm, at least 550 ppm, at least 575 ppm, at least 600 ppm, at least 625 ppm, at least 650 ppm, at least 675 ppm, at least 700 ppm, at least 725 ppm, at least 750 ppm, at least 775 ppm, at least 800 ppm, at least 825 ppm, at least 850 ppm, at least 875 ppm, at least 900 ppm, at least 925 ppm, at least 950 ppm, at least 975 ppm, at least 1,000 ppm, at least 1,025 ppm, at least 1,050 ppm, at least 1075 ppm, at least 1,100 ppm, at least 1,125 ppm, at least 1,150 ppm, at least 1,175 ppm, at least 1,200 ppm, at least 1,225 ppm, at least 1,250 ppm, at least 1,275 ppm, at least 1,300 ppm, at least 1,325 ppm, at least 1,350 ppm, at least 1,375 ppm, at least 1,400 ppm, at least 1,425 ppm, at least 1,450 ppm, at least 1,475 ppm, at least 1,500 ppm, at least 1,600 ppm, at least 1,700 ppm, at least 1,800 ppm, at least 1,900 ppm, or at least 2,000 ppm. In yet other aspects of this embodiment, a composition disclosed herein comprises metallic particles in an amount of, e.g., at most 0.05 ppm, at most 0.10 ppm, at most 0.20 ppm, at most 0.30 ppm, at most 0.40 ppm, at most 0.50 ppm, at most 0.60 ppm, at most 0.70 ppm, at most 0.80 ppm, at most 0.90 ppm, at most 1 ppm, at most 10 ppm, at most 20 ppm, at most 30 ppm, at most 40 ppm, at most 50 ppm, at most 60 ppm, at most 70 ppm, at most 80 ppm, at most 90 ppm, at most 100 ppm, at most 125 ppm, at most 150 ppm, at most 175 ppm, at most 200 ppm, at most 225 ppm, at most 250 ppm, at most 275 ppm, at most 300 ppm, at most 325 ppm, at most 350 ppm, at most 375 ppm, at most 400 ppm, at most 425 ppm, at most 450 ppm, at most 475 ppm, at most 500 ppm, at most 525 ppm, at most 550 ppm, at most 575 ppm, at most 600 ppm, at most 625 ppm, at most 650 ppm, at most 675 ppm, at most 700 ppm, at most 725 ppm, at most 750 ppm, at most 775 ppm, at most 800 ppm, at most 825 ppm, at most 850 ppm, at most 875 ppm, at most 900 ppm, at most 925 ppm, at most 950 ppm, at most 975 ppm, at most 1,000 ppm, at most 1,025 ppm, at most 1,050 ppm, at most 1075 ppm, at most 1,100 ppm, at most 1,125 ppm, at most 1,150 ppm, at most 1,175 ppm, at most 1,200 ppm, at most 1,225 ppm, at most 1,250 ppm, at most 1,275 ppm, at most 1,300 ppm, at most 1,325 ppm, at most 1,350 ppm, at most 1,375 ppm, at most 1,400 ppm, at most 1,425 ppm, at most 1,450 ppm, at most 1,475 ppm, at most 1,500 ppm, at most 1,600 ppm, at most 1,700 ppm, at most 1,800 ppm, at most 1,900 ppm, or at most 2,000 ppm.

In still other aspects of this embodiment, a composition disclosed herein comprises metallic particles in an amount of from, e.g., about 0.5 ppm to about 20 ppm, about 0.5 ppm to about 25 ppm, about 0.5 ppm to about 30 ppm, about 0.5 ppm to about 35 ppm, about 0.5 ppm to about 40 ppm, about 0.5 ppm to about 45 ppm, about 0.5 ppm to about 50 ppm, about 0.5 ppm to about 55 ppm, about 0.5 ppm to about 60 ppm, about 0.5 ppm to about 65 ppm, about 0.5 ppm to about 70 ppm, about 0.5 ppm to about 75 ppm, about 0.5 ppm to about 80 ppm, about 0.5 ppm to about 85 ppm, about 0.5 ppm to about 90 ppm, about 0.5 ppm to about 95 ppm, about 0.5 ppm to about 100 ppm, about 0.75 ppm to about 20 ppm, about 0.75 ppm to about 25 ppm, about 0.75 ppm to about 30 ppm, about 0.75 ppm to about 35 ppm, about 0.75 ppm to about 40 ppm, about 0.75 ppm to about 45 ppm, about 0.75 ppm to about 50 ppm, about 0.75 ppm to about 55 ppm, about 0.75 ppm to about 60 ppm, about 0.75 ppm to about 65 ppm, about 0.75 ppm to about 70 ppm, about 0.75 ppm to about 75 ppm, about 0.75 ppm to about 80 ppm, about 0.75 ppm to about 85 ppm, about 0.75 ppm to about 90 ppm, about 0.75 ppm to about 95 ppm, about 0.75 ppm to about 100 ppm, about 1 ppm to about 20 ppm, about 1 ppm to about 25 ppm, about 1 ppm to about 30 ppm, about 1 ppm to about 35 ppm, about 1 ppm to about 40 ppm, about 1 ppm to about 45 ppm, about 1 ppm to about 50 ppm, about 1 ppm to about 55 ppm, about 1 ppm to about 60 ppm, about 1 ppm to about 65 ppm, about 1 ppm to about 70 ppm, about 1 ppm to about 75 ppm, about 1 ppm to about 80 ppm, about 1 ppm to about 85 ppm, about 1 ppm to about 90 ppm, about 1 ppm to about 95 ppm, about 1 ppm to about 100 ppm, about 5 ppm to about 20 ppm, about 5 ppm to about 25 ppm, about 5 ppm to about 30 ppm, about 5 ppm to about 35 ppm, about 5 ppm to about 40 ppm, about 5 ppm to about 45 ppm, about 5 ppm to about 50 ppm, about 5 ppm to about 55 ppm, about 5 ppm to about 60 ppm, about 5 ppm to about 65 ppm, about 5 ppm to about 70 ppm, about 5 ppm to about 75 ppm, about 5 ppm to about 80 ppm, about 5 ppm to about 85 ppm, about 5 ppm to about 90 ppm, about 5 ppm to about 95 ppm, about 5 ppm to about 100 ppm, about 10 ppm to about 20 ppm, about 10 ppm to about 25 ppm, about 10 ppm to about 30 ppm, about 10 ppm to about 35 ppm, about 10 ppm to about 40 ppm, about 10 ppm to about 45 ppm, about 10 ppm to about 50 ppm, about 10 ppm to about 55 ppm, about 10 ppm to about 60 ppm, about 10 ppm to about 65 ppm, about 10 ppm to about 70 ppm, about 10 ppm to about 75 ppm, about 10 ppm to about 80 ppm, about 10 ppm to about 85 ppm, about 10 ppm to about 90 ppm, about 10 ppm to about 95 ppm, or about 10 ppm to about 100 ppm.

In other aspects of this embodiment, a composition disclosed herein comprises metallic particles in an amount of from, e.g., about 1 ppm to about 25 ppm, about 1 ppm to about 50 ppm, about 1 ppm to about 75 ppm, about 1 ppm to about 100 ppm, about 1 ppm to about 125 ppm, about 1 ppm to about 150 ppm, about 1 ppm to about 175 ppm, about 1 ppm to about 200 ppm, about 1 ppm to about 225 ppm, about 1 ppm to about 250 ppm, about 1 ppm to about 275 ppm, about 1 ppm to about 300 ppm, about 1 ppm to about 325 ppm, about 1 ppm to about 350 ppm, about 1 ppm to about 375 ppm, about 1 ppm to about 400 ppm, about 10 ppm to about 25 ppm, about 10 ppm to about 50 ppm, about 10 ppm to about 75 ppm, about 10 ppm to about 100 ppm, about 10 ppm to about 125 ppm, about 10 ppm to about 150 ppm, about 10 ppm to about 175 ppm, about 10 ppm to about 200 ppm, about 10 ppm to about 225 ppm, about 10 ppm to about 250 ppm, about 10 ppm to about 275 ppm, about 10 ppm to about 300 ppm, about 10 ppm to about 325 ppm, about 10 ppm to about 350 ppm, about 10 ppm to about 375 ppm, about 10 ppm to about 400 ppm, about 25 ppm to about 50 ppm, about 25 ppm to about 75 ppm, about 25 ppm to about 100 ppm, about 25 ppm to about 125 ppm, about 25 ppm to about 150 ppm, about 25 ppm to about 175 ppm, about 25 ppm to about 200 ppm, about 25 ppm to about 225 ppm, about 25 ppm to about 250 ppm, about 25 ppm to about 275 ppm, about 25 ppm to about 300 ppm, about 25 ppm to about 325 ppm, about 25 ppm to about 350 ppm, about 25 ppm to about 375 ppm, about 25 ppm to about 400 ppm, about 50 ppm to about 75 ppm, about 50 ppm to about 100 ppm, about 50 ppm to about 125 ppm, about 50 ppm to about 150 ppm, about 50 ppm to about 175 ppm, about 50 ppm to about 200 ppm, about 50 ppm to about 225 ppm, about 50 ppm to about 250 ppm, about 50 ppm to about 275 ppm, about 50 ppm to about 300 ppm, about 50 ppm to about 325 ppm, about 50 ppm to about 350 ppm, about 50 ppm to about 375 ppm, about 50 ppm to about 400 ppm, about 75 ppm to about 100 ppm, about 75 ppm to about 125 ppm, about 75 ppm to about 150 ppm, about 75 ppm to about 175 ppm, about 75 ppm to about 200 ppm, about 75 ppm to about 225 ppm, about 75 ppm to about 250 ppm, about 75 ppm to about 275 ppm, about 75 ppm to about 300 ppm, about 75 ppm to about 325 ppm, about 75 ppm to about 350 ppm, about 75 ppm to about 375 ppm, about 75 ppm to about 400 ppm, about 100 ppm to about 125 ppm, about 100 ppm to about 150 ppm, about 100 ppm to about 175 ppm, about 100 ppm to about 200 ppm, about 100 ppm to about 225 ppm, about 100 ppm to about 250 ppm, about 100 ppm to about 275 ppm, about 100 ppm to about 300 ppm, about 100 ppm to about 325 ppm, about 100 ppm to about 350 ppm, about 100 ppm to about 375 ppm, about 100 ppm to about 400 ppm, about 150 ppm to about 175 ppm, about 150 ppm to about 200 ppm, about 150 ppm to about 225 ppm, about 150 ppm to about 250 ppm, about 150 ppm to about 275 ppm, about 150 ppm to about 300 ppm, about 150 ppm to about 325 ppm, about 150 ppm to about 350 ppm, about 150 ppm to about 375 ppm, about 150 ppm to about 400 ppm, about 200 ppm to about 225 ppm, about 200 ppm to about 250 ppm, about 200 ppm to about 275 ppm, about 200 ppm to about 300 ppm, about 200 ppm to about 325 ppm, about 200 ppm to about 350 ppm, about 200 ppm to about 375 ppm, about 200 ppm to about 400 ppm, about 250 ppm to about 275 ppm, about 250 ppm to about 300 ppm, about 250 ppm to about 325 ppm, about 250 ppm to about 350 ppm, about 250 ppm to about 375 ppm, about 250 ppm to about 400 ppm, about 300 ppm to about 325 ppm, about 300 ppm to about 350 ppm, about 300 ppm to about 375 ppm, about 300 ppm to about 400 ppm, about 350 ppm to about 375 ppm, about 350 ppm to about 400 ppm, or about 375 ppm to about 400 ppm.

In other aspects of this embodiment, a composition disclosed herein comprises metallic particles in an amount of, e.g., about 400 ppm to about 500 ppm, about 400 ppm to about 600 ppm, about 400 ppm to about 700 ppm, about 400 ppm to about 800 ppm, about 400 ppm to about 900 ppm, about 400 ppm to about 1,000 ppm, about 400 ppm to about 1,100 ppm, about 400 ppm to about 1,200 ppm, about 400 ppm to about 1,300 ppm, about 400 ppm to about 1,400 ppm, about 400 ppm to about 1,500 ppm, about 400 ppm to about 1,600 ppm, about 400 ppm to about 1,700 ppm, about 400 ppm to about 1,800 ppm, about 400 ppm to about 1,900 ppm, about 400 ppm to about 2,000 ppm, about 500 ppm to about 600 ppm, about 500 ppm to about 700 ppm, about 500 ppm to about 800 ppm, about 500 ppm to about 900 ppm, about 500 ppm to about 1,000 ppm, about 500 ppm to about 1,100 ppm, about 500 ppm to about 1,200 ppm, about 500 ppm to about 1,300 ppm, about 500 ppm to about 1,400 ppm, about 500 ppm to about 1,500 ppm, about 500 ppm to about 1,600 ppm, about 500 ppm to about 1,700 ppm, about 500 ppm to about 1,800 ppm, about 500 ppm to about 1,900 ppm, about 500 ppm to about 2,000 ppm, about 600 ppm to about 700 ppm, about 600 ppm to about 800 ppm, about 600 ppm to about 900 ppm, about 600 ppm to about 1,000 ppm, about 600 ppm to about 1,100 ppm, about 600 ppm to about 1,200 ppm, about 600 ppm to about 1,300 ppm, about 600 ppm to about 1,400 ppm, about 600 ppm to about 1,500 ppm, about 600 ppm to about 1,600 ppm, about 600 ppm to about 1,700 ppm, about 600 ppm to about 1,800 ppm, about 600 ppm to about 1,900 ppm, about 600 ppm to about 2,000 ppm, about 700 ppm to about 800 ppm, about 700 ppm to about 900 ppm, about 700 ppm to about 1,000 ppm, about 700 ppm to about 1,100 ppm, about 700 ppm to about 1,200 ppm, about 700 ppm to about 1,300 ppm, about 700 ppm to about 1,400 ppm, about 700 ppm to about 1,500 ppm, about 700 ppm to about 1,600 ppm, about 700 ppm to about 1,700 ppm, about 700 ppm to about 1,800 ppm, about 700 ppm to about 1,900 ppm, about 700 ppm to about 2,000 ppm, about 800 ppm to about 900 ppm, about 800 ppm to about 1,000 ppm, about 800 ppm to about 1,100 ppm, about 800 ppm to about 1,200 ppm, about 800 ppm to about 1,300 ppm, about 800 ppm to about 1,400 ppm, about 800 ppm to about 1,500 ppm, about 800 ppm to about 1,600 ppm, about 800 ppm to about 1,700 ppm, about 800 ppm to about 1,800 ppm, about 800 ppm to about 1,900 ppm, about 800 ppm to about 2,000 ppm, about 900 ppm to about 1,000 ppm, about 900 ppm to about 1,100 ppm, about 900 ppm to about 1,200 ppm, about 900 ppm to about 1,300 ppm, about 900 ppm to about 1,400 ppm, about 900 ppm to about 1,500 ppm, about 900 ppm to about 1,600 ppm, about 900 ppm to about 1,700 ppm, about 900 ppm to about 1,800 ppm, about 900 ppm to about 1,900 ppm, about 900 ppm to about 2,000 ppm, about 1,000 ppm to about 1,100 ppm, about 1,000 ppm to about 1,200 ppm, about 1,000 ppm to about 1,300 ppm, about 1,000 ppm to about 1,400 ppm, about 1,000 ppm to about 1,500 ppm, about 1,000 ppm to about 1,600 ppm, about 1,000 ppm to about 1,700 ppm, about 1,000 ppm to about 1,800 ppm, about 1,000 ppm to about 1,900 ppm, about 1,000 ppm to about 2,000 ppm, about 1,100 ppm to about 1,200 ppm, about 1,100 ppm to about 1,300 ppm, about 1,100 ppm to about 1,400 ppm, about 1,100 ppm to about 1,500 ppm, about 1,100 ppm to about 1,600 ppm, about 1,100 ppm to about 1,700 ppm, about 1,100 ppm to about 1,800 ppm, about 1,100 ppm to about 1,900 ppm, about 1,100 ppm to about 2,000 ppm, about 1,200 ppm to about 1,300 ppm, about 1,200 ppm to about 1,400 ppm, about 1,200 ppm to about 1,500 ppm, about 1,200 ppm to about 1,600 ppm, about 1,200 ppm to about 1,700 ppm, about 1,200 ppm to about 1,800 ppm, about 1,200 ppm to about 1,900 ppm, about 1,200 ppm to about 2,000 ppm, about 1,300 ppm to about 1,400 ppm, about 1,300 ppm to about 1,500 ppm, about 1,300 ppm to about 1,600 ppm, about 1,300 ppm to about 1,700 ppm, about 1,300 ppm to about 1,800 ppm, about 1,300 ppm to about 1,900 ppm, about 1,300 ppm to about 2,000 ppm, about 1,400 ppm to about 1,500 ppm, about 1,400 ppm to about 1,600 ppm, about 1,400 ppm to about 1,700 ppm, about 1,400 ppm to about 1,800 ppm, about 1,400 ppm to about 1,900 ppm, about 1,400 ppm to about 2,000 ppm, about 1,500 ppm to about 1,600 ppm, about 1,500 ppm to about 1,700 ppm, about 1,500 ppm to about 1,800 ppm, about 1,500 ppm to about 1,900 ppm, about 1,500 ppm to about 2,000 ppm, about 1,600 ppm to about 1,700 ppm, about 1,600 ppm to about 1,800 ppm, about 1,600 ppm to about 1,900 ppm, about 1,600 ppm to about 2,000 ppm, about 1,700 ppm to about 1,800 ppm, about 1,700 ppm to about 1,900 ppm, about 1,700 ppm to about 2,000 ppm, about 1,800 ppm to about 1,900 ppm, about 1,800 ppm to about 2,000 ppm, or about 1,900 ppm to about 2,000 ppm.

In aspects of this embodiment, a composition disclosed herein comprises metallic particles in an amount of, e.g., 0.05 mg/L, 0.10 mg/L, 0.15 mg/L, 0.20 mg/L, 0.25 mg/L, 0.30 mg/L, 0.35 mg/L, 0.40 mg/L, 0.45 mg/L, 0.50 mg/L, 0.55 mg/L, 0.60 mg/L, 0.65 mg/L, 0.70 mg/L, 0.75 mg/L, 0.80 mg/L, 0.85 mg/L, 0.90 mg/L, 0.95 mg/L, 1 mg/L, 5 mg/L, 10 mg/L, 15 mg/L, 20 mg/L, 25 mg/L, 30 mg/L, 35 mg/L, 40 mg/L, 45 mg/L, 50 mg/L, 55 mg/L, 60 mg/L, 65 mg/L, 70 mg/L, 75 mg/L, 80 mg/L, 85 mg/L, 90 mg/L, 95 mg/L, 100 mg/L, 125 mg/L, 150 mg/L, 175 mg/L, 200 mg/L, 225 mg/L, 250 mg/L, 275 mg/L, 300 mg/L, 325 mg/L, 350 mg/L, 375 mg/L, 400 mg/L, 425 mg/L, 450 mg/L, 475 mg/L, 500 mg/L, 525 mg/L, 550 mg/L, 575 mg/L, 600 mg/L, 625 mg/L, 650 mg/L, 675 mg/L, 700 mg/L, 725 mg/L, 750 mg/L, 775 mg/L, 800 mg/L, 825 mg/L, 850 mg/L, 875 mg/L, 900 mg/L, 925 mg/L, 950 mg/L, 975 mg/L, 1,000 mg/L, 1,025 mg/L, 1,050 mg/L, 1075 mg/L, 1,100 mg/L, 1,125 mg/L, 1,150 mg/L, 1,175 mg/L, 1,200 mg/L, 1,225 mg/L, 1,250 mg/L, 1,275 mg/L, 1,300 mg/L, 1,325 mg/L, 1,350 mg/L, 1,375 mg/L, 1,400 mg/L, 1,425 mg/L, 1,450 mg/L, 1,475 mg/L, 1,500 mg/L, 1,600 mg/L, 1,700 mg/L, 1,800 mg/L, 1,900 mg/L, or 2,000 mg/L. In other aspects of this embodiment, a composition disclosed herein comprises metallic particles in an amount of, e.g., at least 0.05 mg/L, at least 0.10 mg/L, at least 0.20 mg/L, at least 0.30 mg/L, at least 0.40 mg/L, at least 0.50 mg/L, at least 0.60 mg/L, at least 0.70 mg/L, at least 0.80 mg/L, at least 0.90 mg/L, at least 1 mg/L, at least 10 mg/L, at least 20 mg/L, at least 30 mg/L, at least 40 mg/L, at least 50 mg/L, at least 60 mg/L, at least 70 mg/L, at least 80 mg/L, at least 90 mg/L, at least 100 mg/L, at least 125 mg/L, at least 150 mg/L, at least 175 mg/L, at least 200 mg/L, at least 225 mg/L, at least 250 mg/L, at least 275 mg/L, at least 300 mg/L, at least 325 mg/L, at least 350 mg/L, at least 375 mg/L, at least 400 mg/L, at least 425 mg/L, at least 450 mg/L, at least 475 mg/L, at least 500 mg/L, at least 525 mg/L, at least 550 mg/L, at least 575 mg/L, at least 600 mg/L, at least 625 mg/L, at least 650 mg/L, at least 675 mg/L, at least 700 mg/L, at least 725 mg/L, at least 750 mg/L, at least 775 mg/L, at least 800 mg/L, at least 825 mg/L, at least 850 mg/L, at least 875 mg/L, at least 900 mg/L, at least 925 mg/L, at least 950 mg/L, at least 975 mg/L, at least 1,000 mg/L, at least 1,025 mg/L, at least 1,050 mg/L, at least 1075 mg/L, at least 1,100 mg/L, at least 1,125 mg/L, at least 1,150 mg/L, at least 1,175 mg/L, at least 1,200 mg/L, at least 1,225 mg/L, at least 1,250 mg/L, at least 1,275 mg/L, at least 1,300 mg/L, at least 1,325 mg/L, at least 1,350 mg/L, at least 1,375 mg/L, at least 1,400 mg/L, at least 1,425 mg/L, at least 1,450 mg/L, at least 1,475 mg/L, at least 1,500 mg/L, at least 1,600 mg/L, at least 1,700 mg/L, at least 1,800 mg/L, at least 1,900 mg/L, or at least 2,000 mg/L. In yet other aspects of this embodiment, a composition disclosed herein comprises metallic particles in an amount of, e.g., at most 0.05 mg/L, at most 0.10 mg/L, at most 0.20 mg/L, at most 0.30 mg/L, at most 0.40 mg/L, at most 0.50 mg/L, at most 0.60 mg/L, at most 0.70 mg/L, at most 0.80 mg/L, at most 0.90 mg/L, at most 1 mg/L, at most 10 mg/L, at most 20 mg/L, at most 30 mg/L, at most 40 mg/L, at most 50 mg/L, at most 60 mg/L, at most 70 mg/L, at most 80 mg/L, at most 90 mg/L, at most 100 mg/L, at most 125 mg/L, at most 150 mg/L, at most 175 mg/L, at most 200 mg/L, at most 225 mg/L, at most 250 mg/L, at most 275 mg/L, at most 300 mg/L, at most 325 mg/L, at most 350 mg/L, at most 375 mg/L, at most 400 mg/L, at most 425 mg/L, at most 450 mg/L, at most 475 mg/L, at most 500 mg/L, at most 525 mg/L, at most 550 mg/L, at most 575 mg/L, at most 600 mg/L, at most 625 mg/L, at most 650 mg/L, at most 675 mg/L, at most 700 mg/L, at most 725 mg/L, at most 750 mg/L, at most 775 mg/L, at most 800 mg/L, at most 825 mg/L, at most 850 mg/L, at most 875 mg/L, at most 900 mg/L, at most 925 mg/L, at most 950 mg/L, at most 975 mg/L, at most 1,000 mg/L, at most 1,025 mg/L, at most 1,050 mg/L, at most 1075 mg/L, at most 1,100 mg/L, at most 1,125 mg/L, at most 1,150 mg/L, at most 1,175 mg/L, at most 1,200 mg/L, at most 1,225 mg/L, at most 1,250 mg/L, at most 1,275 mg/L, at most 1,300 mg/L, at most 1,325 mg/L, at most 1,350 mg/L, at most 1,375 mg/L, at most 1,400 mg/L, at most 1,425 mg/L, at most 1,450 mg/L, at most 1,475 mg/L, at most 1,500 mg/L, at most 1,600 mg/L, at most 1,700 mg/L, at most 1,800 mg/L, at most 1,900 mg/L, or at most 2,000 mg/L.

In still other aspects of this embodiment, a composition disclosed herein comprises metallic particles in an amount of from, e.g., about 0.5 mg/L to about 20 mg/L, about 0.5 mg/L to about 25 mg/L, about 0.5 mg/L to about 30 mg/L, about 0.5 mg/L to about 35 mg/L, about 0.5 mg/L to about 40 mg/L, about 0.5 mg/L to about 45 mg/L, about 0.5 mg/L to about 50 mg/L, about 0.5 mg/L to about 55 mg/L, about 0.5 mg/L to about 60 mg/L, about 0.5 mg/L to about 65 mg/L, about 0.5 mg/L to about 70 mg/L, about 0.5 mg/L to about 75 mg/L, about 0.5 mg/L to about 80 mg/L, about 0.5 mg/L to about 85 mg/L, about 0.5 mg/L to about 90 mg/L, about 0.5 mg/L to about 95 mg/L, about 0.5 mg/L to about 100 mg/L, about 0.75 mg/L to about 20 mg/L, about 0.75 mg/L to about 25 mg/L, about 0.75 mg/L to about 30 mg/L, about 0.75 mg/L to about 35 mg/L, about 0.75 mg/L to about 40 mg/L, about 0.75 mg/L to about 45 mg/L, about 0.75 mg/L to about 50 mg/L, about 0.75 mg/L to about 55 mg/L, about 0.75 mg/L to about 60 mg/L, about 0.75 mg/L to about 65 mg/L, about 0.75 mg/L to about 70 mg/L, about 0.75 mg/L to about 75 mg/L, about 0.75 mg/L to about 80 mg/L, about 0.75 mg/L to about 85 mg/L, about 0.75 mg/L to about 90 mg/L, about 0.75 mg/L to about 95 mg/L, about 0.75 mg/L to about 100 mg/L, about 1 mg/L to about 20 mg/L, about 1 mg/L to about 25 mg/L, about 1 mg/L to about 30 mg/L, about 1 mg/L to about 35 mg/L, about 1 mg/L to about 40 mg/L, about 1 mg/L to about 45 mg/L, about 1 mg/L to about 50 mg/L, about 1 mg/L to about 55 mg/L, about 1 mg/L to about 60 mg/L, about 1 mg/L to about 65 mg/L, about 1 mg/L to about 70 mg/L, about 1 mg/L to about 75 mg/L, about 1 mg/L to about 80 mg/L, about 1 mg/L to about 85 mg/L, about 1 mg/L to about 90 mg/L, about 1 mg/L to about 95 mg/L, about 1 mg/L to about 100 mg/L, about 5 mg/L to about 20 mg/L, about 5 mg/L to about 25 mg/L, about 5 mg/L to about 30 mg/L, about 5 mg/L to about 35 mg/L, about 5 mg/L to about 40 mg/L, about 5 mg/L to about 45 mg/L, about 5 mg/L to about 50 mg/L, about 5 mg/L to about 55 mg/L, about 5 mg/L to about 60 mg/L, about 5 mg/L to about 65 mg/L, about 5 mg/L to about 70 mg/L, about 5 mg/L to about 75 mg/L, about 5 mg/L to about 80 mg/L, about 5 mg/L to about 85 mg/L, about 5 mg/L to about 90 mg/L, about 5 mg/L to about 95 mg/L, about 5 mg/L to about 100 mg/L, about 10 mg/L to about 20 mg/L, about 10 mg/L to about 25 mg/L, about 10 mg/L to about 30 mg/L, about 10 mg/L to about 35 mg/L, about 10 mg/L to about 40 mg/L, about 10 mg/L to about 45 mg/L, about 10 mg/L to about 50 mg/L, about 10 mg/L to about 55 mg/L, about 10 mg/L to about 60 mg/L, about 10 mg/L to about 65 mg/L, about 10 mg/L to about 70 mg/L, about 10 mg/L to about 75 mg/L, about 10 mg/L to about 80 mg/L, about 10 mg/L to about 85 mg/L, about 10 mg/L to about 90 mg/L, about 10 mg/L to about 95 mg/L, or about 10 mg/L to about 100 mg/L.

In other aspects of this embodiment, a composition disclosed herein comprises metallic particles in an amount of from, e.g., about 1 mg/L to about 25 mg/L, about 1 mg/L to about 50 mg/L, about 1 mg/L to about 75 mg/L, about 1 mg/L to about 100 mg/L, about 1 mg/L to about 125 mg/L, about 1 mg/L to about 150 mg/L, about 1 mg/L to about 175 mg/L, about 1 mg/L to about 200 mg/L, about 1 mg/L to about 225 mg/L, about 1 mg/L to about 250 mg/L, about 1 mg/L to about 275 mg/L, about 1 mg/L to about 300 mg/L, about 1 mg/L to about 325 mg/L, about 1 mg/L to about 350 mg/L, about 1 mg/L to about 375 mg/L, about 1 mg/L to about 400 mg/L, about 10 mg/L to about 25 mg/L, about 10 mg/L to about 50 mg/L, about 10 mg/L to about 75 mg/L, about 10 mg/L to about 100 mg/L, about 10 mg/L to about 125 mg/L, about 10 mg/L to about 150 mg/L, about 10 mg/L to about 175 mg/L, about 10 mg/L to about 200 mg/L, about 10 mg/L to about 225 mg/L, about 10 mg/L to about 250 mg/L, about 10 mg/L to about 275 mg/L, about 10 mg/L to about 300 mg/L, about 10 mg/L to about 325 mg/L, about 10 mg/L to about 350 mg/L, about 10 mg/L to about 375 mg/L, about 10 mg/L to about 400 mg/L, about 25 mg/L to about 50 mg/L, about 25 mg/L to about 75 mg/L, about 25 mg/L to about 100 mg/L, about 25 mg/L to about 125 mg/L, about 25 mg/L to about 150 mg/L, about 25 mg/L to about 175 mg/L, about 25 mg/L to about 200 mg/L, about 25 mg/L to about 225 mg/L, about 25 mg/L to about 250 mg/L, about 25 mg/L to about 275 mg/L, about 25 mg/L to about 300 mg/L, about 25 mg/L to about 325 mg/L, about 25 mg/L to about 350 mg/L, about 25 mg/L to about 375 mg/L, about 25 mg/L to about 400 mg/L, about 50 mg/L to about 75 mg/L, about 50 mg/L to about 100 mg/L, about 50 mg/L to about 125 mg/L, about 50 mg/L to about 150 mg/L, about 50 mg/L to about 175 mg/L, about 50 mg/L to about 200 mg/L, about 50 mg/L to about 225 mg/L, about 50 mg/L to about 250 mg/L, about 50 mg/L to about 275 mg/L, about 50 mg/L to about 300 mg/L, about 50 mg/L to about 325 mg/L, about 50 mg/L to about 350 mg/L, about 50 mg/L to about 375 mg/L, about 50 mg/L to about 400 mg/L, about 75 mg/L to about 100 mg/L, about 75 mg/L to about 125 mg/L, about 75 mg/L to about 150 mg/L, about 75 mg/L to about 175 mg/L, about 75 mg/L to about 200 mg/L, about 75 mg/L to about 225 mg/L, about 75 mg/L to about 250 mg/L, about 75 mg/L to about 275 mg/L, about 75 mg/L to about 300 mg/L, about 75 mg/L to about 325 mg/L, about 75 mg/L to about 350 mg/L, about 75 mg/L to about 375 mg/L, about 75 mg/L to about 400 mg/L, about 100 mg/L to about 125 mg/L, about 100 mg/L to about 150 mg/L, about 100 mg/L to about 175 mg/L, about 100 mg/L to about 200 mg/L, about 100 mg/L to about 225 mg/L, about 100 mg/L to about 250 mg/L, about 100 mg/L to about 275 mg/L, about 100 mg/L to about 300 mg/L, about 100 mg/L to about 325 mg/L, about 100 mg/L to about 350 mg/L, about 100 mg/L to about 375 mg/L, about 100 mg/L to about 400 mg/L, about 150 mg/L to about 175 mg/L, about 150 mg/L to about 200 mg/L, about 150 mg/L to about 225 mg/L, about 150 mg/L to about 250 mg/L, about 150 mg/L to about 275 mg/L, about 150 mg/L to about 300 mg/L, about 150 mg/L to about 325 mg/L, about 150 mg/L to about 350 mg/L, about 150 mg/L to about 375 mg/L, about 150 mg/L to about 400 mg/L, about 200 mg/L to about 225 mg/L, about 200 mg/L to about 250 mg/L, about 200 mg/L to about 275 mg/L, about 200 mg/L to about 300 mg/L, about 200 mg/L to about 325 mg/L, about 200 mg/L to about 350 mg/L, about 200 mg/L to about 375 mg/L, about 200 mg/L to about 400 mg/L, about 250 mg/L to about 275 mg/L, about 250 mg/L to about 300 mg/L, about 250 mg/L to about 325 mg/L, about 250 mg/L to about 350 mg/L, about 250 mg/L to about 375 mg/L, about 250 mg/L to about 400 mg/L, about 300 mg/L to about 325 mg/L, about 300 mg/L to about 350 mg/L, about 300 mg/L to about 375 mg/L, about 300 mg/L to about 400 mg/L, about 350 mg/L to about 375 mg/L, about 350 mg/L to about 400 mg/L, or about 375 mg/L to about 400 mg/L.

In other aspects of this embodiment, a composition disclosed herein comprises metallic particles in an amount of, e.g., about 400 mg/L to about 500 mg/L, about 400 mg/L to about 600 mg/L, about 400 mg/L to about 700 mg/L, about 400 mg/L to about 800 mg/L, about 400 mg/L to about 900 mg/L, about 400 mg/L to about 1,000 mg/L, about 400 mg/L to about 1,100 mg/L, about 400 mg/L to about 1,200 mg/L, about 400 mg/L to about 1,300 mg/L, about 400 mg/L to about 1,400 mg/L, about 400 mg/L to about 1,500 mg/L, about 400 mg/L to about 1,600 mg/L, about 400 mg/L to about 1,700 mg/L, about 400 mg/L to about 1,800 mg/L, about 400 mg/L to about 1,900 mg/L, about 400 mg/L to about 2,000 mg/L, about 500 mg/L to about 600 mg/L, about 500 mg/L to about 700 mg/L, about 500 mg/L to about 800 mg/L, about 500 mg/L to about 900 mg/L, about 500 mg/L to about 1,000 mg/L, about 500 mg/L to about 1,100 mg/L, about 500 mg/L to about 1,200 mg/L, about 500 mg/L to about 1,300 mg/L, about 500 mg/L to about 1,400 mg/L, about 500 mg/L to about 1,500 mg/L, about 500 mg/L to about 1,600 mg/L, about 500 mg/L to about 1,700 mg/L, about 500 mg/L to about 1,800 mg/L, about 500 mg/L to about 1,900 mg/L, about 500 mg/L to about 2,000 mg/L, about 600 mg/L to about 700 mg/L, about 600 mg/L to about 800 mg/L, about 600 mg/L to about 900 mg/L, about 600 mg/L to about 1,000 mg/L, about 600 mg/L to about 1,100 mg/L, about 600 mg/L to about 1,200 mg/L, about 600 mg/L to about 1,300 mg/L, about 600 mg/L to about 1,400 mg/L, about 600 mg/L to about 1,500 mg/L, about 600 mg/L to about 1,600 mg/L, about 600 mg/L to about 1,700 mg/L, about 600 mg/L to about 1,800 mg/L, about 600 mg/L to about 1,900 mg/L, about 600 mg/L to about 2,000 mg/L, about 700 mg/L to about 800 mg/L, about 700 mg/L to about 900 mg/L, about 700 mg/L to about 1,000 mg/L, about 700 mg/L to about 1,100 mg/L, about 700 mg/L to about 1,200 mg/L, about 700 mg/L to about 1,300 mg/L, about 700 mg/L to about 1,400 mg/L, about 700 mg/L to about 1,500 mg/L, about 700 mg/L to about 1,600 mg/L, about 700 mg/L to about 1,700 mg/L, about 700 mg/L to about 1,800 mg/L, about 700 mg/L to about 1,900 mg/L, about 700 mg/L to about 2,000 mg/L, about 800 mg/L to about 900 mg/L, about 800 mg/L to about 1,000 mg/L, about 800 mg/L to about 1,100 mg/L, about 800 mg/L to about 1,200 mg/L, about 800 mg/L to about 1,300 mg/L, about 800 mg/L to about 1,400 mg/L, about 800 mg/L to about 1,500 mg/L, about 800 mg/L to about 1,600 mg/L, about 800 mg/L to about 1,700 mg/L, about 800 mg/L to about 1,800 mg/L, about 800 mg/L to about 1,900 mg/L, about 800 mg/L to about 2,000 mg/L, about 900 mg/L to about 1,000 mg/L, about 900 mg/L to about 1,100 mg/L, about 900 mg/L to about 1,200 mg/L, about 900 mg/L to about 1,300 mg/L, about 900 mg/L to about 1,400 mg/L, about 900 mg/L to about 1,500 mg/L, about 900 mg/L to about 1,600 mg/L, about 900 mg/L to about 1,700 mg/L, about 900 mg/L to about 1,800 mg/L, about 900 mg/L to about 1,900 mg/L, about 900 mg/L to about 2,000 mg/L, about 1,000 mg/L to about 1,100 mg/L, about 1,000 mg/L to about 1,200 mg/L, about 1,000 mg/L to about 1,300 mg/L, about 1,000 mg/L to about 1,400 mg/L, about 1,000 mg/L to about 1,500 mg/L, about 1,000 mg/L to about 1,600 mg/L, about 1,000 mg/L to about 1,700 mg/L, about 1,000 mg/L to about 1,800 mg/L, about 1,000 mg/L to about 1,900 mg/L, about 1,000 mg/L to about 2,000 mg/L, about 1,100 mg/L to about 1,200 mg/L, about 1,100 mg/L to about 1,300 mg/L, about 1,100 mg/L to about 1,400 mg/L, about 1,100 mg/L to about 1,500 mg/L, about 1,100 mg/L to about 1,600 mg/L, about 1,100 mg/L to about 1,700 mg/L, about 1,100 mg/L to about 1,800 mg/L, about 1,100 mg/L to about 1,900 mg/L, about 1,100 mg/L to about 2,000 mg/L, about 1,200 mg/L to about 1,300 mg/L, about 1,200 mg/L to about 1,400 mg/L, about 1,200 mg/L to about 1,500 mg/L, about 1,200 mg/L to about 1,600 mg/L, about 1,200 mg/L to about 1,700 mg/L, about 1,200 mg/L to about 1,800 mg/L, about 1,200 mg/L to about 1,900 mg/L, about 1,200 mg/L to about 2,000 mg/L, about 1,300 mg/L to about 1,400 mg/L, about 1,300 mg/L to about 1,500 mg/L, about 1,300 mg/L to about 1,600 mg/L, about 1,300 mg/L to about 1,700 mg/L, about 1,300 mg/L to about 1,800 mg/L, about 1,300 mg/L to about 1,900 mg/L, about 1,300 mg/L to about 2,000 mg/L, about 1,400 mg/L to about 1,500 mg/L, about 1,400 mg/L to about 1,600 mg/L, about 1,400 mg/L to about 1,700 mg/L, about 1,400 mg/L to about 1,800 mg/L, about 1,400 mg/L to about 1,900 mg/L, about 1,400 mg/L to about 2,000 mg/L, about 1,500 mg/L to about 1,600 mg/L, about 1,500 mg/L to about 1,700 mg/L, about 1,500 mg/L to about 1,800 mg/L, about 1,500 mg/L to about 1,900 mg/L, about 1,500 mg/L to about 2,000 mg/L, about 1,600 mg/L to about 1,700 mg/L, about 1,600 mg/L to about 1,800 mg/L, about 1,600 mg/L to about 1,900 mg/L, about 1,600 mg/L to about 2,000 mg/L, about 1,700 mg/L to about 1,800 mg/L, about 1,700 mg/L to about 1,900 mg/L, about 1,700 mg/L to about 2,000 mg/L, about 1,800 mg/L to about 1,900 mg/L, about 1,800 mg/L to about 2,000 mg/L, or about 1,900 mg/L to about 2,000 mg/L.

In aspects of this embodiment, a composition disclosed herein comprises metallic particles in an amount of, e.g., about 0.001%, about 0.005%, about 0.01%, about 0.05%, about 0.10%, about 0.11%, about 0.12%, about 0.13%, about 0.14%, about 0.15%, about 0.16%, about 0.17%, about 0.18%, about 0.19%, about 0.2%, about 0.25%, about 0.3%, about 0.35%, about 0.4%, about 0.45%, or about 0.5% by weight of the composition. In other aspects of this embodiment, a composition disclosed herein comprises metallic particles in an amount of, e.g., at least 0.001%, at least 0.005%, at least 0.01%, at least 0.05%, at least 0.10%, at least 0.11%, at least 0.12%, at least 0.13%, at least 0.14%, at least 0.15%, at least 0.16%, at least 0.17%, at least 0.18%, at least 0.19%, at least 0.2%, at least 0.25%, at least 0.3%, at least 0.35%, at least 0.4%, at least 0.45%, or at least 0.5% by weight of the composition. In yet other aspects of this embodiment, a composition disclosed herein comprises metallic particles in an amount of, e.g., at most 0.001%, at most 0.005%, at most 0.01%, at most 0.05%, at most 0.10%, at most 0.11%, at most 0.12%, at most 0.13%, at most 0.14%, at most 0.15%, at most 0.16%, at most 0.17%, at most 0.18%, at most 0.19%, at most 0.2%, at most 0.25%, at most 0.3%, at most 0.35%, at most 0.4%, at most 0.45%, or at most 0.5% by weight of the composition. In still other aspects of this embodiment, a composition disclosed herein comprises metallic particles in an amount of, e.g., about 0.0010% to about 0.005%, about 0.0010% to about 0.010%, about 0.0010% to about 0.050%, about 0.0010% to about 0.10%, about 0.0010% to about 0.15%, about 0.0010% to about 0.20%, about 0.0010% to about 0.25%, about 0.0010% to about 0.30%, about 0.0010% to about 0.35%, about 0.0010% to about 0.40%, about 0.0010% to about 0.45%, about 0.0010% to about 0.50%, about 0.010% to about 0.050%, about 0.010% to about 0.10%, about 0.010% to about 0.15%, about 0.010% to about 0.20%, about 0.010% to about 0.25%, about 0.010% to about 0.30%, about 0.010% to about 0.35%, about 0.010% to about 0.40%, about 0.010% to about 0.45%, about 0.010% to about 0.50%, about 0.10% to about 0.15%, about 0.10% to about 0.20%, about 0.10% to about 0.25%, about 0.10% to about 0.30%, about 0.10% to about 0.35%, about 0.10% to about 0.40%, about 0.10% to about 0.45%, about 0.10% to about 0.50%, about 0.20% to about 0.25%, about 0.20% to about 0.30%, about 0.20% to about 0.35%, about 0.20% to about 0.40%, about 0.20% to about 0.45%, about 0.20% to about 0.50%, about 0.30% to about 0.35%, about 0.30% to about 0.40%, about 0.30% to about 0.45%, about 0.30% to about 0.50%, about 0.40% to about 0.45%, about 0.40% to about 0.50%, or about 0.40% to about 0.50% by weight of the composition.

In an embodiment, metallic particles can be of any size that provides a desired beneficial effect to a composition disclosed herein. In aspects of this embodiment, metallic particles disclosed herein have a mean diameter of, e.g., about 10 nm, about 20 nm, about 30 nm, about 40 nm, about 50 nm, about 60 nm, about 70 nm, about 80 nm, about 90 nm, about 100 nm. In other aspects of this embodiment, metallic particles disclosed herein have a mean diameter of, e.g., at least 10 nm, at least 20 nm, at least 30 nm, at least 40 nm, at least 50 nm, at least 60 nm, at least 70 nm, at least 80 nm, at least 90 nm, at least 100 nm. In yet other aspects of this embodiment, metallic particles disclosed herein have a mean diameter of, e.g., at most 10 nm, at most 20 nm, at most 30 nm, at most 40 nm, at most 50 nm, at most 60 nm, at most 70 nm, at most 80 nm, at most 90 nm, at most 100 nm. In yet other aspects of this embodiment, metallic particles disclosed herein have a mean diameter of, e.g., about 10 nm to about 20 nm, about 10 nm to about 30 nm, about 10 nm to about 40 nm, about 10 nm to about 50 nm, about 10 nm to about 60 nm, about 10 nm to about 70 nm, about 10 nm to about 80 nm, about 10 nm to about 90 nm, about 10 nm to about 100 nm, about 20 nm to about 30 nm, about 20 nm to about 40 nm, about 20 nm to about 50 nm, about 20 nm to about 60 nm, about 20 nm to about 70 nm, about 20 nm to about 80 nm, about 20 nm to about 90 nm, about 20 nm to about 100 nm, about 30 nm to about 40 nm, about 30 nm to about 50 nm, about 30 nm to about 60 nm, about 30 nm to about 70 nm, about 30 nm to about 80 nm, about 30 nm to about 90 nm, about 30 nm to about 100 nm, about 40 nm to about 50 nm, about 40 nm to about 60 nm, about 40 nm to about 70 nm, about 40 nm to about 80 nm, about 40 nm to about 90 nm, about 40 nm to about 100 nm, about 50 nm to about 60 nm, about 50 nm to about 70 nm, about 50 nm to about 80 nm, about 50 nm to about 90 nm, about 50 nm to about 100 nm, about 60 nm to about 70 nm, about 60 nm to about 80 nm, about 60 nm to about 90 nm, about 60 nm to about 100 nm, about 70 nm to about 80 nm, about 70 nm to about 90 nm, about 70 nm to about 100 nm, about 80 nm to about 90 nm, about 80 nm to about 100 nm, or about 90 nm to about 100 nm.

In an embodiment, a composition disclosed herein does not comprise metallic particles.

A composition disclosed herein may comprise a metal salt. A metal salt disclosed herein can be an alkali metal salt, an alkaline earth metal salt, a base metal salt, or a transition metal salt. An alkali metal salt includes a lithium salt, a sodium salt, a potassium salt, a rubidium salt, a cesium salt, and a francium salt. An alkaline earth metal salt includes a beryllium salt, a magnesium salt, a calcium salt, a strontium salt, a barium salt, and a radium salt. A basic metal salt includes an aluminum salt, a gallium salt, an indium salt, a tin salt, a thallium salt, a lead salt, and a bismuth salt. A transition metal salt includes a scandium salt, a titanium salt, a vanadium salt, a chromium salt, a manganese salt, an iron salt, a cobalt salt, a palladium salt, a silver salt, m a nickel salt, a copper salt, a zinc salt, a yttrium salt, Non-limiting examples of a metal salt includes a calcium salt, a copper salt, iron salt, a potassium salt, silver salt, titanium salt, and zinc salt.

Other non-limiting examples of a metal salt includes a metal acetate salt, a metal chloride salt, a metal nitrate salt, a metal oxide salt, and a metal sulfate salt. A metal acetate salt includes, without limitation, calcium acetate, copper acetate, iron acetate, e.g., iron (II) acetate and iron (III) acetate, potassium acetate, silver acetate, titanium acetate, and zinc acetate. A metal chloride salt includes, without limitation, calcium chloride salt, copper chloride, iron chloride, e.g., iron (II) chloride or iron (III) chloride, potassium chloride, silver chloride, titanium chloride, and zinc chloride. A metal nitrate salt includes, without limitation, calcium nitrate salt, copper nitrate, iron nitrate, e.g., iron (II) nitrate, iron (III) nitrate, potassium nitrate, silver nitrate, titanium nitrate, and zinc nitrate. A metal oxide salt includes, without limitation, calcium oxide salt, copper oxide salt, iron oxide salt, e.g., iron (II) oxide salt, iron (III) oxide salt, potassium oxide salt, silver oxide salt, titanium oxide salt, and zinc oxide salt. A metal sulfate salt includes, without limitation, calcium sulfate, copper sulfate, iron sulfate, e.g., iron (II) sulfate, iron (III) sulfate, potassium sulfate, silver sulfate, titanium sulfate, and zinc sulfate.

In some embodiments, a composition may include calcium oxide obtained from an organic source like shells from oceanic organisms. In some embodiment, a composition may include calcium oxide obtain from an inorganic source like by a synthetic chemical process.

In an embodiment, a composition disclosed herein comprises an amount of a metal salt that provides a desired beneficial effect to a composition disclosed herein. For example, when a composition comprising metallic salts is formulated to be used in an undiluted state, the amount of metallic salts used is a microbiostatic effective amount. A microbiostatic effective amount of metallic salts is an amount that restrains, prohibits, or otherwise prevents microbial population growth without eradicating the microbes. In an aspect of this embodiment, a microbiostatic effective amount of metallic salts is an amount that restrains, prohibits, or otherwise prevents microbial population growth without eradicating the microbes to a degree that achieves disinfection or sterilization. Alternatively, when a composition comprising metallic salts is formulated to be used in methods where the composition will be diluted, the amount of metallic salts used in such concentrated formulations can be any amount that does not cause precipitation of components in the composition disclosed herein or otherwise create unwanted effects that make the composition inoperable.

In aspects of this embodiment, a composition disclosed herein comprises a metal salt in an amount of, e.g., 0.05 ppm, 0.10 ppm, 0.15 ppm, 0.20 ppm, 0.25 ppm, 0.30 ppm, 0.35 ppm, 0.40 ppm, 0.45 ppm, 0.50 ppm, 0.55 ppm, 0.60 ppm, 0.65 ppm, 0.70 ppm, 0.75 ppm, 0.80 ppm, 0.85 ppm, 0.90 ppm, 0.95 ppm, 1 ppm, 5 ppm, 10 ppm, 15 ppm, 20 ppm, 25 ppm, 30 ppm, 35 ppm, 40 ppm, 45 ppm, 50 ppm, 55 ppm, 60 ppm, 65 ppm, 70 ppm, 75 ppm, 80 ppm, 85 ppm, 90 ppm, 95 ppm, 100 ppm, 125 ppm, 150 ppm, 175 ppm, 200 ppm, 225 ppm, 250 ppm, 275 ppm, 300 ppm, 325 ppm, 350 ppm, 375 ppm, 400 ppm, 425 ppm, 450 ppm, 475 ppm, 500 ppm, 525 ppm, 550 ppm, 575 ppm, 600 ppm, 625 ppm, 650 ppm, 675 ppm, 700 ppm, 725 ppm, 750 ppm, 775 ppm, 800 ppm, 825 ppm, 850 ppm, 875 ppm, 900 ppm, 925 ppm, 950 ppm, 975 ppm, 1,000 ppm, 1,025 ppm, 1,050 ppm, 1075 ppm, 1,100 ppm, 1,125 ppm, 1,150 ppm, 1,175 ppm, 1,200 ppm, 1,225 ppm, 1,250 ppm, 1,275 ppm, 1,300 ppm, 1,325 ppm, 1,350 ppm, 1,375 ppm, 1,400 ppm, 1,425 ppm, 1,450 ppm, 1,475 ppm, 1,500 ppm, 1,600 ppm, 1,700 ppm, 1,800 ppm, 1,900 ppm, or 2,000 ppm. In other aspects of this embodiment, a composition disclosed herein comprises a metal salt in an amount of, e.g., at least 0.05 ppm, at least 0.10 ppm, at least 0.20 ppm, at least 0.30 ppm, at least 0.40 ppm, at least 0.50 ppm, at least 0.60 ppm, at least 0.70 ppm, at least 0.80 ppm, at least 0.90 ppm, at least 1 ppm, at least 10 ppm, at least 20 ppm, at least 30 ppm, at least 40 ppm, at least 50 ppm, at least 60 ppm, at least 70 ppm, at least 80 ppm, at least 90 ppm, at least 100 ppm, at least 125 ppm, at least 150 ppm, at least 175 ppm, at least 200 ppm, at least 225 ppm, at least 250 ppm, at least 275 ppm, at least 300 ppm, at least 325 ppm, at least 350 ppm, at least 375 ppm, at least 400 ppm, at least 425 ppm, at least 450 ppm, at least 475 ppm, at least 500 ppm, at least 525 ppm, at least 550 ppm, at least 575 ppm, at least 600 ppm, at least 625 ppm, at least 650 ppm, at least 675 ppm, at least 700 ppm, at least 725 ppm, at least 750 ppm, at least 775 ppm, at least 800 ppm, at least 825 ppm, at least 850 ppm, at least 875 ppm, at least 900 ppm, at least 925 ppm, at least 950 ppm, at least 975 ppm, at least 1,000 ppm, at least 1,025 ppm, at least 1,050 ppm, at least 1075 ppm, at least 1,100 ppm, at least 1,125 ppm, at least 1,150 ppm, at least 1,175 ppm, at least 1,200 ppm, at least 1,225 ppm, at least 1,250 ppm, at least 1,275 ppm, at least 1,300 ppm, at least 1,325 ppm, at least 1,350 ppm, at least 1,375 ppm, at least 1,400 ppm, at least 1,425 ppm, at least 1,450 ppm, at least 1,475 ppm, at least 1,500 ppm, at least 1,600 ppm, at least 1,700 ppm, at least 1,800 ppm, at least 1,900 ppm, or at least 2,000 ppm. In yet other aspects of this embodiment, a composition disclosed herein comprises a metal salt in an amount of, e.g., at most 0.05 ppm, at most 0.10 ppm, at most 0.20 ppm, at most 0.30 ppm, at most 0.40 ppm, at most 0.50 ppm, at most 0.60 ppm, at most 0.70 ppm, at most 0.80 ppm, at most 0.90 ppm, at most 1 ppm, at most 10 ppm, at most 20 ppm, at most 30 ppm, at most 40 ppm, at most 50 ppm, at most 60 ppm, at most 70 ppm, at most 80 ppm, at most 90 ppm, at most 100 ppm, at most 125 ppm, at most 150 ppm, at most 175 ppm, at most 200 ppm, at most 225 ppm, at most 250 ppm, at most 275 ppm, at most 300 ppm, at most 325 ppm, at most 350 ppm, at most 375 ppm, at most 400 ppm, at most 425 ppm, at most 450 ppm, at most 475 ppm, at most 500 ppm, at most 525 ppm, at most 550 ppm, at most 575 ppm, at most 600 ppm, at most 625 ppm, at most 650 ppm, at most 675 ppm, at most 700 ppm, at most 725 ppm, at most 750 ppm, at most 775 ppm, at most 800 ppm, at most 825 ppm, at most 850 ppm, at most 875 ppm, at most 900 ppm, at most 925 ppm, at most 950 ppm, at most 975 ppm, at most 1,000 ppm, at most 1,025 ppm, at most 1,050 ppm, at most 1075 ppm, at most 1,100 ppm, at most 1,125 ppm, at most 1,150 ppm, at most 1,175 ppm, at most 1,200 ppm, at most 1,225 ppm, at most 1,250 ppm, at most 1,275 ppm, at most 1,300 ppm, at most 1,325 ppm, at most 1,350 ppm, at most 1,375 ppm, at most 1,400 ppm, at most 1,425 ppm, at most 1,450 ppm, at most 1,475 ppm, at most 1,500 ppm, at most 1,600 ppm, at most 1,700 ppm, at most 1,800 ppm, at most 1,900 ppm, or at most 2,000 ppm.

In still other aspects of this embodiment, a composition disclosed herein comprises a metal salt in an amount of from, e.g., about 0.5 ppm to about 20 ppm, about 0.5 ppm to about 25 ppm, about 0.5 ppm to about 30 ppm, about 0.5 ppm to about 35 ppm, about 0.5 ppm to about 40 ppm, about 0.5 ppm to about 45 ppm, about 0.5 ppm to about 50 ppm, about 0.5 ppm to about 55 ppm, about 0.5 ppm to about 60 ppm, about 0.5 ppm to about 65 ppm, about 0.5 ppm to about 70 ppm, about 0.5 ppm to about 75 ppm, about 0.5 ppm to about 80 ppm, about 0.5 ppm to about 85 ppm, about 0.5 ppm to about 90 ppm, about 0.5 ppm to about 95 ppm, about 0.5 ppm to about 100 ppm, about 0.75 ppm to about 20 ppm, about 0.75 ppm to about 25 ppm, about 0.75 ppm to about 30 ppm, about 0.75 ppm to about 35 ppm, about 0.75 ppm to about 40 ppm, about 0.75 ppm to about 45 ppm, about 0.75 ppm to about 50 ppm, about 0.75 ppm to about 55 ppm, about 0.75 ppm to about 60 ppm, about 0.75 ppm to about 65 ppm, about 0.75 ppm to about 70 ppm, about 0.75 ppm to about 75 ppm, about 0.75 ppm to about 80 ppm, about 0.75 ppm to about 85 ppm, about 0.75 ppm to about 90 ppm, about 0.75 ppm to about 95 ppm, about 0.75 ppm to about 100 ppm, about 1 ppm to about 20 ppm, about 1 ppm to about 25 ppm, about 1 ppm to about 30 ppm, about 1 ppm to about 35 ppm, about 1 ppm to about 40 ppm, about 1 ppm to about 45 ppm, about 1 ppm to about 50 ppm, about 1 ppm to about 55 ppm, about 1 ppm to about 60 ppm, about 1 ppm to about 65 ppm, about 1 ppm to about 70 ppm, about 1 ppm to about 75 ppm, about 1 ppm to about 80 ppm, about 1 ppm to about 85 ppm, about 1 ppm to about 90 ppm, about 1 ppm to about 95 ppm, about 1 ppm to about 100 ppm, about 5 ppm to about 20 ppm, about 5 ppm to about 25 ppm, about 5 ppm to about 30 ppm, about 5 ppm to about 35 ppm, about 5 ppm to about 40 ppm, about 5 ppm to about 45 ppm, about 5 ppm to about 50 ppm, about 5 ppm to about 55 ppm, about 5 ppm to about 60 ppm, about 5 ppm to about 65 ppm, about 5 ppm to about 70 ppm, about 5 ppm to about 75 ppm, about 5 ppm to about 80 ppm, about 5 ppm to about 85 ppm, about 5 ppm to about 90 ppm, about 5 ppm to about 95 ppm, about 5 ppm to about 100 ppm, about 10 ppm to about 20 ppm, about 10 ppm to about 25 ppm, about 10 ppm to about 30 ppm, about 10 ppm to about 35 ppm, about 10 ppm to about 40 ppm, about 10 ppm to about 45 ppm, about 10 ppm to about 50 ppm, about 10 ppm to about 55 ppm, about 10 ppm to about 60 ppm, about 10 ppm to about 65 ppm, about 10 ppm to about 70 ppm, about 10 ppm to about 75 ppm, about 10 ppm to about 80 ppm, about 10 ppm to about 85 ppm, about 10 ppm to about 90 ppm, about 10 ppm to about 95 ppm, or about 10 ppm to about 100 ppm.

In other aspects of this embodiment, a composition disclosed herein comprises a metal salt in an amount of from, e.g., about 1 ppm to about 25 ppm, about 1 ppm to about 50 ppm, about 1 ppm to about 75 ppm, about 1 ppm to about 100 ppm, about 1 ppm to about 125 ppm, about 1 ppm to about 150 ppm, about 1 ppm to about 175 ppm, about 1 ppm to about 200 ppm, about 1 ppm to about 225 ppm, about 1 ppm to about 250 ppm, about 1 ppm to about 275 ppm, about 1 ppm to about 300 ppm, about 1 ppm to about 325 ppm, about 1 ppm to about 350 ppm, about 1 ppm to about 375 ppm, about 1 ppm to about 400 ppm, about 1 ppm to about 425 ppm, about 1 ppm to about 450 ppm, about 1 ppm to about 475 ppm, about 1 ppm to about 500 ppm, about 5 ppm to about 25 ppm, about 5 ppm to about 50 ppm, about 5 ppm to about 75 ppm, about 5 ppm to about 100 ppm, about 5 ppm to about 125 ppm, about 5 ppm to about 150 ppm, about 5 ppm to about 175 ppm, about 5 ppm to about 200 ppm, about 5 ppm to about 225 ppm, about 5 ppm to about 250 ppm, about 5 ppm to about 275 ppm, about 1 ppm to about 300 ppm, about 5 ppm to about 325 ppm, about 5 ppm to about 350 ppm, about 5 ppm to about 375 ppm, about 5 ppm to about 400 ppm, about 5 ppm to about 425 ppm, about 5 ppm to about 450 ppm, about 5 ppm to about 475 ppm, about 5 ppm to about 500 ppm, about 10 ppm to about 25 ppm, about 10 ppm to about 50 ppm, about 10 ppm to about 75 ppm, about 10 ppm to about 100 ppm, about 10 ppm to about 125 ppm, about 10 ppm to about 150 ppm, about 10 ppm to about 175 ppm, about 10 ppm to about 200 ppm, about 10 ppm to about 225 ppm, about 10 ppm to about 250 ppm, about 10 ppm to about 275 ppm, about 10 ppm to about 300 ppm, about 10 ppm to about 325 ppm, about 10 ppm to about 350 ppm, about 10 ppm to about 375 ppm, about 10 ppm to about 400 ppm, about 10 ppm to about 425 ppm, about 10 ppm to about 450 ppm, about 10 ppm to about 475 ppm, about 10 ppm to about 500 ppm, about 25 ppm to about 50 ppm, about 25 ppm to about 75 ppm, about 25 ppm to about 100 ppm, about 25 ppm to about 125 ppm, about 25 ppm to about 150 ppm, about 25 ppm to about 175 ppm, about 25 ppm to about 200 ppm, about 25 ppm to about 225 ppm, about 25 ppm to about 250 ppm, about 25 ppm to about 275 ppm, about 25 ppm to about 300 ppm, about 25 ppm to about 325 ppm, about 25 ppm to about 350 ppm, about 25 ppm to about 375 ppm, about 25 ppm to about 400 ppm, about 25 ppm to about 425 ppm, about 25 ppm to about 450 ppm, about 25 ppm to about 475 ppm, about 25 ppm to about 500 ppm, about 50 ppm to about 75 ppm, about 50 ppm to about 100 ppm, about 50 ppm to about 125 ppm, about 50 ppm to about 150 ppm, about 50 ppm to about 175 ppm, about 50 ppm to about 200 ppm, about 50 ppm to about 225 ppm, about 50 ppm to about 250 ppm, about 50 ppm to about 275 ppm, about 50 ppm to about 300 ppm, about 50 ppm to about 325 ppm, about 50 ppm to about 350 ppm, about 50 ppm to about 375 ppm, about 50 ppm to about 400 ppm, about 50 ppm to about 425 ppm, about 50 ppm to about 450 ppm, about 50 ppm to about 475 ppm, about 50 ppm to about 500 ppm, about 75 ppm to about 100 ppm, about 75 ppm to about 125 ppm, about 75 ppm to about 150 ppm, about 75 ppm to about 175 ppm, about 75 ppm to about 200 ppm, about 75 ppm to about 225 ppm, about 75 ppm to about 250 ppm, about 75 ppm to about 275 ppm, about 75 ppm to about 300 ppm, about 75 ppm to about 325 ppm, about 75 ppm to about 350 ppm, about 75 ppm to about 375 ppm, about 75 ppm to about 400 ppm, about 75 ppm to about 425 ppm, about 75 ppm to about 450 ppm, about 75 ppm to about 475 ppm, about 75 ppm to about 500 ppm, about 100 ppm to about 125 ppm, about 100 ppm to about 150 ppm, about 100 ppm to about 175 ppm, about 100 ppm to about 200 ppm, about 100 ppm to about 225 ppm, about 100 ppm to about 250 ppm, about 100 ppm to about 275 ppm, about 100 ppm to about 300 ppm, about 100 ppm to about 325 ppm, about 100 ppm to about 350 ppm, about 100 ppm to about 375 ppm, about 100 ppm to about 400 ppm, about 100 ppm to about 425 ppm, about 100 ppm to about 450 ppm, about 100 ppm to about 475 ppm, about 100 ppm to about 500 ppm, about 150 ppm to about 175 ppm, about 150 ppm to about 200 ppm, about 150 ppm to about 225 ppm, about 150 ppm to about 250 ppm, about 150 ppm to about 275 ppm, about 150 ppm to about 300 ppm, about 150 ppm to about 325 ppm, about 150 ppm to about 350 ppm, about 150 ppm to about 375 ppm, about 150 ppm to about 400 ppm, about 150 ppm to about 425 ppm, about 150 ppm to about 450 ppm, about 150 ppm to about 475 ppm, about 150 ppm to about 500 ppm, about 200 ppm to about 225 ppm, about 200 ppm to about 250 ppm, about 200 ppm to about 275 ppm, about 200 ppm to about 300 ppm, about 200 ppm to about 325 ppm, about 200 ppm to about 350 ppm, about 200 ppm to about 375 ppm, about 200 ppm to about 400 ppm, about 200 ppm to about 425 ppm, about 200 ppm to about 450 ppm, about 200 ppm to about 475 ppm, about 200 ppm to about 500 ppm, about 250 ppm to about 275 ppm, about 250 ppm to about 300 ppm, about 250 ppm to about 325 ppm, about 250 ppm to about 350 ppm, about 250 ppm to about 375 ppm, about 250 ppm to about 400 ppm, about 250 ppm to about 425 ppm, about 250 ppm to about 450 ppm, about 250 ppm to about 475 ppm, about 250 ppm to about 500 ppm, about 300 ppm to about 325 ppm, about 300 ppm to about 350 ppm, about 300 ppm to about 375 ppm, about 300 ppm to about 400 ppm, about 300 ppm to about 425 ppm, about 300 ppm to about 450 ppm, about 300 ppm to about 475 ppm, about 300 ppm to about 500 ppm, about 350 ppm to about 375 ppm, about 350 ppm to about 400 ppm, about 350 ppm to about 425 ppm, about 350 ppm to about 450 ppm, about 350 ppm to about 475 ppm, about 350 ppm to about 500 ppm, about 400 ppm to about 425 ppm, about 400 ppm to about 450 ppm, about 400 ppm to about 475 ppm, about 400 ppm to about 500 ppm, about 450 ppm to about 475 ppm, or about 450 ppm to about 500 ppm.

In other aspects of this embodiment, a composition disclosed herein comprises a metal salt in an amount of, e.g., about 400 ppm to about 500 ppm, about 400 ppm to about 600 ppm, about 400 ppm to about 700 ppm, about 400 ppm to about 800 ppm, about 400 ppm to about 900 ppm, about 400 ppm to about 1,000 ppm, about 400 ppm to about 1,100 ppm, about 400 ppm to about 1,200 ppm, about 400 ppm to about 1,300 ppm, about 400 ppm to about 1,400 ppm, about 400 ppm to about 1,500 ppm, about 400 ppm to about 1,600 ppm, about 400 ppm to about 1,700 ppm, about 400 ppm to about 1,800 ppm, about 400 ppm to about 1,900 ppm, about 400 ppm to about 2,000 ppm, about 500 ppm to about 600 ppm, about 500 ppm to about 700 ppm, about 500 ppm to about 800 ppm, about 500 ppm to about 900 ppm, about 500 ppm to about 1,000 ppm, about 500 ppm to about 1,100 ppm, about 500 ppm to about 1,200 ppm, about 500 ppm to about 1,300 ppm, about 500 ppm to about 1,400 ppm, about 500 ppm to about 1,500 ppm, about 500 ppm to about 1,600 ppm, about 500 ppm to about 1,700 ppm, about 500 ppm to about 1,800 ppm, about 500 ppm to about 1,900 ppm, about 500 ppm to about 2,000 ppm, about 600 ppm to about 700 ppm, about 600 ppm to about 800 ppm, about 600 ppm to about 900 ppm, about 600 ppm to about 1,000 ppm, about 600 ppm to about 1,100 ppm, about 600 ppm to about 1,200 ppm, about 600 ppm to about 1,300 ppm, about 600 ppm to about 1,400 ppm, about 600 ppm to about 1,500 ppm, about 600 ppm to about 1,600 ppm, about 600 ppm to about 1,700 ppm, about 600 ppm to about 1,800 ppm, about 600 ppm to about 1,900 ppm, about 600 ppm to about 2,000 ppm, about 700 ppm to about 800 ppm, about 700 ppm to about 900 ppm, about 700 ppm to about 1,000 ppm, about 700 ppm to about 1,100 ppm, about 700 ppm to about 1,200 ppm, about 700 ppm to about 1,300 ppm, about 700 ppm to about 1,400 ppm, about 700 ppm to about 1,500 ppm, about 700 ppm to about 1,600 ppm, about 700 ppm to about 1,700 ppm, about 700 ppm to about 1,800 ppm, about 700 ppm to about 1,900 ppm, about 700 ppm to about 2,000 ppm, about 800 ppm to about 900 ppm, about 800 ppm to about 1,000 ppm, about 800 ppm to about 1,100 ppm, about 800 ppm to about 1,200 ppm, about 800 ppm to about 1,300 ppm, about 800 ppm to about 1,400 ppm, about 800 ppm to about 1,500 ppm, about 800 ppm to about 1,600 ppm, about 800 ppm to about 1,700 ppm, about 800 ppm to about 1,800 ppm, about 800 ppm to about 1,900 ppm, about 800 ppm to about 2,000 ppm, about 900 ppm to about 1,000 ppm, about 900 ppm to about 1,100 ppm, about 900 ppm to about 1,200 ppm, about 900 ppm to about 1,300 ppm, about 900 ppm to about 1,400 ppm, about 900 ppm to about 1,500 ppm, about 900 ppm to about 1,600 ppm, about 900 ppm to about 1,700 ppm, about 900 ppm to about 1,800 ppm, about 900 ppm to about 1,900 ppm, about 900 ppm to about 2,000 ppm, about 1,000 ppm to about 1,100 ppm, about 1,000 ppm to about 1,200 ppm, about 1,000 ppm to about 1,300 ppm, about 1,000 ppm to about 1,400 ppm, about 1,000 ppm to about 1,500 ppm, about 1,000 ppm to about 1,600 ppm, about 1,000 ppm to about 1,700 ppm, about 1,000 ppm to about 1,800 ppm, about 1,000 ppm to about 1,900 ppm, about 1,000 ppm to about 2,000 ppm, about 1,100 ppm to about 1,200 ppm, about 1,100 ppm to about 1,300 ppm, about 1,100 ppm to about 1,400 ppm, about 1,100 ppm to about 1,500 ppm, about 1,100 ppm to about 1,600 ppm, about 1,100 ppm to about 1,700 ppm, about 1,100 ppm to about 1,800 ppm, about 1,100 ppm to about 1,900 ppm, about 1,100 ppm to about 2,000 ppm, about 1,200 ppm to about 1,300 ppm, about 1,200 ppm to about 1,400 ppm, about 1,200 ppm to about 1,500 ppm, about 1,200 ppm to about 1,600 ppm, about 1,200 ppm to about 1,700 ppm, about 1,200 ppm to about 1,800 ppm, about 1,200 ppm to about 1,900 ppm, about 1,200 ppm to about 2,000 ppm, about 1,300 ppm to about 1,400 ppm, about 1,300 ppm to about 1,500 ppm, about 1,300 ppm to about 1,600 ppm, about 1,300 ppm to about 1,700 ppm, about 1,300 ppm to about 1,800 ppm, about 1,300 ppm to about 1,900 ppm, about 1,300 ppm to about 2,000 ppm, about 1,400 ppm to about 1,500 ppm, about 1,400 ppm to about 1,600 ppm, about 1,400 ppm to about 1,700 ppm, about 1,400 ppm to about 1,800 ppm, about 1,400 ppm to about 1,900 ppm, about 1,400 ppm to about 2,000 ppm, about 1,500 ppm to about 1,600 ppm, about 1,500 ppm to about 1,700 ppm, about 1,500 ppm to about 1,800 ppm, about 1,500 ppm to about 1,900 ppm, about 1,500 ppm to about 2,000 ppm, about 1,600 ppm to about 1,700 ppm, about 1,600 ppm to about 1,800 ppm, about 1,600 ppm to about 1,900 ppm, about 1,600 ppm to about 2,000 ppm, about 1,700 ppm to about 1,800 ppm, about 1,700 ppm to about 1,900 ppm, about 1,700 ppm to about 2,000 ppm, about 1,800 ppm to about 1,900 ppm, about 1,800 ppm to about 2,000 ppm, or about 1,900 ppm to about 2,000 ppm.

In aspects of this embodiment, a composition disclosed herein comprises a metal salt in an amount of, e.g., about 0.001%, about 0.005%, about 0.01%, about 0.05%, about 0.10%, about 0.11%, about 0.12%, about 0.13%, about 0.14%, about 0.15%, about 0.16%, about 0.17%, about 0.18%, about 0.19%, about 0.2%, about 0.25%, about 0.3%, about 0.35%, about 0.4%, about 0.45%, or about 0.5% by weight of the composition. In other aspects of this embodiment, a composition disclosed herein comprises a metal salt in an amount of, e.g., at least 0.001%, at least 0.005%, at least 0.01%, at least 0.05%, at least 0.10%, at least 0.11%, at least 0.12%, at least 0.13%, at least 0.14%, at least 0.15%, at least 0.16%, at least 0.17%, at least 0.18%, at least 0.19%, at least 0.2%, at least 0.25%, at least 0.3%, at least 0.35%, at least 0.4%, at least 0.45%, or at least 0.5% by weight of the composition. In yet other aspects of this embodiment, a composition disclosed herein comprises a metal salt in an amount of, e.g., at most 0.001%, at most 0.005%, at most 0.01%, at most 0.05%, at most 0.10%, at most 0.11%, at most 0.12%, at most 0.13%, at most 0.14%, at most 0.15%, at most 0.16%, at most 0.17%, at most 0.18%, at most 0.19%, at most 0.2%, at most 0.25%, at most 0.3%, at most 0.35%, at most 0.4%, at most 0.45%, or at most 0.5% by weight of the composition. In still other aspects of this embodiment, a composition disclosed herein comprises a metal salt in an amount of, e.g., about 0.0010% to about 0.005%, about 0.0010% to about 0.010%, about 0.0010% to about 0.050%, about 0.0010% to about 0.10%, about 0.0010% to about 0.15%, about 0.0010% to about 0.20%, about 0.0010% to about 0.25%, about 0.0010% to about 0.30%, about 0.0010% to about 0.35%, about 0.0010% to about 0.40%, about 0.0010% to about 0.45%, about 0.0010% to about 0.50%, about 0.010% to about 0.050%, about 0.010% to about 0.10%, about 0.010% to about 0.15%, about 0.010% to about 0.20%, about 0.010% to about 0.25%, about 0.010% to about 0.30%, about 0.010% to about 0.35%, about 0.010% to about 0.40%, about 0.010% to about 0.45%, about 0.010% to about 0.50%, about 0.10% to about 0.15%, about 0.10% to about 0.20%, about 0.10% to about 0.25%, about 0.10% to about 0.30%, about 0.10% to about 0.35%, about 0.10% to about 0.40%, about 0.10% to about 0.45%, about 0.10% to about 0.50%, about 0.20% to about 0.25%, about 0.20% to about 0.30%, about 0.20% to about 0.35%, about 0.20% to about 0.40%, about 0.20% to about 0.45%, about 0.20% to about 0.50%, about 0.30% to about 0.35%, about 0.30% to about 0.40%, about 0.30% to about 0.45%, about 0.30% to about 0.50%, about 0.40% to about 0.45%, about 0.40% to about 0.50%, or about 0.40% to about 0.50% by weight of the composition.

In an embodiment, a composition disclosed herein does not comprise metal salts.

A composition disclosed herein may comprise a phosphate buffer. A phosphate buffer or phosphate buffer solution) is especially sensitive to pH changes, and thus is useful for biological applications. A phosphate buffer can be adapted to a variety of pH levels, including isotonic. This wide range is due to phosphoric acid having 3 dissociation constants (pKa) allowing for formulation of buffers near each of the pH levels of 2.15, 7.21, or 12.32. Monosodium phosphate and its conjugate base, disodium phosphate, are usually used to generate buffers of pH values around 7. Non-limiting examples of a phosphate buffer include a calcium phosphate buffer and a sodium phosphate buffer.

In an embodiment, a composition disclosed herein comprises an amount of a phosphate buffer that provides a desired beneficial effect to a composition disclosed herein. In aspects of this embodiment, a composition disclosed herein comprises a phosphate buffer in an amount of, e.g., 0.05 ppm, 0.10 ppm, 0.15 ppm, 0.20 ppm, 0.25 ppm, 0.30 ppm, 0.35 ppm, 0.40 ppm, 0.45 ppm, 0.50 ppm, 0.55 ppm, 0.60 ppm, 0.65 ppm, 0.70 ppm, 0.75 ppm, 0.80 ppm, 0.85 ppm, 0.90 ppm, 0.95 ppm, 1 ppm, 5 ppm, 10 ppm, 15 ppm, 20 ppm, 25 ppm, 30 ppm, 35 ppm, 40 ppm, 45 ppm, 50 ppm, 55 ppm, 60 ppm, 65 ppm, 70 ppm, 75 ppm, 80 ppm, 85 ppm, 90 ppm, 95 ppm, 100 ppm, 125 ppm, 150 ppm, 175 ppm, 200 ppm, 225 ppm, 250 ppm, 275 ppm, 300 ppm, 325 ppm, 350 ppm, 375 ppm, 400 ppm, 425 ppm, 450 ppm, 475 ppm, 500 ppm, 525 ppm, 550 ppm, 575 ppm, 600 ppm, 625 ppm, 650 ppm, 675 ppm, 700 ppm, 725 ppm, 750 ppm, 775 ppm, 800 ppm, 825 ppm, 850 ppm, 875 ppm, 900 ppm, 925 ppm, 950 ppm, 975 ppm, 1,000 ppm, 1,025 ppm, 1,050 ppm, 1075 ppm, 1,100 ppm, 1,125 ppm, 1,150 ppm, 1,175 ppm, 1,200 ppm, 1,225 ppm, 1,250 ppm, 1,275 ppm, 1,300 ppm, 1,325 ppm, 1,350 ppm, 1,375 ppm, 1,400 ppm, 1,425 ppm, 1,450 ppm, 1,475 ppm, 1,500 ppm, 1,600 ppm, 1,700 ppm, 1,800 ppm, 1,900 ppm, or 2,000 ppm. In other aspects of this embodiment, a composition disclosed herein comprises a phosphate buffer in an amount of, e.g., at least 0.05 ppm, at least 0.10 ppm, at least 0.20 ppm, at least 0.30 ppm, at least 0.40 ppm, at least 0.50 ppm, at least 0.60 ppm, at least 0.70 ppm, at least 0.80 ppm, at least 0.90 ppm, at least 1 ppm, at least 10 ppm, at least 20 ppm, at least 30 ppm, at least 40 ppm, at least 50 ppm, at least 60 ppm, at least 70 ppm, at least 80 ppm, at least 90 ppm, at least 100 ppm, at least 125 ppm, at least 150 ppm, at least 175 ppm, at least 200 ppm, at least 225 ppm, at least 250 ppm, at least 275 ppm, at least 300 ppm, at least 325 ppm, at least 350 ppm, at least 375 ppm, at least 400 ppm, at least 425 ppm, at least 450 ppm, at least 475 ppm, at least 500 ppm, at least 525 ppm, at least 550 ppm, at least 575 ppm, at least 600 ppm, at least 625 ppm, at least 650 ppm, at least 675 ppm, at least 700 ppm, at least 725 ppm, at least 750 ppm, at least 775 ppm, at least 800 ppm, at least 825 ppm, at least 850 ppm, at least 875 ppm, at least 900 ppm, at least 925 ppm, at least 950 ppm, at least 975 ppm, at least 1,000 ppm, at least 1,025 ppm, at least 1,050 ppm, at least 1075 ppm, at least 1,100 ppm, at least 1,125 ppm, at least 1,150 ppm, at least 1,175 ppm, at least 1,200 ppm, at least 1,225 ppm, at least 1,250 ppm, at least 1,275 ppm, at least 1,300 ppm, at least 1,325 ppm, at least 1,350 ppm, at least 1,375 ppm, at least 1,400 ppm, at least 1,425 ppm, at least 1,450 ppm, at least 1,475 ppm, at least 1,500 ppm, at least 1,600 ppm, at least 1,700 ppm, at least 1,800 ppm, at least 1,900 ppm, or at least 2,000 ppm. In yet other aspects of this embodiment, a composition disclosed herein comprises a phosphate buffer in an amount of, e.g., at most 0.05 ppm, at most 0.10 ppm, at most 0.20 ppm, at most 0.30 ppm, at most 0.40 ppm, at most 0.50 ppm, at most 0.60 ppm, at most 0.70 ppm, at most 0.80 ppm, at most 0.90 ppm, at most 1 ppm, at most 10 ppm, at most 20 ppm, at most 30 ppm, at most 40 ppm, at most 50 ppm, at most 60 ppm, at most 70 ppm, at most 80 ppm, at most 90 ppm, at most 100 ppm, at most 125 ppm, at most 150 ppm, at most 175 ppm, at most 200 ppm, at most 225 ppm, at most 250 ppm, at most 275 ppm, at most 300 ppm, at most 325 ppm, at most 350 ppm, at most 375 ppm, at most 400 ppm, at most 425 ppm, at most 450 ppm, at most 475 ppm, at most 500 ppm, at most 525 ppm, at most 550 ppm, at most 575 ppm, at most 600 ppm, at most 625 ppm, at most 650 ppm, at most 675 ppm, at most 700 ppm, at most 725 ppm, at most 750 ppm, at most 775 ppm, at most 800 ppm, at most 825 ppm, at most 850 ppm, at most 875 ppm, at most 900 ppm, at most 925 ppm, at most 950 ppm, at most 975 ppm, at most 1,000 ppm, at most 1,025 ppm, at most 1,050 ppm, at most 1075 ppm, at most 1,100 ppm, at most 1,125 ppm, at most 1,150 ppm, at most 1,175 ppm, at most 1,200 ppm, at most 1,225 ppm, at most 1,250 ppm, at most 1,275 ppm, at most 1,300 ppm, at most 1,325 ppm, at most 1,350 ppm, at most 1,375 ppm, at most 1,400 ppm, at most 1,425 ppm, at most 1,450 ppm, at most 1,475 ppm, at most 1,500 ppm, at most 1,600 ppm, at most 1,700 ppm, at most 1,800 ppm, at most 1,900 ppm, or at most 2,000 ppm.

In still other aspects of this embodiment, a composition disclosed herein comprises a phosphate buffer in an amount of from, e.g., about 0.5 ppm to about 20 ppm, about 0.5 ppm to about 25 ppm, about 0.5 ppm to about 30 ppm, about 0.5 ppm to about 35 ppm, about 0.5 ppm to about 40 ppm, about 0.5 ppm to about 45 ppm, about 0.5 ppm to about 50 ppm, about 0.5 ppm to about 55 ppm, about 0.5 ppm to about 60 ppm, about 0.5 ppm to about 65 ppm, about 0.5 ppm to about 70 ppm, about 0.5 ppm to about 75 ppm, about 0.5 ppm to about 80 ppm, about 0.5 ppm to about 85 ppm, about 0.5 ppm to about 90 ppm, about 0.5 ppm to about 95 ppm, about 0.5 ppm to about 100 ppm, about 0.75 ppm to about 20 ppm, about 0.75 ppm to about 25 ppm, about 0.75 ppm to about 30 ppm, about 0.75 ppm to about 35 ppm, about 0.75 ppm to about 40 ppm, about 0.75 ppm to about 45 ppm, about 0.75 ppm to about 50 ppm, about 0.75 ppm to about 55 ppm, about 0.75 ppm to about 60 ppm, about 0.75 ppm to about 65 ppm, about 0.75 ppm to about 70 ppm, about 0.75 ppm to about 75 ppm, about 0.75 ppm to about 80 ppm, about 0.75 ppm to about 85 ppm, about 0.75 ppm to about 90 ppm, about 0.75 ppm to about 95 ppm, about 0.75 ppm to about 100 ppm, about 1 ppm to about 20 ppm, about 1 ppm to about 25 ppm, about 1 ppm to about 30 ppm, about 1 ppm to about 35 ppm, about 1 ppm to about 40 ppm, about 1 ppm to about 45 ppm, about 1 ppm to about 50 ppm, about 1 ppm to about 55 ppm, about 1 ppm to about 60 ppm, about 1 ppm to about 65 ppm, about 1 ppm to about 70 ppm, about 1 ppm to about 75 ppm, about 1 ppm to about 80 ppm, about 1 ppm to about 85 ppm, about 1 ppm to about 90 ppm, about 1 ppm to about 95 ppm, about 1 ppm to about 100 ppm, about 5 ppm to about 20 ppm, about 5 ppm to about 25 ppm, about 5 ppm to about 30 ppm, about 5 ppm to about 35 ppm, about 5 ppm to about 40 ppm, about 5 ppm to about 45 ppm, about 5 ppm to about 50 ppm, about 5 ppm to about 55 ppm, about 5 ppm to about 60 ppm, about 5 ppm to about 65 ppm, about 5 ppm to about 70 ppm, about 5 ppm to about 75 ppm, about 5 ppm to about 80 ppm, about 5 ppm to about 85 ppm, about 5 ppm to about 90 ppm, about 5 ppm to about 95 ppm, about 5 ppm to about 100 ppm, about 10 ppm to about 20 ppm, about 10 ppm to about 25 ppm, about 10 ppm to about 30 ppm, about 10 ppm to about 35 ppm, about 10 ppm to about 40 ppm, about 10 ppm to about 45 ppm, about 10 ppm to about 50 ppm, about 10 ppm to about 55 ppm, about 10 ppm to about 60 ppm, about 10 ppm to about 65 ppm, about 10 ppm to about 70 ppm, about 10 ppm to about 75 ppm, about 10 ppm to about 80 ppm, about 10 ppm to about 85 ppm, about 10 ppm to about 90 ppm, about 10 ppm to about 95 ppm, or about 10 ppm to about 100 ppm.

In other aspects of this embodiment, a composition disclosed herein comprises a phosphate buffer in an amount of from, e.g., about 1 ppm to about 25 ppm, about 1 ppm to about 50 ppm, about 1 ppm to about 75 ppm, about 1 ppm to about 100 ppm, about 1 ppm to about 125 ppm, about 1 ppm to about 150 ppm, about 1 ppm to about 175 ppm, about 1 ppm to about 200 ppm, about 1 ppm to about 225 ppm, about 1 ppm to about 250 ppm, about 1 ppm to about 275 ppm, about 1 ppm to about 300 ppm, about 1 ppm to about 325 ppm, about 1 ppm to about 350 ppm, about 1 ppm to about 375 ppm, about 1 ppm to about 400 ppm, about 1 ppm to about 425 ppm, about 1 ppm to about 450 ppm, about 1 ppm to about 475 ppm, about 1 ppm to about 500 ppm, about 5 ppm to about 25 ppm, about 5 ppm to about 50 ppm, about 5 ppm to about 75 ppm, about 5 ppm to about 100 ppm, about 5 ppm to about 125 ppm, about 5 ppm to about 150 ppm, about 5 ppm to about 175 ppm, about 5 ppm to about 200 ppm, about 5 ppm to about 225 ppm, about 5 ppm to about 250 ppm, about 5 ppm to about 275 ppm, about 1 ppm to about 300 ppm, about 5 ppm to about 325 ppm, about 5 ppm to about 350 ppm, about 5 ppm to about 375 ppm, about 5 ppm to about 400 ppm, about 5 ppm to about 425 ppm, about 5 ppm to about 450 ppm, about 5 ppm to about 475 ppm, about 5 ppm to about 500 ppm, about 10 ppm to about 25 ppm, about 10 ppm to about 50 ppm, about 10 ppm to about 75 ppm, about 10 ppm to about 100 ppm, about 10 ppm to about 125 ppm, about 10 ppm to about 150 ppm, about 10 ppm to about 175 ppm, about 10 ppm to about 200 ppm, about 10 ppm to about 225 ppm, about 10 ppm to about 250 ppm, about 10 ppm to about 275 ppm, about 10 ppm to about 300 ppm, about 10 ppm to about 325 ppm, about 10 ppm to about 350 ppm, about 10 ppm to about 375 ppm, about 10 ppm to about 400 ppm, about 10 ppm to about 425 ppm, about 10 ppm to about 450 ppm, about 10 ppm to about 475 ppm, about 10 ppm to about 500 ppm, about 25 ppm to about 50 ppm, about 25 ppm to about 75 ppm, about 25 ppm to about 100 ppm, about 25 ppm to about 125 ppm, about 25 ppm to about 150 ppm, about 25 ppm to about 175 ppm, about 25 ppm to about 200 ppm, about 25 ppm to about 225 ppm, about 25 ppm to about 250 ppm, about 25 ppm to about 275 ppm, about 25 ppm to about 300 ppm, about 25 ppm to about 325 ppm, about 25 ppm to about 350 ppm, about 25 ppm to about 375 ppm, about 25 ppm to about 400 ppm, about 25 ppm to about 425 ppm, about 25 ppm to about 450 ppm, about 25 ppm to about 475 ppm, about 25 ppm to about 500 ppm, about 50 ppm to about 75 ppm, about 50 ppm to about 100 ppm, about 50 ppm to about 125 ppm, about 50 ppm to about 150 ppm, about 50 ppm to about 175 ppm, about 50 ppm to about 200 ppm, about 50 ppm to about 225 ppm, about 50 ppm to about 250 ppm, about 50 ppm to about 275 ppm, about 50 ppm to about 300 ppm, about 50 ppm to about 325 ppm, about 50 ppm to about 350 ppm, about 50 ppm to about 375 ppm, about 50 ppm to about 400 ppm, about 50 ppm to about 425 ppm, about 50 ppm to about 450 ppm, about 50 ppm to about 475 ppm, about 50 ppm to about 500 ppm, about 75 ppm to about 100 ppm, about 75 ppm to about 125 ppm, about 75 ppm to about 150 ppm, about 75 ppm to about 175 ppm, about 75 ppm to about 200 ppm, about 75 ppm to about 225 ppm, about 75 ppm to about 250 ppm, about 75 ppm to about 275 ppm, about 75 ppm to about 300 ppm, about 75 ppm to about 325 ppm, about 75 ppm to about 350 ppm, about 75 ppm to about 375 ppm, about 75 ppm to about 400 ppm, about 75 ppm to about 425 ppm, about 75 ppm to about 450 ppm, about 75 ppm to about 475 ppm, about 75 ppm to about 500 ppm, about 100 ppm to about 125 ppm, about 100 ppm to about 150 ppm, about 100 ppm to about 175 ppm, about 100 ppm to about 200 ppm, about 100 ppm to about 225 ppm, about 100 ppm to about 250 ppm, about 100 ppm to about 275 ppm, about 100 ppm to about 300 ppm, about 100 ppm to about 325 ppm, about 100 ppm to about 350 ppm, about 100 ppm to about 375 ppm, about 100 ppm to about 400 ppm, about 100 ppm to about 425 ppm, about 100 ppm to about 450 ppm, about 100 ppm to about 475 ppm, about 100 ppm to about 500 ppm, about 150 ppm to about 175 ppm, about 150 ppm to about 200 ppm, about 150 ppm to about 225 ppm, about 150 ppm to about 250 ppm, about 150 ppm to about 275 ppm, about 150 ppm to about 300 ppm, about 150 ppm to about 325 ppm, about 150 ppm to about 350 ppm, about 150 ppm to about 375 ppm, about 150 ppm to about 400 ppm, about 150 ppm to about 425 ppm, about 150 ppm to about 450 ppm, about 150 ppm to about 475 ppm, about 150 ppm to about 500 ppm, about 200 ppm to about 225 ppm, about 200 ppm to about 250 ppm, about 200 ppm to about 275 ppm, about 200 ppm to about 300 ppm, about 200 ppm to about 325 ppm, about 200 ppm to about 350 ppm, about 200 ppm to about 375 ppm, about 200 ppm to about 400 ppm, about 200 ppm to about 425 ppm, about 200 ppm to about 450 ppm, about 200 ppm to about 475 ppm, about 200 ppm to about 500 ppm, about 250 ppm to about 275 ppm, about 250 ppm to about 300 ppm, about 250 ppm to about 325 ppm, about 250 ppm to about 350 ppm, about 250 ppm to about 375 ppm, about 250 ppm to about 400 ppm, about 250 ppm to about 425 ppm, about 250 ppm to about 450 ppm, about 250 ppm to about 475 ppm, about 250 ppm to about 500 ppm, about 300 ppm to about 325 ppm, about 300 ppm to about 350 ppm, about 300 ppm to about 375 ppm, about 300 ppm to about 400 ppm, about 300 ppm to about 425 ppm, about 300 ppm to about 450 ppm, about 300 ppm to about 475 ppm, about 300 ppm to about 500 ppm, about 350 ppm to about 375 ppm, about 350 ppm to about 400 ppm, about 350 ppm to about 425 ppm, about 350 ppm to about 450 ppm, about 350 ppm to about 475 ppm, about 350 ppm to about 500 ppm, about 400 ppm to about 425 ppm, about 400 ppm to about 450 ppm, about 400 ppm to about 475 ppm, about 400 ppm to about 500 ppm, about 450 ppm to about 475 ppm, or about 450 ppm to about 500 ppm.

In an embodiment, a composition disclosed herein does not comprise a phosphate buffer. In an embodiment, a composition disclosed herein does not comprise a calcium phosphate buffer. In an embodiment, a composition disclosed herein does not comprise a sodium phosphate buffer.

A composition disclosed herein may comprise a surfactant. Surfactants are compounds that lower the surface tension of a liquid, allowing easier spreading, and lowering of the interfacial tension between two liquids, or between a liquid and a solid. By acting at the interface of two surfaces, surfactants modulate the interfacial tension resulting in the formation of the foam. Either a single surfactant may be mixed with the buffered solution disclosed herein, or a plurality of surfactants may be mixed with the buffered solution disclosed herein. Useful surfactants, include, without limitation, ionic surfactants, zwitterionic (amphoteric) surfactants, non-ionic surfactants, or any combination therein. The surfactant used in a composition disclosed herein can be varied as appropriate by one skilled in the art and generally depends, in part, on the particular buffer being used, the protein being eluted, and the conductivity values being employed.

Ionic surfactants include anionic surfactants. Anionic surfactants include ones based on permanent functional groups attached to the head, such as, e.g., sulfate, sulfonate, phosphate carboxylates) or pH dependent anionic surfactants. Anionic surfactants include, without limitation, alkyl sulfates like ammonium lauryl sulfate and sodium lauryl sulfate (SDS); alkyl ether sulfates like sodium laureth sulfate and sodium myreth sulfate; docusates like dioctyl sodium sulfosuccinate; sulfonate fluorosurfactants like perfluorooctanesulfonate (PFOS) and perfluorobutanesulfonate; alkyldiphenyloxide Disulfonates like DOWFAX™ 2A1 (Disodium Lauryl Phenyl Ether Disulfonate), DOWFAX™ 3B2 (Disodium Decyl Phenyl Ether Disulfonate), DOWFAX™ C10L (Disodium Decyl Phenyl Ether Disulfonate), DOWFAX™ 2EP, and DOWFAX™ 8390 (Disodium Cetyl Phenyl Ether Disulfonate); potassium phosphate polyether esters like TRITON™ H-55 and TRITON™ H-66; alkyl benzene sulfonates; alkyl aryl ether phosphates; alkyl ether phosphates; alkyl carboxylates like fatty acid salts and sodium stearate; sodium lauroyl sarcosinate; carboxylate fluorosurfactants like perfluorononanoate and perfluorooctanoate; and Sodium Hexyldiphenyl Ether Sulfonate (DOWFAX™ C6L).

Ionic surfactants also include cationic surfactants. Cationic surfactants include ones based on permanent or pH dependent cationic surfactants, such as, e.g., primary, secondary or tertiary amines. Cationic surfactants include, without limitation, quaternary ammonium surfactants like alkyl-trimethylammonium salts and alkyl-benzyldimethylammonium salts as well as pH-dependent primary, secondary or tertiary amines like surfactants where the primary amines become positively charged at pH greater than 10, or the secondary amines become charged at pH less than 4, like octenidine dihydrochloride. Non-limiting examples of quaternary ammonium surfactants include first generation quaternary ammonium surfactants comprising C12-18 alkyl chains, second generation quaternary ammonium surfactants comprising aromatic ring groups, third generation quaternary ammonium surfactants comprising dual first generation quaternary ammonium compounds and/or mixtures of alkyl dimethyl ammonium chloride, fourth generation quaternary ammonium surfactants comprising dialkylmethyl aminos with twin chains, fifth generation quaternary ammonium surfactants comprising synergistic combinations of dual quaternary ammonium compounds, six generation quaternary ammonium surfactants comprising polymeric quaternary ammonium compounds, and seventh generation quaternary ammonium surfactants comprising bis-quaternary ammonium compounds with polymeric quaternary ammonium compounds. Quaternary ammonium surfactants include, without limitation, benzalkonium chloride (BAC), benzethonium chloride (BZT), benzododecinium bromide (or dimethyldodecylbenzylammonium bromide), bronidox, (or 5-bromo-5-nitro-1,3-dioxane), carbethopendecinium bromide, cetalkonium chloride (CKC), cetrimonium, cetrimide, cetylpyridinium chloride (CPC), cetylpyridinium bromide, cetyl trimethylammonium bromide (CTAB or cetrimonium bromide) and cetyl trimethylammonium chloride (CTAC or cetrimonium chloride), didecyldimethylammonium chloride (DDAC), dioctadecyldimethylammonium bromide (DODAB or dimethyldioctadecylammonium bromide), dioctadecyldimethylammonium chloride (DODAC or dimethyldioctadecylammonium chloride), docosyltrimethylammonium chloride (DCTAC or behentrimonium chloride), dofanium chloride, domiphen bromide, methylbenzethonium chloride, octenidine dihydrochloride, polidronium chloride, stearalkonium chloride (or dimethylbenzyloctadecylammonium), tetraethylammonium bromide, tetramethylammonium hydroxide (TMAH) and thonzonium bromide. Other useful anionic surfactants include bio-based anionic surfactants, including, without limitation, STEPONOL® AM 30-KE, an ammonium lauryl sulfate, and STEPONOL® EHS, a sodium 2-ethyl hexyl sulfate. Such bio-based surfactants are not synthetic molecules, but instead are anionic biosurfactants derived from organic matter such as plants.

Zwitterionic surfactants are based on primary, secondary or tertiary amines or quaternary ammonium cation with a sulfonate, a carboxylate, or a phosphate. Zwitterionic surfactants include, without limitation, 3-[(3-Cholamidopropyl)dimethylammonio]-1-propanesulfonate (CHAPS); sultaines like cocamidopropyl hydroxysultaine; betaines like cocamidopropyl betaine; or lecithins.

Non-ionic surfactants are less denaturing and as such are useful to solubilize membrane proteins and lipids while retaining protein-protein interactions. Nonionic surfactants include polyether nonionic surfactants, polyhydroxyl nonionic surfactants and biosurfactants. Nonionic surfactants include alcohol ethoxylates, alkylphenol ethoxylates, phenol ethoxylates, amide ethoxylates, glyceride ethoxylates, fatty acid ethoxylates, fatty amine ethoxylates and polyethoxylated tallow amine (POEA). Other useful non-ionic surfactants include bio-based non-ionic surfactants, including, without limitation, STEPOSOL® MET-10U, a metathesis-derived, nonionic surfactant that is an unsaturated, short chain amide. Such bio-based surfactants are not synthetic molecules, but instead are non-ionic biosurfactants derived from organic matter such as plants.

Non-limiting examples of surfactants include polyoxyethylene glycol sorbitan alkyl esters (or ethoxylated sorbital esters) like polysorbate 20 sorbitan monooleate (TWEEN® 20), polysorbate 40 sorbitan monooleate (TWEEN® 40), polysorbate 60 sorbitan monooleate (TWEEN® 60), polysorbate 61 sorbitan monooleate (TWEEN® 61), polysorbate 65 sorbitan monooleate (TWEEN® 65), polysorbate 80 sorbitan monooleate (TWEEN® 80), polysorbate 81 sorbitan monooleate (TWEEN® 81) and polysorbate 85 sorbitan monooleate (TWEEN® 85); sorbital esters like sorbitan monooleate, sorbitan monolaurate, sorbitan monopalmitate, sorbitan monostearate and sorbitan tristearate; polyglycerol esters like glycerol monooleate, glycerol monolaurate, glycerol monopalmitate, glycerol monostearate, glycerol trioleate, glycerol ricinoleate, glycerol tristearate, mono diglycerides and glycerol triacetate; ethoxylated polyglycerol esters; alkyl glucosides like arachidyl glucoside, C₁₂₋₂₀ alkyl glucoside, caprylyl/capryl glucoside, cetearyl glucoside, coco-glucoside, ethyl glucoside and lauryl glucoside. decyl glucoside; ethoxylated alkyl glucosides; sucrose esters like sucrose monooleate, sucrose monolaurate, sucrose monopalmitate, sucrose monostearate, sucrose trioleate, sucrose ricinoleate, sucrose tristearate, sucrose diglycerides and sucrose triacetate; ethoxylated sucrose ester; amine oxides; ethoxylated alcohols; ethoxylated aliphatic alcohols; alkylamines; ethoxylated alkylamines; ethoxylated alkyl phenols like ethoxylated nonyl phenol and ethoxylated octyl phenol; alkyl polysaccharides; ethoxylated alkyl polysaccharides; ethoxylated fatty acids like ethoxylated castor oil; ethoxylated fatty alcohols like ethoxylated ceto-oleyl alcohol, ethoxylated ceto-stearyl alcohol, ethoxylated decyl alcohol, ethoxylated dodecyl alcohol and ethoxylated tridecyl alcohol; ethoxylated fatty amines; poloxamers (polyethylene-polypropylene copolymers), like Poloxamer 124 (PLURONIC® L44), Poloxamer 181 (PLURONIC® L61), Poloxamer 182 (PLURONIC® L62), Poloxamer 184 (PLURONIC®L64), Poloxamer 188 (PLURONIC® F68), Poloxamer 237 (PLURONIC® F87), Poloxamer 338 (PLURONIC® L108), and Poloxamer 407 (PLURONIC® F127); linear secondary alcohol ethoxylates like TERGITOL™ 15-S-5, TERGITOL™ 15-S-7, TERGITOL™ 15-S-9, TERGITOL™ 15-S-12, TERGITOL™ 15-S-15, TERGITOL™ 15-S-20, TERGITOL™ 15-S-30 and TERGITOL™ 15-S-40; alkyl phenol polyglycol ethers; polyethylene glycol alkyl aryl ethers; polyoxyethylene glycol alkyl ethers, like octaethylene glycol monododecyl ether, pentaethylene glycol monododecyl ether, BRIJ® 30, and BRIJ® 35; 2-dodecoxyethanol (LUBROL®-PX); polyoxyethylene glycol octylphenol ethers like polyoxyethylene (4-5) p-t-octyl phenol (TRITON® X-45) and polyoxyethylene octyl phenyl ether (TRITON® X-100); polyoxyethylene glycol alkylphenol ethers like Nonoxynol-9; phenoxypolyethoxylethanols like nonylphenoxypolyethoxylethanol and octylphenoxypolyethoxylethanol (IGEPAL® CA-630 or NONIDET™ P-40); glucoside alkyl ethers like octyl glucopyranoside; maltoside alkyl ethers like dodecyl maltopyranoside; thioglucoside alkyl ethers like heptyl thioglucopyranoside; digitonins; glycerol alkyl esters like glyceryl laurate; alkyl aryl polyether sulfates; alcohol sulfonates; sorbitan alkyl esters; cocamide ethanolamines like cocamide monoethanolamine and cocamide diethanolamine; sucrose monolaurate; dodecyl dimethylamine oxide, and sodium cholate. Other non-limiting examples of surfactants useful in the methods disclosed herein can be found in, e.g., Winslow, et al., Methods and Compositions for Simultaneously Isolating Hemoglobin from Red Blood Cells and Inactivating Viruses, U.S. 2008/0138790; Pharmaceutical Dosage Forms and Drug Delivery Systems (Howard C. Ansel et al., eds., Lippincott Williams & Wilkins Publishers, 7^{th} ed. 1999); Remington: The Science and Practice of Pharmacy (Alfonso R. Gennaro ed., Lippincott, Williams & Wilkins, 20th ed. 2000); Goodman & Gilman's The Pharmacological Basis of Therapeutics (Joel G. Hardman et al., eds., McGraw-Hill Professional, 10^{th} ed. 2001); and Handbook of Pharmaceutical Excipients (Raymond C. Rowe et al., APhA Publications, 4^{th} edition 2003), each of which is hereby incorporated by reference in its entirety.

In one embodiment, a single surfactant is present in a composition disclosed herein. In another embodiment, a plurality of surfactants is present in a composition disclosed herein. In aspects of this embodiment, a composition disclosed herein comprises, e.g., one or more surfactants, two or more surfactants, three or more surfactants, four or more surfactants or five or more surfactants. In other aspects of this embodiment, a composition disclosed herein comprises, e.g., only one surfactant, at most two surfactants, at most three surfactants, at most four surfactants, or at most five surfactants. In yet other aspects of this embodiment, a composition disclosed herein comprises from, e.g., 1 to 2 surfactants, 1 to 3 surfactants, 1 to 4 surfactants, 1 to 5 surfactants, 2 to 3 surfactants, 2 to 4 surfactants, 2 to 5 surfactants, 3 to 4 surfactants, 3 to 5 surfactants or 4 to 5 surfactants.

Any amount of a surfactant disclosed herein may be used, with the proviso that the amount is useful to practice the methods disclosed herein. For example, when a composition comprising a surfactant is formulated to be used in an undiluted state, the amount of a surfactant used is a microbiostatic effective amount. A microbiostatic effective amount of a surfactant is an amount that restrains, prohibits, or otherwise prevents microbial population growth without eradicating the microbes. In an aspect of this embodiment, a microbiostatic effective amount of a surfactant is an amount that restrains, prohibits, or otherwise prevents microbial population growth without eradicating the microbes to a degree that achieves disinfection or sterilization. Alternatively, when a composition comprising a surfactant is formulated to be used in methods where the composition will be diluted, the amount of a surfactant used in such concentrated formulations can be any amount that does not cause precipitation of components in the composition disclosed herein or otherwise create unwanted effects that make the composition inoperable.

In aspects of this embodiment, a composition disclosed herein comprises a surfactant in an amount of, e.g., about 0.001%, about 0.005%, about 0.0075%, about 0.01%, about 0.05%, about 0.075%, about 0.1%, about 0.2%, about 0.3%, about 0.4%, about 0.5%, about 0.6%, about 0.7%, about 0.8%, about 0.9%, about 1.0%, about 1.5%, about 2.0%, about 2.5%, about 3.0%, about 4.0%, about 5.0%, about 6.0%, about 7.0%, about 7.5%, about 8.0%, about 9.0% or about 10.0% by weight of the composition. In other aspects of this embodiment, a composition disclosed herein comprises a surfactant in an amount of, e.g., at least 0.001%, at least 0.005%, at least 0.0075%, at least 0.01%, at least 0.05%, at least 0.075%, at least 0.1%, at least 0.25%, at least 0.5%, at least 0.75%, at least 1.0%, at least 1.5%, at least 2.0%, at least 2.5%, at least 3.0%, at least 4.0%, at least 5.0%, at least 6.0%, at least 7.0%, at least 7.5%, at least 8.0%, at least 9.0%, or at least 10.0% by weight of the composition. In yet other aspects of this embodiment, a composition disclosed herein comprises a surfactant in an amount of, e.g., at most 0.001%, at most 0.005%, at most 0.0075%, at most 0.01%, at most 0.05%, at most 0.075%, at most 0.1%, at most 0.25%, at most 0.5%, at most 0.75%, at most 1.0%, at most 1.5%, at most 2.0%, at most 2.5%, at most 3.0%, at most 4.0%, at most 5.0%, at most 6.0%, at most 7.5%, at most 8.0%, at most 9.0% or at most 10.0% by weight of the composition.

In still other aspects of this embodiment, a composition disclosed herein comprises a surfactant in an amount of, e.g., about 0.001% to about 0.005%, about 0.001% to about 0.0075%, about 0.001% to about 0.01%, about 0.001% to about 0.025%, about 0.001% to about 0.05%, about 0.001% to about 0.075%, about 0.001% to about 0.1%, about 0.001% to about 0.25%, about 0.001% to about 0.75%, about 0.001% to about 1.0%, about 0.005% to about 0.0075%, about 0.005% to about 0.01%, about 0.005% to about 0.025%, about 0.005% to about 0.05%, about 0.005% to about 0.075%, about 0.005% to about 0.1%, about 0.005% to about 0.25%, about 0.005% to about 0.75%, about 0.005% to about 1.0%, about 0.01% to about 0.05%, about 0.01% to about 0.075%, about 0.01% to about 0.1%, about 0.01% to about 0.25%, about 0.01% to about 0.5%, about 0.01% to about 0.75%, about 0.01% to about 1.0%, about 0.01% to about 1.5%, about 0.01% to about 2.0%, about 0.01% to about 2.5%, about 0.05% to about 0.075%, about 0.05% to about 0.1%, about 0.05% to about 0.25%, about 0.05% to about 0.5%, about 0.05% to about 0.75%, about 0.05% to about 1.0%, about 0.05% to about 1.5%, about 0.05% to about 2.0%, about 0.05% to about 2.5%, about 0.1% to about 0.5%, about 0.1% to about 0.75%, about 0.1% to about 1.0%, about 0.1% to about 1.5%, about 0.1% to about 2.0%, about 0.1% to about 2.5%, about 0.1% to about 3.0%, about 0.1% to about 4.0%, about 0.1% to about 5.0%, about 0.2% to about 0.5%, about 0.2% to about 0.75%, about 0.2% to about 1.0%, about 0.2% to about 1.5%, about 0.2% to about 2.0%, about 0.2% to about 2.5%, about 0.2% to about 3.0%, about 0.2% to about 4.0%, about 0.2% to about 5.0%, about 0.5% to about 1.0%, about 0.5% to about 1.5%, about 0.5% to about 2.0%, about 0.5% to about 2.5%, about 0.5% to about 3.0%, about 0.5% to about 4.0%, about 0.5% to about 5.0%, about 0.5% to about 6.0%, about 0.5% to about 7.0%, about 0.5% to about 8.0%, about 0.5% to about 9.0%, about 0.5% to about 10.0%, about 1.0% to about 2.5%, about 1.0% to about 3.0%, about 1.0% to about 4.0%, about 1.0% to about 5.0%, about 1.0% to about 6.0%, about 1.0% to about 7.0%, about 1.0% to about 7.5%, about 1.0% to about 8.0%, about 1.0% to about 9.0%, about 1.0% to about 10.0%, about 2.0% to about 2.5%, about 2.0% to about 3.0%, about 2.0% to about 4.0%, about 2.0% to about 5.0%, about 2.0% to about 6.0%, about 2.0% to about 7.0%, about 2.0% to about 7.5%, about 2.0% to about 8.0%, about 2.0% to about 9.0%, about 2.0% to about 10.0%, about 5.0% to about 6.0%, about 5.0% to about 7.0%, about 5.0% to about 7.5%, about 5.0% to about 8.0%, about 5.0% to about 9.0%, about 5.0% to about 10.0%, about 5.0% to about 11.0%, about 5.0% to about 12.0%, about 5.0% to about 13.0%, about 5.0% to about 14.0%, about 5.0% to about 15.0%, about 6.0% to about 7.0%, about 6.0% to about 7.5%, about 6.0% to about 8.0%, about 6.0% to about 9.0%, about 6.0% to about 10.0%, about 6.0% to about 11.0%, about 6.0% to about 12.0%, about 6.0% to about 13.0%, about 6.0% to about 14.0% or about 6.0% to about 15.0% by weight of the composition.

In another embodiment, a composition disclosed herein does not comprise a surfactant.

A composition disclosed herein may comprise a carrier. A carrier can be any material typically known in the skin care, cosmetic and medical arts that is used as a base to formulate a composition disclosed herein. A carrier may be an aqueous carrier, a semi-solid carrier or a solid carrier. A carrier can also provide a skin care benefit as disclosed herein. A carrier has substantially no long term or permanent detrimental effect when administered and encompasses terms such as pharmacologically acceptable vehicle, stabilizer, diluent, additive, auxiliary, or excipient. Such a carrier generally is mixed with one or more active compounds or permitted to dilute or enclose one or more active compounds and can be a solid, semi-solid, or liquid. It is understood that the one or more active ingredients can be soluble or can be delivered as a suspension in the desired carrier or diluent. A carrier includes, without limitation, water, a vegetable oil, a mineral oil, an ester oil, an ether, an alcohol, a fatty alcohol, an isoparaffin, a hydrocarbon oil, a polyol, and a wax. Non-limiting examples of an ester oil include octal palmitate, isopropyl myristate and isopropyl palmitate. Non-limiting examples of an ether includes dicapryl ether and dimethyl isosorbide. Non-limiting examples of an alcohol includes ethanol and isopropanol. Non-limiting examples of a fatty alcohol include cetyl alcohol, cetearyl alcohol, stearyl alcohol and behenyl alcohol. Non-limiting examples of an isoparaffin include isooctane, isododecane (IDD) and isohexadecane. Non-limiting examples of a hydrocarbon oil include mineral oil, petrolatum, isoeicosane and a polyolefin, including (hydrogenated) polyisobutene. Non-limiting examples of a polyol include propylene glycol, glycerin, butylene glycol, pentylene glycol, hexylene glycol, caprylyl glycol. Non-limiting examples of a wax include beeswax, carnauba, ozokerite, microcrystalline wax, polyethylene wax, and a botanical wax.

In one embodiment, a single carrier is present in a composition disclosed herein. In another embodiment, a plurality of carriers is present in a composition disclosed herein. In aspects of this embodiment, a composition disclosed herein comprises, e.g., one or more carriers, two or more carriers, three or more carriers, four or more carriers or five or more carriers. In other aspects of this embodiment, a composition disclosed herein comprises, e.g., only one carrier, at most two carriers, at most three carriers, at most four carriers, or at most five carriers. In yet other aspects of this embodiment, a composition disclosed herein comprises from, e.g., 1 to 2 carriers, 1 to 3 carriers, 1 to 4 carriers, 1 to 5 carriers, 2 to 3 carriers, 2 to 4 carriers, 2 to 5 carriers, 3 to 4 carriers, 3 to 5 carriers or 4 to 5 carriers.

In another embodiment, a composition disclosed herein comprises an amount of carrier that provides a desired formulative or beneficial effect to a composition disclosed herein. In aspects of this embodiment, a composition disclosed herein comprises a carrier in an amount of, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98% or at least 99% by weight of the composition. In other aspects of this embodiment, a composition disclosed herein comprises a carrier in an amount of, e.g., at most 5%, at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95%, at most 96%, at most 97%, at most 98% or at most 99% by weight of the composition. In yet other aspects of this embodiment, a composition disclosed herein comprises a carrier in an amount of from, e.g., about 5% to about 25%, about 5% to about 50%, about 5% to about 75%, about 5% to about 90%, about 5% to about 95%, about 5% to about 96%, about 5% to about 97%, about 5% to about 98%, about 5% to about 99%, about 25% to about 50%, about 25% to about 75%, about 25% to about 90%, about 25% to about 95%, about 25% to about 96%, about 25% to about 97%, about 25% to about 98%, about 25% to about 99%, about 50% to about 75%, about 50% to about 90%, about 50% to about 95%, about 50% to about 96%, about 50% to about 97%, about 50% to about 98%, about 50% to about 99%, about 75% to about 80%, about 75% to about 85%, about 75% to about 90%, about 75% to about 95%, about 75% to about 96%, about 75% to about 97%, about 75% to about 98%, about 75% to about 99%, about 80% to about 85%, about 80% to about 90%, about 80% to about 95%, about 80% to about 96%, about 80% to about 97%, about 80% to about 98%, about 80% to about 99%, about 85% to about 90%, about 85% to about 95%, about 85% to about 96%, about 85% to about 97%, about 85% to about 98%, about 85% to about 99%, about 90% to about 95%, about 90% to about 96%, about 90% to about 97%, about 90% to about 98%, about 90% to about 99%, or about 95% to about 99%, by weight of the composition.

In another embodiment, a composition disclosed herein comprises a carrier which is not an alcohol. In aspects of this embodiment, when a composition disclosed herein comprises hypochlorous acid or free available chlorine, then the composition may comprise a carrier where the carrier is not an alcohol.

A composition disclosed herein may further optionally include additional ingredients. An additional ingredient is one known to be useful in finishing a composition disclosed herein. An additional ingredient includes, without limitation, a buffer, a hydrophilic clay, a disinfectant, an antiseptic, a preservative, or a chelating agent. An additional ingredient disclosed herein is known in the art.

Various buffers and means for adjusting pH can be used to prepare a microbiostatic composition disclosed herein, provided that the resulting preparation is pharmacologically acceptable. Such buffers include, without limitation, acetate buffers, citrate buffers, phosphate buffers, neutral buffered saline, phosphate buffered saline and borate buffers. It is understood that acids or bases can be used to adjust the pH of a composition as needed. Additional acceptable antioxidants include, without limitation, sodium metabisulfite, sodium thiosulfate, acetylcysteine, butylated hydroxyanisole and butylated hydroxytoluene.

A hydrophilic clay is a synthetic or naturally occurring smectic clay that forms three-dimensional colloidal structures when hydrated leading to increased viscosity and improved suspension control components in a composition. A hydrophilic clay includes hydrous phyllosilicate clays. A hydrophilic clay can have thixotrophic properties. Non-limiting examples of a hydrophilic clay include a bentonite clay, a hectorite clay, a kaolinite clay, and a silicate clay. A bentonite clay is a phyllosilicate clay and includes, without limitation, a potassium bentonite clay, a sodium bentonite clay, a calcium bentonite clay, and an aluminum bentonite clay. A hectorite clay is a phyllosilicate clay and includes those commercially sold as the BENTONE® product line, including hectorite clay (BENTONE® EW), hectorite clay (BENTONE® MA), and hectorite clay and hydroxy ethyl cellulose (BENTONE® LT). A kaolinite clay is a phyllosilicate clay. A silicate clay is a synthetic layered silicate clay and includes those commercially sold as the LAPONITE® product line, including lithium magnesium sodium silicate (LAPONITE® XLG), lithium magnesium sodium silicate and tetrasodium pyrophosphate (LAPONITE® XLS), sodium magnesium fluorosilicate (LAPONITE® XL), and sodium magnesium fluorosilicate (LAPONITE® XL21).

In one embodiment, a single hydrophilic clay is present in a composition disclosed herein. In another embodiment, a plurality of hydrophilic clays is present in a composition disclosed herein. In aspects of this embodiment, a composition disclosed herein comprises, e.g., one or more hydrophilic clays, two or more hydrophilic clays, three or more hydrophilic clays, four or more hydrophilic clays or five or more hydrophilic clays. In other aspects of this embodiment, a composition disclosed herein comprises, e.g., only one hydrophilic clay, at most two hydrophilic clays, at most three hydrophilic clays, at most four hydrophilic clays, or at most five hydrophilic clays. In yet other aspects of this embodiment, a composition disclosed herein comprises from, e.g., 1 to 2 hydrophilic clays, 1 to 3 hydrophilic clays, 1 to 4 hydrophilic clays, 1 to 5 hydrophilic clays, 2 to 3 hydrophilic clays, 2 to 4 hydrophilic clays, 2 to 5 hydrophilic clays, 3 to 4 hydrophilic clays, 3 to 5 hydrophilic clays or 4 to 5 hydrophilic clays.

In another embodiment, a composition disclosed herein comprises an amount of a hydrophilic clay that provides a desired beneficial effect to a composition disclosed herein. In aspects of this embodiment, a composition disclosed herein comprises a hydrophilic clay in an amount of, e.g., 0.01%, 0.025%, 0.05%, 0.075%, 0.1%, 0.2%, 0.25%, 0.3%, 0.4%, 0.5%, 0.6%, 0.7%, 0.75%, 0.8%, 0.9%, 1%, 2%, 3%, 4%, 5%, 6%, 7%, 8%. 9%, or 10% by weight of the composition. In other aspects of this embodiment, a composition disclosed herein comprises a hydrophilic clay in an amount of, e.g., at least 0.01%, at least 0.025%, at least 0.05%, at least 0.075%, at least 0.1%, at least 0.25%, at least 0.5%, at least 0.75%, at least 1%, at least 2%, at least 3%, at least 4%, at least 5%, at least 6%, at least 7%, at least 8%, at least 9%, or at least 10% by weight of the composition. In yet other aspects of this embodiment, a composition disclosed herein comprises a hydrophilic clay in an amount of, e.g., at most 0.01%, at most 0.025%, at most 0.05%, at most 0.075%, at most 0.1%, at most 0.25%, at most 0.5%, at most 0.75%, at most 1%, at most 2%, at most 3%, at most 4%, at most 5%, at most 6%, at most 7%, at most 8%, at most 9%, or at most 10% by weight of the composition.

In still other aspects of this embodiment, a composition disclosed herein comprises a hydrophilic clay in an amount of from, e.g., about 0.01% to about 0.05%, 0.01% to about 0.075%, about 0.01% to about 0.1%, about 0.1% to about 0.5%, about 0.1% to about 0.75%, about 0.1% to about 1%, about 0.1% to about 2%, about 0.1% to about 3%, about 0.1% to about 4%, about 0.1% to about 5%, about 0.1% to about 6%, about 0.1% to about 7%, about 0.1% to about 8%, about 0.1% to about 9%, about 0.1% to about 10%, about 0.25% to about 0.5%, about 0.25% to about 0.75%, about 0.25% to about 1%, about 0.25% to about 2%, about 0.25% to about 3%, about 0.25% to about 4%, about 0.25% to about 5%, about 0.25% to about 6%, about 0.25% to about 7%, about 0.25% to about 8%, about 0.25% to about 9%, about 0.25% to about 10%, about 0.5% to about 0.75%, about 0.5% to about 1%, about 0.5% to about 2%, about 0.5% to about 3%, about 0.5% to about 4%, about 0.5% to about 5%, about 0.5% to about 6%, about 0.5% to about 7%, about 0.5% to about 8%, about 0.5% to about 9%, about 0.5% to about 10%, about 0.75% to about 1%, about 0.75% to about 2%, about 0.75% to about 3%, about 0.75% to about 4%, about 0.75% to about 5%, about 0.75% to about 6%, about 0.75% to about 7%, about 0.75% to about 8%, about 0.75% to about 9%, about 0.75% to about 10%, about 1% to about 2%, about 1% to about 3%, about 1% to about 4%, about 1% to about 5%, about 1% to about 6%, about 1% to about 7%, about 1% to about 8%, about 1% to about 9%, about 1% to about 10%, about 2% to about 3%, about 2% to about 4%, about 2% to about 5%, about 2% to about 6%, about 2% to about 7%, about 2% to about 8%, about 2% to about 9%, about 2% to about 10%, about 3% to about 4%, about 3% to about 5%, about 3% to about 6%, about 3% to about 7%, about 3% to about 8%, about 3% to about 9%, about 3% to about 10%, about 4% to about 5%, about 4% to about 6%, about 4% to about 7%, about 4% to about 8%, about 4% to about 9%, about 4% to about 10%, about 5% to about 6%, about 5% to about 7%, about 5% to about 8%, about 5% to about 9%, about 5% to about 10%, about 6% to about 7%, about 6% to about 8%, about 6% to about 9%, about 6% to about 10%, about 7% to about 8%, about 7% to about 9%, about 7% to about 10%, about 8% to about 9%, about 8% to about 10% or about 9% to about 10% by weight of the composition.

In another embodiment, a composition disclosed herein comprises an amount of a silicate clay that provides a desired beneficial effect to a composition disclosed herein. In aspects of this embodiment, a composition disclosed herein comprises a silicate clay in an amount of, e.g., 0.001%, 0.0025%, 0.005%, 0.0075%, 0.01%, 0.025%, 0.05%, 0.075%, 0.1%, 0.2%, 0.25%, 0.3%, 0.4%, 0.5%, 0.6%, 0.7%, 0.75%, 0.8%, 0.9%, 1%, 2%, 3%, 4%, 5%, 6%, 7%, 8%. 9%, or 10% by weight of the composition. In other aspects of this embodiment, a composition disclosed herein comprises a silicate clay in an amount of, e.g., at least 0.001%, at least 0.0025%, at least 0.005%, at least 0.0075%, at least 0.01%, at least 0.025%, at least 0.05%, at least 0.075%, at least 0.1%, at least 0.25%, at least 0.5%, at least 0.75%, at least 1%, at least 2%, at least 3%, at least 4%, at least 5%, at least 6%, at least 7%, at least 8%, at least 9%, or at least 10% by weight of the composition. In yet other aspects of this embodiment, a composition disclosed herein comprises a silicate clay in an amount of, e.g., at most 0.001%, at most 0.0025%, at most 0.005%, at most 0.0075%, at most 0.01%, at most 0.025%, at most 0.05%, at most 0.075%, at most 0.1%, at most 0.25%, at most 0.5%, at most 0.75%, at most 1%, at most 2%, at most 3%, at most 4%, at most 5%, at most 6%, at most 7%, at most 8%, at most 9%, or at most 10% by weight of the composition.

In still other aspects of this embodiment, a composition disclosed herein comprises a silicate clay in an amount of from, e.g., about 0.001% to about 0.005%, 0.001% to about 0.0075%, about 0.001% to about 0.01%, about 0.001% to about 0.05%, about 0.001% to about 0.075%, about 0.001% to about 0.1%, about 0.001% to about 0.25%, about 0.001% to about 0.5%, about 0.001% to about 0.75%, about 0.005% to about 0.0075%, about 0.005% to about 0.01%, about 0.005% to about 0.05%, about 0.005% to about 0.075%, about 0.005% to about 0.1%, about 0.005% to about 0.25%, about 0.005% to about 0.5%, about 0.005% to about 0.75%, about 0.01% to about 0.05%, 0.01% to about 0.075%, about 0.01% to about 0.1%, about 0.1% to about 0.5%, about 0.1% to about 0.75%, about 0.1% to about 1%, about 0.1% to about 2%, about 0.1% to about 3%, about 0.1% to about 4%, about 0.1% to about 5%, about 0.1% to about 6%, about 0.1% to about 7%, about 0.1% to about 8%, about 0.1% to about 9%, about 0.1% to about 10%, about 0.25% to about 0.5%, about 0.25% to about 0.75%, about 0.25% to about 1%, about 0.25% to about 2%, about 0.25% to about 3%, about 0.25% to about 4%, about 0.25% to about 5%, about 0.25% to about 6%, about 0.25% to about 7%, about 0.25% to about 8%, about 0.25% to about 9%, about 0.25% to about 10%, about 0.5% to about 0.75%, about 0.5% to about 1%, about 0.5% to about 2%, about 0.5% to about 3%, about 0.5% to about 4%, about 0.5% to about 5%, about 0.5% to about 6%, about 0.5% to about 7%, about 0.5% to about 8%, about 0.5% to about 9%, about 0.5% to about 10%, about 0.75% to about 1%, about 0.75% to about 2%, about 0.75% to about 3%, about 0.75% to about 4%, about 0.75% to about 5%, about 0.75% to about 6%, about 0.75% to about 7%, about 0.75% to about 8%, about 0.75% to about 9%, about 0.75% to about 10%, about 1% to about 2%, about 1% to about 3%, about 1% to about 4%, about 1% to about 5%, about 1% to about 6%, about 1% to about 7%, about 1% to about 8%, about 1% to about 9%, about 1% to about 10%, about 2% to about 3%, about 2% to about 4%, about 2% to about 5%, about 2% to about 6%, about 2% to about 7%, about 2% to about 8%, about 2% to about 9%, about 2% to about 10%, about 3% to about 4%, about 3% to about 5%, about 3% to about 6%, about 3% to about 7%, about 3% to about 8%, about 3% to about 9%, about 3% to about 10%, about 4% to about 5%, about 4% to about 6%, about 4% to about 7%, about 4% to about 8%, about 4% to about 9%, about 4% to about 10%, about 5% to about 6%, about 5% to about 7%, about 5% to about 8%, about 5% to about 9%, about 5% to about 10%, about 6% to about 7%, about 6% to about 8%, about 6% to about 9%, about 6% to about 10%, about 7% to about 8%, about 7% to about 9%, about 7% to about 10%, about 8% to about 9%, about 8% to about 10% or about 9% to about 10% by weight of the composition.

A preservative preserves the stability of a composition disclosed herein. A preservative can also prevent the growth of microbial organisms in a composition disclosed herein. Non-limiting examples of a preservative include methylparaben, phenoxyethanol, capryl glycol, glyceryl caprylate, benzoic acid, sorbic acid, gallic acid, and propylparaben.

A composition disclosed herein may be adjusted to any pH that enables a composition disclosed herein to achieve a desired beneficial effect. In aspects of this embodiment, the pH of a composition disclosed herein is, e.g., at least 2, at least 3, at least 4, at least 5, at least 6, at least 7, at least 8, at least 9, at least 10, or at least 11. In other aspects of this embodiment, the pH of a composition disclosed herein is, e.g., at most 2, at most 3, at most 4, at most 5, at most 6, at most 7, at most 8, at most 9, at most 10, or at most 11.

In yet other aspects of this embodiment, the pH of a composition disclosed herein is between, e.g., about 2 to about 5, about 2 to about 5.5, about 2 to about 6, about 2 to about 6.5, about 2 to about 7, about 2 to about 7.5, about 2 to about 8, about 2 to about 8.5, about 2 to about 9, about 2.5 to about 5, about 2.5 to about 5.5, about 2.5 to about 6, about 2.5 to about 6.5, about 2.5 to about 7, about 2.5 to about 7.5, about 2.5 to about 8, about 2.5 to about 8.5, about 2.5 to about 9, about 3 to about 5, about 3 to about 5.5, about 3 to about 6, about 3 to about 6.5, about 3 to about 7, about 3 to about 7.5, about 3 to about 8, about 3 to about 8.5, about 3 to about 9, about 3.5 to about 5, about 3.5 to about 5.5, about 3.5 to about 6, about 3.5 to about 6.5, about 3.5 to about 7, about 3.5 to about 7.5, about 3.5 to about 8, about 3.5 to about 8.5, about 3.5 to about 9, about 4 to about 5, about 4 to about 5.5, about 4 to about 6, about 4 to about 6.5, about 4 to about 7, about 4 to about 7.5, about 4 to about 8, about 4 to about 8.5, about 4 to about 9, about 4.5 to about 5, about 4.5 to about 5.5, about 4.5 to about 6, about 4.5 to about 6.5, about 4.5 to about 7, about 4.5 to about 7.5, about 4.5 to about 8, about 4.5 to about 8.5, about 4.5 to about 9, about 5 to about 5.5, about 5 to about 6, about 5 to about 6.5, about 5 to about 7, about 5 to about 7.5, about 5 to about 8, about 5 to about 8.5, about 5 to about 9, about 5.5 to about 6, about 5.5 to about 6.5, about 5.5 to about 7, about 5.5 to about 7.5, about 5.5 to about 8, about 5.5 to about 8.5, about 5.5 to about 9, about 6 to about 6.5, about 6 to about 7, about 6 to about 7.5, about 6 to about 8, about 6 to about 8.5, about 6 to about 9, about 6.5 to about 7, about 6.5 to about 7.5, about 6.5 to about 8, about 6.5 to about 8.5, about 6.5 to about 9, about 7 to about 7.5, about 7 to about 8, about 7 to about 8.5, about 7 to about 9, about 7.5 to about 8, about 7.5 to about 8.5, about 7.5 to about 9, about 8 to about 8.5, about 8 to about 9, or about 8.5 to about 9.

In another embodiment, a calcium oxide powder is used to pH a composition disclosed herein. In some embodiments, a calcium oxide powder obtained from an organic source like shells from oceanic organisms. In some embodiment, a calcium oxide powder obtain from an inorganic source like by a synthetic chemical process. In some embodiments, about 160 ppm of a calcium oxide powder results in a composition having a pH of about 5.0 to about 5.2. In some embodiments, about 240 ppm of a calcium oxide powder results in a composition having a pH of about 7.5 to about 7.8. In aspects of this embodiment, a calcium oxide powder is used to pH a composition disclosed herein to, e.g., at least 2, at least 3, at least 4, at least 5, at least 6, at least 7, at least 8, at least 9, at least 10, or at least 11. In other aspects of this embodiment, a calcium oxide powder is used to pH a composition disclosed herein to, e.g., at most 2, at most 3, at most 4, at most 5, at most 6, at most 7, at most 8, at most 9, at most 10, or at most 11.

In yet other aspects of this embodiment, a calcium oxide powder is used to pH a composition disclosed herein to, e.g., about 2 to about 5, about 2 to about 5.5, about 2 to about 6, about 2 to about 6.5, about 2 to about 7, about 2 to about 7.5, about 2 to about 8, about 2 to about 8.5, about 2 to about 9, about 2.5 to about 5, about 2.5 to about 5.5, about 2.5 to about 6, about 2.5 to about 6.5, about 2.5 to about 7, about 2.5 to about 7.5, about 2.5 to about 8, about 2.5 to about 8.5, about 2.5 to about 9, about 3 to about 5, about 3 to about 5.5, about 3 to about 6, about 3 to about 6.5, about 3 to about 7, about 3 to about 7.5, about 3 to about 8, about 3 to about 8.5, about 3 to about 9, about 3.5 to about 5, about 3.5 to about 5.5, about 3.5 to about 6, about 3.5 to about 6.5, about 3.5 to about 7, about 3.5 to about 7.5, about 3.5 to about 8, about 3.5 to about 8.5, about 3.5 to about 9, about 4 to about 5, about 4 to about 5.5, about 4 to about 6, about 4 to about 6.5, about 4 to about 7, about 4 to about 7.5, about 4 to about 8, about 4 to about 8.5, about 4 to about 9, about 4.5 to about 5, about 4.5 to about 5.5, about 4.5 to about 6, about 4.5 to about 6.5, about 4.5 to about 7, about 4.5 to about 7.5, about 4.5 to about 8, about 4.5 to about 8.5, about 4.5 to about 9, about 5 to about 5.5, about 5 to about 6, about 5 to about 6.5, about 5 to about 7, about 5 to about 7.5, about 5 to about 8, about 5 to about 8.5, about 5 to about 9, about 5.5 to about 6, about 5.5 to about 6.5, about 5.5 to about 7, about 5.5 to about 7.5, about 5.5 to about 8, about 5.5 to about 8.5, about 5.5 to about 9, about 6 to about 6.5, about 6 to about 7, about 6 to about 7.5, about 6 to about 8, about 6 to about 8.5, about 6 to about 9, about 6.5 to about 7, about 6.5 to about 7.5, about 6.5 to about 8, about 6.5 to about 8.5, about 6.5 to about 9, about 7 to about 7.5, about 7 to about 8, about 7 to about 8.5, about 7 to about 9, about 7.5 to about 8, about 7.5 to about 8.5, about 7.5 to about 9, about 8 to about 8.5, about 8 to about 9, or about 8.5 to about 9.

A composition disclosed herein is resistant to inactivation when exposed to a bodily fluid. A bodily fluid is one containing charged molecules and includes, without limitation, blood, and a blood byproduct like plasma and serum. In one embodiment, a composition disclosed herein is resistant to inactivation when it shows, e.g., at most 1% inactivation, at most 2% inactivation, at most 3% inactivation, at most 4% inactivation, at most 5% inactivation, at most 6% inactivation, at most 7% inactivation, at most 8% inactivation, at most 9% inactivation, or at most 10% inactivation upon exposure to a bodily fluid. In another embodiment, a composition disclosed herein is resistant to inactivation when it shows, e.g., at most 10% inactivation, at most 15% inactivation, at most 20% inactivation, at most 25% inactivation, at most 30% inactivation, at most 35% inactivation, at most 40% inactivation, at most 45% inactivation, at most 50% inactivation upon exposure to a bodily fluid. In another embodiment, a composition disclosed herein is resistant to inactivation when it shows, e.g., about 1% to about 10% inactivation, about 1% to about 20% inactivation, about 1% to about 30% inactivation, about 1% to about 40% inactivation, about 1% to about 50% inactivation, about 5% to about 10% inactivation, about 5% to about 20% inactivation, about 5% to about 30% inactivation, about 5% to about 40% inactivation, about 5% to about 50% inactivation, about 10% to about 20% inactivation, about 10% to about 30% inactivation, about 10% to about 40% inactivation, about 10% to about 50% inactivation, 20% to about 30% inactivation, about 20% to about 40% inactivation, about 20% to about 50% inactivation, about 30% to about 40% inactivation, about 30% to about 50% inactivation, or about 40% to about 50% inactivation upon exposure to a bodily fluid.

In one embodiment, a composition disclosed herein is resistant to inactivation when it shows, e.g., at most 1% degradation, at most 2% degradation, at most 3% degradation, at most 4% degradation, at most 5% degradation, at most 6% degradation, at most 7% degradation, at most 8% degradation, at most 9% degradation, or at most 10% degradation of hypochlorous acid upon exposure to a bodily fluid. In another embodiment, a composition disclosed herein is resistant to inactivation when it shows, e.g., at most 10% degradation, at most 15% degradation, at most 20% degradation, at most 25% degradation, at most 30% degradation, at most 35% degradation, at most 40% degradation, at most 45% degradation, at most 50% degradation of hypochlorous acid upon exposure to a bodily fluid. In another embodiment, a composition disclosed herein is resistant to inactivation when it shows, e.g., about 1% to about 10% degradation, about 1% to about 20% degradation, about 1% to about 30% degradation, about 1% to about 40% degradation, about 1% to about 50% degradation, about 5% to about 10% degradation, about 5% to about 20% degradation, about 5% to about 30% degradation, about 5% to about 40% degradation, about 5% to about 50% degradation, about 10% to about 20% degradation, about 10% to about 30% degradation, about 10% to about 40% degradation, about 10% to about 50% degradation, 20% to about 30% degradation, about 20% to about 40% degradation, about 20% to about 50% degradation, about 30% to about 40% degradation, about 30% to about 50% degradation, or about 40% to about 50% degradation of hypochlorous acid upon exposure to a bodily fluid.

A composition disclosed herein is stable. A composition disclosed herein is stable, or has stability, in two different aspects. First, a composition disclosed herein has a shelf-life stability. Shelf-life stability is the length of time that a composition disclosed herein may be stored prior to its first use without becoming unfit for its intended purpose of restraining, inhibiting or otherwise preventing microbial population growth. Shelf-life stability is the time from when a composition disclosed herein is manufactured and packaged in a container until the time the composition becomes unfit for its intended purpose while remaining unopened in the container. Second, a composition disclosed herein has an effective-use stability. Effective-use stability is the length of time that a composition disclosed herein is effective for its intended purpose of restraining, inhibiting or otherwise preventing microbial population growth after its first use. Effective-use stability is the time from when a container storing a composition disclosed herein is first open or manufactured on site for immediate use until the time the composition becomes unfit for its intended purpose.

In one embodiment, a composition disclosed herein is stable when it shows, e.g., at most 1% degradation, at most 2% degradation, at most 3% degradation, at most 4% degradation, at most 5% degradation, at most 6% degradation, at most 7% degradation, at most 8% degradation, at most 9% degradation, or at most 10% degradation of hypochlorous acid. In another embodiment, a composition disclosed herein is stable when it shows, e.g., at most 10% degradation, at most 15% degradation, at most 20% degradation, at most 25% degradation, at most 30% degradation, at most 35% degradation, at most 40% degradation, at most 45% degradation, at most 50% degradation, at most 55% degradation, at most 60% degradation, at most 65% degradation, at most 70% degradation, at most 75% degradation, at most 80% degradation, at most 85% degradation, at most 90% degradation, or at most 95% degradation of hypochlorous acid. In another embodiment, a composition disclosed herein is stable when it shows, e.g., about 1% to about 10% degradation, about 1% to about 20% degradation, about 1% to about 30% degradation, about 1% to about 40% degradation, about 1% to about 50% degradation, about 1% to about 60% degradation, about 1% to about 70% degradation, about 1% to about 80% degradation, about 1% to about 90% degradation, about 1% to about 95% degradation, about 5% to about 10% degradation, about 5% to about 20% degradation, about 5% to about 30% degradation, about 5% to about 40% degradation, about 5% to about 50% degradation, about 5% to about 60% degradation, about 5% to about 70% degradation, about 5% to about 80% degradation, about 5% to about 90% degradation, about 5% to about 95% degradation, about 10% to about 20% degradation, about 10% to about 30% degradation, about 10% to about 40% degradation, about 10% to about 50% degradation, about 10% to about 60% degradation, about 10% to about 70% degradation, about 10% to about 80% degradation, about 10% to about 90% degradation, about 10% to about 95% degradation, 20% to about 30% degradation, about 20% to about 40% degradation, about 20% to about 50% degradation, about 20% to about 60% degradation, about 20% to about 70% degradation, about 20% to about 80% degradation, about 20% to about 90% degradation, about 20% to about 95% degradation, about 30% to about 40% degradation, about 30% to about 50% degradation, about 30% to about 60% degradation, about 30% to about 70% degradation, about 30% to about 80% degradation, about 30% to about 90% degradation, about 30% to about 95% degradation, about 40% to about 50% degradation, about 40% to about 60% degradation, about 40% to about 70% degradation, about 40% to about 80% degradation, about 40% to about 90% degradation, about 40% to about 95% degradation, about 50% to about 60% degradation, about 50% to about 70% degradation, about 50% to about 80% degradation, about 50% to about 90% degradation, about 50% to about 95% degradation, about 60% to about 70% degradation, about 60% to about 80% degradation, about 60% to about 90% degradation, about 60% to about 95% degradation, about 70% to about 80% degradation, about 70% to about 90% degradation, about 70% to about 95% degradation, about 80% to about 90% degradation, about 80% to about 95% degradation, or about 90% to about 95% degradation of hypochlorous acid.

In one embodiment, a composition disclosed herein is stable when it shows, e.g., at most 1% degradation, at most 2% degradation, at most 3% degradation, at most 4% degradation, at most 5% degradation, at most 6% degradation, at most 7% degradation, at most 8% degradation, at most 9% degradation, or at most 10% degradation of free available chlorine. In another embodiment, a composition disclosed herein is stable when it shows, e.g., at most 10% degradation, at most 15% degradation, at most 20% degradation, at most 25% degradation, at most 30% degradation, at most 35% degradation, at most 40% degradation, at most 45% degradation, at most 50% degradation, at most 55% degradation, at most 60% degradation, at most 65% degradation, at most 70% degradation, at most 75% degradation, at most 80% degradation, at most 85% degradation, at most 90% degradation, or at most 95% degradation of free available chlorine. In another embodiment, a composition disclosed herein is stable when it shows, e.g., about 1% to about 10% degradation, about 1% to about 20% degradation, about 1% to about 30% degradation, about 1% to about 40% degradation, about 1% to about 50% degradation, about 1% to about 60% degradation, about 1% to about 70% degradation, about 1% to about 80% degradation, about 1% to about 90% degradation, about 1% to about 95% degradation, about 5% to about 10% degradation, about 5% to about 20% degradation, about 5% to about 30% degradation, about 5% to about 40% degradation, about 5% to about 50% degradation, about 5% to about 60% degradation, about 5% to about 70% degradation, about 5% to about 80% degradation, about 5% to about 90% degradation, about 5% to about 95% degradation, about 10% to about 20% degradation, about 10% to about 30% degradation, about 10% to about 40% degradation, about 10% to about 50% degradation, about 10% to about 60% degradation, about 10% to about 70% degradation, about 10% to about 80% degradation, about 10% to about 90% degradation, about 10% to about 95% degradation, 20% to about 30% degradation, about 20% to about 40% degradation, about 20% to about 50% degradation, about 20% to about 60% degradation, about 20% to about 70% degradation, about 20% to about 80% degradation, about 20% to about 90% degradation, about 20% to about 95% degradation, about 30% to about 40% degradation, about 30% to about 50% degradation, about 30% to about 60% degradation, about 30% to about 70% degradation, about 30% to about 80% degradation, about 30% to about 90% degradation, about 30% to about 95% degradation, about 40% to about 50% degradation, about 40% to about 60% degradation, about 40% to about 70% degradation, about 40% to about 80% degradation, about 40% to about 90% degradation, about 40% to about 95% degradation, about 50% to about 60% degradation, about 50% to about 70% degradation, about 50% to about 80% degradation, about 50% to about 90% degradation, about 50% to about 95% degradation, about 60% to about 70% degradation, about 60% to about 80% degradation, about 60% to about 90% degradation, about 60% to about 95% degradation, about 70% to about 80% degradation, about 70% to about 90% degradation, about 70% to about 95% degradation, about 80% to about 90% degradation, about 80% to about 95% degradation, or about 90% to about 95% degradation of free available chlorine.

In aspects of this embodiment, a composition disclosed herein has an effective-use stability of e.g., about 1 day, about 2 days, 3 days, 4 days, 5 days, 6, days, 7 days, 8 days, 9 days, 10 days, 11 days, 12 days, 13 days, 14 days, 15 days, about 20 days, about 25 days, or about 30 days. In other aspects of this embodiment, a composition disclosed herein has an effective-use stability of, e.g., at least 1 day, at least 2 days, at least 3 days, at least 4 days, at least 5 days, at least 6, days, at least 7 days, at least 8 days, at least 9 days, at least 10 days, at least 11 days, at least 12 days, at least 13 days, at least 14 days, at least 15 days, at least 20 days, at least 25 days, or at least 30 days. In yet other aspects of this embodiment, a composition disclosed herein has an effective-use stability of, e.g., at most 1 day, at most 2 days, at most 3 days, at most 4 days, at most 5 days, at most 6, days, at most 7 days, at most 8 days, at most 9 days, at most 10 days, at most 11 days, at most 12 days, at most 13 days, at most 14 days, at most 15 days, at most 20 days, at most 25 days, or at most 30 days. In still other aspects of this embodiment, a composition disclosed herein has an effective-use stability of, e.g., about 1 hour to about 4 hours, about 1 hour to about 8 hours, about 1 hour to about 12 hours, about 1 hour to about 16 hours, about 1 hour to about 20 hours, or about 1 hour to about 24 hours.

In still aspects of this embodiment, a composition disclosed herein has an effective-use stability of, e.g., about 1 day to about 2 days, about 1 day to about 3 days, about 1 day to about 4 days, about 1 day to about 5 days, about 1 day to about 6 days, about 1 day to about 7 days, about 1 day to about 8 days, about 1 day to about 9 days, about 1 day to about 10 days, about 1 day to about 11 days, about 1 day to about 12 days, about 1 day to about 13 days, about 1 day to about 14 days, about 1 day to about 15 days, about 1 day to about 20 days, about 1 day to about 25 days, about 1 day to about 30 days, about 2 days to about 3 days, about 2 days to about 4 days, about 2 days to about 5 days, about 2 days to about 6 days, about 2 days to about 7 days, about 2 days to about 8 days, about 2 days to about 9 days, about 2 days to about 10 days, about 2 days to about 11 days, about 2 days to about 12 days, about 2 days to about 13 days, about 2 days to about 14 days, about 2 days to about 15 days, about 2 days to about 20 days, about 2 days to about 25 days, about 2 days to about 30 days, about 3 days to about 4 days, about 3 days to about 5 days, about 3 days to about 6 days, about 3 days to about 7 days, about 3 days to about 8 days, about 3 days to about 9 days, about 3 days to about 10 days, about 3 days to about 11 days, about 3 days to about 12 days, about 3 days to about 13 days, about 3 days to about 14 days, about 3 days to about 15 days, about 3 days to about 20 days, about 3 days to about 25 days, about 3 days to about 30 days, about 4 days to about 5 days, about 4 days to about 6 days, about 4 days to about 7 days, about 4 days to about 8 days, about 4 days to about 9 days, about 4 days to about 10 days, about 4 days to about 11 days, about 4 days to about 12 days, about 4 days to about 13 days, about 4 days to about 14 days, about 4 days to about 15 days, about 4 days to about 20 days, about 4 days to about 25 days, about 4 days to about 30 days, about 5 days to about 6 days, about 5 days to about 7 days, about 5 days to about 8 days, about 5 days to about 9 days, about 5 days to about 10 days, about 5 days to about 11 days, about 5 days to about 12 days, about 5 days to about 13 days, about 5 days to about 14 days, about 5 days to about 15 days, about 5 days to about 20 days, about 5 days to about 25 days, about 5 days to about 30 days, about 6 days to about 7 days, about 6 days to about 8 days, about 6 days to about 9 days, about 6 days to about 10 days, about 6 days to about 11 days, about 6 days to about 12 days, about 6 days to about 13 days, about 6 days to about 14 days, about 6 days to about 15 days, about 6 days to about 20 days, about 6 days to about 25 days, about 6 days to about 30 days, about 7 days to about 8 days, about 7 days to about 9 days, about 7 days to about 10 days, about 7 days to about 11 days, about 7 days to about 12 days, about 7 days to about 13 days, about 7 days to about 14 days, about 7 days to about 15 days, about 7 days to about 20 days, about 7 days to about 25 days, about 7 days to about 30 days, about 8 days to about 9 days, about 8 days to about 10 days, about 8 days to about 11 days, about 8 days to about 12 days, about 8 days to about 13 days, about 8 days to about 14 days, about 8 days to about 15 days, about 8 days to about 20 days, about 8 days to about 25 days, about 8 days to about 30 days, about 9 days to about 10 days, about 9 days to about 11 days, about 9 days to about 12 days, about 9 days to about 13 days, about 9 days to about 14 days, about 9 days to about 15 days, about 9 days to about 20 days, about 9 days to about 25 days, about 9 days to about 30 days, about 10 days to about 11 days, about 10 days to about 12 days, about 10 days to about 13 days, about 10 days to about 14 days, about 10 days to about 15 days, about 10 days to about 20 days, about 10 days to about 25 days, about 10 days to about 30 days, about 11 days to about 12 days, about 11 days to about 13 days, about 11 days to about 14 days, about 11 days to about 15 days, about 11 days to about 20 days, about 11 days to about 25 days, about 11 days to about 30 days, about 12 days to about 13 days, about 12 days to about 14 days, about 12 days to about 15 days, about 12 days to about 20 days, about 12 days to about 25 days, about 12 days to about 30 days, about 13 days to about 14 days, about 13 days to about 15 days, about 13 days to about 20 days, about 13 days to about 25 days, about 13 days to about 30 days, about 14 days to about 15 days, about 14 days to about 20 days, about 14 days to about 25 days, about 14 days to about 30 days, about 15 days to about 20 days, about 15 days to about 25 days, about 15 days to about 30 days, about 20 days to about 25 days, about 20 days to about 30 days, or about 25 days to about 30 days.

In other aspects of this embodiment, a composition disclosed herein has an effective-use stability of, e.g., about 1 week, about 2 weeks, 3 weeks, 4 weeks, about 5 weeks, about 6 weeks, about 7 weeks, about 8 weeks, about 9 weeks, about 10 weeks, about 11 weeks, or about 12 weeks. In yet other aspects of this embodiment, a composition disclosed herein has an effective-use stability of, e.g., at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, or at least 12 weeks. In still other aspects of this embodiment, a composition disclosed herein has an effective-use stability of, e.g., at most 1 week, at most 2 weeks, at most 3 weeks, at most 4 weeks, at most 5 weeks, at most 6 weeks, at most 7 weeks, at most 8 weeks, at most 9 weeks, at most 10 weeks, at most 11 weeks, or at most 12 weeks. In aspects of this embodiment, a composition disclosed herein has an effective-use stability of, e.g., about 1 week to about 2 weeks, about 1 week to about 3 weeks, about 1 week to about 4 weeks, about 1 week to about 5 weeks, about 1 week to about 6 weeks, about 1 week to about 7 weeks, about 1 week to about 8 weeks, about 1 week to about 9 weeks, about 1 week to about 10 weeks, about 1 week to about 11 weeks, about 1 week to about 12 weeks, about 2 weeks to about 3 weeks, about 2 weeks to about 4 weeks, about 2 weeks to about 5 weeks, about 2 weeks to about 6 weeks, about 2 weeks to about 7 weeks, about 2 weeks to about 8 weeks, about 2 weeks to about 9 weeks, about 2 weeks to about 10 weeks, about 2 weeks to about 11 weeks, about 2 weeks to about 12 weeks, about 3 weeks to about 4 weeks, about 3 weeks to about 5 weeks, about 3 weeks to about 6 weeks, about 3 weeks to about 7 weeks, about 3 weeks to about 8 weeks, about 3 weeks to about 9 weeks, about 3 weeks to about 10 weeks, about 3 weeks to about 11 weeks, about 3 weeks to about 12 weeks, about 4 weeks to about 5 weeks, about 4 weeks to about 6 weeks, about 4 weeks to about 7 weeks, about 4 weeks to about 8 weeks, about 4 weeks to about 9 weeks, about 4 weeks to about 10 weeks, about 4 weeks to about 11 weeks, about 4 weeks to about 12 weeks, about 5 weeks to about 6 weeks, about 5 weeks to about 7 weeks, about 5 weeks to about 8 weeks, about 5 weeks to about 9 weeks, about 5 weeks to about 10 weeks, about 5 weeks to about 11 weeks, about 5 weeks to about 12 weeks, about 6 weeks to about 7 weeks, about 6 weeks to about 8 weeks, about 6 weeks to about 9 weeks, about 6 weeks to about 10 weeks, about 6 weeks to about 11 weeks, about 6 weeks to about 12 weeks, about 7 weeks to about 8 weeks, about 7 weeks to about 9 weeks, about 7 weeks to about 10 weeks, about 7 weeks to about 11 weeks, about 7 weeks to about 12 weeks, about 8 weeks to about 9 weeks, about 8 weeks to about 10 weeks, about 8 weeks to about 11 weeks, about 8 weeks to about 12 weeks, about 9 weeks to about 10 weeks, about 9 weeks to about 11 weeks, about 9 weeks to about 12 weeks, about 10 weeks to about 11 weeks, about 10 weeks to about 12 weeks, or about 11 weeks to about 12 weeks.

In other aspects of this embodiment, a composition disclosed herein has a shelf-life stability of, e.g., about 1 month, about 2 months, about 3 months, about 4 months, about 5 months, about 6, months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, or about 24 months. In yet other aspects of this embodiment, a composition disclosed herein has a shelf-life stability o, e.g., at least 1 month, at least 2 months, at least 3 months, at least 4 months, at least 5 months, at least 6, months, at least 7 months, at least 8 months, at least 9 months, at least 10 months, at least 11 months, at least 12 months, at least 13 months, at least 14 months, at least 15 months, at least 16 months, at least 17 months, at least 18 months, at least 19 months, at least 20 months, at least 21 months, at least 22 months, at least 23 months, or at least 24 months. In still other aspects of this embodiment, a composition disclosed herein has a shelf-life stability of, e.g., at most 1 month, at most 2 months, at most 3 months, at most 4 months, at most 5 months, at most 6, months, at most 7 months, at most 8 months, at most 9 months, at most 10 months, at most 11 months, at most 12 months, at most 13 months, at most 14 months, at most 15 months, at most 16 months, at most 17 months, at most 18 months, at most 19 months, at most 20 months, at most 21 months, at most 22 months, at most 23 months, or at most 24 months.

In aspects of this embodiment, a composition disclosed herein has a shelf-life stability of, e.g., about 1 month to about 2 months, about 1 month to about 3 months, about 1 month to about 4 months, about 1 month to about 5 months, about 1 month to about 6 months, about 1 month to about 7 months, about 1 month to about 8 months, about 1 month to about 9 months, about 1 month to about 10 months, about 1 month to about 11 months, about 1 month to about 12 months, about 1 month to about 13 months, about 1 month to about 14 months, about 1 month to about 15 months, about 1 month to about 16 months, about 1 month to about 17 months, about 1 month to about 18 months, about 1 month to about 19 months, about 1 month to about 20 months, about 1 month to about 21 months, about 1 month to about 22 months, about 1 month to about 23 months, about 1 month to about 24 months, about 2 months to about 3 months, about 2 months to about 4 months, about 2 months to about 5 months, about 2 months to about 6 months, about 2 months to about 7 months, about 2 months to about 8 months, about 2 months to about 9 months, about 2 months to about 10 months, about 2 months to about 11 months, about 2 months to about 12 months, about 2 months to about 13 months, about 2 months to about 14 months, about 2 months to about 15 months, about 2 months to about 16 months, about 2 months to about 17 months, about 2 months to about 18 months, about 2 months to about 19 months, about 2 months to about 20 months, about 2 months to about 21 months, about 2 months to about 22 months, about 2 months to about 23 months, about 2 months to about 24 months, about 3 months to about 4 months, about 3 months to about 5 months, about 3 months to about 6 months, about 3 months to about 7 months, about 3 months to about 8 months, about 3 months to about 9 months, about 3 months to about 10 months, about 3 months to about 11 months, about 3 months to about 12 months, about 3 months to about 13 months, about 3 months to about 14 months, about 3 months to about 15 months, about 3 months to about 16 months, about 3 months to about 17 months, about 3 months to about 18 months, about 3 months to about 19 months, about 3 months to about 20 months, about 3 months to about 21 months, about 3 months to about 22 months, about 3 months to about 23 months, about 3 months to about 24 months, about 4 months to about 5 months, about 4 months to about 6 months, about 4 months to about 7 months, about 4 months to about 8 months, about 4 months to about 9 months, about 4 months to about 10 months, about 4 months to about 11 months, about 4 months to about 12 months, about 4 months to about 13 months, about 4 months to about 14 months, about 4 months to about 15 months, about 4 months to about 16 months, about 4 months to about 17 months, about 4 months to about 18 months, about 4 months to about 19 months, about 4 months to about 20 months, about 4 months to about 21 months, about 4 months to about 22 months, about 4 months to about 23 months, about 4 months to about 24 months, about 5 months to about 6 months, about 5 months to about 7 months, about 5 months to about 8 months, about 5 months to about 9 months, about 5 months to about 10 months, about 5 months to about 11 months, about 5 months to about 12 months, about 5 months to about 13 months, about 5 months to about 14 months, about 5 months to about 15 months, about 5 months to about 16 months, about 5 months to about 17 months, about 5 months to about 18 months, about 5 months to about 19 months, about 5 months to about 20 months, about 5 months to about 21 months, about 5 months to about 22 months, about 5 months to about 23 months, about 5 months to about 24 months, about 6 months to about 7 months, about 6 months to about 8 months, about 6 months to about 9 months, about 6 months to about 10 months, about 6 months to about 11 months, about 6 months to about 12 months, about 6 months to about 13 months, about 6 months to about 14 months, about 6 months to about 15 months, about 6 months to about 16 months, about 6 months to about 17 months, about 6 months to about 18 months, about 6 months to about 19 months, about 6 months to about 20 months, about 6 months to about 21 months, about 6 months to about 22 months, about 6 months to about 23 months, about 6 months to about 24 months, about 7 months to about 8 months, about 7 months to about 9 months, about 7 months to about 10 months, about 7 months to about 11 months, about 7 months to about 12 months, about 7 months to about 13 months, about 7 months to about 14 months, about 7 months to about 15 months, about 7 months to about 16 months, about 7 months to about 17 months, about 7 months to about 18 months, about 7 months to about 19 months, about 7 months to about 20 months, about 7 months to about 21 months, about 7 months to about 22 months, about 7 months to about 23 months, about 7 months to about 24 months, about 8 months to about 9 months, about 8 months to about 10 months, about 8 months to about 11 months, about 8 months to about 12 months, about 8 months to about 13 months, about 8 months to about 14 months, about 8 months to about 15 months, about 8 months to about 16 months, about 8 months to about 17 months, about 8 months to about 18 months, about 8 months to about 19 months, about 8 months to about 20 months, about 8 months to about 21 months, about 8 months to about 22 months, about 8 months to about 23 months, about 8 months to about 24 months, about 9 months to about 10 months, about 9 months to about 11 months, about 9 months to about 12 months, about 9 months to about 13 months, about 9 months to about 14 months, about 9 months to about 15 months, about 9 months to about 16 months, about 9 months to about 17 months, about 9 months to about 18 months, about 9 months to about 19 months, about 9 months to about 20 months, about 9 months to about 21 months, about 9 months to about 22 months, about 9 months to about 23 months, about 9 months to about 24 months, about 10 months to about 11 months, about 10 months to about 12 months, about 10 months to about 13 months, about 10 months to about 14 months, about 10 months to about 15 months, about 10 months to about 16 months, about 10 months to about 17 months, about 10 months to about 18 months, about 10 months to about 19 months, about 10 months to about 20 months, about 10 months to about 21 months, about 10 months to about 22 months, about 10 months to about 23 months, about 10 months to about 24 months, about 11 months to about 12 months, about 11 months to about 13 months, about 11 months to about 14 months, about 11 months to about 15 months, about 11 months to about 16 months, about 11 months to about 17 months, about 11 months to about 18 months, about 11 months to about 19 months, about 11 months to about 20 months, about 11 months to about 21 months, about 11 months to about 22 months, about 11 months to about 23 months, about 11 months to about 24 months, about 12 months to about 13 months, about 12 months to about 14 months, about 12 months to about 15 months, about 12 months to about 16 months, about 12 months to about 17 months, about 12 months to about 18 months, about 12 months to about 19 months, about 12 months to about 20 months, about 12 months to about 21 months, about 12 months to about 22 months, about 12 months to about 23 months, about 12 months to about 24 months, about 13 months to about 14 months, about 13 months to about 15 months, about 13 months to about 16 months, about 13 months to about 17 months, about 13 months to about 18 months, about 13 months to about 19 months, about 13 months to about 20 months, about 13 months to about 21 months, about 13 months to about 22 months, about 13 months to about 23 months, about 13 months to about 24 months, about 14 months to about 15 months, about 14 months to about 16 months, about 14 months to about 17 months, about 14 months to about 18 months, about 14 months to about 19 months, about 14 months to about 20 months, about 14 months to about 21 months, about 14 months to about 22 months, about 14 months to about 23 months, about 14 months to about 24 months, about 15 months to about 16 months, about 15 months to about 17 months, about 15 months to about 18 months, about 15 months to about 19 months, about 15 months to about 20 months, about 15 months to about 21 months, about 15 months to about 22 months, about 15 months to about 23 months, about 15 months to about 24 months, about 16 months to about 17 months, about 16 months to about 18 months, about 16 months to about 19 months, about 16 months to about 20 months, about 16 months to about 21 months, about 16 months to about 22 months, about 16 months to about 23 months, about 16 months to about 24 months, about 17 months to about 18 months, about 17 months to about 19 months, about 17 months to about 20 months, about 17 months to about 21 months, about 17 months to about 22 months, about 17 months to about 23 months, about 17 months to about 24 months, about 18 months to about 19 months, about 18 months to about 20 months, about 18 months to about 21 months, about 18 months to about 22 months, about 18 months to about 23 months, about 18 months to about 24 months, about 19 months to about 20 months, about 19 months to about 21 months, about 19 months to about 22 months, about 19 months to about 23 months, about 19 months to about 24 months, about 20 months to about 22 months, about 20 months to about 23 months, about 20 months to about 24 months, about 21 months to about 22 months, about 21 months to about 23 months, about 21 months to about 24 months, about 22 months to about 23 months, about 22 months to about 24 months, or about 23 months to about 24 months.

In other aspects of this embodiment, a composition disclosed herein has a shelf-life stability of, e.g., about 1 year, about 2 years, about 3 years, about 4 years, or about 5 years. In yet other aspects of this embodiment, a composition disclosed herein has a shelf-life stability of, e.g., at least 1 year, at least 2 years, at least 3 years, at least 4 years, or at least 5 years. In still other aspects of this embodiment, a composition disclosed herein has a shelf-life stability of, e.g., at most 1 year, at most 2 years, at most 3 years, at most 4 years, or at most 5 years. In other aspects of this embodiment, a composition disclosed herein has a shelf-life stability of, e.g., about 1 year to about 2 years, about 1 year to about 3 years, about 1 year to about 4 years, about 1 year to about 5 years, about 2 years to about 3 years, about 2 years to about 4 years, about 2 years to about 5 years, about 3 years to about 4 years, about 3 years to about 5 years, or about 4 years to about 5 years.

A composition disclosed herein can be formulated into any form that enables application of a composition disclosed herein in a manner that achieves a desired beneficial effect. In one embodiment, a composition disclosed herein can be formulated into, e.g., a single-phase formulation or a biphasic formulation comprising a medium phase and a dispersed phase. In another embodiment, a composition disclosed herein can be formulated into, e.g., a liquid composition, a colloidal composition, a semi-solid composition, or a solid composition. In another embodiment, a composition disclosed herein can be formulated into, e.g., a liquid aerosol, a foam, an emulsion, a gel, a sol, or a solid sol. In another embodiment, a composition disclosed herein can be formulated into, e.g., a spray, a liquid aerosol, a soap, or a suspension.

In an embodiment, a composition disclosed herein comprises 5 ppm to 100 ppm hypochlorous acid or free available chlorine. In an aspect of this embodiment, a composition disclosed herein comprises 10 ppm to 90 ppm hypochlorous acid or free available chlorine. In an aspect of this embodiment, a composition disclosed herein comprises 15 ppm to 85 ppm hypochlorous acid or free available chlorine. In an aspect of this embodiment, a composition disclosed herein comprises 20 ppm to 80 ppm hypochlorous acid or free available chlorine. In an aspect of this embodiment, a composition disclosed herein comprises 25 ppm to 75 ppm hypochlorous acid or free available chlorine.

In an embodiment, a composition disclosed herein comprises 5 ppm to 90 ppm hypochlorous acid or free available chlorine. In an aspect of this embodiment, a composition disclosed herein comprises 10 ppm to 85 ppm hypochlorous acid or free available chlorine. In an aspect of this embodiment, a composition disclosed herein comprises 15 ppm to 80 ppm hypochlorous acid or free available chlorine. In an aspect of this embodiment, a composition disclosed herein comprises 20 ppm to 75 ppm hypochlorous acid or free available chlorine. In an aspect of this embodiment, a composition disclosed herein comprises 25 ppm to 70 ppm hypochlorous acid or free available chlorine.

In an embodiment, a composition disclosed herein comprises 5 ppm to 80 ppm hypochlorous acid or free available chlorine. In an aspect of this embodiment, a composition disclosed herein comprises 10 ppm to 75 ppm hypochlorous acid or free available chlorine. In an aspect of this embodiment, a composition disclosed herein comprises 15 ppm to 70 ppm hypochlorous acid or free available chlorine. In an aspect of this embodiment, a composition disclosed herein comprises 20 ppm to 65 ppm hypochlorous acid or free available chlorine. In an aspect of this embodiment, a composition disclosed herein comprises 25 ppm to 60 ppm hypochlorous acid or free available chlorine.

In an embodiment, a composition disclosed herein comprises 5 ppm to 70 ppm hypochlorous acid or free available chlorine. In an aspect of this embodiment, a composition disclosed herein comprises 10 ppm to 65 ppm hypochlorous acid or free available chlorine. In an aspect of this embodiment, a composition disclosed herein comprises 15 ppm to 60 ppm hypochlorous acid or free available chlorine. In an aspect of this embodiment, a composition disclosed herein comprises 20 ppm to 55 ppm hypochlorous acid or free available chlorine. In an aspect of this embodiment, a composition disclosed herein comprises 25 ppm to 50 ppm hypochlorous acid or free available chlorine.

In an embodiment, a composition disclosed herein comprises 5 ppm to 60 ppm hypochlorous acid or free available chlorine. In an aspect of this embodiment, a composition disclosed herein comprises 10 ppm to 55 ppm hypochlorous acid or free available chlorine. In an aspect of this embodiment, a composition disclosed herein comprises 15 ppm to 50 ppm hypochlorous acid or free available chlorine. In an aspect of this embodiment, a composition disclosed herein comprises 20 ppm to 45 ppm hypochlorous acid or free available chlorine. In an aspect of this embodiment, a composition disclosed herein comprises 25 ppm to 40 ppm hypochlorous acid or free available chlorine.

In an embodiment, a composition disclosed herein comprises 5 ppm to 50 ppm hypochlorous acid or free available chlorine. In an aspect of this embodiment, a composition disclosed herein comprises 10 ppm to 45 ppm hypochlorous acid or free available chlorine. In an aspect of this embodiment, a composition disclosed herein comprises 15 ppm to 40 ppm hypochlorous acid or free available chlorine. In an aspect of this embodiment, a composition disclosed herein comprises 20 ppm to 35 ppm hypochlorous acid or free available chlorine. In an aspect of this embodiment, a composition disclosed herein comprises 25 ppm to 30 ppm hypochlorous acid or free available chlorine.

In an embodiment, a composition disclosed herein comprises 90 ppm to 140 ppm hypochlorous acid or free available chlorine. In an aspect of this embodiment, a composition disclosed herein comprises 95 ppm to 135 ppm hypochlorous acid or free available chlorine. In an aspect of this embodiment, a composition disclosed herein comprises 100 ppm to 130 ppm hypochlorous acid or free available chlorine. In an aspect of this embodiment, a composition disclosed herein comprises 105 ppm to 125 ppm hypochlorous acid or free available chlorine. In an aspect of this embodiment, a composition disclosed herein comprises 110 ppm to 120 ppm hypochlorous acid or free available chlorine. In an aspect of this embodiment, a composition disclosed herein comprises 115 ppm hypochlorous acid or free available chlorine.

In an embodiment, a composition disclosed herein comprises 75 ppm to 125 ppm hypochlorous acid or free available chlorine. In an aspect of this embodiment, a composition disclosed herein comprises 80 ppm to 120 ppm hypochlorous acid or free available chlorine. In an aspect of this embodiment, a composition disclosed herein comprises 85 ppm to 115 ppm hypochlorous acid or free available chlorine. In an aspect of this embodiment, a composition disclosed herein comprises 90 ppm to 110 ppm hypochlorous acid or free available chlorine. In an aspect of this embodiment, a composition disclosed herein comprises 95 ppm to 105 ppm hypochlorous acid or free available chlorine. In an aspect of this embodiment, a composition disclosed herein comprises 100 ppm hypochlorous acid or free available chlorine.

In an embodiment, a composition disclosed herein comprises 50 ppm to 100 ppm hypochlorous acid or free available chlorine. In an aspect of this embodiment, a composition disclosed herein comprises 55 ppm to 95 ppm hypochlorous acid or free available chlorine. In an aspect of this embodiment, a composition disclosed herein comprises 60 ppm to 90 ppm hypochlorous acid or free available chlorine. In an aspect of this embodiment, a composition disclosed herein comprises 65 ppm to 85 ppm hypochlorous acid or free available chlorine. In an aspect of this embodiment, a composition disclosed herein comprises 70 ppm to 80 ppm hypochlorous acid or free available chlorine. In an aspect of this embodiment, a composition disclosed herein comprises 75 ppm hypochlorous acid or free available chlorine.

In an embodiment, a composition disclosed herein comprises 45 ppm to 95 ppm hypochlorous acid or free available chlorine. In an aspect of this embodiment, a composition disclosed herein comprises 50 ppm to 90 ppm hypochlorous acid or free available chlorine. In an aspect of this embodiment, a composition disclosed herein comprises 55 ppm to 85 ppm hypochlorous acid or free available chlorine. In an aspect of this embodiment, a composition disclosed herein comprises 60 ppm to 80 ppm hypochlorous acid or free available chlorine. In an aspect of this embodiment, a composition disclosed herein comprises 65 ppm to 75 ppm hypochlorous acid or free available chlorine. In an aspect of this embodiment, a composition disclosed herein comprises 70 ppm hypochlorous acid or free available chlorine.

In an embodiment, a composition disclosed herein comprises 40 ppm to 90 ppm hypochlorous acid or free available chlorine. In an aspect of this embodiment, a composition disclosed herein comprises 45 ppm to 85 ppm hypochlorous acid or free available chlorine. In an aspect of this embodiment, a composition disclosed herein comprises 50 ppm to 80 ppm hypochlorous acid or free available chlorine. In an aspect of this embodiment, a composition disclosed herein comprises 55 ppm to 75 ppm hypochlorous acid or free available chlorine. In an aspect of this embodiment, a composition disclosed herein comprises 60 ppm to 70 ppm hypochlorous acid or free available chlorine. In an aspect of this embodiment, a composition disclosed herein comprises 65 ppm hypochlorous acid or free available chlorine.

In an embodiment, a composition disclosed herein comprises 25 ppm to 85 ppm hypochlorous acid or free available chlorine. In an aspect of this embodiment, a composition disclosed herein comprises 40 ppm to 80 ppm hypochlorous acid or free available chlorine. In an aspect of this embodiment, a composition disclosed herein comprises 45 ppm to 75 ppm hypochlorous acid or free available chlorine. In an aspect of this embodiment, a composition disclosed herein comprises 50 ppm to 70 ppm hypochlorous acid or free available chlorine. In an aspect of this embodiment, a composition disclosed herein comprises 55 ppm to 65 ppm hypochlorous acid or free available chlorine. In an aspect of this embodiment, a composition disclosed herein comprises 60 ppm hypochlorous acid or free available chlorine.

In an embodiment, a composition disclosed herein comprises 30 ppm to 80 ppm hypochlorous acid or free available chlorine. In an aspect of this embodiment, a composition disclosed herein comprises 35 ppm to 75 ppm hypochlorous acid or free available chlorine. In an aspect of this embodiment, a composition disclosed herein comprises 40 ppm to 70 ppm hypochlorous acid or free available chlorine. In an aspect of this embodiment, a composition disclosed herein comprises 45 ppm to 65 ppm hypochlorous acid or free available chlorine. In an aspect of this embodiment, a composition disclosed herein comprises 50 ppm to 60 ppm hypochlorous acid or free available chlorine. In an aspect of this embodiment, a composition disclosed herein comprises 55 ppm hypochlorous acid or free available chlorine.

In an embodiment, a composition disclosed herein comprises 25 ppm to 75 ppm hypochlorous acid or free available chlorine. In an aspect of this embodiment, a composition disclosed herein comprises 30 ppm to 70 ppm hypochlorous acid or free available chlorine. In an aspect of this embodiment, a composition disclosed herein comprises 35 ppm to 65 ppm hypochlorous acid or free available chlorine. In an aspect of this embodiment, a composition disclosed herein comprises 40 ppm to 60 ppm hypochlorous acid or free available chlorine. In an aspect of this embodiment, a composition disclosed herein comprises 45 ppm to 55 ppm hypochlorous acid or free available chlorine. In an aspect of this embodiment, a composition disclosed herein comprises 50 ppm hypochlorous acid or free available chlorine.

In an embodiment, a composition disclosed herein comprises 20 ppm to 70 ppm hypochlorous acid or free available chlorine. In an aspect of this embodiment, a composition disclosed herein comprises 25 ppm to 65 ppm hypochlorous acid or free available chlorine. In an aspect of this embodiment, a composition disclosed herein comprises 30 ppm to 60 ppm hypochlorous acid or free available chlorine. In an aspect of this embodiment, a composition disclosed herein comprises 35 ppm to 55 ppm hypochlorous acid or free available chlorine. In an aspect of this embodiment, a composition disclosed herein comprises 40 ppm to 50 ppm hypochlorous acid or free available chlorine. In an aspect of this embodiment, a composition disclosed herein comprises 45 ppm hypochlorous acid or free available chlorine.

In an embodiment, a composition disclosed herein comprises 15 ppm to 65 ppm hypochlorous acid or free available chlorine. In an aspect of this embodiment, a composition disclosed herein comprises 20 ppm to 60 ppm hypochlorous acid or free available chlorine. In an aspect of this embodiment, a composition disclosed herein comprises 25 ppm to 55 ppm hypochlorous acid or free available chlorine. In an aspect of this embodiment, a composition disclosed herein comprises 30 ppm to 50 ppm hypochlorous acid or free available chlorine. In an aspect of this embodiment, a composition disclosed herein comprises 35 ppm to 45 ppm hypochlorous acid or free available chlorine. In an aspect of this embodiment, a composition disclosed herein comprises 40 ppm hypochlorous acid or free available chlorine.

In an embodiment, a composition disclosed herein comprises 10 ppm to 60 ppm hypochlorous acid or free available chlorine. In an aspect of this embodiment, a composition disclosed herein comprises 15 ppm to 55 ppm hypochlorous acid or free available chlorine. In an aspect of this embodiment, a composition disclosed herein comprises 20 ppm to 50 ppm hypochlorous acid or free available chlorine. In an aspect of this embodiment, a composition disclosed herein comprises 25 ppm to 45 ppm hypochlorous acid or free available chlorine. In an aspect of this embodiment, a composition disclosed herein comprises 30 ppm to 40 ppm hypochlorous acid or free available chlorine. In an aspect of this embodiment, a composition disclosed herein comprises 35 ppm hypochlorous acid or free available chlorine.

In an embodiment, a composition disclosed herein comprises 5 ppm to 55 ppm hypochlorous acid or free available chlorine. In an aspect of this embodiment, a composition disclosed herein comprises 10 ppm to 50 ppm hypochlorous acid or free available chlorine. In an aspect of this embodiment, a composition disclosed herein comprises 15 ppm to 45 ppm hypochlorous acid or free available chlorine. In an aspect of this embodiment, a composition disclosed herein comprises 20 ppm to 40 ppm hypochlorous acid or free available chlorine. In an aspect of this embodiment, a composition disclosed herein comprises 25 ppm to 35 ppm hypochlorous acid or free available chlorine. In an aspect of this embodiment, a composition disclosed herein comprises 30 ppm hypochlorous acid or free available chlorine.

In an aspect of this embodiment, a composition disclosed herein comprises 1 ppm to 50 ppm hypochlorous acid or free available chlorine. In an aspect of this embodiment, a composition disclosed herein comprises 5 ppm to 45 ppm hypochlorous acid or free available chlorine. In an aspect of this embodiment, a composition disclosed herein comprises 10 ppm to 40 ppm hypochlorous acid or free available chlorine. In an aspect of this embodiment, a composition disclosed herein comprises 15 ppm to 35 ppm hypochlorous acid or free available chlorine. In an aspect of this embodiment, a composition disclosed herein comprises 20 ppm to 30 ppm hypochlorous acid or free available chlorine. In an aspect of this embodiment, a composition disclosed herein comprises 25 ppm hypochlorous acid or free available chlorine.

In an embodiment, a composition disclosed herein comprises 1 ppm to 100 ppm hypochlorous acid or free available chlorine, and 1 ppm to 200 ppm one or more metal salts. In an aspect of this embodiment, a composition disclosed herein comprises 1 ppm to 100 ppm hypochlorous acid or free available chlorine, and 1 ppm to 50 ppm of a copper salt, 1 ppm to 50 ppm of a zinc salt, 1 ppm to 50 ppm of an iron salt, or any combination thereof. In an aspect of this embodiment, a composition disclosed herein comprises 5 ppm to 75 ppm hypochlorous acid or free available chlorine, and 5 ppm to 40 ppm of a copper salt, 5 ppm to 40 ppm of a zinc salt and 5 ppm to 40 ppm of an iron salt. In an aspect of this embodiment, a composition disclosed herein comprises 40 ppm to 60 ppm hypochlorous acid or free available chlorine, and 10 ppm to 30 ppm of a copper salt, 10 ppm to 30 ppm of a zinc salt and 10 ppm to 30 ppm of an iron salt. In an aspect of this embodiment, a composition disclosed herein comprises 50 ppm hypochlorous acid or free available chlorine, and 20 ppm of a copper salt, 20 ppm of a zinc salt and 20 ppm of an iron salt.

In an embodiment, a composition disclosed herein comprises 1 ppm to 100 ppm hypochlorous acid or free available chlorine, and 1 ppm to 200 ppm one or more metal salts. In an aspect of this embodiment, a composition disclosed herein comprises 1 ppm to 100 ppm hypochlorous acid or free available chlorine, and 1 ppm to 50 ppm of a copper salt, 1 ppm to 50 ppm of a zinc salt, 1 ppm to 50 ppm of a calcium salt, or any combination thereof. In an aspect of this embodiment, a composition disclosed herein comprises 5 ppm to 75 ppm hypochlorous acid or free available chlorine, and 5 ppm to 40 ppm of a copper salt, 5 ppm to 40 ppm of a zinc salt and 5 ppm to 40 ppm of a calcium salt. In an aspect of this embodiment, a composition disclosed herein comprises 40 ppm to 60 ppm hypochlorous acid or free available chlorine, and 10 ppm to 30 ppm of a copper salt, 10 ppm to 30 ppm of a zinc salt and 10 ppm to 30 ppm of a calcium salt. In an aspect of this embodiment, a composition disclosed herein comprises 50 ppm hypochlorous acid or free available chlorine, and 20 ppm of a copper salt, 20 ppm of a zinc salt and 20 ppm of a calcium salt.

In an aspect of this embodiment, a composition disclosed herein comprises 1 ppm to 100 ppm hypochlorous acid or free available chlorine, and 1 ppm to 50 ppm of a copper chloride, 1 ppm to 50 ppm of a zinc chloride, 1 ppm to 50 ppm of an iron chloride, or any combination thereof. In an aspect of this embodiment, a composition disclosed herein comprises 5 ppm to 75 ppm hypochlorous acid or free available chlorine, and 5 ppm to 40 ppm of a copper chloride, 5 ppm to 40 ppm of a zinc chloride and 5 ppm to 40 ppm of an iron chloride. In an aspect of this embodiment, a composition disclosed herein comprises 40 ppm to 60 ppm hypochlorous acid or free available chlorine, and 10 ppm to 30 ppm of a copper chloride, 10 ppm to 30 ppm of a zinc chloride and 10 ppm to 30 ppm of an iron chloride. In an aspect of this embodiment, a composition disclosed herein comprises 50 ppm hypochlorous acid or free available chlorine, and 20 ppm of a copper chloride, 20 ppm of a zinc chloride and 20 ppm of an iron chloride. The iron chloride can be iron (II) chloride or iron (III) chloride.

In an aspect of this embodiment, a composition disclosed herein comprises 1 ppm to 100 ppm hypochlorous acid or free available chlorine, and 1 ppm to 50 ppm of a copper chloride, 1 ppm to 50 ppm of a zinc chloride, 1 ppm to 50 ppm of a calcium oxide salt, or any combination thereof. In an aspect of this embodiment, a composition disclosed herein comprises 5 ppm to 75 ppm hypochlorous acid or free available chlorine, and 5 ppm to 40 ppm of a copper chloride, 5 ppm to 40 ppm of a zinc chloride and 5 ppm to 40 ppm of a calcium oxide salt. In an aspect of this embodiment, a composition disclosed herein comprises 40 ppm to 60 ppm hypochlorous acid or free available chlorine, and 10 ppm to 30 ppm of a copper chloride, 10 ppm to 30 ppm of a zinc chloride and 10 ppm to 30 ppm of a calcium oxide salt. In an aspect of this embodiment, a composition disclosed herein comprises 50 ppm hypochlorous acid or free available chlorine, and 20 ppm of a copper chloride, 20 ppm of a zinc chloride and 20 ppm of a calcium oxide salt.

In an embodiment, a composition disclosed herein comprises 0.1 ppm to 10 ppm hypochlorous acid or free available chlorine, and 0.1 ppm to 20 ppm one or more metal salts. In an aspect of this embodiment, a composition disclosed herein comprises 0.1 ppm to 10 ppm hypochlorous acid or free available chlorine, and 0.1 ppm to 5 ppm of a copper salt, 0.1 ppm to 5 ppm of a zinc salt, 0.1 ppm to 5 ppm of an iron salt, or any combination thereof. In an aspect of this embodiment, a composition disclosed herein comprises 0.5 ppm to 7.5 ppm hypochlorous acid or free available chlorine, and 0.5 ppm to 4 ppm of a copper salt, 0.5 ppm to 4 ppm of a zinc salt and 0.5 ppm to 4 ppm of an iron salt. In an aspect of this embodiment, a composition disclosed herein comprises 4 ppm to 6 ppm hypochlorous acid or free available chlorine, and 1 ppm to 3 ppm of a copper salt, 1 ppm to 3 ppm of a zinc salt and 1 ppm to 3 ppm of an iron salt. In an aspect of this embodiment, a composition disclosed herein comprises 5 ppm hypochlorous acid or free available chlorine, and 2 ppm of a copper salt, 2 ppm of a zinc salt and 2 ppm of an iron salt.

In an embodiment, a composition disclosed herein comprises 0.1 ppm to 10 ppm hypochlorous acid or free available chlorine, and 0.1 ppm to 20 ppm one or more metal salts. In an aspect of this embodiment, a composition disclosed herein comprises 0.1 ppm to 10 ppm hypochlorous acid or free available chlorine, and 0.1 ppm to 5 ppm of a copper salt, 0.1 ppm to 5 ppm of a zinc salt, 0.1 ppm to 5 ppm of a calcium salt, or any combination thereof. In an aspect of this embodiment, a composition disclosed herein comprises 0.5 ppm to 7.5 ppm hypochlorous acid or free available chlorine, and 0.5 ppm to 4 ppm of a copper salt, 0.5 ppm to 4 ppm of a zinc salt and 0.5 ppm to 4 ppm of a calcium salt. In an aspect of this embodiment, a composition disclosed herein comprises 4 ppm to 6 ppm hypochlorous acid or free available chlorine, and 1 ppm to 3 ppm of a copper salt, 1 ppm to 3 ppm of a zinc salt and 1 ppm to 3 ppm of a calcium salt. In an aspect of this embodiment, a composition disclosed herein comprises 5 ppm hypochlorous acid or free available chlorine, and 2 ppm of a copper salt, 2 ppm of a zinc salt and 2 ppm of a calcium salt.

In an aspect of this embodiment, a composition disclosed herein comprises 0.1 ppm to 10 ppm hypochlorous acid or free available chlorine, and 0.1 ppm to 5 ppm of a copper chloride, 0.1 ppm to 5 ppm of a zinc chloride, 0.1 ppm to 5 ppm of an iron chloride, or any combination thereof. In an aspect of this embodiment, a composition disclosed herein comprises 0.5 ppm to 7.5 ppm hypochlorous acid or free available chlorine, and 0.5 ppm to 4 ppm of a copper chloride, 0.5 ppm to 4 ppm of a zinc chloride and 0.5 ppm to 4 ppm of an iron chloride. In an aspect of this embodiment, a composition disclosed herein comprises 4 ppm to 6 ppm hypochlorous acid or free available chlorine, and 1 ppm to 3 ppm of a copper chloride, 1 ppm to 3 ppm of a zinc chloride and 1 ppm to 3 ppm of an iron chloride. In an aspect of this embodiment, a composition disclosed herein comprises 5 ppm hypochlorous acid or free available chlorine, and 2 ppm of a copper chloride, 2 ppm of a zinc chloride and 2 ppm of an iron chloride. The iron chloride can be iron (II) chloride or iron (III) chloride.

In an aspect of this embodiment, a composition disclosed herein comprises 0.1 ppm to 10 ppm hypochlorous acid or free available chlorine, and 0.1 ppm to 5 ppm of a copper chloride, 0.1 ppm to 5 ppm of a zinc chloride, 0.1 ppm to 5 ppm of a calcium oxide salt, or any combination thereof. In an aspect of this embodiment, a composition disclosed herein comprises 0.5 ppm to 7.5 ppm hypochlorous acid or free available chlorine, and 0.5 ppm to 4 ppm of a copper chloride, 0.5 ppm to 4 ppm of a zinc chloride and 0.5 ppm to 4 ppm of a calcium oxide salt. In an aspect of this embodiment, a composition disclosed herein comprises 4 ppm to 6 ppm hypochlorous acid or free available chlorine, and 1 ppm to 3 ppm of a copper chloride, 1 ppm to 3 ppm of a zinc chloride and 1 ppm to 3 ppm of a calcium oxide salt. In an aspect of this embodiment, a composition disclosed herein comprises 5 ppm hypochlorous acid or free available chlorine, and 2 ppm of a copper chloride, 2 ppm of a zinc chloride and 2 ppm of a calcium oxide salt.

In an embodiment, a composition disclosed herein comprises 0.01 ppm to 5 ppm hypochlorous acid or free available chlorine, and 0.01 ppm to 5 ppm one or more metal salts. In an aspect of this embodiment, a composition disclosed herein comprises 0.05 ppm to 1 ppm hypochlorous acid or free available chlorine, and 0.01 ppm to 2 ppm one or more metal salts. In an aspect of this embodiment, a composition disclosed herein comprises 0.01 ppm to 1 ppm hypochlorous acid or free available chlorine, and 0.01 ppm to 0.5 ppm of a copper salt, 0.01 ppm to 0.5 ppm of a zinc salt, 0.01 ppm to 0.5 ppm of an iron salt, or any combination thereof. In an aspect of this embodiment, a composition disclosed herein comprises 0.05 ppm to 0.75 ppm hypochlorous acid or free available chlorine, and 0.05 ppm to 0.4 ppm of a copper salt, 0.05 ppm to 0.4 ppm of a zinc salt and 0.05 ppm to 0.4 ppm of an iron salt. In an aspect of this embodiment, a composition disclosed herein comprises 0.4 ppm to 0.6 ppm hypochlorous acid or free available chlorine, and 0.1 ppm to 0.3 ppm of a copper salt, 0.1 ppm to 0.3 ppm of a zinc salt and 0.1 ppm to 0.3 ppm of an iron salt. In an aspect of this embodiment, a composition disclosed herein comprises 0.5 ppm hypochlorous acid or free available chlorine, and 0.2 ppm of a copper salt, 0.2 ppm of a zinc salt and 0.2 ppm of an iron salt.

In an embodiment, a composition disclosed herein comprises 0.01 ppm to 5 ppm hypochlorous acid or free available chlorine, and 0.01 ppm to 2 ppm one or more metal salts. In an aspect of this embodiment, a composition disclosed herein comprises 0.05 ppm to 1 ppm hypochlorous acid or free available chlorine, and 0.01 ppm to 2 ppm one or more metal salts. In an aspect of this embodiment, a composition disclosed herein comprises 0.01 ppm to 1 ppm hypochlorous acid or free available chlorine, and 0.01 ppm to 0.5 ppm of a copper salt, 0.01 ppm to 0.5 ppm of a zinc salt, 0.01 ppm to 0.5 ppm of a calcium salt, or any combination thereof. In an aspect of this embodiment, a composition disclosed herein comprises 0.05 ppm to 0.75 ppm hypochlorous acid or free available chlorine, and 0.05 ppm to 0.4 ppm of a copper salt, 0.05 ppm to 0.4 ppm of a zinc salt and 0.05 ppm to 0.4 ppm of a calcium salt. In an aspect of this embodiment, a composition disclosed herein comprises 0.4 ppm to 0.6 ppm hypochlorous acid or free available chlorine, and 0.1 ppm to 0.3 ppm of a copper salt, 0.1 ppm to 0.3 ppm of a zinc salt and 0.1 ppm to 0.3 ppm of a calcium salt. In an aspect of this embodiment, a composition disclosed herein comprises 0.5 ppm hypochlorous acid or free available chlorine, and 0.2 ppm of a copper salt, 0.2 ppm of a zinc salt and 0.2 ppm of a calcium salt.

In an aspect of this embodiment, a composition disclosed herein comprises 0.01 ppm to 5 ppm hypochlorous acid or free available chlorine, and 0.01 ppm to 1 ppm of a copper chloride, 0.01 ppm to 1 ppm of a zinc chloride, 0.01 ppm to 1 ppm of an iron chloride, or any combination thereof. In an aspect of this embodiment, a composition disclosed herein comprises 0.05 ppm to 1 ppm hypochlorous acid or free available chlorine, and 0.01 ppm to 0.5 ppm of a copper chloride, 0.01 ppm to 0.5 ppm of a zinc chloride, 0.01 ppm to 0.5 ppm of an iron chloride, or any combination thereof. In an aspect of this embodiment, a composition disclosed herein comprises 0.05 ppm to 0.75 ppm hypochlorous acid or free available chlorine, and 0.05 ppm to 0.4 ppm of a copper chloride, 0.05 ppm to 0.4 ppm of a zinc chloride and 0.05 ppm to 0.4 ppm of an iron chloride. In an aspect of this embodiment, a composition disclosed herein comprises 0.4 ppm to 0.6 ppm hypochlorous acid or free available chlorine, and 0.1 ppm to 0.3 ppm of a copper chloride, 0.1 ppm to 0.3 ppm of a zinc chloride and 0.1 ppm to 0.3 ppm of an iron chloride. In an aspect of this embodiment, a composition disclosed herein comprises 0.5 ppm hypochlorous acid or free available chlorine, and 0.2 ppm of a copper chloride, 0.2 ppm of a zinc chloride and 0.2 ppm of an iron chloride. The iron chloride can be iron (II) chloride or iron (III) chloride.

In an aspect of this embodiment, a composition disclosed herein comprises 0.01 ppm to 5 ppm hypochlorous acid or free available chlorine, and 0.01 ppm to 1 ppm of a copper chloride, 0.01 ppm to 1 ppm of a zinc chloride, 0.01 ppm to 1 ppm of a calcium oxide salt, or any combination thereof. In an aspect of this embodiment, a composition disclosed herein comprises 0.01 ppm to 1 ppm hypochlorous acid or free available chlorine, and 0.01 ppm to 0.5 ppm of a copper chloride, 0.01 ppm to 0.5 ppm of a zinc chloride, 0.01 ppm to 0.5 ppm of a calcium oxide salt, or any combination thereof. In an aspect of this embodiment, a composition disclosed herein comprises 0.05 ppm to 0.75 ppm hypochlorous acid or free available chlorine, and 0.05 ppm to 0.4 ppm of a copper chloride, 0.05 ppm to 0.4 ppm of a zinc chloride and 0.05 ppm to 0.4 ppm of a calcium oxide salt. In an aspect of this embodiment, a composition disclosed herein comprises 0.4 ppm to 0.6 ppm hypochlorous acid or free available chlorine, and 0.1 ppm to 0.3 ppm of a copper chloride, 0.1 ppm to 0.3 ppm of a zinc chloride and 0.1 ppm to 0.3 ppm of a calcium oxide salt. In an aspect of this embodiment, a composition disclosed herein comprises 0.5 ppm hypochlorous acid or free available chlorine, and 0.2 ppm of a copper chloride, 0.2 ppm of a zinc chloride and 0.2 ppm of a calcium oxide salt.

In an embodiment, a composition disclosed herein comprises 1 ppm to 100 ppm hypochlorous acid or free available chlorine and 1 ppm to 100 ppm one or more metal particles. In an aspect of this embodiment, a composition disclosed herein comprises 1 ppm to 100 ppm hypochlorous acid or free available chlorine and 1 ppm to 100 ppm of metal oxide particles. In an aspect of this embodiment, a composition disclosed herein comprises 5 ppm to 75 ppm hypochlorous acid or free available chlorine and 5 ppm to 75 ppm of metal oxide particles. In an aspect of this embodiment, a composition disclosed herein comprises 40 ppm to 60 ppm hypochlorous acid or free available chlorine, and 10 ppm to 30 ppm of metal oxide particles. In an aspect of this embodiment, a composition disclosed herein comprises 50 ppm hypochlorous acid or free available chlorine and 20 ppm of metal oxide particles.

In an aspect of this embodiment, a composition disclosed herein comprises 1 ppm to 100 ppm hypochlorous acid or free available chlorine and 1 ppm to 100 ppm of copper zinc iron oxide particles. In an aspect of this embodiment, a composition disclosed herein comprises 5 ppm to 75 ppm hypochlorous acid or free available chlorine and 5 ppm to 75 ppm of copper zinc iron oxide particles. In an aspect of this embodiment, a composition disclosed herein comprises 40 ppm to 60 ppm hypochlorous acid or free available chlorine, and 10 ppm to 30 ppm of copper zinc iron oxide particles. In an aspect of this embodiment, a composition disclosed herein comprises 50 ppm hypochlorous acid or free available chlorine and 20 ppm of copper zinc iron oxide particles.

In an embodiment, a composition disclosed herein comprises 0.1 ppm to 10 ppm hypochlorous acid or free available chlorine and 0.1 ppm to 10 ppm one or more metal particles. In an aspect of this embodiment, a composition disclosed herein comprises 0.1 ppm to 10 ppm hypochlorous acid or free available chlorine and 0.1 ppm to 10 ppm of metal oxide particles. In an aspect of this embodiment, a composition disclosed herein comprises 0.5 ppm to 7.5 ppm hypochlorous acid or free available chlorine and 0.5 ppm to 7.5 ppm of metal oxide particles. In an aspect of this embodiment, a composition disclosed herein comprises 4 ppm to 6 ppm hypochlorous acid or free available chlorine, and 1 ppm to 3 ppm of metal oxide particles. In an aspect of this embodiment, a composition disclosed herein comprises 5 ppm hypochlorous acid or free available chlorine and 2 ppm of metal oxide particles.

In an aspect of this embodiment, a composition disclosed herein comprises 0.1 ppm to 10 ppm hypochlorous acid or free available chlorine and 0.1 ppm to 10 ppm of copper zinc iron oxide particles. In an aspect of this embodiment, a composition disclosed herein comprises 0.5 ppm to 7.5 ppm hypochlorous acid or free available chlorine and 0.5 ppm to 7.5 ppm of copper zinc iron oxide particles. In an aspect of this embodiment, a composition disclosed herein comprises 4 ppm to 6 ppm hypochlorous acid or free available chlorine, and 1 ppm to 3 ppm of copper zinc iron oxide particles. In an aspect of this embodiment, a composition disclosed herein comprises 5 ppm hypochlorous acid or free available chlorine and 2 ppm of copper zinc iron oxide particles.

In an embodiment, a composition disclosed herein comprises 0.01 ppm to 1 ppm hypochlorous acid or free available chlorine and 0.01 ppm to 1 ppm one or more metal particles. In an aspect of this embodiment, a composition disclosed herein comprises 0.01 ppm to 1 ppm hypochlorous acid or free available chlorine and 0.01 ppm to 1 ppm of metal oxide particles. In an aspect of this embodiment, a composition disclosed herein comprises 0.05 ppm to 0.75 ppm hypochlorous acid or free available chlorine and 0.05 ppm to 0.75 ppm of metal oxide particles. In an aspect of this embodiment, a composition disclosed herein comprises 0.4 ppm to 0.6 ppm hypochlorous acid or free available chlorine, and 0.1 ppm to 0.3 ppm of metal oxide particles. In an aspect of this embodiment, a composition disclosed herein comprises 0.5 ppm hypochlorous acid or free available chlorine and 0.2 ppm of metal oxide particles.

In an aspect of this embodiment, a composition disclosed herein comprises 0.01 ppm to 1 ppm hypochlorous acid or free available chlorine and 0.01 ppm to 1 ppm of copper zinc iron oxide particles. In an aspect of this embodiment, a composition disclosed herein comprises 0.05 ppm to 0.75 ppm hypochlorous acid or free available chlorine and 0.05 ppm to 0.75 ppm of copper zinc iron oxide particles. In an aspect of this embodiment, a composition disclosed herein comprises 0.4 ppm to 0.6 ppm hypochlorous acid or free available chlorine, and 0.1 ppm to 0.3 ppm of copper zinc iron oxide particles. In an aspect of this embodiment, a composition disclosed herein comprises 0.5 ppm hypochlorous acid or free available chlorine and 0.2 ppm of copper zinc iron oxide particles.

In an embodiment, a composition disclosed herein comprises about 0.1% to about 2% one or more biguanide-containing compounds and about 1% to about 20% one or more alcohols. In an aspect of this embodiment, a composition disclosed herein comprises about 0.25% to about 1.75% one or more biguanide-containing compounds and about 2% to about 15% one or more alcohols. In an aspect of this embodiment, a composition disclosed herein comprises about 0.5% to about 1.5% one or more biguanide-containing compounds and about 3% to about 12% one or more alcohols. In an aspect of this embodiment, a composition disclosed herein comprises about 0.75% to about 1.25% one or more biguanide-containing compounds and about 4% to about 10% one or more alcohols. In an aspect of this embodiment, a composition disclosed herein comprises 1% one or more biguanide-containing compounds and about 5% to about 7% one or more alcohols.

In an aspect of this embodiment, a composition disclosed herein comprises about 0.25% to about 1.75% polyhexamethylene biguanide and about 2% to about 15% isopropanol. In an aspect of this embodiment, a composition disclosed herein comprises about 0.5% to about 1.5% polyhexamethylene biguanide and about 3% to about 12% isopropanol. In an aspect of this embodiment, a composition disclosed herein comprises about 0.75% to about 1.25% polyhexamethylene biguanide and about 4% to about 10% isopropanol. In an aspect of this embodiment, a composition disclosed herein comprises about 1% polyhexamethylene biguanide and about 5% to about 7% isopropanol.

In an embodiment, a composition disclosed herein comprises about 0.01% to about 0.2% one or more biguanide-containing compounds and about 0.1% to about 2% one or more alcohols. In an aspect of this embodiment, a composition disclosed herein comprises about 0.025% to about 0.175% one or more biguanide-containing compounds and about 0.2% to about 1.5% one or more alcohols. In an aspect of this embodiment, a composition disclosed herein comprises about 0.05% to about 0.15% one or more biguanide-containing compounds and about 0.3% to about 1.2% one or more alcohols. In an aspect of this embodiment, a composition disclosed herein comprises about 0.075% to about 0.125% one or more biguanide-containing compounds and about 0.4% to about 1% one or more alcohols. In an aspect of this embodiment, a composition disclosed herein comprises 0.1% one or more biguanide-containing compounds and about 0.5% to about 0.7% one or more alcohols.

In an aspect of this embodiment, a composition disclosed herein comprises about 0.025% to about 0.175% polyhexamethylene biguanide and about 0.2% to about 1.5% isopropanol. In an aspect of this embodiment, a composition disclosed herein comprises about 0.05% to about 0.15% polyhexamethylene biguanide and about 0.3% to about 1.2% isopropanol. In an aspect of this embodiment, a composition disclosed herein comprises about 0.075% to about 0.125% polyhexamethylene biguanide and about 0.4% to about 1% isopropanol. In an aspect of this embodiment, a composition disclosed herein comprises about 0.1% polyhexamethylene biguanide and about 0.5% to about 0.7% isopropanol.

In an embodiment, a composition disclosed herein comprises about 0.001% to about 0.02% one or more biguanide-containing compounds and about 0.01% to about 0.2% one or more alcohols. In an aspect of this embodiment, a composition disclosed herein comprises about 0.0025% to about 0.0175% one or more biguanide-containing compounds and about 0.02% to about 0.15% one or more alcohols. In an aspect of this embodiment, a composition disclosed herein comprises about 0.005% to about 0.015% one or more biguanide-containing compounds and about 0.03% to about 0.12% one or more alcohols. In an aspect of this embodiment, a composition disclosed herein comprises about 0.0075% to about 0.0125% one or more biguanide-containing compounds and about 0.04% to about 0.1% one or more alcohols. In an aspect of this embodiment, a composition disclosed herein comprises 0.01% one or more biguanide-containing compounds and about 0.05% to about 0.07% one or more alcohols.

In an aspect of this embodiment, a composition disclosed herein comprises about 0.0025% to about 0.0175% polyhexamethylene biguanide and about 0.02% to about 0.15% isopropanol. In an aspect of this embodiment, a composition disclosed herein comprises about 0.005% to about 0.015% polyhexamethylene biguanide and about 0.03% to about 0.12% isopropanol. In an aspect of this embodiment, a composition disclosed herein comprises about 0.0075% to about 0.0125% polyhexamethylene biguanide and about 0.04% to about 0.1% isopropanol. In an aspect of this embodiment, a composition disclosed herein comprises about 0.01% polyhexamethylene biguanide and about 0.05% to about 0.07% isopropanol.

In an embodiment, a composition disclosed herein comprises about 0.1% to about 2% one or more biguanide-containing compounds, about 1% to about 20% one or more alcohols, and about 0.1% to about 2% of one or more quaternary ammonium salts. In an aspect of this embodiment, a composition disclosed herein comprises about 0.25% to about 1.75% one or more biguanide-containing compounds, about 2% to about 15% one or more alcohols, and about 0.2% to about 1.5% of one or more quaternary ammonium salts. In an aspect of this embodiment, a composition disclosed herein comprises about 0.5% to about 1.5% one or more biguanide-containing compounds, about 3% to about 12% one or more alcohols, and about 0.3% to about 1% of one or more quaternary ammonium salts. In an aspect of this embodiment, a composition disclosed herein comprises about 0.75% to about 1.25% one or more biguanide-containing compounds, about 4% to about 10% one or more alcohols, and about 0.4% to about 0.6% of one or more quaternary ammonium salts. In an aspect of this embodiment, a composition disclosed herein comprises 1% one or more biguanide-containing compounds, about 5% to about 7% one or more alcohols, and about 0.5% one or more quaternary ammonium salts.

In an embodiment, a composition disclosed herein comprises about 0.1% to about 2% polyhexamethylene biguanide, about 1% to about 20% isopropanol, and about 0.1% to about 2% of one or more cetylpyridinium salts. In an aspect of this embodiment, a composition disclosed herein comprises about 0.25% to about 1.75% polyhexamethylene biguanide, about 2% to about 15% isopropanol, and about 0.2% to about 1.5% of one or more cetylpyridinium salts. In an aspect of this embodiment, a composition disclosed herein comprises about 0.5% to about 1.5% polyhexamethylene biguanide, about 3% to about 12% isopropanol, and about 0.3% to about 1% of one or more cetylpyridinium salts. In an aspect of this embodiment, a composition disclosed herein comprises about 0.75% to about 1.25% polyhexamethylene biguanide, about 4% to about 10% isopropanol, and about 0.4% to about 0.6% of one or more cetylpyridinium salts. In an aspect of this embodiment, a composition disclosed herein comprises 1% polyhexamethylene biguanide, about 5% to about 7% isopropanol, and about 0.5% one or more cetylpyridinium salts.

In an embodiment, a composition disclosed herein comprises about 0.01% to about 0.2% one or more biguanide-containing compounds, about 0.1% to about 2% one or more alcohols, and about 0.01% to about 0.2% of one or more quaternary ammonium salts. In an aspect of this embodiment, a composition disclosed herein comprises about 0.025% to about 0.175% one or more biguanide-containing compounds, about 0.2% to about 1.5% one or more alcohols, and about 0.02% to about 0.15% of one or more quaternary ammonium salts. In an aspect of this embodiment, a composition disclosed herein comprises about 0.05% to about 0.15% one or more biguanide-containing compounds, about 0.3% to about 1.2% one or more alcohols, and about 0.03% to about 0.1% of one or more quaternary ammonium salts. In an aspect of this embodiment, a composition disclosed herein comprises about 0.075% to about 0.125% one or more biguanide-containing compounds, about 0.4% to about 1% one or more alcohols, and about 0.04% to about 0.06% of one or more quaternary ammonium salts. In an aspect of this embodiment, a composition disclosed herein comprises 0.1% one or more biguanide-containing compounds, about 0.5% to about 0.7% one or more alcohols, and about 0.05% one or more quaternary ammonium salts.

In an embodiment, a composition disclosed herein comprises about 0.01% to about 0.2% polyhexamethylene biguanide, about 0.1% to about 2% isopropanol, and about 0.01% to about 0.2% of one or more cetylpyridinium salts. In an aspect of this embodiment, a composition disclosed herein comprises about 0.025% to about 0.175% polyhexamethylene biguanide, about 0.2% to about 1.5% isopropanol, and about 0.02% to about 0.15% of one or more cetylpyridinium salts. In an aspect of this embodiment, a composition disclosed herein comprises about 0.05% to about 0.15% polyhexamethylene biguanide, about 0.3% to about 1.2% isopropanol, and about 0.03% to about 0.1% of one or more cetylpyridinium salts. In an aspect of this embodiment, a composition disclosed herein comprises about 0.075% to about 0.125% polyhexamethylene biguanide, about 0.4% to about 1% isopropanol, and about 0.04% to about 0.06% of one or more cetylpyridinium salts. In an aspect of this embodiment, a composition disclosed herein comprises 0.1% polyhexamethylene biguanide, about 0.5% to about 0.7% isopropanol, and about 0.05% one or more cetylpyridinium salts.

In an embodiment, a composition disclosed herein comprises about 0.001% to about 0.02% one or more biguanide-containing compounds, about 0.01% to about 0.2% one or more alcohols, and about 0.001% to about 0.02% of one or more quaternary ammonium salts. In an aspect of this embodiment, a composition disclosed herein comprises about 0.0025% to about 0.0175% one or more biguanide-containing compounds, about 0.02% to about 0.15% one or more alcohols, and about 0.002% to about 0.015% of one or more quaternary ammonium salts. In an aspect of this embodiment, a composition disclosed herein comprises about 0.005% to about 0.015% one or more biguanide-containing compounds, about 0.03% to about 0.12% one or more alcohols, and about 0.003% to about 0.01% of one or more quaternary ammonium salts. In an aspect of this embodiment, a composition disclosed herein comprises about 0.0075% to about 0.0125% one or more biguanide-containing compounds, about 0.04% to about 0.1% one or more alcohols, and about 0.004% to about 0.006% of one or more quaternary ammonium salts. In an aspect of this embodiment, a composition disclosed herein comprises 0.01% one or more biguanide-containing compounds, about 0.05% to about 0.07% one or more alcohols, and about 0.005% one or more quaternary ammonium salts.

In an embodiment, a composition disclosed herein comprises about 0.001% to about 0.02% polyhexamethylene biguanide, about 0.01% to about 0.2% isopropanol, and about 0.001% to about 0.02% of one or more cetylpyridinium salts. In an aspect of this embodiment, a composition disclosed herein comprises about 0.0025% to about 0.0175% polyhexamethylene biguanide, about 0.02% to about 0.15% isopropanol, and about 0.002% to about 0.015% of one or more cetylpyridinium salts. In an aspect of this embodiment, a composition disclosed herein comprises about 0.005% to about 0.015% polyhexamethylene biguanide, about 0.03% to about 0.12% isopropanol, and about 0.003% to about 0.01% of one or more cetylpyridinium salts. In an aspect of this embodiment, a composition disclosed herein comprises about 0.0075% to about 0.0125% polyhexamethylene biguanide, about 0.04% to about 0.1% isopropanol, and about 0.004% to about 0.006% of one or more cetylpyridinium salts. In an aspect of this embodiment, a composition disclosed herein comprises 0.01% polyhexamethylene biguanide, about 0.05% to about 0.07% isopropanol, and about 0.005% one or more cetylpyridinium salts.

In an embodiment, a composition disclosed herein comprises 1 ppm to 100 ppm hypochlorous acid or free available chlorine and 1 ppm to 100 ppm one or more quaternary compounds or silicone quaternary compounds. In an aspect of this embodiment, a composition disclosed herein comprises 1 ppm to 100 ppm hypochlorous acid or free available chlorine and 1 ppm to 100 ppm of one or more silicone dialkyl quaternary compounds. In an aspect of this embodiment, a composition disclosed herein comprises 5 ppm to 75 ppm hypochlorous acid or free available chlorine and 5 ppm to 75 ppm of one or more silicone dialkyl quaternary compounds. In an aspect of this embodiment, a composition disclosed herein comprises 40 ppm to 60 ppm hypochlorous acid or free available chlorine, and 40 ppm to 60 ppm of one or more silicone dialkyl quaternary compounds. In an aspect of this embodiment, a composition disclosed herein comprises 50 ppm hypochlorous acid or free available chlorine and 50 ppm of one or more silicone dialkyl quaternary compounds.

In an aspect of this embodiment, a composition disclosed herein comprises 1 ppm to 100 ppm hypochlorous acid or free available chlorine and 1 ppm to 100 ppm of dimethyloctadecyl[3-(trimethoxysilyl)propyl]ammonium chloride. In an aspect of this embodiment, a composition disclosed herein comprises 5 ppm to 75 ppm hypochlorous acid or free available chlorine and 5 ppm to 75 ppm of dimethyloctadecyl[3-(trimethoxysilyl)propyl]ammonium chloride. In an aspect of this embodiment, a composition disclosed herein comprises 40 ppm to 60 ppm hypochlorous acid or free available chlorine, and 40 ppm to 60 ppm of dimethyloctadecyl[3-(trimethoxysilyl)propyl]ammonium chloride. In an aspect of this embodiment, a composition disclosed herein comprises 50 ppm hypochlorous acid or free available chlorine and 50 ppm of dimethyloctadecyl[3-(trimethoxysilyl)propyl]ammonium chloride.

In an embodiment, a composition disclosed herein comprises 0.1 ppm to 10 ppm hypochlorous acid or free available chlorine and 0.1 ppm to 10 ppm one or more metal particles. In an aspect of this embodiment, a composition disclosed herein comprises 0.1 ppm to 10 ppm hypochlorous acid or free available chlorine and 0.1 ppm to 10 ppm of one or more silicone dialkyl quaternary compounds. In an aspect of this embodiment, a composition disclosed herein comprises 0.5 ppm to 7.5 ppm hypochlorous acid or free available chlorine and 0.5 ppm to 7.5 ppm of one or more silicone dialkyl quaternary compounds. In an aspect of this embodiment, a composition disclosed herein comprises 4 ppm to 6 ppm hypochlorous acid or free available chlorine, and 4 ppm to 6 ppm of one or more silicone dialkyl quaternary compounds. In an aspect of this embodiment, a composition disclosed herein comprises 5 ppm hypochlorous acid or free available chlorine and 5 ppm of one or more silicone dialkyl quaternary compounds.

In an aspect of this embodiment, a composition disclosed herein comprises 0.1 ppm to 10 ppm hypochlorous acid or free available chlorine and 0.1 ppm to 10 ppm of dimethyloctadecyl[3-(trimethoxysilyl)propyl]ammonium chloride. In an aspect of this embodiment, a composition disclosed herein comprises 0.5 ppm to 7.5 ppm hypochlorous acid or free available chlorine and 0.5 ppm to 7.5 ppm of dimethyloctadecyl[3-(trimethoxysilyl)propyl]ammonium chloride. In an aspect of this embodiment, a composition disclosed herein comprises 4 ppm to 6 ppm hypochlorous acid or free available chlorine, and 4 ppm to 6 ppm of dimethyloctadecyl[3-(trimethoxysilyl)propyl]ammonium chloride. In an aspect of this embodiment, a composition disclosed herein comprises 5 ppm hypochlorous acid or free available chlorine and 5 ppm of dimethyloctadecyl[3-(trimethoxysilyl)propyl]ammonium chloride.

In an embodiment, a composition disclosed herein comprises 0.01 ppm to 1 ppm hypochlorous acid or free available chlorine and 0.01 ppm to 1 ppm one or more metal particles. In an aspect of this embodiment, a composition disclosed herein comprises 0.01 ppm to 1 ppm hypochlorous acid or free available chlorine and 0.01 ppm to 1 ppm of one or more silicone dialkyl quaternary compounds. In an aspect of this embodiment, a composition disclosed herein comprises 0.05 ppm to 0.75 ppm hypochlorous acid or free available chlorine and 0.05 ppm to 0.75 ppm of one or more silicone dialkyl quaternary compounds. In an aspect of this embodiment, a composition disclosed herein comprises 0.4 ppm to 0.6 ppm hypochlorous acid or free available chlorine, and 0.4 ppm to 0.6 ppm of one or more silicone dialkyl quaternary compounds. In an aspect of this embodiment, a composition disclosed herein comprises 0.5 ppm hypochlorous acid or free available chlorine and 0.5 ppm of one or more silicone dialkyl quaternary compounds.

In an aspect of this embodiment, a composition disclosed herein comprises 0.01 ppm to 1 ppm hypochlorous acid or free available chlorine and 0.01 ppm to 1 ppm of dimethyloctadecyl[3-(trimethoxysilyl)propyl]ammonium chloride. In an aspect of this embodiment, a composition disclosed herein comprises 0.05 ppm to 0.75 ppm hypochlorous acid or free available chlorine and 0.05 ppm to 0.75 ppm of dimethyloctadecyl[3-(trimethoxysilyl)propyl]ammonium chloride. In an aspect of this embodiment, a composition disclosed herein comprises 0.4 ppm to 0.6 ppm hypochlorous acid or free available chlorine, and 0.4 ppm to 0.6 ppm of dimethyloctadecyl[3-(trimethoxysilyl)propyl]ammonium chloride. In an aspect of this embodiment, a composition disclosed herein comprises 0.5 ppm hypochlorous acid or free available chlorine and 0.5 ppm of dimethyloctadecyl[3-(trimethoxysilyl)propyl]ammonium chloride.

Aspects of the present specification disclose a kit. In one embodiment, the kit can comprise a container including a composition disclosed herein. In one embodiment, the kit can comprise a container including a microbiostatic agent disclosed herein. In another embodiment, a kit can comprise one or more containers, each container including an individual component like a microbiostatic agent disclosed herein or more than one individual components like more than one microbiostatic agents disclosed herein in combination. For example, a kit can comprise one container including hypochlorous acid or free available chlorine and a second container including one or more metallic particles. As another example, a kit can comprise one container including hypochlorous acid or free available chlorine and a second container including copper zinc iron oxide particles. As yet another example, a kit can comprise one container including one or more alcohols and a second container including one or more guanide-containing compounds. As still another example, a kit can comprise one container including isopropanol and a second container including polyhexamethylene biguanide. As a further example, a kit can comprise one container including hypochlorous acid or free available chlorine and a second container including one or more quaternary compounds or silicon quaternary compounds. As another example, a kit can comprise one container including hypochlorous acid or free available chlorine and a second container including dimethyloctadecyl[3-(trimethoxysilyl)propyl]ammonium chloride. The remainder of the components of a composition disclosed herein may be included in either the first or second container, or may be separately including in at least a third container. For example, a third container including a rinse solution disclosed herein can be included in the kit. Packaging of individual components into separate container can assist in prolonging the stability of the individual components, and thus shelf life of the product.

In another example, a kit can comprise one container including hypochlorous acid and/or free available chlorine, a second container including one or more metallic particles, and a third container including a rinse solution. As another example, a kit can comprise one container including hypochlorous acid and/or free available chlorine, a second container including copper zinc iron oxide particles, and a third container including a rinse solution. As yet another example, a kit can comprise one container including one or more alcohols, a second container including one or more guanide-containing compounds, and a third container including a rinse solution. As still another example, a kit can comprise one container including isopropanol, a second container including polyhexamethylene biguanide, and a third container including a rinse solution. As a further example, a kit can comprise one container including hypochlorous acid or free available chlorine, a second container including one or more quaternary compounds or silicon quaternary compounds, and a third container including a rinse solution. As another example, a kit can comprise one container including hypochlorous acid or free available chlorine, a second container including dimethyloctadecyl[3-(trimethoxysilyl)propyl]ammonium chloride, and a third container including a rinse solution. As another example, a kit can comprise one container including isopropanol, a second container including polyhexamethylene biguanide, a third container including one or more cationic surfactants, and a fourth container including a rinse solution.

In another embodiment, a kit can comprise one container including a composition disclosed herein and a second container including a rinse solution disclosed herein. In another embodiment, a kit can comprise one container including a microbiostatic agent disclosed herein and a second container including a rinse solution disclosed herein. For example, a kit can comprise one container including hypochlorous acid and/or free available chlorine and one or more metallic particles and a second container including a rinse solution. As another example, a kit can comprise one container including hypochlorous acid and/or free available chlorine and copper zinc iron oxide particles and a second container including a rinse solution. As yet another example, a kit can comprise one container including one or more alcohols and one or more guanide-containing compounds and a second container including a rinse solution. As still another example, a kit can comprise one container including isopropanol and polyhexamethylene biguanide and a second container including a rinse solution. As a further example, a kit can comprise one container including hypochlorous acid and/or free available chlorine and one or more quaternary compounds or silicon quaternary compounds and a second container including a rinse solution. As another example, a kit can comprise one container including hypochlorous acid and/or free available chlorine and dimethyloctadecyl[3-(trimethoxysilyl)propyl]ammonium chloride and a second container including a rinse solution. The remainder of the components of a composition disclosed herein may be included in either the first container or may be separately including in at least a third container. Packaging of individual components into separate containers can assist in prolonging the stability of the individual components, and thus shelf life of the product.

A kit disclosed herein can comprise a delivery or application system. The delivery or application system of the kit are useful for applying a composition disclosed herein, and/or individual components disclosed herein to a site of interest, such as, e.g., a surface of a device disclosed herein. A delivery or application system disclosed herein, includes, without limitation, one or more of an applicator brush, porous foam swab or pad, hollow tube, dipstick, or a combination thereof. In an embodiment, a kit comprises a single delivery or application system. In another embodiment, a kit comprises a plurality delivery or application systems. For example, in a 30-day supply kit, there can be 30 delivery or application systems, such that there is one delivery or application system per day for 30 days. Alternately, there can be 2, 10, 20, 30, 40, 50, 60, 90, 120, etc. delivery or application systems per kit. Within the kit, the delivery or application system may be packaged individually, or in sets of 2 or more. The delivery or application system can be packaged such that it remains sterile until use. In certain embodiments, a delivery or application system disclosed herein can be packaged in plastic sheaths. Further, to prevent contamination, delivery or application system disclosed herein is preferably single-use, disposable delivery or application system.

The kit can also comprise a set of instructions. The instructions may include information useful to the end user such as how to use a delivery or application system to apply a composition and/or individual components disclosed herein, and/or how often to apply a composition and/or individual components disclosed herein. In addition, such instructions may include information regarding how to mix the individual components disclosed herein to form a composition disclosed herein. Such instructions can indicate that mixing should be done at a certain time before application, such as, e.g., just prior to use. Instructions disclosed herein may also include information regarding how to apply the individual components disclosed herein directly to a site of interest, such as, e.g., a surface of a device disclosed herein, and in what order the individual components should be applied to such sites of interest.

The contents of the kit, including the container including a composition, a microbiostatic agent, or other component disclosed herein, the delivery or application system, and instructions, are enclosed in an outer casing. The outer casing can be a box, a sealed bag, a foil pouch, etc. In certain embodiments, the delivery system, container and instructions are enclosed in a box. In other embodiments of the kit, the container and instructions are contained in a first box, the delivery system is contained in a second box, and the first and second box are contained together in a third box.

Although water does not provide an environment favorable for microbial growth, water can become subsequently contaminated with organic matter that can then support microbial growth in the water. As such, the presently disclosed compositions are useful in any application involving the restraining or inhibition of microbial growth in water or other liquid exposed to microbes or organic matter which supports microbial growth. In one embodiment, a composition disclosed herein is used to reduce or inhibit microbial growth in water or other liquid present in a device during use or storage of the device. The water or other liquid can be manufactured as sterile or non-sterile. In one embodiment, a composition disclosed herein is used to reduce or inhibit microbial growth in water or other liquid present in a medical device during use or storage of the medical device. In one embodiment, a composition disclosed herein is used to reduce or inhibit microbial growth in water or other liquid present in a non-medical device during use or storage of the non-medical device. In one embodiment, a composition disclosed herein is used to reduce or inhibit microbial growth in water or other liquid present in a residential, commercial or industrial device during use or storage of the residential, commercial or industrial device.

In one embodiment, a composition disclosed herein is used to reduce or inhibit microbial growth in water or other liquid. In one embodiment, a composition disclosed herein is used to reduce or inhibit microbial growth in water or other liquid present in a water reservoir or other component of a device. In one embodiment, a composition disclosed herein is used to reduce or inhibit microbial growth in water or other liquid present in a water reservoir or other component of a medical device. In one embodiment, a composition disclosed herein is used to reduce or inhibit microbial growth in water or other liquid present in a water reservoir or other component of a non-medical device. In one embodiment, a composition disclosed herein is used to reduce or inhibit microbial growth in water or other liquid present in a water reservoir or other component of a residential, commercial or industrial device.

In one embodiment, a composition disclosed herein is used to wash or irrigate a surface area. In one embodiment, a composition disclosed herein is used to wash or irrigate a biological surface, a tissue, a skin, or a body cavity.

In one embodiment, a composition disclosed herein is used to wash or irrigate a wound in an individual. In one embodiment, a composition disclosed herein is used to wash or irrigate an infection in an individual. In one embodiment, a composition disclosed herein is used to treat and/or provide relief from a wound or infection in an individual.

Aspects of the present specification disclose a making a microbiostatic water or other liquid. Such methods are designed to restrain, prohibit, or otherwise prevents population growth of microbes pre-existing in water or other liquid or subsequently introduced to the water or other liquid. In aspects of this embodiment, a method disclosed herein comprises applying one or more microbiostatic agents disclosed herein to water or other liquid in a microbiostatic effective amount. In other aspects of this embodiment, a method disclosed herein comprises applying one or more microbiostatic agents disclosed herein to water or other liquid in an amount that restrains, prohibits, or otherwise prevents microbial population growth without eradicating the microbes. In yet other aspects of this embodiment, a method disclosed herein comprises applying one or more microbiostatic agents disclosed herein to water or other liquid in an amount that restrains, prohibits, or otherwise prevents microbial population growth without eradicating the microbes to a degree that achieves disinfection or sterilization.

Aspects of the present specification disclose a method of reducing or inhibiting microbial growth in water or other liquid. Such methods are designed to restrain, prohibit, or otherwise prevent population growth of microbes pre-existing in water or other liquid or subsequently introduced to the water or other liquid. In aspects of this embodiment, a method disclosed herein comprises applying a composition disclosed herein to water or other liquid in a microbiostatic effective amount, wherein application of the composition reduces or inhibits microbial growth. In other aspects of this embodiment, a method disclosed herein comprises applying a composition disclosed herein to water or other liquid in an amount that restrains, prohibits, or otherwise prevents microbial population growth without eradicating the microbes, wherein application of the composition reduces or inhibits microbial growth. In yet other aspects of this embodiment, a method disclosed herein comprises applying a composition disclosed herein to water or other liquid in an amount that restrains, prohibits, or otherwise prevents microbial population growth without eradicating the microbes to a degree that achieves disinfection or sterilization, wherein application of the composition reduces or inhibits microbial growth.

In aspects of this embodiment, a composition disclosed herein is for use in reducing or inhibiting microbial growth in water or other liquid. In other aspects of this embodiment, a use of a disclosed composition is to reduce or inhibit microbial growth in water or other liquid. In yet other aspects of this embodiment, a use of a composition disclosed herein is to manufacture a medicament of water or other liquid to reduce or inhibit microbial growth.

Aspects of the present specification disclose a method of reducing or inhibiting microbial growth in a medical device. Such methods are designed to restrain, prohibit, or otherwise prevent population growth of microbes present in a medical device, prevent the spread of such microbial contamination to other areas or components of the medical device as well as reduce or inhibit microbial growth of microbes subsequently introduced to the medical device.

In an embodiment, a method disclosed herein applies a composition disclosed herein or one or more microbiostatic agents disclosed herein directly to water or other liquid. In this embodiment, composition disclosed herein or one or more microbiostatic agents disclosed herein is used as a concentrate that is diluted in water or other liquid.

In aspects of this embodiment, a method disclosed herein applies a composition disclosed herein to water or other liquid already present in a medical device, or a component of a medical device, such as, e.g., a reservoir or other storage container for water or other liquid. A method disclosed herein can also apply a composition disclosed herein to water or other liquid prior to introduction to a medical device, and then adding this admixture to a medical device, or a component of a medical device, such as, e.g., a reservoir or other storage container for water or other liquid. In aspects of this embodiment, a disclosed method comprises applying a composition disclosed herein to water or other liquid present in or to be added to a medical device in a microbiostatic effective amount, wherein application of the composition reduces or inhibits microbial growth in the water or other liquid and/or in the medical device. In other aspects of this embodiment, a disclosed method comprises applying a composition disclosed herein to water or other liquid present in or to be added to a medical device in an amount that restrains, prohibits, or otherwise prevents microbial population growth without eradicating the microbes, wherein application of the composition reduces or inhibits microbial growth in the water or other liquid and/or in the medical device. In yet other aspects of this embodiment, a disclosed method comprises applying a composition disclosed herein to water or other liquid present in or to be added to a medical device in an amount that restrains, prohibits, or otherwise prevents microbial population growth without eradicating the microbes to a degree that achieves disinfection or sterilization, wherein application of the composition reduces or inhibits microbial growth in the water or other liquid and/or in the medical device.

In aspects of this embodiment, a disclosed method comprises applying one or more microbiostatic agents disclosed herein to water or other liquid present in or to be added to a medical device in a microbiostatic effective amount, wherein application of the one or more microbiostatic agents reduces or inhibits microbial growth in the water or other liquid and/or in the medical device. In other aspects of this embodiment, a disclosed method comprises applying one or more microbiostatic agents disclosed herein to water or other liquid present in or to be added to a medical device in an amount that restrains, prohibits, or otherwise prevents microbial population growth without eradicating the microbes, wherein application of the one or more microbiostatic agents reduces or inhibits microbial growth in the water or other liquid and/or in the medical device. In yet other aspects of this embodiment, a disclosed method comprises applying one or more microbiostatic agents disclosed herein to water or other liquid present in or to be added to a medical device in an amount that restrains, prohibits, or otherwise prevents microbial population growth without eradicating the microbes to a degree that achieves disinfection or sterilization, wherein application of the one or more microbiostatic agents reduces or inhibits microbial growth in the water or other liquid and/or in the medical device.

In another embodiment, a method disclosed herein applies a composition disclosed herein in an undiluted which is added directly to a medical device, or a component of a medical device, such as, e.g., a reservoir or other storage container for water or other liquid. In this embodiment, a composition disclosed herein is used as a substitute or replacement for water or other liquid and would typically be used in an undiluted form. In aspects of this embodiment, a disclosed method comprises applying a composition disclosed herein directly to a medical device, or a component of a medical device, such as, e.g., a reservoir or other storage container for water or other liquid, wherein application of the composition reduces or inhibits microbial growth prior and during operation of the device.

In aspects of this embodiment, a composition disclosed herein is for use in reducing or inhibiting microbial growth in a medical device. In other aspects of this embodiment, a use of a composition disclosed herein is to reduce or inhibit microbial growth in a medical device. In yet other aspects of this embodiment, a use of a composition disclosed herein is to manufacture a medicament to reduce or inhibit microbial growth in a medical device.

Aspects of the present specification disclose a method of reducing or inhibiting microbial growth in a residential, commercial or industrial device. Such methods are designed to restrain, prohibit, or otherwise prevent population growth of microbes present in a residential, commercial or industrial device, prevent the spread of such microbial contamination to other areas or components of the residential, commercial or industrial device as well as restrain, prohibit, or otherwise prevent population growth of microbes subsequently introduced to the residential, commercial or industrial device.

In an embodiment, a method disclosed herein applies a composition disclosed herein or one or more microbiostatic agents disclosed herein directly to water or other liquid. In this embodiment, composition disclosed herein or one or more microbiostatic agents disclosed herein is used as a concentrate that is diluted in water or other liquid.

In aspects of this embodiment, a method disclosed herein applies a composition disclosed herein to water or other liquid already present in a residential, commercial or industrial device, or a component of a residential, commercial or industrial device, such as, e.g., a reservoir or other storage container for water or other liquid. A method disclosed herein can also apply a composition disclosed herein to water or other liquid prior to introduction to a residential, commercial or industrial device, and then adding this admixture to a residential, commercial or industrial device, or a component of a residential, commercial or industrial device, such as, e.g., a reservoir or other storage container for water or other liquid. In aspects of this embodiment, a method disclosed herein comprises applying a composition disclosed herein to water or other liquid present in or to be added to a residential, commercial or industrial device in a microbiostatic effective amount, wherein application of the composition reduces or inhibits microbial growth in the water or other liquid and/or in the residential, commercial or industrial device. In other aspects of this embodiment, a disclosed method comprises applying a composition disclosed herein to water or other liquid present in or to be added to a residential, commercial or industrial device in an amount that restrains, prohibits, or otherwise prevents microbial population growth without eradicating the microbes, wherein application of the composition reduces or inhibits microbial growth in the water or other liquid and/or in the residential, commercial or industrial device. In yet other aspects of this embodiment, a disclosed method comprises applying a composition disclosed herein to water or other liquid present in or to be added to a residential, commercial or industrial device in an amount that restrains, prohibits, or otherwise prevents microbial population growth without eradicating the microbes to a degree that achieves disinfection or sterilization, wherein application of the composition reduces or inhibits microbial growth in the water or other liquid and/or in the residential, commercial or industrial device.

In aspects of this embodiment, a disclosed method comprises applying one or more microbiostatic agents disclosed herein to water or other liquid present in or to be added to a residential, commercial or industrial device in a microbiostatic effective amount, wherein application of the one or more microbiostatic agents reduces or inhibits microbial growth in the water or other liquid and/or in the residential, commercial or industrial device. In other aspects of this embodiment, a disclosed method comprises applying one or more microbiostatic agents disclosed herein to water or other liquid present in or to be added to a residential, commercial or industrial device in an amount that restrains, prohibits, or otherwise prevents microbial population growth without eradicating the microbes, wherein application of the one or more microbiostatic agents reduces or inhibits microbial growth in the water or other liquid and/or in the residential, commercial or industrial device. In yet other aspects of this embodiment, a disclosed method comprises applying one or more microbiostatic agents disclosed herein to water or other liquid present in or to be added to a residential, commercial or industrial device in an amount that restrains, prohibits, or otherwise prevents microbial population growth without eradicating the microbes to a degree that achieves disinfection or sterilization, wherein application of the one or more microbiostatic agents reduces or inhibits microbial growth in the water or other liquid and/or in the residential, commercial or industrial device.

In another embodiment, a method disclosed herein applies a composition disclosed herein in an undiluted which is added directly to a residential, commercial or industrial device, or a component of a residential, commercial or industrial device, such as, e.g., a reservoir or other storage container for water or other liquid. In this embodiment, a composition disclosed herein is used as a substitute or replacement for water or other liquid and would typically be used in an undiluted form. In other aspects, a disclosed method comprises applying a composition disclosed herein directly to a residential, commercial or industrial device, wherein application of the composition reduces or inhibits microbial growth prior and during operation of the device.

In aspects of this embodiment, a composition disclosed herein is for use in reducing or inhibiting microbial growth in a residential, commercial or industrial device. In other aspects of this embodiment, a use of a composition disclosed herein is to reduce or inhibit microbial growth in a residential, commercial or industrial device. In yet other aspects of this embodiment, a use of a composition disclosed herein is to manufacture a medicament to reduce or inhibit microbial growth in a residential, commercial or industrial device.

In an embodiment, a method disclosed herein does not disinfect or sterilize water or other liquid. Both sterilization and disinfection kill or otherwise eradicate microbes. Sterilization is a process that destroys or eliminates all forms of a microbe including microbial spores. Water or other liquid disclosed herein is considered to be sterilized when there is a 1×10⁶ log reduction of microbes. Disinfection destroys or eliminates most recognized pathogenic microbes but not necessarily all microbial forms (e.g., microbial spores). Water or other liquid disclosed herein is considered to be sterilized when there is a 1×10⁴ reduction of microbes. Since the amount of a composition disclosed herein is used in a microbiostatic effective amount in a method disclosed herein, the method does not sterilize or disinfect the water or other liquid. In aspects of this embodiment, treating a water or other liquid with a composition disclosed herein or one or more microbiostatic agents disclosed herein does not result in a 1×10⁴ or greater reduction of microbes. In other aspects of this embodiment, treating a water or other liquid with a composition disclosed herein or one or more microbiostatic agents disclosed herein does not result in a 1×10⁵ or greater reduction of microbes. In yet other aspects of this embodiment, treating a water or other liquid with a composition disclosed herein or one or more microbiostatic agents disclosed herein does not result in a 1×10⁶ or greater reduction of microbes.

In aspects of this embodiment, treating a water or other liquid with a composition disclosed herein or one or more microbiostatic agents disclosed herein results in, e.g., about 1×10⁰ reduction in microbes, about 1×10¹ reduction in microbes, about 1×10² reduction in microbes, or about 1×10³ reduction in microbes, In other aspects of this embodiment, treating a water or other liquid with a composition disclosed herein or one or more microbiostatic agents disclosed herein results in, e.g., at most 1×10⁰ reduction in microbes, at most 1×10¹ reduction in microbes, at most 1×10² reduction in microbes, or at most 1×10³ reduction in microbes, In yet other aspects of this embodiment, treating a water or other liquid with a composition disclosed herein or one or more microbiostatic agents disclosed herein results in, e.g., about 1×10⁰ to about 1×10¹ reduction in microbes, about 1×10⁰ to about 1×10² reduction in microbes, about 1×10⁰ to about 1×10³ reduction in microbes, about 1×10¹ to about 1×10² reduction in microbes, about 1×10¹ to about 1×10³ reduction in microbes, or about 1×10² to about 1×10³ reduction in microbes.

The term “other liquid” refers to any single or multi-phase fluid that can support or is susceptible to microbial contamination and growth. In aspects of this embodiment, a liquid or “other liquid” is single or multi-phase liquid comprising water. A liquid or “other liquid” can be a pharmaceutical carrier, such as, e.g., vehicle, stabilizer, diluent, additive, auxiliary, or excipient, used to formulate a pharmaceutical drug or prepare such a drug for administration to an individual. A liquid or “other liquid” can be a buffered solution, a medical solution, a contact solution, a dental solution, a mouthwash solution, or a denture solution. A liquid or “other liquid” can be a fluid used as a coolant, a flushing agent, a washing agent or other fluid susceptible to microbial contamination when stored or used in devices, equipment, appliances, machinery, or other apparatus.

A medical device is an instrument, apparatus, material, or other article, whether used alone or in combination, including software necessary for its application, intended by the manufacturer to be used for human beings for diagnosis, prevention, monitoring treatment, or alleviation of disease; diagnosis, monitoring, treatment, or alleviation of or compensation for an injury or handicap; investigation, replacement, or modification of the anatomy or of a physiologic process; or control of conception, and that does not achieve its primary intended action in or on the human body by pharmacologic, immunologic, or metabolic means but might be assisted in its function by such means. A medical device includes, without limitation, a surgical instrument, a dental instrument, a respiratory therapy instrument including positive-pressure ventilators and negative-pressure ventilators, an anesthesia instrument, a catheter, an implant, a probe, an endoscope, an arthroscope, a laparoscope, a blade, a cystoscope, a spirometer, a continuous positive airway pressure (CPAP) mask and tubing, a positive end-expiratory pressure (PEEP) mask and tubing, dialysis instrument and accessories, a heart-lung machine and accessories, a heart-lung bypass machine and accessories, and a diaphragm fitting ring. Non-limiting examples of a probe includes an ultrasound probe and an esophageal manometry probe. Non-limiting examples of a catheter includes a cardiac catheter, an urinary catheter, an anorectal manometry catheter. Non-limiting examples of an endoscope includes a gastrointestinal endoscope, a bronchoscope, and a nasopharyngoscope. Non-limiting examples of a blade includes a laryngoscope blade.

Aspects of the present specification disclose a method to wash or irrigate a surface area. Such methods are designed to prevent introduction of microbes to a surface area due to adverse levels of contamination in a composition disclosed herein. In aspects of this embodiment, a method disclosed herein comprises applying a composition disclosed herein to a surface area, wherein application of the composition washes or irrigates the surface area.

In other aspects of this embodiment, a composition disclosed herein is for use in washing or irrigating a surface area. In other aspects of this embodiment, a use of a disclosed composition is to wash or irrigate a surface area. A surface area can be a porous surface area or a non-porous surface area including a skin surface of an individual or a hard surface of a medical or a non-medical device, equipment, appliance, machinery, or other apparatus.

In an embodiment, a method disclosed herein does not disinfect or sterilize a surface area.

In another aspect of this embodiment, a method disclosed herein comprises applying one or more individual components disclosed herein to a hard surface area for a specified amount of time, wherein application results in the washing or irrigation of the hard surface area. In an aspect of this embodiment, the one or more individual components include a first component including hypochlorous acid or free available chlorine and a second component including one or more disinfectants. In another aspect, application of the one or more individual components occur in a specific order, such as. e.g., first applying a first component including hypochlorous acid or free available chlorine and then applying a second component including one or more disinfectants. A hard surface area can be a porous surface area or a non-porous surface area.

A hard surface area can include any items present in a residence or a commercial, industrial and/or agricultural facility, such as, e.g., a hospital, a laboratory, a restaurant, an educational center, a food-processing facility, a dairy-processing facility, an airport, an oil field system, a sport facility, a shipping dock, a freight transport center, or any other commercial or industrial setting. A surface area can include any type of transportation carrier, such as, e.g., a water vessel like a boat, barge or ship, an aircraft like an airplane or helicopter, a ground vehicle like a motorcycle, car, truck or train, A surface area may be made of any material including brass, copper, aluminum, stainless steel, carbon steel, rubber, plastic, glass, wood, painted surface, or any combination thereof. A surface area includes, without limitation, a table top, counter top, floor, wall, ceiling, window, bed, gurney, door, door handle, shower, bath, sink, faucet, toilet, toilet seat, drain, equipment, machinery, personal protective gear, personal biohazard gear, and the like. A surface area may comprise a medical, dental, pharmaceutical, veterinary or mortuary device. A surface area may comprise human skin.

A composition or component disclosed herein can be applied to a hard surface area according to a method disclosed herein as often as needed and/or desired. A composition disclosed herein can be applied to a hard surface area daily, every other day, every third of day, once a week, multiple times per week, once a month, multiple times per month, once a year or multiple times per year, as desired. A composition disclosed herein can be applied to a hard surface area multiple times per day, e.g., twice a day, three times a day, four time a day, five times a day, six times a day or as often as desired.

The presently disclosed compositions are useful in any application involving treating and/or providing relief to an individual. The presently disclosed compositions are useful in any application involving medical use, veterinarian use, or both. In one embodiment, a composition disclosed herein is useful in any application were hypochlorous acid is applied or administered.

In one embodiment, the presently disclosed compositions are useful in any application involving treating and/or providing relief to a tissue in an individual. For example, the compositions disclosed herein can be used to treat and/or provide relief from a wound by washing or irrigating the tissue that minimizes the introduction of microbes to the tissue thereby facilitating the healing of the tissue.

In one embodiment, the presently disclosed compositions are useful in any application involving treating and/or providing relief from a wound in an individual. For example, the compositions disclosed herein can be used to treat and/or provide relief from a wound by washing or irrigating the wound that minimizes the introduction of microbes to the wound thereby facilitating the healing of the wound. A wound can be an open wound, a closed wound or a burn. Non-limiting examples, of an open wound include a laceration, an abrasion, an incision, a puncture, an avulsion, an ulcer, and a tear. Non-limiting examples, of a closed wound includes a bruise, a contusion and a hematoma.

Aspects of the present specification disclose a method to wash or irrigate a wound in an individual. Such methods are designed to prevent introduction of microbes to a wound due to adverse levels of contamination in a composition disclosed herein. In aspects of this embodiment, a method disclosed herein comprises applying a composition disclosed herein to the wound of an individual, wherein application of the composition washes or irrigates the wound.

In aspects of this embodiment, a composition disclosed herein is for use in washing or irrigating a wound in an individual. In other aspects of this embodiment, a use of a disclosed composition is to wash or irrigate a wound in an individual. In yet aspects of this embodiment, a use of a disclosed composition is in the manufacture of a medicament to wash or irrigate a wound in an individual.

In aspects of this embodiment, a composition disclosed herein is for use for a therapeutic effect, such as angiogenesis, reduction of inflammation, pain, itch, irritation, or pressure, reduction of histidine production, oxygenation of tissues, or soothing. As such, methods of treatment are also disclosed.

Other aspects of the present specification disclose a method to treat and/or provide relief from a wound in an individual. Such methods are designed to prevent introduction of microbes to a wound due to adverse levels of contamination in a composition disclosed herein. In aspects of this embodiment, a method disclosed herein comprises applying a composition disclosed herein to an individual, wherein application of the composition promotes healing of a wound.

In aspects of this embodiment, a composition disclosed herein is for use in treating and/or providing relief from a wound in an individual. In other aspects of this embodiment, a use of a disclosed composition is to treat and/or provide relief from a wound in an individual. In still aspects of this embodiment, a use of a disclosed composition is in the manufacture of a medicament to treat and/or provide relief from a wound in an individual.

In an embodiment, a method disclosed herein does not disinfect or sterilize a wound.

In one embodiment, the presently disclosed compositions are useful in any application involving a microbial infection in an individual. A microbial infection includes a viral infection, a bacterial infection and a fungal infection. A microbial infection can be an external infection on, e.g., a surface area of an individual or an internal infection, e.g., a mercer infection or sepsis, located in the body or body cavity of an individual.

Aspects of the present specification disclose a method to wash or irrigate an infection in an individual. Such methods are designed to prevent introduction of microbes to an infection due to adverse levels of contamination in a composition disclosed herein. In aspects of this embodiment, a method disclosed herein comprises applying a composition disclosed herein to an individual, wherein application of the composition washes or irrigates the infection.

In aspects of this embodiment, a composition disclosed herein is for use in washing or irrigating an infection in an individual. In other aspects of this embodiment, a use of a disclosed composition is to wash or irrigate an infection in an individual. In yet aspects of this embodiment, a use of a disclosed composition is in the manufacture of a medicament to wash or irrigate an infection in an individual. An infection can be a microbial infection.

Aspects of the present specification disclose a method of treating and/or providing relief from an infection in an individual. Such methods are designed to prevent introduction of microbes to an infection due to adverse levels of contamination in a composition disclosed herein. In aspects of this embodiment, a method disclosed herein comprises applying a composition disclosed herein to an individual, wherein application of the composition treats and/or provides relief from the infection.

In aspects of this embodiment, a composition disclosed herein is for use in treating and/or providing relief from an infection in an individual. In other aspects of this embodiment, a use of a disclosed composition is to treat and/or provide relief from an infection in an individual. In yet aspects of this embodiment, a use of a disclosed composition is in the manufacture of a medicament to treat and/or provide relief from an infection in an infection.

In an embodiment, a method disclosed herein does not disinfect or sterilize an infection.

In one embodiment, a composition disclosed herein is used to wash or irrigate or treat and/or provide relief for a body part of an individual. Non-limiting examples of uses for a composition disclosed herein include washing or irrigating or treating and/or providing relief to tissue in wound care, pre-operative preparation, surgery or other invasive procedure, and post-operative care, washing or irrigating or treating and/or providing relief to a skin region in dermatological or other healthcare applications, washing or irrigating or treating and/or providing relief to a body cavity, including, without limitation, an oral cavity, a nasal cavity, an otic cavity and a pulmonary cavity, and an abdominal cavity, washing or irrigating or treating and/or providing relief to gums or tooth in dental applications, and washing or irrigating or treating and/or providing relief to the eye in ophthalmological applications.

A dermatological application refers to washing or irrigating or treating and/or providing relief to a skin region of an individual of a microbial infection, such as, e.g., a viral, bacterial or fungal infection. Non-limiting examples of a microbial infection of a skin region include urinary tract infection, eye lid wash, cataract treatment, warts, cutaneous leishmaniasis, candidal vulvovagititis, derrmatophytoses, bromhidrosis, pityriasis versicolor, acne vulgarish, rosasea, hydradenitis suppurativa, psoriasis, eczema, alopecia areata, oral lichen planus, xeroderma pigmentosum, actinic keratoses, melasma, keloids, and anti-aging.

An ophthalmological application refers to washing or irrigating or treating and/or providing relief to an eye of an individual of a microbial infection, such as, e.g., a viral, bacterial or fungal infection. Non-limiting examples of a microbial infection of an eye include a bacterial conjunctivitis, a viral conjunctivitis, an epidemic keratoconjunctivitis, a pharyngoconjunctival fever, a stye, a blephartis, an episcleritis, a keratitis, a trachoma, and a corneal ulcer.

The presently disclosed compositions are useful in any application involving treating and/or providing relief from of an inflammation, an itch, an irritation, a pressure, an ache and/or a pain in an individual.

Aspects of the present specification disclose a method to wash or irrigate an inflammation in an individual. Such methods are designed to prevent introduction of microbes to an inflamed site due to adverse levels of contamination in a composition disclosed herein. In aspects of this embodiment, a method disclosed herein comprises applying a composition disclosed herein to an individual, wherein application of the composition washes or irrigates the inflammation.

In aspects of this embodiment, a composition disclosed herein is for use in washing or treating and/or providing relief from an inflammation in an individual. In other aspects of this embodiment, a use of a disclosed composition is to wash or treat and/or provide relief from an inflammation in an individual. In yet aspects of this embodiment, a use of a disclosed composition is in the manufacture of a medicament to wash or irrigate an inflammation.

Aspects of the present specification disclose a method of treating and/or providing relief from an inflammation in an individual. Such methods are designed to prevent introduction of microbes to an inflamed site due to adverse levels of contamination in a composition disclosed herein. In aspects of this embodiment, a method disclosed herein comprises applying a composition disclosed herein to an individual, wherein application of the composition treats and/or provides relief from the inflammation.

In aspects of this embodiment, a composition disclosed herein is for use in treating and/or providing relief from an inflammation in an individual. In other aspects of this embodiment, a use of a disclosed composition is to treat and/or provide relief from an inflammation in an individual. In yet aspects of this embodiment, a use of a disclosed composition is in the manufacture of a medicament to treat and/or provide relief from an inflammation.

In an embodiment, a method disclosed herein does not disinfect or sterilize an inflammation.

The disclosed composition use active ingredients in a microbiostatic effective amount that are non-toxic and non-irritating to an individual. As such, the presently disclosed compositions are useful in enteral and parenteral applications, including oral, injectable and topical applications. For example, the compositions disclosed herein can be used to wash or irrigate a body region injured by a wound or infected with a microbe.

In an aspect of this embodiment, a method disclosed herein comprises applying a composition disclosed herein to an individual for a specified amount of time, wherein application results in the reduction or inhibition of microbial growth in a microbial infection in the individual. Other aspects of the present specification disclose a composition disclosed herein for use to reduce or inhibit microbial growth in a microbial infection in an individual. Yet other aspects of the present specification disclose a use of a disclosed composition to reduce or inhibit microbial growth in a microbial infection in an individual. Still other aspects of the present specification disclose a composition disclosed herein for the manufacture of a medicament to reduce or inhibit microbial growth in a microbial infection in an individual.

A method and/or use disclosed herein applies a composition disclosed herein to an individual. An individual refers to any animal including, without limitation, a fish, an amphibian, a bird and a mammal. A mammal includes a human, a horse, a cow, a sheep, a dog and a cat. As such, a method disclosed herein is for human use as well as veterinarian use.

In applications to an individual, a composition disclosed herein can be applied to a skin surface or can be internally administered. In one embodiment, a composition disclosed herein is applied topically to a skin region of an individual in order to reduce or inhibit microbial growth in the skin region. A skin region, includes, without limitation, the face, forehead, lips, scalp, neck, shoulder, arms, hands, thighs, legs, knees, feet, chest, breast, back, groin, buttocks, and the like.

In one embodiment, a composition disclosed herein is internally administered to an individual. Such routes of administration include enteral routes of administration and parenteral routes of administration.

A composition disclosed herein can be applied according to a method disclosed herein to a skin region. Application of a composition disclosed herein can be by rubbing, pouring, sprinkling, or spraying on, or otherwise applied to the human body. A composition disclosed herein can be applied by introducing the composition into or onto a solid support such as, e.g., a wipe, a towelette, a towel, a mitt, a glove, or a mask and then applying the composition to a skin region. A composition disclosed herein can be applied by using a delivery device, such as, e.g., an aerosol dispenser, a pump spray, a trigger spray, a squeeze bottle, a topical patch, a transdermal patch, or a dermal implant to apply the composition to a skin region.

A composition disclosed herein can be applied to an individual according to a method disclosed herein as often as needed and/or desired. A composition disclosed herein can be applied to an individual daily, every other day, every third of day, once a week, multiple times per week, once a month, multiple times per month, once a year or multiple times per year, as desired. A composition disclosed herein can be applied to an individual multiple times per day, e.g., twice a day, three times a day, four time a day, five times a day, six times a day or as often as desired.

The presently disclosed compositions are useful in food product, poultry, meat, vegetable production. For example, the compositions disclosed herein can be used to reduce or inhibit microbial growth on or in equipment, appliances or machinery, tables, rooms, including floors, ceilings and walls and any other hard surface from microbial contamination.

The presently disclosed compositions are useful in residential, commercial or industrial settings using equipment, appliances, machinery or any other apparatus using water in the course of its operation. For example, the compositions disclosed herein can be used to reduce or inhibit microbial growth on or in equipment, appliances or machinery, such as, e.g., refrigerators, freezers, dishwashers, clothes washers, water heaters, air conditioners, cooling towers or any apparatus using a humidifier.

The presently disclosed compositions are useful in formulations on plants as a preservative or pesticide. For example, the compositions disclosed herein can be used to reduce or inhibit microbial growth on or in a plant injured by a wound or infected with a microbe.

The presently disclosed compositions are useful in formulations on cut flowers to prolong freshness and health. For example, the compositions disclosed herein can be used to reduce or inhibit microbial growth on or in a cut flower at a wound or to prevent microbial infection.

A disclosed method and/or use applies a composition disclosed herein for specified amount of time. In one embodiment, a specified amount of time is a time sufficient to reduce or inhibit microbial growth on or in a medical device, a surface, or an individual. In another embodiment, a specified amount of time is a time sufficient to wash or irrigate a medical device, a surface, or an individual.

In aspects of this embodiment, a composition disclosed herein is applied to a device, like a medical device, a surface, or an individual for, e.g., about 1 minute, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 70 minutes, about 80 minutes, about 90 minutes, about 100 minutes, about 110 minutes, or about 120 minutes. In other aspects of this embodiment, a composition disclosed herein is applied to a device, like a medical device, a surface, or an individual for, e.g., at least 1 minute, at least 5 minutes, at least 10 minutes, at least 15 minutes, at least 20 minutes, at least 25 minutes, at least 30 minutes, at least 35 minutes, at least 40 minutes, at least 45 minutes, at least 50 minutes, at least 55 minutes, at least 60 minutes, at least 70 minutes, at least 80 minutes, at least 90 minutes, at least 100 minutes, at least 110 minutes, or at least 120 minutes. In yet other aspects of this embodiment, a composition disclosed herein is applied to device, like a medical device, a surface, or an individual for, e.g., at most 1 minute, at most 5 minutes, at most 10 minutes, at most 15 minutes, at most 20 minutes, at most 25 minutes, at most 30 minutes, at most 35 minutes, at most 40 minutes, at most 45 minutes, at most 50 minutes, at most 55 minutes, at most 60 minutes, at most 70 minutes, at most 80 minutes, at most 90 minutes, at most 100 minutes, at most 110 minutes, or at most 120 minutes.

In still other aspects of this embodiment, a composition disclosed herein is applied to a device, like a medical device, a surface, or an individual for, e.g., about 1 minute to about 5 minutes, about 1 minute to about 10 minutes, about 1 minute to about 15 minutes, about 1 minute to about 20 minutes, about 1 minute to about 25 minutes, about 1 minute to about 30 minutes, about 1 minute to about 35 minutes, about 1 minute to about 40 minutes, about 1 minute to about 45 minutes, about 1 minute to about 50 minutes, about 1 minute to about 55 minutes, about 1 minute to about 60 minutes, about 5 minutes to about 10 minutes, about 5 minutes to about 15 minutes, about 5 minutes to about 20 minutes, about 5 minutes to about 25 minutes, about 5 minutes to about 30 minutes, about 5 minutes to about 35 minutes, about 5 minutes to about 40 minutes, about 5 minutes to about 45 minutes, about 5 minutes to about 50 minutes, about 5 minutes to about 55 minutes, about 5 minutes to about 60 minutes, about 5 minutes to about 70 minutes, about 5 minutes to about 80 minutes, about 5 minutes to about 90 minutes, about 5 minutes to about 100 minutes, about 5 minutes to about 110 minutes, about 5 minutes to about 120 minutes, about 10 minutes to about 15 minutes, about 10 minutes to about 20 minutes, about 10 minutes to about 25 minutes, about 10 minutes to about 30 minutes, about 10 minutes to about 35 minutes, about 10 minutes to about 40 minutes, about 10 minutes to about 45 minutes, about 10 minutes to about 50 minutes, about 10 minutes to about 55 minutes, about 10 minutes to about 60 minutes, about 10 minutes to about 70 minutes, about 10 minutes to about 80 minutes, about 10 minutes to about 90 minutes, about 10 minutes to about 100 minutes, about 10 minutes to about 110 minutes, about 10 minutes to about 120 minutes, about 15 minutes to about 20 minutes, about 15 minutes to about 25 minutes, about 15 minutes to about 30 minutes, about 15 minutes to about 35 minutes, about 15 minutes to about 40 minutes, about 15 minutes to about 45 minutes, about 15 minutes to about 50 minutes, about 15 minutes to about 55 minutes, about 15 minutes to about 60 minutes, about 15 minutes to about 70 minutes, about 15 minutes to about 80 minutes, about 15 minutes to about 90 minutes, about 15 minutes to about 100 minutes, about 15 minutes to about 110 minutes, about 15 minutes to about 120 minutes, about 20 minutes to about 25 minutes, about 20 minutes to about 30 minutes, about 20 minutes to about 35 minutes, about 20 minutes to about 40 minutes, about 20 minutes to about 45 minutes, about 20 minutes to about 50 minutes, about 20 minutes to about 55 minutes, about 20 minutes to about 60 minutes, about 20 minutes to about 70 minutes, about 20 minutes to about 80 minutes, about 20 minutes to about 90 minutes, about 20 minutes to about 100 minutes, about 20 minutes to about 110 minutes, about 20 minutes to about 120 minutes, about 25 minutes to about 30 minutes, about 25 minutes to about 35 minutes, about 25 minutes to about 40 minutes, about 25 minutes to about 45 minutes, about 25 minutes to about 50 minutes, about 25 minutes to about 55 minutes, about 25 minutes to about 60 minutes, about 25 minutes to about 70 minutes, about 25 minutes to about 80 minutes, about 25 minutes to about 90 minutes, about 25 minutes to about 100 minutes, about 25 minutes to about 110 minutes, about 25 minutes to about 120 minutes, about 30 minutes to about 35 minutes, about 30 minutes to about 40 minutes, about 30 minutes to about 45 minutes, about 30 minutes to about 50 minutes, about 30 minutes to about 55 minutes, about 30 minutes to about 60 minutes, about 30 minutes to about 70 minutes, about 30 minutes to about 80 minutes, about 30 minutes to about 90 minutes, about 30 minutes to about 100 minutes, about 30 minutes to about 110 minutes, about 30 minutes to about 120 minutes, about 35 minutes to about 40 minutes, about 35 minutes to about 45 minutes, about 35 minutes to about 50 minutes, about 35 minutes to about 55 minutes, about 35 minutes to about 60 minutes, about 35 minutes to about 70 minutes, about 35 minutes to about 80 minutes, about 35 minutes to about 90 minutes, about 35 minutes to about 100 minutes, about 35 minutes to about 110 minutes, about 35 minutes to about 120 minutes, about 40 minutes to about 45 minutes, about 40 minutes to about 50 minutes, about 40 minutes to about 55 minutes, about 40 minutes to about 60 minutes, about 40 minutes to about 70 minutes, about 40 minutes to about 80 minutes, about 40 minutes to about 90 minutes, about 40 minutes to about 100 minutes, about 40 minutes to about 110 minutes, about 40 minutes to about 120 minutes, about 45 minutes to about 50 minutes, about 45 minutes to about 55 minutes, about 45 minutes to about 60 minutes, about 45 minutes to about 70 minutes, about 45 minutes to about 80 minutes, about 45 minutes to about 90 minutes, about 45 minutes to about 100 minutes, about 45 minutes to about 110 minutes, about 45 minutes to about 120 minutes, about 50 minutes to about 55 minutes, about 50 minutes to about 60 minutes, about 50 minutes to about 70 minutes, about 50 minutes to about 80 minutes, about 50 minutes to about 90 minutes, about 50 minutes to about 100 minutes, about 50 minutes to about 110 minutes, about 50 minutes to about 120 minutes, about 55 minutes to about 60 minutes, about 55 minutes to about 70 minutes, about 55 minutes to about 80 minutes, about 55 minutes to about 90 minutes, about 55 minutes to about 100 minutes, about 55 minutes to about 110 minutes, about 55 minutes to about 120 minutes, about 60 minutes to about 70 minutes, about 60 minutes to about 80 minutes, about 60 minutes to about 90 minutes, about 60 minutes to about 100 minutes, about 60 minutes to about 110 minutes, about 60 minutes to about 120 minutes, about 70 minutes to about 80 minutes, about 70 minutes to about 90 minutes, about 70 minutes to about 100 minutes, about 70 minutes to about 110 minutes, about 70 minutes to about 120 minutes, about 80 minutes to about 90 minutes, about 80 minutes to about 100 minutes, about 80 minutes to about 110 minutes, about 80 minutes to about 120 minutes, about 90 minutes to about 100 minutes, about 90 minutes to about 110 minutes, about 90 minutes to about 120 minutes, about 100 minutes to about 110 minutes, about 100 minutes to about 120 minutes, or about 110 minutes to about 120 minutes.

The disclosed compositions, methods and uses are less harsh than conventional compositions, methods and uses resulting in a longer lifetime use of a surface and/or device, equipment, machinery or other apparatus.

In aspects of this embodiment, a surface and/or device, equipment, machinery or other apparatus can be exposed to a composition disclosed herein, e.g., about 50 times, about 60 times, about 70 times, about 80 times, about 90 times, about 100 times, about 110 times, about 120 times, about 130 times, about 140 times, about 150 times, about 160 times, about 170 times, about 180 times, about 190 times, about 200 times, about 210 times, about 220 times, about 230 times, about 240 times, about 250 times, about 260 times, about 270 times, about 280 times, about 290 times, or about 300 times. In other aspects of this embodiment, a surface and/or device, equipment, machinery or other apparatus can be exposed to a composition disclosed herein, e.g., at least 50 times, at least 60 times, at least 70 times, at least 80 times, at least 90 times, at least 100 times, at least 110 times, at least 120 times, at least 130 times, at least 140 times, at least 150 times, at least 160 times, at least 170 times, at least 180 times, at least 190 times, at least 200 times, at least 210 times, at least 220 times, at least 230 times, at least 240 times, at least 250 times, at least 260 times, at least 270 times, at least 280 times, at least 290 times, or at least 300 times. In yet other aspects of this embodiment, a medical device can be cleaned, disinfected and/or sterilized, e.g., at most 50 times, at most 60 times, at most 70 times, at most 80 times, at most 90 times, at most 100 times, at most 110 times, at most 120 times, at most 130 times, at most 140 times, at most 150 times, at most 160 times, at most 170 times, at most 180 times, at most 190 times, at most 200 times, at most 210 times, at most 220 times, at most 230 times, at most 240 times, at most 250 times, at most 260 times, at most 270 times, at most 280 times, at most 290 times, or at most 300 times.

In still other aspects of this embodiment, a surface and/or device, equipment, machinery or other apparatus can be exposed to a composition disclosed herein, e.g., about 50 times to about 60 times, about 50 times to about 70 times, about 50 times to about 80 times, about 50 times to about 90 times, about 50 times to about 100 times, about 50 times to about 110 times, about 50 times to about 120 times, about 50 times to about 130 times, about 50 times to about 140 times, about 50 times to about 150 times, about 50 times to about 175 times, about 50 times to about 200 times, about 50 times to about 225 times, about 50 times to about 250 times, about 50 times to about 275 times, about 50 times to about 300 times, about 75 times to about 90 times, about 75 times to about 100 times, about 75 times to about 110 times, about 75 times to about 120 times, about 75 times to about 130 times, about 75 times to about 140 times, about 75 times to about 150 times, about 75 times to about 175 times, about 75 times to about 200 times, about 75 times to about 225 times, about 75 times to about 250 times, about 75 times to about 275 times, about 75 times to about 300 times, about 100 times to about 110 times, about 100 times to about 120 times, about 10⁰ times to about 130 times, about 100 times to about 140 times, about 100 times to about 150 times, about 100 times to about 175 times, about 100 times to about 200 times, about 100 times to about 225 times, about 100 times to about 250 times, about 100 times to about 275 times, about 100 times to about 300 times, about 125 times to about 150 times, about 125 times to about 175 times, about 125 times to about 200 times, about 125 times to about 225 times, about 125 times to about 250 times, about 125 times to about 275 times, about 125 times to about 300 times, about 150 times to about 175 times, about 150 times to about 200 times, about 150 times to about 225 times, about 150 times to about 250 times, about 150 times to about 275 times, about 150 times to about 300 times, about 175 times to about 200 times, about 175 times to about 225 times, about 175 times to about 250 times, about 175 times to about 275 times, about 175 times to about 300 times, about 200 times to about 225 times, about 200 times to about 250 times, about 200 times to about 275 times, about 200 times to about 300 times, about 225 times to about 250 times, about 225 times to about 275 times, about 225 times to about 300 times, about 250 times to about 275 times, about 250 times to about 300 times, or about 275 times to about 300 times.

Aspects of the present specification can also be described as follows:
1. A composition comprising, consisting essentially of, or consisting of hypochlorous acid or free available chlorine.
2. A composition comprising, consisting essentially of, or consisting of hypochlorous acid or free available chlorine and one or more quaternary compounds or silicon quaternary compounds.
3. A composition comprising, consisting essentially of, or consisting of hypochlorous acid or free available chlorine and one or more guanide-containing compounds.
4. A composition comprising, consisting essentially of, or consisting of hypochlorous acid or free available chlorine and one or more guanide-containing compounds and one or more surfactants.
5. A composition comprising, consisting essentially of, or consisting of hypochlorous acid or free available chlorine, one or more quaternary compounds or silicon quaternary compounds. and one or more guanide-containing compounds.
6. A composition comprising, consisting essentially of, or consisting of hypochlorous acid or free available chlorine and one or more alcohols.
7. A composition comprising, consisting essentially of, or consisting of one or more alcohols and one or more quaternary compounds or silicon quaternary compounds.
8. A composition comprising, consisting essentially of, or consisting of one or more alcohols and one or more guanide-containing compounds.
9. The composition according to any one of embodiments 1-8 further comprising, consisting essentially of, or consisting of one or more metallic particles.
10. The composition according to any one of embodiments 1-9 further comprising, consisting essentially of, or consisting of one or more metal salts.
11. A composition comprising, consisting essentially of, or consisting of hypochlorous acid or free available chlorine and one or more metallic particles.
12. A composition comprising, consisting essentially of, or consisting of hypochlorous acid or free available chlorine and one or more metal salts.
13. A composition comprising, consisting essentially of, or consisting of hypochlorous acid or free available chlorine, one or more metallic particles and one or more metal salts.
14. The composition according to embodiments 1-6 and 11-13, wherein the hypochlorous acid or free available chlorine is in an amount of 0.01 ppm, 0.05 ppm, 0.10 ppm, 0.15 ppm, 0.20 ppm, 0.25 ppm, 0.30 ppm, 0.35 ppm, 0.40 ppm, 0.45 ppm, 0.50 ppm, 0.55 ppm, 0.60 ppm, 0.65 ppm, 0.70 ppm, 0.75 ppm, 0.80 ppm, 0.85 ppm, 0.90 ppm, 0.95 ppm, 1 ppm, 5 ppm, 10 ppm, 15 ppm, 20 ppm, 25 ppm, 30 ppm, 35 ppm, 40 ppm, 45 ppm, 50 ppm, 55 ppm, 60 ppm, 65 ppm, 70 ppm, 75 ppm, 80 ppm, 85 ppm, 90 ppm, 95 ppm, 100 ppm, 125 ppm, 150 ppm, 175 ppm, 200 ppm, 225 ppm, 250 ppm, 275 ppm, 300 ppm, 325 ppm, 350 ppm, 375 ppm, 400 ppm, 425 ppm, 450 ppm, 475 ppm, 500 ppm, 525 ppm, 550 ppm, 575 ppm, 600 ppm, 625 ppm, 650 ppm, 675 ppm, 700 ppm, 725 ppm, 750 ppm, 775 ppm, 800 ppm, 825 ppm, 850 ppm, 875 ppm, 900 ppm, 925 ppm, 950 ppm, 975 ppm, 1,000 ppm, 1,025 ppm, 1,050 ppm, 1075 ppm, 1,100 ppm, 1,125 ppm, 1,150 ppm, 1,175 ppm, 1,200 ppm, 1,225 ppm, 1,250 ppm, 1,275 ppm, 1,300 ppm, 1,325 ppm, 1,350 ppm, 1,375 ppm, 1,400 ppm, 1,425 ppm, 1,450 ppm, 1,475 ppm, 1,500 ppm, 1,600 ppm, 1,700 ppm, 1,800 ppm, 1,900 ppm, or 2,000 ppm or at least 0.01 ppm, at least 0.05 ppm, at least 0.10 ppm, at least 0.20 ppm, at least 0.30 ppm, at least 0.40 ppm, at least 0.50 ppm, at least 0.60 ppm, at least 0.70 ppm, at least 0.80 ppm, at least 0.90 ppm, at least 1 ppm, at least 10 ppm, at least 20 ppm, at least 30 ppm, at least 40 ppm, at least 50 ppm, at least 60 ppm, at least 70 ppm, at least 80 ppm, at least 90 ppm, at least 100 ppm, at least 125 ppm, at least 150 ppm, at least 175 ppm, at least 200 ppm, at least 225 ppm, at least 250 ppm, at least 275 ppm, at least 300 ppm, at least 325 ppm, at least 350 ppm, at least 375 ppm, at least 400 ppm, at least 425 ppm, at least 450 ppm, at least 475 ppm, at least 500 ppm, at least 525 ppm, at least 550 ppm, at least 575 ppm, at least 600 ppm, at least 625 ppm, at least 650 ppm, at least 675 ppm, at least 700 ppm, at least 725 ppm, at least 750 ppm, at least 775 ppm, at least 800 ppm, at least 825 ppm, at least 850 ppm, at least 875 ppm, at least 900 ppm, at least 925 ppm, at least 950 ppm, at least 975 ppm, at least 1,000 ppm, at least 1,025 ppm, at least 1,050 ppm, at least 1075 ppm, at least 1,100 ppm, at least 1,125 ppm, at least 1,150 ppm, at least 1,175 ppm, at least 1,200 ppm, at least 1,225 ppm, at least 1,250 ppm, at least 1,275 ppm, at least 1,300 ppm, at least 1,325 ppm, at least 1,350 ppm, at least 1,375 ppm, at least 1,400 ppm, at least 1,425 ppm, at least 1,450 ppm, at least 1,475 ppm, at least 1,500 ppm, at least 1,600 ppm, at least 1,700 ppm, at least 1,800 ppm, at least 1,900 ppm, or at least 2,000 ppm or at most 0.01 ppm, at most 0.05 ppm, at most 0.10 ppm, at most 0.20 ppm, at most 0.30 ppm, at most 0.40 ppm, at most 0.50 ppm, at most 0.60 ppm, at most 0.70 ppm, at most 0.80 ppm, at most 0.90 ppm, at most 1 ppm, at most 10 ppm, at most 20 ppm, at most 30 ppm, at most 40 ppm, at most 50 ppm, at most 60 ppm, at most 70 ppm, at most 80 ppm, at most 90 ppm, at most 100 ppm, at most 125 ppm, at most 150 ppm, at most 175 ppm, at most 200 ppm, at most 225 ppm, at most 250 ppm, at most 275 ppm, at most 300 ppm, at most 325 ppm, at most 350 ppm, at most 375 ppm, at most 400 ppm, at most 425 ppm, at most 450 ppm, at most 475 ppm, at most 500 ppm, at most 525 ppm, at most 550 ppm, at most 575 ppm, at most 600 ppm, at most 625 ppm, at most 650 ppm, at most 675 ppm, at most 700 ppm, at most 725 ppm, at most 750 ppm, at most 775 ppm, at most 800 ppm, at most 825 ppm, at most 850 ppm, at most 875 ppm, at most 900 ppm, at most 925 ppm, at most 950 ppm, at most 975 ppm, at most 1,000 ppm, at most 1,025 ppm, at most 1,050 ppm, at most 1075 ppm, at most 1,100 ppm, at most 1,125 ppm, at most 1,150 ppm, at most 1,175 ppm, at most 1,200 ppm, at most 1,225 ppm, at most 1,250 ppm, at most 1,275 ppm, at most 1,300 ppm, at most 1,325 ppm, at most 1,350 ppm, at most 1,375 ppm, at most 1,400 ppm, at most 1,425 ppm, at most 1,450 ppm, at most 1,475 ppm, at most 1,500 ppm, at most 1,600 ppm, at most 1,700 ppm, at most 1,800 ppm, at most 1,900 ppm, or at most 2,000 ppm or about 0.01 ppm to about 0.1 ppm, about 0.01 ppm to about 0.2 ppm, about 0.01 ppm to about 0.25 ppm, about 0.01 ppm to about 0.3 ppm, about 0.01 ppm to about 0.4 ppm, about 0.01 ppm to about 0.5 ppm, about 0.01 ppm to about 0.1 ppm, about 0.01 ppm to about 0.5 ppm, about 0.01 ppm to about 1 ppm, about 0.01 ppm to about 5 ppm, about 0.01 ppm to about 10 ppm, about 0.01 ppm to about 15 ppm, about 0.01 ppm to about 20 ppm, about 0.01 ppm to about 25 ppm, about 0.01 ppm to about 30 ppm, about 0.01 ppm to about 35 ppm, about 0.01 ppm to about 40 ppm, about 0.01 ppm to about 45 ppm, about 0.01 ppm to about 50 ppm, about 0.05 ppm to about 0.1 ppm, about 0.05 ppm to about 0.5 ppm, about 0.05 ppm to about 1 ppm, about 0.05 ppm to about 5 ppm, about 0.05 ppm to about 10 ppm, about 0.05 ppm to about 15 ppm, about 0.05 ppm to about 20 ppm, about 0.05 ppm to about 25 ppm, about 0.05 ppm to about 30 ppm, about 0.05 ppm to about 35 ppm, about 0.05 ppm to about 40 ppm, about 0.05 ppm to about 45 ppm, about 0.05 ppm to about 50 ppm, about 0.1 ppm to about 1 ppm, about 0.1 ppm to about 5 ppm, about 0.1 ppm to about 10 ppm, about 0.1 ppm to about 15 ppm, about 0.1 ppm to about 20 ppm, about 0.1 ppm to about 25 ppm, about 0.1 ppm to about 30 ppm, about 0.1 ppm to about 35 ppm, about 0.1 ppm to about 40 ppm, about 0.1 ppm to about 45 ppm, about 0.1 ppm to about 50 ppm, about 0.5 ppm to about 1 ppm, about 0.5 ppm to about 5 ppm, about 0.5 ppm to about 10 ppm, about 0.5 ppm to about 15 ppm, about 0.5 ppm to about 20 ppm, about 0.5 ppm to about 25 ppm, about 0.5 ppm to about 30 ppm, about 0.5 ppm to about 35 ppm, about 0.5 ppm to about 40 ppm, about 0.5 ppm to about 45 ppm, about 0.5 ppm to about 50 ppm, about 0.5 ppm to about 55 ppm, about 0.5 ppm to about 60 ppm, about 0.5 ppm to about 65 ppm, about 0.5 ppm to about 70 ppm, about 0.5 ppm to about 75 ppm, about 0.5 ppm to about 80 ppm, about 0.5 ppm to about 85 ppm, about 0.5 ppm to about 90 ppm, about 0.5 ppm to about 95 ppm, about 0.5 ppm to about 100 ppm, about 0.75 ppm to about 20 ppm, about 0.75 ppm to about 25 ppm, about 0.75 ppm to about 30 ppm, about 0.75 ppm to about 35 ppm, about 0.75 ppm to about 40 ppm, about 0.75 ppm to about 45 ppm, about 0.75 ppm to about 50 ppm, about 0.75 ppm to about 55 ppm, about 0.75 ppm to about 60 ppm, about 0.75 ppm to about 65 ppm, about 0.75 ppm to about 70 ppm, about 0.75 ppm to about 75 ppm, about 0.75 ppm to about 80 ppm, about 0.75 ppm to about 85 ppm, about 0.75 ppm to about 90 ppm, about 0.75 ppm to about 95 ppm, about 0.75 ppm to about 100 ppm, about 1 ppm to about 20 ppm, about 1 ppm to about 25 ppm, about 1 ppm to about 30 ppm, about 1 ppm to about 35 ppm, about 1 ppm to about 40 ppm, about 1 ppm to about 45 ppm, about 1 ppm to about 50 ppm, about 1 ppm to about 55 ppm, about 1 ppm to about 60 ppm, about 1 ppm to about 65 ppm, about 1 ppm to about 70 ppm, about 1 ppm to about 75 ppm, about 1 ppm to about 80 ppm, about 1 ppm to about 85 ppm, about 1 ppm to about 90 ppm, about 1 ppm to about 95 ppm, about 1 ppm to about 100 ppm, about 5 ppm to about 20 ppm, about 5 ppm to about 25 ppm, about 5 ppm to about 30 ppm, about 5 ppm to about 35 ppm, about 5 ppm to about 40 ppm, about 5 ppm to about 45 ppm, about 5 ppm to about 50 ppm, about 5 ppm to about 55 ppm, about 5 ppm to about 60 ppm, about 5 ppm to about 65 ppm, about 5 ppm to about 70 ppm, about 5 ppm to about 75 ppm, about 5 ppm to about 80 ppm, about 5 ppm to about 85 ppm, about 5 ppm to about 90 ppm, about 5 ppm to about 95 ppm, about 5 ppm to about 100 ppm, about 10 ppm to about 20 ppm, about 10 ppm to about 25 ppm, about 10 ppm to about 30 ppm, about 10 ppm to about 35 ppm, about 10 ppm to about 40 ppm, about 10 ppm to about 45 ppm, about 10 ppm to about 50 ppm, about 10 ppm to about 55 ppm, about 10 ppm to about 60 ppm, about 10 ppm to about 65 ppm, about 10 ppm to about 70 ppm, about 10 ppm to about 75 ppm, about 10 ppm to about 80 ppm, about 10 ppm to about 85 ppm, about 10 ppm to about 90 ppm, about 10 ppm to about 95 ppm, or about 10 ppm to about 100 ppm, about 1 ppm to about 25 ppm, about 1 ppm to about 50 ppm, about 1 ppm to about 75 ppm, about 1 ppm to about 100 ppm, about 1 ppm to about 125 ppm, about 1 ppm to about 150 ppm, about 1 ppm to about 175 ppm, about 1 ppm to about 200 ppm, about 1 ppm to about 225 ppm, about 1 ppm to about 250 ppm, about 1 ppm to about 275 ppm, about 1 ppm to about 300 ppm, about 1 ppm to about 325 ppm, about 1 ppm to about 350 ppm, about 1 ppm to about 375 ppm, about 1 ppm to about 400 ppm, about 10 ppm to about 25 ppm, about 10 ppm to about 50 ppm, about 10 ppm to about 75 ppm, about 10 ppm to about 100 ppm, about 10 ppm to about 125 ppm, about 10 ppm to about 150 ppm, about 10 ppm to about 175 ppm, about 10 ppm to about 200 ppm, about 10 ppm to about 225 ppm, about 10 ppm to about 250 ppm, about 10 ppm to about 275 ppm, about 10 ppm to about 300 ppm, about 10 ppm to about 325 ppm, about 10 ppm to about 350 ppm, about 10 ppm to about 375 ppm, about 10 ppm to about 400 ppm, about 25 ppm to about 50 ppm, about 25 ppm to about 75 ppm, about 25 ppm to about 100 ppm, about 25 ppm to about 125 ppm, about 25 ppm to about 150 ppm, about 25 ppm to about 175 ppm, about 25 ppm to about 200 ppm, about 25 ppm to about 225 ppm, about 25 ppm to about 250 ppm, about 25 ppm to about 275 ppm, about 25 ppm to about 300 ppm, about 25 ppm to about 325 ppm, about 25 ppm to about 350 ppm, about 25 ppm to about 375 ppm, about 25 ppm to about 400 ppm, about 50 ppm to about 75 ppm, about 50 ppm to about 100 ppm, about 50 ppm to about 125 ppm, about 50 ppm to about 150 ppm, about 50 ppm to about 175 ppm, about 50 ppm to about 200 ppm, about 50 ppm to about 225 ppm, about 50 ppm to about 250 ppm, about 50 ppm to about 275 ppm, about 50 ppm to about 300 ppm, about 50 ppm to about 325 ppm, about 50 ppm to about 350 ppm, about 50 ppm to about 375 ppm, about 50 ppm to about 400 ppm, about 75 ppm to about 100 ppm, about 75 ppm to about 125 ppm, about 75 ppm to about 150 ppm, about 75 ppm to about 175 ppm, about 75 ppm to about 200 ppm, about 75 ppm to about 225 ppm, about 75 ppm to about 250 ppm, about 75 ppm to about 275 ppm, about 75 ppm to about 300 ppm, about 75 ppm to about 325 ppm, about 75 ppm to about 350 ppm, about 75 ppm to about 375 ppm, about 75 ppm to about 400 ppm, about 100 ppm to about 125 ppm, about 100 ppm to about 150 ppm, about 100 ppm to about 175 ppm, about 100 ppm to about 200 ppm, about 100 ppm to about 225 ppm, about 100 ppm to about 250 ppm, about 100 ppm to about 275 ppm, about 100 ppm to about 300 ppm, about 100 ppm to about 325 ppm, about 100 ppm to about 350 ppm, about 100 ppm to about 375 ppm, about 100 ppm to about 400 ppm, about 150 ppm to about 175 ppm, about 150 ppm to about 200 ppm, about 150 ppm to about 225 ppm, about 150 ppm to about 250 ppm, about 150 ppm to about 275 ppm, about 150 ppm to about 300 ppm, about 150 ppm to about 325 ppm, about 150 ppm to about 350 ppm, about 150 ppm to about 375 ppm, about 150 ppm to about 400 ppm, about 200 ppm to about 225 ppm, about 200 ppm to about 250 ppm, about 200 ppm to about 275 ppm, about 200 ppm to about 300 ppm, about 200 ppm to about 325 ppm, about 200 ppm to about 350 ppm, about 200 ppm to about 375 ppm, about 200 ppm to about 400 ppm, about 200 ppm to about 425 ppm, about 200 ppm to about 450 ppm, about 200 ppm to about 475 ppm, about 200 ppm to about 500 ppm, about 200 ppm to about 525 ppm, about 200 ppm to about 550 ppm, about 200 ppm to about 575 ppm, about 200 ppm to about 600 ppm, about 250 ppm to about 275 ppm, about 250 ppm to about 300 ppm, about 250 ppm to about 325 ppm, about 250 ppm to about 350 ppm, about 250 ppm to about 375 ppm, about 250 ppm to about 400 ppm, about 250 ppm to about 425 ppm, about 250 ppm to about 450 ppm, about 250 ppm to about 475 ppm, about 250 ppm to about 500 ppm, about 250 ppm to about 525 ppm, about 250 ppm to about 550 ppm, about 250 ppm to about 575 ppm, about 250 ppm to about 600 ppm, about 300 ppm to about 325 ppm, about 300 ppm to about 350 ppm, about 300 ppm to about 375 ppm, about 300 ppm to about 400 ppm, about 300 ppm to about 425 ppm, about 300 ppm to about 450 ppm, about 300 ppm to about 475 ppm, about 300 ppm to about 500 ppm, about 300 ppm to about 525 ppm, about 300 ppm to about 550 ppm, about 300 ppm to about 575 ppm, about 300 ppm to about 600 ppm, about 350 ppm to about 375 ppm, about 350 ppm to about 400 ppm, about 375 ppm to about 400 ppm, about 350 ppm to about 425 ppm, about 350 ppm to about 450 ppm, about 350 ppm to about 475 ppm, about 350 ppm to about 500 ppm, about 350 ppm to about 525 ppm, about 350 ppm to about 550 ppm, about 350 ppm to about 575 ppm, about 350 ppm to about 600 ppm, about 400 ppm to about 500 ppm, about 400 ppm to about 600 ppm, about 400 ppm to about 700 ppm, about 400 ppm to about 800 ppm, about 400 ppm to about 900 ppm, about 400 ppm to about 1,000 ppm, about 400 ppm to about 1,100 ppm, about 400 ppm to about 1,200 ppm, about 400 ppm to about 1,300 ppm, about 400 ppm to about 1,400 ppm, about 400 ppm to about 1,500 ppm, about 400 ppm to about 1,600 ppm, about 400 ppm to about 1,700 ppm, about 400 ppm to about 1,800 ppm, about 400 ppm to about 1,900 ppm, about 400 ppm to about 2,000 ppm, about 500 ppm to about 600 ppm, about 500 ppm to about 700 ppm, about 500 ppm to about 800 ppm, about 500 ppm to about 900 ppm, about 500 ppm to about 1,000 ppm, about 500 ppm to about 1,100 ppm, about 500 ppm to about 1,200 ppm, about 500 ppm to about 1,300 ppm, about 500 ppm to about 1,400 ppm, about 500 ppm to about 1,500 ppm, about 500 ppm to about 1,600 ppm, about 500 ppm to about 1,700 ppm, about 500 ppm to about 1,800 ppm, about 500 ppm to about 1,900 ppm, about 500 ppm to about 2,000 ppm, about 600 ppm to about 700 ppm, about 600 ppm to about 800 ppm, about 600 ppm to about 900 ppm, about 600 ppm to about 1,000 ppm, about 600 ppm to about 1,100 ppm, about 600 ppm to about 1,200 ppm, about 600 ppm to about 1,300 ppm, about 600 ppm to about 1,400 ppm, about 600 ppm to about 1,500 ppm, about 600 ppm to about 1,600 ppm, about 600 ppm to about 1,700 ppm, about 600 ppm to about 1,800 ppm, about 600 ppm to about 1,900 ppm, about 600 ppm to about 2,000 ppm, about 700 ppm to about 800 ppm, about 700 ppm to about 900 ppm, about 700 ppm to about 1,000 ppm, about 700 ppm to about 1,100 ppm, about 700 ppm to about 1,200 ppm, about 700 ppm to about 1,300 ppm, about 700 ppm to about 1,400 ppm, about 700 ppm to about 1,500 ppm, about 700 ppm to about 1,600 ppm, about 700 ppm to about 1,700 ppm, about 700 ppm to about 1,800 ppm, about 700 ppm to about 1,900 ppm, about 700 ppm to about 2,000 ppm, about 800 ppm to about 900 ppm, about 800 ppm to about 1,000 ppm, about 800 ppm to about 1,100 ppm, about 800 ppm to about 1,200 ppm, about 800 ppm to about 1,300 ppm, about 800 ppm to about 1,400 ppm, about 800 ppm to about 1,500 ppm, about 800 ppm to about 1,600 ppm, about 800 ppm to about 1,700 ppm, about 800 ppm to about 1,800 ppm, about 800 ppm to about 1,900 ppm, about 800 ppm to about 2,000 ppm, about 900 ppm to about 1,000 ppm, about 900 ppm to about 1,100 ppm, about 900 ppm to about 1,200 ppm, about 900 ppm to about 1,300 ppm, about 900 ppm to about 1,400 ppm, about 900 ppm to about 1,500 ppm, about 900 ppm to about 1,600 ppm, about 900 ppm to about 1,700 ppm, about 900 ppm to about 1,800 ppm, about 900 ppm to about 1,900 ppm, about 900 ppm to about 2,000 ppm, about 1,000 ppm to about 1,100 ppm, about 1,000 ppm to about 1,200 ppm, about 1,000 ppm to about 1,300 ppm, about 1,000 ppm to about 1,400 ppm, about 1,000 ppm to about 1,500 ppm, about 1,000 ppm to about 1,600 ppm, about 1,000 ppm to about 1,700 ppm, about 1,000 ppm to about 1,800 ppm, about 1,000 ppm to about 1,900 ppm, about 1,000 ppm to about 2,000 ppm, about 1,100 ppm to about 1,200 ppm, about 1,100 ppm to about 1,300 ppm, about 1,100 ppm to about 1,400 ppm, about 1,100 ppm to about 1,500 ppm, about 1,100 ppm to about 1,600 ppm, about 1,100 ppm to about 1,700 ppm, about 1,100 ppm to about 1,800 ppm, about 1,100 ppm to about 1,900 ppm, about 1,100 ppm to about 2,000 ppm, about 1,200 ppm to about 1,300 ppm, about 1,200 ppm to about 1,400 ppm, about 1,200 ppm to about 1,500 ppm, about 1,200 ppm to about 1,600 ppm, about 1,200 ppm to about 1,700 ppm, about 1,200 ppm to about 1,800 ppm, about 1,200 ppm to about 1,900 ppm, about 1,200 ppm to about 2,000 ppm, about 1,300 ppm to about 1,400 ppm, about 1,300 ppm to about 1,500 ppm, about 1,300 ppm to about 1,600 ppm, about 1,300 ppm to about 1,700 ppm, about 1,300 ppm to about 1,800 ppm, about 1,300 ppm to about 1,900 ppm, about 1,300 ppm to about 2,000 ppm, about 1,400 ppm to about 1,500 ppm, about 1,400 ppm to about 1,600 ppm, about 1,400 ppm to about 1,700 ppm, about 1,400 ppm to about 1,800 ppm, about 1,400 ppm to about 1,900 ppm, about 1,400 ppm to about 2,000 ppm, about 1,500 ppm to about 1,600 ppm, about 1,500 ppm to about 1,700 ppm, about 1,500 ppm to about 1,800 ppm, about 1,500 ppm to about 1,900 ppm, about 1,500 ppm to about 2,000 ppm, about 1,600 ppm to about 1,700 ppm, about 1,600 ppm to about 1,800 ppm, about 1,600 ppm to about 1,900 ppm, about 1,600 ppm to about 2,000 ppm, about 1,700 ppm to about 1,800 ppm, about 1,700 ppm to about 1,900 ppm, about 1,700 ppm to about 2,000 ppm, about 1,800 ppm to about 1,900 ppm, about 1,800 ppm to about 2,000 ppm, or about 1,900 ppm to about 2,000 ppm. 15. The composition according to any one of embodiments 1-6 and 11-13, wherein the hypochlorous acid or free available chlorine is in an amount of about 0.001%, about 0.005%, about 0.01%, about 0.05%, about 0.10%, about 0.11%, about 0.12%, about 0.13%, about 0.14%, about 0.15%, about 0.16%, about 0.17%, about 0.18%, about 0.19%, about 0.2%, about 0.25%, about 0.3%, about 0.35%, about 0.4%, about 0.45%, or about 0.5% by weight of the composition, or at least 0.001%, at least 0.005%, at least 0.01%, at least 0.05%, at least 0.10%, at least 0.11%, at least 0.12%, at least 0.13%, at least 0.14%, at least 0.15%, at least 0.16%, at least 0.17%, at least 0.18%, at least 0.19%, at least 0.2%, at least 0.25%, at least 0.3%, at least 0.35%, at least 0.4%, at least 0.45%, or at least 0.5% by weight of the composition, or at most 0.001%, at most 0.005%, at most 0.01%, at most 0.05%, at most 0.10%, at most 0.11%, at most 0.12%, at most 0.13%, at most 0.14%, at most 0.15%, at most 0.16%, at most 0.17%, at most 0.18%, at most 0.19%, at most 0.2%, at most 0.25%, at most 0.3%, at most 0.35%, at most 0.4%, at most 0.45%, or at most 0.5% by weight of the composition, or about 0.0010% to about 0.005%, about 0.0010% to about 0.010%, about 0.0010% to about 0.050%, about 0.0010% to about 0.10%, about 0.0010% to about 0.15%, about 0.0010% to about 0.20%, about 0.0010% to about 0.25%, about 0.0010% to about 0.30%, about 0.0010% to about 0.35%, about 0.0010% to about 0.40%, about 0.0010% to about 0.45%, about 0.0010% to about 0.50%, about 0.010% to about 0.050%, about 0.010% to about 0.10%, about 0.010% to about 0.15%, about 0.010% to about 0.20%, about 0.010% to about 0.25%, about 0.010% to about 0.30%, about 0.010% to about 0.35%, about 0.010% to about 0.40%, about 0.010% to about 0.45%, about 0.010% to about 0.50%, about 0.10% to about 0.15%, about 0.10% to about 0.20%, about 0.10% to about 0.25%, about 0.10% to about 0.30%, about 0.10% to about 0.35%, about 0.10% to about 0.40%, about 0.10% to about 0.45%, about 0.10% to about 0.50%, about 0.20% to about 0.25%, about 0.20% to about 0.30%, about 0.20% to about 0.35%, about 0.20% to about 0.40%, about 0.20% to about 0.45%, about 0.20% to about 0.50%, about 0.30% to about 0.35%, about 0.30% to about 0.40%, about 0.30% to about 0.45%, about 0.30% to about 0.50%, about 0.40% to about 0.45%, about 0.40% to about 0.50%, or about 0.40% to about 0.50% by weight of the composition.
16. The composition according to any one of embodiments 2, 5, 7, 14 or 15, wherein the one or more quaternary compounds include one or more dialkyl quaternary compounds and/or one or more polyether fatty quaternary compounds or wherein the one or more silicon quaternary compounds include one or more silicone dialkyl quaternary compounds and/or one or more silicone polyether fatty quaternary compounds.
17. The composition according to embodiment 15, wherein the one or more dialkyl quaternary compounds include didodecyldimethylammonium chloride and di-n-alkyldimethyl ammonium chloride and/or wherein the one or more silicone dialkyl quaternary compounds include dimethyloctadecyl[3-(trimethoxysilyl)propyl]ammonium chloride and tetradecyldimethyl-(3-trimethoxysilyl)propyl ammonium chloride.
18. The composition according to any one of embodiments 2, 5, 7, or 14-17, wherein the one or more quaternary compounds or silicon quaternary compounds are in an amount of about 0.01%, about 0.05%, about 0.075%, about 0.1%, about 0.2%, about 0.3%, about 0.4%, about 0.5%, about 0.6%, about 0.7%, about 0.8%, about 0.9%, about 1.0%, about 1.5%, about 2.0%, about 2.5%, about 3.0%, about 4.0%, about 5.0%, about 6.0%, about 7.0%, about 7.5%, about 8.0%, about 9.0%, about 10.0%, about 11.0%, about 12.0%, about 13.0%, about 14.0%, about 15.0%, about 16.0%, about 17.0%, 18.0%, about 19.0%, or about 20.0% by weight of the composition, or at least 0.01%, at least 0.05%, at least 0.075%, at least 0.1%, at least 0.25%, at least 0.5%, at least 0.75%, at least 1.0%, at least 1.5%, at least 2.0%, at least 2.5%, at least 3.0%, at least 4.0%, at least 5.0%, at least 6.0%, at least 7.0%, at least 7.5%, at least 8.0%, at least 9.0%, at least 10.0%, at least 11.0%, at least 12.0%, at least 13.0%, at least 14.0%, at least 15.0%, at least 16.0%, at least 17.0%, at least 18.0%, at least 19.0%, or at least 20.0% by weight of the composition by weight of the composition, or at most 0.01%, at most 0.05%, at most 0.075%, at most 0.1%, at most 0.25%, at most 0.5%, at most 0.75%, at most 1.0%, at most 1.5%, at most 2.0%, at most 2.5%, at most 3.0%, at most 4.0%, at most 5.0%, at most 6.0%, at most 7.5%, at most 8.0%, at most 9.0%, at most 10.0%, at most 11.0%, at most 12.0%, at most 13.0%, at most 14.0%, at most 15.0%, at most 16.0%, at most 17.0%, at most 18.0%, at most 19.0%, or at most 20.0% by weight of the composition by weight of the composition, or about 0.1% to about 0.5%, about 0.1% to about 0.75%, about 0.1% to about 1.0%, about 0.1% to about 1.5%, about 0.1% to about 2.0%, about 0.1% to about 2.5%, about 0.2% to about 0.5%, about 0.2% to about 0.75%, about 0.2% to about 1.0%, about 0.2% to about 1.5%, about 0.2% to about 2.0%, about 0.2% to about 2.5%, about 0.5% to about 1.0%, about 0.5% to about 1.5%, about 0.5% to about 2.0%, about 0.5% to about 2.5%, about 0.5% to about 3.0%, about 0.5% to about 4.0%, about 0.5% to about 5.0%, about 1.0% to about 2.0%, about 1.0% to about 3.0%, about 1.0% to about 4.0%, about 1.0% to about 5.0%, about 1.0% to about 6.0%, about 1.0% to about 7.0%, about 1.0% to about 8.0%, about 1.0% to about 9.0%, about 1.0% to about 10%, about 1.0% to about 11%, about 1.0% to about 12%, about 1.0% to about 13%, about 1.0% to about 14%, about 1.0% to about 15%, about 2.0% to about 3.0%, about 2.0% to about 4.0%, about 2.0% to about 5.0%, about 2.0% to about 6.0%, about 2.0% to about 7.0%, about 2.0% to about 8.0%, about 2.0% to about 9.0%, about 2.0% to about 10%, about 2.0% to about 11%, about 2.0% to about 12%, about 2.0% to about 13%, about 2.0% to about 14%, about 2.0% to about 15%, about 3.0% to about 4.0%, about 3.0% to about 5.0%, about 3.0% to about 6.0%, about 3.0% to about 7.0%, about 3.0% to about 8.0%, about 3.0% to about 9.0%, about 3.0% to about 10%, about 3.0% to about 11%, about 3.0% to about 12%, about 3.0% to about 13%, about 3.0% to about 14%, about 3.0% to about 15%, about 4.0% to about 5.0%, about 4.0% to about 6.0%, about 4.0% to about 7.0%, about 4.0% to about 8.0%, about 4.0% to about 9.0%, about 4.0% to about 10%, about 4.0% to about 11%, about 4.0% to about 12%, about 4.0% to about 13%, about 4.0% to about 14%, about 4.0% to about 15%, about 5.0% to about 6.0%, about 5.0% to about 7.0%, about 5.0% to about 8.0%, about 5.0% to about 9.0%, about 5.0% to about 10%, about 5.0% to about 11%, about 5.0% to about 12%, about 5.0% to about 13%, about 5.0% to about 14%, about 5.0% to about 15%, about 6.0% to about 7.0%, about 6.0% to about 8.0%, about 6.0% to about 9.0%, about 6.0% to about 10%, about 6.0% to about 11%, about 6.0% to about 12%, about 6.0% to about 13%, about 6.0% to about 14%, about 6.0% to about 15%, about 7.0% to about 8.0%, about 7.0% to about 9.0%, about 7.0% to about 10%, about 7.0% to about 11%, about 7.0% to about 12%, about 7.0% to about 13%, about 7.0% to about 14%, about 7.0% to about 15%, about 8.0% to about 9.0%, about 8.0% to about 10%, about 8.0% to about 11%, about 8.0% to about 12%, about 8.0% to about 13%, about 8.0% to about 14%, about 8.0% to about 15%, about 9.0% to about 10%, about 9.0% to about 11%, about 9.0% to about 12%, about 9.0% to about 13%, about 9.0% to about 14%, about 9.0% to about 15%, about 10% to about 11%, about 10% to about 12%, about 10% to about 13%, about 10% to about 14%, about 10% to about 15%, about 11% to about 12%, about 11% to about 13%, about 11% to about 14%, about 11% to about 15%, about 12% to about 13%, about 12% to about 14%, about 12% to about 15%, about 13% to about 14%, about 13% to about 15%, or 14% to about 15% by weight of the composition.
19. The composition according to any one of embodiments 2, 5, 7, or 14-17, wherein the one or more quaternary compounds or silicon quaternary compounds is in an amount of 0.05 ppm, 0.10 ppm, 0.15 ppm, 0.20 ppm, 0.25 ppm, 0.30 ppm, 0.35 ppm, 0.40 ppm, 0.45 ppm, 0.50 ppm, 0.55 ppm, 0.60 ppm, 0.65 ppm, 0.70 ppm, 0.75 ppm, 0.80 ppm, 0.85 ppm, 0.90 ppm, 0.95 ppm, 1 ppm, 5 ppm, 10 ppm, 15 ppm, 20 ppm, 25 ppm, 30 ppm, 35 ppm, 40 ppm, 45 ppm, 50 ppm, 55 ppm, 60 ppm, 65 ppm, 70 ppm, 75 ppm, 80 ppm, 85 ppm, 90 ppm, 95 ppm, 100 ppm, 125 ppm, 150 ppm, 175 ppm, 200 ppm, 225 ppm, 250 ppm, 275 ppm, 300 ppm, 325 ppm, 350 ppm, 375 ppm, 400 ppm, 425 ppm, 450 ppm, 475 ppm, 500 ppm, 525 ppm, 550 ppm, 575 ppm, 600 ppm, 625 ppm, 650 ppm, 675 ppm, 700 ppm, 725 ppm, 750 ppm, 775 ppm, 800 ppm, 825 ppm, 850 ppm, 875 ppm, 900 ppm, 925 ppm, 950 ppm, 975 ppm, 1,000 ppm, 1,025 ppm, 1,050 ppm, 1075 ppm, 1,100 ppm, 1,125 ppm, 1,150 ppm, 1,175 ppm, 1,200 ppm, 1,225 ppm, 1,250 ppm, 1,275 ppm, 1,300 ppm, 1,325 ppm, 1,350 ppm, 1,375 ppm, 1,400 ppm, 1,425 ppm, 1,450 ppm, 1,475 ppm, or 1,500 ppm or at least 0.05 ppm, at least 0.10 ppm, at least 0.20 ppm, at least 0.30 ppm, at least 0.40 ppm, at least 0.50 ppm, at least 0.60 ppm, at least 0.70 ppm, at least 0.80 ppm, at least 0.90 ppm, at least 1 ppm, at least 10 ppm, at least 20 ppm, at least 30 ppm, at least 40 ppm, at least 50 ppm, at least 60 ppm, at least 70 ppm, at least 80 ppm, at least 90 ppm, at least 100 ppm, at least 125 ppm, at least 150 ppm, at least 175 ppm, at least 200 ppm, at least 225 ppm, at least 250 ppm, at least 275 ppm, at least 300 ppm, at least 325 ppm, at least 350 ppm, at least 375 ppm, at least 400 ppm, at least 425 ppm, at least 450 ppm, at least 475 ppm, at least 500 ppm, at least 525 ppm, at least 550 ppm, at least 575 ppm, at least 600 ppm, at least 625 ppm, at least 650 ppm, at least 675 ppm, at least 700 ppm, at least 725 ppm, at least 750 ppm, at least 775 ppm, at least 800 ppm, at least 825 ppm, at least 850 ppm, at least 875 ppm, at least 900 ppm, at least 925 ppm, at least 950 ppm, at least 975 ppm, at least 1,000 ppm, at least 1,025 ppm, at least 1,050 ppm, at least 1075 ppm, at least 1,100 ppm, at least 1,125 ppm, at least 1,150 ppm, at least 1,175 ppm, at least 1,200 ppm, at least 1,225 ppm, at least 1,250 ppm, at least 1,275 ppm, at least 1,300 ppm, at least 1,325 ppm, at least 1,350 ppm, at least 1,375 ppm, at least 1,400 ppm, at least 1,425 ppm, at least 1,450 ppm, at least 1,475 ppm, or at least 1,500 ppm or at most 0.05 ppm, at most 0.10 ppm, at most 0.20 ppm, at most 0.30 ppm, at most 0.40 ppm, at most 0.50 ppm, at most 0.60 ppm, at most 0.70 ppm, at most 0.80 ppm, at most 0.90 ppm, at most 1 ppm, at most 10 ppm, at most 20 ppm, at most 30 ppm, at most 40 ppm, at most 50 ppm, at most 60 ppm, at most 70 ppm, at most 80 ppm, at most 90 ppm, at most 100 ppm, at most 125 ppm, at most 150 ppm, at most 175 ppm, at most 200 ppm, at most 225 ppm, at most 250 ppm, at most 275 ppm, at most 300 ppm, at most 325 ppm, at most 350 ppm, at most 375 ppm, at most 400 ppm, at most 425 ppm, at most 450 ppm, at most 475 ppm, at most 500 ppm, at most 525 ppm, at most 550 ppm, at most 575 ppm, at most 600 ppm, at most 625 ppm, at most 650 ppm, at most 675 ppm, at most 700 ppm, at most 725 ppm, at most 750 ppm, at most 775 ppm, at most 800 ppm, at most 825 ppm, at most 850 ppm, at most 875 ppm, at most 900 ppm, at most 925 ppm, at most 950 ppm, at most 975 ppm, at most 1,000 ppm, at most 1,025 ppm, at most 1,050 ppm, at most 1075 ppm, at most 1,100 ppm, at most 1,125 ppm, at most 1,150 ppm, at most 1,175 ppm, at most 1,200 ppm, at most 1,225 ppm, at most 1,250 ppm, at most 1,275 ppm, at most 1,300 ppm, at most 1,325 ppm, at most 1,350 ppm, at most 1,375 ppm, at most 1,400 ppm, at most 1,425 ppm, at most 1,450 ppm, at most 1,475 ppm, or at most 1,500 ppm or about 0.5 ppm to about 20 ppm, about 0.5 ppm to about 25 ppm, about 0.5 ppm to about 30 ppm, about 0.5 ppm to about 35 ppm, about 0.5 ppm to about 40 ppm, about 0.5 ppm to about 45 ppm, about 0.5 ppm to about 50 ppm, about 0.5 ppm to about 55 ppm, about 0.5 ppm to about 60 ppm, about 0.5 ppm to about 65 ppm, about 0.5 ppm to about 70 ppm, about 0.5 ppm to about 75 ppm, about 0.5 ppm to about 80 ppm, about 0.5 ppm to about 85 ppm, about 0.5 ppm to about 90 ppm, about 0.5 ppm to about 95 ppm, about 0.5 ppm to about 10⁰ ppm, about 0.75 ppm to about 20 ppm, about 0.75 ppm to about 25 ppm, about 0.75 ppm to about 30 ppm, about 0.75 ppm to about 35 ppm, about 0.75 ppm to about 40 ppm, about 0.75 ppm to about 45 ppm, about 0.75 ppm to about 50 ppm, about 0.75 ppm to about 55 ppm, about 0.75 ppm to about 60 ppm, about 0.75 ppm to about 65 ppm, about 0.75 ppm to about 70 ppm, about 0.75 ppm to about 75 ppm, about 0.75 ppm to about 80 ppm, about 0.75 ppm to about 85 ppm, about 0.75 ppm to about 90 ppm, about 0.75 ppm to about 95 ppm, about 0.75 ppm to about 100 ppm, about 1 ppm to about 20 ppm, about 1 ppm to about 25 ppm, about 1 ppm to about 30 ppm, about 1 ppm to about 35 ppm, about 1 ppm to about 40 ppm, about 1 ppm to about 45 ppm, about 1 ppm to about 50 ppm, about 1 ppm to about 55 ppm, about 1 ppm to about 60 ppm, about 1 ppm to about 65 ppm, about 1 ppm to about 70 ppm, about 1 ppm to about 75 ppm, about 1 ppm to about 80 ppm, about 1 ppm to about 85 ppm, about 1 ppm to about 90 ppm, about 1 ppm to about 95 ppm, about 1 ppm to about 100 ppm, about 5 ppm to about 20 ppm, about 5 ppm to about 25 ppm, about 5 ppm to about 30 ppm, about 5 ppm to about 35 ppm, about 5 ppm to about 40 ppm, about 5 ppm to about 45 ppm, about 5 ppm to about 50 ppm, about 5 ppm to about 55 ppm, about 5 ppm to about 60 ppm, about 5 ppm to about 65 ppm, about 5 ppm to about 70 ppm, about 5 ppm to about 75 ppm, about 5 ppm to about 80 ppm, about 5 ppm to about 85 ppm, about 5 ppm to about 90 ppm, about 5 ppm to about 95 ppm, about 5 ppm to about 100 ppm, about 10 ppm to about 20 ppm, about 10 ppm to about 25 ppm, about 10 ppm to about 30 ppm, about 10 ppm to about 35 ppm, about 10 ppm to about 40 ppm, about 10 ppm to about 45 ppm, about 10 ppm to about 50 ppm, about 10 ppm to about 55 ppm, about 10 ppm to about 60 ppm, about 10 ppm to about 65 ppm, about 10 ppm to about 70 ppm, about 10 ppm to about 75 ppm, about 10 ppm to about 80 ppm, about 10 ppm to about 85 ppm, about 10 ppm to about 90 ppm, about 10 ppm to about 95 ppm, or about 10 ppm to about 100 ppm or about 1 ppm to about 25 ppm, about 1 ppm to about 50 ppm, about 1 ppm to about 75 ppm, about 1 ppm to about 100 ppm, about 1 ppm to about 125 ppm, about 1 ppm to about 150 ppm, about 1 ppm to about 175 ppm, about 1 ppm to about 200 ppm, about 1 ppm to about 225 ppm, about 1 ppm to about 250 ppm, about 1 ppm to about 275 ppm, about 1 ppm to about 300 ppm, about 1 ppm to about 325 ppm, about 1 ppm to about 350 ppm, about 1 ppm to about 375 ppm, about 1 ppm to about 400 ppm, about 10 ppm to about 25 ppm, about 10 ppm to about 50 ppm, about 10 ppm to about 75 ppm, about 10 ppm to about 100 ppm, about 10 ppm to about 125 ppm, about 10 ppm to about 150 ppm, about 10 ppm to about 175 ppm, about 10 ppm to about 200 ppm, about 10 ppm to about 225 ppm, about 10 ppm to about 250 ppm, about 10 ppm to about 275 ppm, about 10 ppm to about 300 ppm, about 10 ppm to about 325 ppm, about 10 ppm to about 350 ppm, about 10 ppm to about 375 ppm, about 10 ppm to about 400 ppm, about 25 ppm to about 50 ppm, about 25 ppm to about 75 ppm, about 25 ppm to about 100 ppm, about 25 ppm to about 125 ppm, about 25 ppm to about 150 ppm, about 25 ppm to about 175 ppm, about 25 ppm to about 200 ppm, about 25 ppm to about 225 ppm, about 25 ppm to about 250 ppm, about 25 ppm to about 275 ppm, about 25 ppm to about 300 ppm, about 25 ppm to about 325 ppm, about 25 ppm to about 350 ppm, about 25 ppm to about 375 ppm, about 25 ppm to about 400 ppm, about 50 ppm to about 75 ppm, about 50 ppm to about 100 ppm, about 50 ppm to about 125 ppm, about 50 ppm to about 150 ppm, about 50 ppm to about 175 ppm, about 50 ppm to about 200 ppm, about 50 ppm to about 225 ppm, about 50 ppm to about 250 ppm, about 50 ppm to about 275 ppm, about 50 ppm to about 300 ppm, about 50 ppm to about 325 ppm, about 50 ppm to about 350 ppm, about 50 ppm to about 375 ppm, about 50 ppm to about 400 ppm, about 75 ppm to about 100 ppm, about 75 ppm to about 125 ppm, about 75 ppm to about 150 ppm, about 75 ppm to about 175 ppm, about 75 ppm to about 200 ppm, about 75 ppm to about 225 ppm, about 75 ppm to about 250 ppm, about 75 ppm to about 275 ppm, about 75 ppm to about 300 ppm, about 75 ppm to about 325 ppm, about 75 ppm to about 350 ppm, about 75 ppm to about 375 ppm, about 75 ppm to about 400 ppm, about 100 ppm to about 125 ppm, about 100 ppm to about 150 ppm, about 100 ppm to about 175 ppm, about 100 ppm to about 200 ppm, about 100 ppm to about 225 ppm, about 100 ppm to about 250 ppm, about 100 ppm to about 275 ppm, about 100 ppm to about 300 ppm, about 100 ppm to about 325 ppm, about 100 ppm to about 350 ppm, about 100 ppm to about 375 ppm, about 100 ppm to about 400 ppm, about 150 ppm to about 175 ppm, about 150 ppm to about 200 ppm, about 150 ppm to about 225 ppm, about 150 ppm to about 250 ppm, about 150 ppm to about 275 ppm, about 150 ppm to about 300 ppm, about 150 ppm to about 325 ppm, about 150 ppm to about 350 ppm, about 150 ppm to about 375 ppm, about 150 ppm to about 400 ppm, about 200 ppm to about 225 ppm, about 200 ppm to about 250 ppm, about 200 ppm to about 275 ppm, about 200 ppm to about 300 ppm, about 200 ppm to about 325 ppm, about 200 ppm to about 350 ppm, about 200 ppm to about 375 ppm, about 200 ppm to about 400 ppm, about 250 ppm to about 275 ppm, about 250 ppm to about 300 ppm, about 250 ppm to about 325 ppm, about 250 ppm to about 350 ppm, about 250 ppm to about 375 ppm, about 250 ppm to about 400 ppm, about 300 ppm to about 325 ppm, about 300 ppm to about 350 ppm, about 300 ppm to about 375 ppm, about 300 ppm to about 400 ppm, about 350 ppm to about 375 ppm, about 350 ppm to about 400 ppm, about 375 ppm to about 400 ppm, about 400 ppm to about 500 ppm, about 400 ppm to about 600 ppm, about 400 ppm to about 700 ppm, about 400 ppm to about 800 ppm, about 400 ppm to about 900 ppm, about 400 ppm to about 1,000 ppm, about 400 ppm to about 1,100 ppm, about 400 ppm to about 1,200 ppm, about 400 ppm to about 1,300 ppm, about 400 ppm to about 1,400 ppm, about 400 ppm to about 1,500 ppm, about 500 ppm to about 600 ppm, about 500 ppm to about 700 ppm, about 500 ppm to about 800 ppm, about 500 ppm to about 900 ppm, about 500 ppm to about 1,000 ppm, about 500 ppm to about 1,100 ppm, about 500 ppm to about 1,200 ppm, about 500 ppm to about 1,300 ppm, about 500 ppm to about 1,400 ppm, about 500 ppm to about 1,500 ppm, about 600 ppm to about 700 ppm, about 600 ppm to about 800 ppm, about 600 ppm to about 900 ppm, about 600 ppm to about 1,000 ppm, about 600 ppm to about 1,100 ppm, about 600 ppm to about 1,200 ppm, about 600 ppm to about 1,300 ppm, about 600 ppm to about 1,400 ppm, about 600 ppm to about 1,500 ppm, about 700 ppm to about 800 ppm, about 700 ppm to about 900 ppm, about 700 ppm to about 1,000 ppm, about 700 ppm to about 1,100 ppm, about 700 ppm to about 1,200 ppm, about 700 ppm to about 1,300 ppm, about 700 ppm to about 1,400 ppm, about 700 ppm to about 1,500 ppm, about 800 ppm to about 900 ppm, about 800 ppm to about 1,000 ppm, about 800 ppm to about 1,100 ppm, about 800 ppm to about 1,200 ppm, about 800 ppm to about 1,300 ppm, about 800 ppm to about 1,400 ppm, about 800 ppm to about 1,500 ppm, about 900 ppm to about 1,000 ppm, about 900 ppm to about 1,100 ppm, about 900 ppm to about 1,200 ppm, about 900 ppm to about 1,300 ppm, about 900 ppm to about 1,400 ppm, about 900 ppm to about 1,500 ppm, about 1,000 ppm to about 1,100 ppm, about 1,000 ppm to about 1,200 ppm, about 1,000 ppm to about 1,300 ppm, about 1,000 ppm to about 1,400 ppm, about 1,000 ppm to about 1,500 ppm, about 1,100 ppm to about 1,200 ppm, about 1,100 ppm to about 1,300 ppm, about 1,100 ppm to about 1,400 ppm, about 1,100 ppm to about 1,500 ppm, about 1,200 ppm to about 1,300 ppm, about 1,200 ppm to about 1,400 ppm, about 1,200 ppm to about 1,500 ppm, about 1,300 ppm to about 1,400 ppm, about 1,300 ppm to about 1,500 ppm, or about 1,400 ppm to about 1,500 ppm.
20. The composition according to any one of embodiment 3-5, 8, or 14-19, wherein the one or more guanide-containing compounds comprise, consist essentially of, or consist of a biguanide, a biguanide-containing compound, a biguanidine, a biguanidine-containing compound, a triguanide, a triguanide-containing compound, or any combination thereof.
21. The composition according to embodiment 20, wherein the one or more biguanide-containing compounds include a polyhexamethylene biguanide (PHMB), a polyaminopropyl biguanide (PAPB), a 1,1′-(1,6-Hexanediyl)bis{2-[N′-(2-ethylhexyl)carbamimidoyl]guanidine} (alexidine), a chlorhexidine, a chlorhexidine gluconate, or any combination thereof.
22. The composition according to any one of embodiments 3-5, 8, or 14-21, wherein the one or more guanide-containing compounds are in an amount of about 0.01%, about 0.05%, about 0.075%, about 0.1%, about 0.2%, about 0.3%, about 0.4%, about 0.5%, about 0.6%, about 0.7%, about 0.8%, about 0.9%, about 1.0%, about 1.5%, about 2.0%, about 2.5%, about 3.0%, about 4.0%, about 5.0%, about 6.0%, about 7.0%, about 7.5%, about 8.0%, about 9.0%, about 10.0%. about 11%, about 12%, about 13%, about 14%, about 15%, about 20%, about 25%, or about 30% by weight of the composition, or at least 0.01%, at least 0.05%, at least 0.075%, at least 0.1%, at least 0.25%, at least 0.5%, at least 0.75%, at least 1.0%, at least 1.5%, at least 2.0%, at least 2.5%, at least 3.0%, at least 4.0%, at least 5.0%, at least 6.0%, at least 7.0%, at least 7.5%, at least 8.0%, at least 9.0%, at least 10.0%, at least 11%, at least 12%, at least 13%, at least 14%, at least 15%, at least 20%, at least 25%, or at least 30% by weight of the composition, or at most 0.01%, at most 0.05%, at most 0.075%, at most 0.1%, at most 0.25%, at most 0.5%, at most 0.75%, at most 1.0%, at most 1.5%, at most 2.0%, at most 2.5%, at most 3.0%, at most 4.0%, at most 5.0%, at most 6.0%, at most 7.5%, at most 8.0%, at most 9.0%, at most 10.0%, at most 11%, at most 12%, at most 13%, at most 14%, at most 15%, at most 20%, at most 25%, or at most 30% by weight of the composition, or about 0.1% to about 0.5%, about 0.1% to about 0.75%, about 0.1% to about 1.0%, about 0.1% to about 1.5%, about 0.1% to about 2.0%, about 0.1% to about 2.5%, about 0.1% to about 3.0%, about 0.1% to about 3.5%, about 0.1% to about 4.0%, about 0.1% to about 4.5%, about 0.1% to about 5.0%, about 0.2% to about 0.5%, about 0.2% to about 0.75%, about 0.2% to about 1.0%, about 0.2% to about 1.5%, about 0.2% to about 2.0%, about 0.2% to about 2.5%, about 0.2% to about 3.0%, about 0.2% to about 3.5%, about 0.2% to about 4.0%, about 0.2% to about 4.5%, about 0.2% to about 5.0%, about 0.5% to about 1.0%, about 0.5% to about 1.5%, about 0.5% to about 2.0%, about 0.5% to about 2.5%, about 0.5% to about 3.0%, about 0.5% to about 4.0%, about 0.5% to about 5.0%, about 0.5% to about 6.0%, about 0.5% to about 7.0%, about 0.5% to about 8.0%, about 0.5% to about 9.0%, about 0.5% to about 10.0%, about 1.0% to about 2.5%, about 1.0% to about 3.0%, about 1.0% to about 4.0%, about 1.0% to about 5.0%, about 1.0% to about 6.0%, about 1.0% to about 7.0%, about 1.0% to about 7.5%, about 1.0% to about 8.0%, about 1.0% to about 9.0%, about 1.0% to about 10.0%, about 1.0% to about 15.0%, about 1.0% to about 20.0%, about 1.0% to about 25.0%, about 1.0% to about 30.0%, about 2.0% to about 2.5%, about 2.0% to about 3.0%, about 2.0% to about 4.0%, about 2.0% to about 5.0%, about 2.0% to about 6.0%, about 2.0% to about 7.0%, about 2.0% to about 7.5%, about 2.0% to about 8.0%, about 2.0% to about 9.0%, about 2.0% to about 10.0%, about 2.0% to about 15.0%, about 2.0% to about 20.0%, about 2.0% to about 25.0%, about 2.0% to about 30.0%, about 5.0% to about 6.0%, about 5.0% to about 7.0%, about 5.0% to about 7.5%, about 5.0% to about 8.0%, about 5.0% to about 9.0%, about 5.0% to about 10.0%, about 5.0% to about 11.0%, about 5.0% to about 12.0%, about 5.0% to about 13.0%, about 5.0% to about 14.0%, about 5.0% to about 15.0%, about 5.0% to about 20.0%, about 5.0% to about 25.0%, about 5.0% to about 30.0%, about 10.0% to about 15.0%, about 10.0% to about 20.0%, about 10.0% to about 25.0%, about 10.0% to about 30.0%, about 15.0% to about 20.0%, about 15.0% to about 25.0%, about 15.0% to about 30.0%, about 20.0% to about 25.0%, about 20.0% to about 30.0%, or about 20.0% to about 30.0%, by weight of the composition.
23. The composition according to any one of embodiments 6-8 or 14-22, wherein the one or more alcohols include methanol, ethanol, propanol, isopropanol, butanol, pentanol, and/or 1-hexadecanol.
24. The composition according any one of embodiments 6-8 or 14-23, wherein the one or more alcohols are in an amount of about 0.5%, about 1%, about 2%, about 3%, about 4%, about 5%, about 6%, about 7%, about 8%, about 9%, about 10%, about 11%, about 12%, about 13%, about 14%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, or about 99%, by weight of the composition, or at least 0.5%, at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99%, by weight of the composition, or at most 0.5%, at most 1%, at most 2%, at most 3%, at most 4%, at most 5%, at most 6%, at most 7%, at most 8%, at most 9%, at most 10%, at most 11%, at most 12%, at most 13%, at most 14%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95%, at most 96%, at most 97%, at most 98%, or at most 99%, by weight of the composition, about 0.05% to about 0.75%, about 0.05% to about 1.0%, about 0.05% to about 1.5%, about 0.05% to about 2.0%, about 0.05% to about 2.5%, about 0.1% to about 0.5%, about 0.1% to about 0.75%, about 0.1% to about 1.0%, about 0.1% to about 1.5%, about 0.1% to about 2.0%, about 0.1% to about 2.5%, about 0.1% to about 3.0%, about 0.1% to about 4.0%, about 0.1% to about 5.0%, about 0.2% to about 0.5%, about 0.2% to about 0.75%, about 0.2% to about 1.0%, about 0.2% to about 1.5%, about 0.2% to about 2.0%, about 0.2% to about 2.5%, about 0.2% to about 3.0%, about 0.2% to about 4.0%, about 0.2% to about 5.0%, about 0.5% to about 1.0%, about 0.5% to about 1.5%, about 0.5% to about 2.0%, about 0.5% to about 2.5%, about 0.5% to about 3.0%, about 0.5% to about 4.0%, about 0.5% to about 5.0%, about 0.5% to about 6.0%, about 0.5% to about 7.0%, about 0.5% to about 8.0%, about 0.5% to about 9.0%, about 0.5% to about 10.0%, about 1.0% to about 2.5%, about 1.0% to about 3.0%, about 1.0% to about 4.0%, about 1.0% to about 5.0%, about 1.0% to about 6.0%, about 1.0% to about 7.0%, about 1.0% to about 7.5%, about 1.0% to about 8.0%, about 1.0% to about 9.0%, about 1.0% to about 10.0%, about 2.0% to about 2.5%, about 2.0% to about 3.0%, about 2.0% to about 4.0%, about 2.0% to about 5.0%, about 2.0% to about 6.0%, about 2.0% to about 7.0%, about 2.0% to about 7.5%, about 2.0% to about 8.0%, about 2.0% to about 9.0%, about 2.0% to about 10.0%, about 5.0% to about 6.0%, about 5.0% to about 7.0%, about 5.0% to about 7.5%, about 5.0% to about 8.0%, about 5.0% to about 9.0%, about 5.0% to about 10.0%, about 5.0% to about 11.0%, about 5.0% to about 12.0%, about 5.0% to about 13.0%, about 5.0% to about 14.0% or about 5.0% to about 15.0% by weight of the composition, or about 10% to about 20%, about 10% to about 30%, about 10% to about 40%, about 10% to about 50%, about 10% to about 60%, about 10% to about 70%, about 10% to about 80%, about 10% to about 90%, about 10% to about 95%, about 10% to about 97%, about 10% to about 99%, about 20% to about 30%, about 20% to about 40%, about 20% to about 50%, about 20% to about 60%, about 20% to about 70%, about 20% to about 80%, about 20% to about 90%, about 20% to about 95%, about 20% to about 97%, about 20% to about 99%, about 30% to about 40%, about 30% to about 50%, about 30% to about 60%, about 30% to about 70%, about 30% to about 80%, about 30% to about 90%, about 30% to about 95%, about 30% to about 97%, about 30% to about 99%, about 40% to about 50%, about 40% to about 60%, about 40% to about 70%, about 40% to about 80%, about 40% to about 90%, about 40% to about 95%, about 40% to about 97%, about 40% to about 99%, about 50% to about 60%, about 50% to about 70%, about 50% to about 80%, about 50% to about 90%, about 50% to about 95%, about 50% to about 97%, about 50% to about 99%, about 60% to about 70%, about 60% to about 80%, about 60% to about 90%, about 60% to about 95%, about 60% to about 97%, about 60% to about 99%, about 70% to about 80%, about 70% to about 90%, about 70% to about 95%, about 70% to about 97%, about 70% to about 99%, about 80% to about 90%, about 80% to about 95%, about 80% to about 97%, about 80% to about 99%, about 90% to about 95%, about 90% to about 97%, about 90% to about 99%, by weight of the composition.
25. The composition according to any one of embodiments 9, 11, or 13-24, wherein the one or more metallic particles include a metal acetate particle, a metal chloride particle, a metal nitrate particle, or a metal oxide particle, or any combination thereof.
26. The composition according to any one of embodiments 9, 11 or 13-25, wherein the one or more metallic particles are in an amount of 0.05 ppm, 0.10 ppm, 0.15 ppm, 0.20 ppm, 0.25 ppm, 0.30 ppm, 0.35 ppm, 0.40 ppm, 0.45 ppm, 0.50 ppm, 0.55 ppm, 0.60 ppm, 0.65 ppm, 0.70 ppm, 0.75 ppm, 0.80 ppm, 0.85 ppm, 0.90 ppm, 0.95 ppm, 1 ppm, 5 ppm, 10 ppm, 15 ppm, 20 ppm, 25 ppm, 30 ppm, 35 ppm, 40 ppm, 45 ppm, 50 ppm, 55 ppm, 60 ppm, 65 ppm, 70 ppm, 75 ppm, 80 ppm, 85 ppm, 90 ppm, 95 ppm, 100 ppm, 125 ppm, 150 ppm, 175 ppm, 200 ppm, 225 ppm, 250 ppm, 275 ppm, 300 ppm, 325 ppm, 350 ppm, 375 ppm, 400 ppm, 425 ppm, 450 ppm, 475 ppm, 500 ppm, 525 ppm, 550 ppm, 575 ppm, 600 ppm, 625 ppm, 650 ppm, 675 ppm, 700 ppm, 725 ppm, 750 ppm, 775 ppm, 800 ppm, 825 ppm, 850 ppm, 875 ppm, 900 ppm, 925 ppm, 950 ppm, 975 ppm, 1,000 ppm, 1,025 ppm, 1,050 ppm, 1075 ppm, 1,100 ppm, 1,125 ppm, 1,150 ppm, 1,175 ppm, 1,200 ppm, 1,225 ppm, 1,250 ppm, 1,275 ppm, 1,300 ppm, 1,325 ppm, 1,350 ppm, 1,375 ppm, 1,400 ppm, 1,425 ppm, 1,450 ppm, 1,475 ppm, 1,500 ppm, 1,600 ppm, 1,700 ppm, 1,800 ppm, 1,900 ppm, or 2,000 ppm or at least 0.05 ppm, at least 0.10 ppm, at least 0.20 ppm, at least 0.30 ppm, at least 0.40 ppm, at least 0.50 ppm, at least 0.60 ppm, at least 0.70 ppm, at least 0.80 ppm, at least 0.90 ppm, at least 1 ppm, at least 10 ppm, at least 20 ppm, at least 30 ppm, at least 40 ppm, at least 50 ppm, at least 60 ppm, at least 70 ppm, at least 80 ppm, at least 90 ppm, at least 100 ppm, at least 125 ppm, at least 150 ppm, at least 175 ppm, at least 200 ppm, at least 225 ppm, at least 250 ppm, at least 275 ppm, at least 300 ppm, at least 325 ppm, at least 350 ppm, at least 375 ppm, at least 400 ppm, at least 425 ppm, at least 450 ppm, at least 475 ppm, at least 500 ppm, at least 525 ppm, at least 550 ppm, at least 575 ppm, at least 600 ppm, at least 625 ppm, at least 650 ppm, at least 675 ppm, at least 700 ppm, at least 725 ppm, at least 750 ppm, at least 775 ppm, at least 800 ppm, at least 825 ppm, at least 850 ppm, at least 875 ppm, at least 900 ppm, at least 925 ppm, at least 950 ppm, at least 975 ppm, at least 1,000 ppm, at least 1,025 ppm, at least 1,050 ppm, at least 1075 ppm, at least 1,100 ppm, at least 1,125 ppm, at least 1,150 ppm, at least 1,175 ppm, at least 1,200 ppm, at least 1,225 ppm, at least 1,250 ppm, at least 1,275 ppm, at least 1,300 ppm, at least 1,325 ppm, at least 1,350 ppm, at least 1,375 ppm, at least 1,400 ppm, at least 1,425 ppm, at least 1,450 ppm, at least 1,475 ppm, at least 1,500 ppm, at least 1,600 ppm, at least 1,700 ppm, at least 1,800 ppm, at least 1,900 ppm, or at least 2,000 ppm or at most 0.05 ppm, at most 0.10 ppm, at most 0.20 ppm, at most 0.30 ppm, at most 0.40 ppm, at most 0.50 ppm, at most 0.60 ppm, at most 0.70 ppm, at most 0.80 ppm, at most 0.90 ppm, at most 1 ppm, at most 10 ppm, at most 20 ppm, at most 30 ppm, at most 40 ppm, at most 50 ppm, at most 60 ppm, at most 70 ppm, at most 80 ppm, at most 90 ppm, at most 100 ppm, at most 125 ppm, at most 150 ppm, at most 175 ppm, at most 200 ppm, at most 225 ppm, at most 250 ppm, at most 275 ppm, at most 300 ppm, at most 325 ppm, at most 350 ppm, at most 375 ppm, at most 400 ppm, at most 425 ppm, at most 450 ppm, at most 475 ppm, at most 500 ppm, at most 525 ppm, at most 550 ppm, at most 575 ppm, at most 600 ppm, at most 625 ppm, at most 650 ppm, at most 675 ppm, at most 700 ppm, at most 725 ppm, at most 750 ppm, at most 775 ppm, at most 800 ppm, at most 825 ppm, at most 850 ppm, at most 875 ppm, at most 900 ppm, at most 925 ppm, at most 950 ppm, at most 975 ppm, at most 1,000 ppm, at most 1,025 ppm, at most 1,050 ppm, at most 1075 ppm, at most 1,100 ppm, at most 1,125 ppm, at most 1,150 ppm, at most 1,175 ppm, at most 1,200 ppm, at most 1,225 ppm, at most 1,250 ppm, at most 1,275 ppm, at most 1,300 ppm, at most 1,325 ppm, at most 1,350 ppm, at most 1,375 ppm, at most 1,400 ppm, at most 1,425 ppm, at most 1,450 ppm, at most 1,475 ppm, at most 1,500 ppm, at most 1,600 ppm, at most 1,700 ppm, at most 1,800 ppm, at most 1,900 ppm, or at most 2,000 ppm or about 0.5 ppm to about 20 ppm, about 0.5 ppm to about 25 ppm, about 0.5 ppm to about 30 ppm, about 0.5 ppm to about 35 ppm, about 0.5 ppm to about 40 ppm, about 0.5 ppm to about 45 ppm, about 0.5 ppm to about 50 ppm, about 0.5 ppm to about 55 ppm, about 0.5 ppm to about 60 ppm, about 0.5 ppm to about 65 ppm, about 0.5 ppm to about 70 ppm, about 0.5 ppm to about 75 ppm, about 0.5 ppm to about 80 ppm, about 0.5 ppm to about 85 ppm, about 0.5 ppm to about 90 ppm, about 0.5 ppm to about 95 ppm, about 0.5 ppm to about 100 ppm, about 0.75 ppm to about 20 ppm, about 0.75 ppm to about 25 ppm, about 0.75 ppm to about 30 ppm, about 0.75 ppm to about 35 ppm, about 0.75 ppm to about 40 ppm, about 0.75 ppm to about 45 ppm, about 0.75 ppm to about 50 ppm, about 0.75 ppm to about 55 ppm, about 0.75 ppm to about 60 ppm, about 0.75 ppm to about 65 ppm, about 0.75 ppm to about 70 ppm, about 0.75 ppm to about 75 ppm, about 0.75 ppm to about 80 ppm, about 0.75 ppm to about 85 ppm, about 0.75 ppm to about 90 ppm, about 0.75 ppm to about 95 ppm, about 0.75 ppm to about 100 ppm, about 1 ppm to about 20 ppm, about 1 ppm to about 25 ppm, about 1 ppm to about 30 ppm, about 1 ppm to about 35 ppm, about 1 ppm to about 40 ppm, about 1 ppm to about 45 ppm, about 1 ppm to about 50 ppm, about 1 ppm to about 55 ppm, about 1 ppm to about 60 ppm, about 1 ppm to about 65 ppm, about 1 ppm to about 70 ppm, about 1 ppm to about 75 ppm, about 1 ppm to about 80 ppm, about 1 ppm to about 85 ppm, about 1 ppm to about 90 ppm, about 1 ppm to about 95 ppm, about 1 ppm to about 100 ppm, about 5 ppm to about 20 ppm, about 5 ppm to about 25 ppm, about 5 ppm to about 30 ppm, about 5 ppm to about 35 ppm, about 5 ppm to about 40 ppm, about 5 ppm to about 45 ppm, about 5 ppm to about 50 ppm, about 5 ppm to about 55 ppm, about 5 ppm to about 60 ppm, about 5 ppm to about 65 ppm, about 5 ppm to about 70 ppm, about 5 ppm to about 75 ppm, about 5 ppm to about 80 ppm, about 5 ppm to about 85 ppm, about 5 ppm to about 90 ppm, about 5 ppm to about 95 ppm, about 5 ppm to about 100 ppm, about 10 ppm to about 20 ppm, about 10 ppm to about 25 ppm, about 10 ppm to about 30 ppm, about 10 ppm to about 35 ppm, about 10 ppm to about 40 ppm, about 10 ppm to about 45 ppm, about 10 ppm to about 50 ppm, about 10 ppm to about 55 ppm, about 10 ppm to about 60 ppm, about 10 ppm to about 65 ppm, about 10 ppm to about 70 ppm, about 10 ppm to about 75 ppm, about 10 ppm to about 80 ppm, about 10 ppm to about 85 ppm, about 10 ppm to about 90 ppm, about 10 ppm to about 95 ppm, or about 10 ppm to about 100 ppm, about 1 ppm to about 25 ppm, about 1 ppm to about 50 ppm, about 1 ppm to about 75 ppm, about 1 ppm to about 100 ppm, about 1 ppm to about 125 ppm, about 1 ppm to about 150 ppm, about 1 ppm to about 175 ppm, about 1 ppm to about 200 ppm, about 1 ppm to about 225 ppm, about 1 ppm to about 250 ppm, about 1 ppm to about 275 ppm, about 1 ppm to about 300 ppm, about 1 ppm to about 325 ppm, about 1 ppm to about 350 ppm, about 1 ppm to about 375 ppm, about 1 ppm to about 400 ppm, about 10 ppm to about 25 ppm, about 10 ppm to about 50 ppm, about 10 ppm to about 75 ppm, about 10 ppm to about 100 ppm, about 10 ppm to about 125 ppm, about 10 ppm to about 150 ppm, about 10 ppm to about 175 ppm, about 10 ppm to about 200 ppm, about 10 ppm to about 225 ppm, about 10 ppm to about 250 ppm, about 10 ppm to about 275 ppm, about 10 ppm to about 300 ppm, about 10 ppm to about 325 ppm, about 10 ppm to about 350 ppm, about 10 ppm to about 375 ppm, about 10 ppm to about 400 ppm, about 25 ppm to about 50 ppm, about 25 ppm to about 75 ppm, about 25 ppm to about 100 ppm, about 25 ppm to about 125 ppm, about 25 ppm to about 150 ppm, about 25 ppm to about 175 ppm, about 25 ppm to about 200 ppm, about 25 ppm to about 225 ppm, about 25 ppm to about 250 ppm, about 25 ppm to about 275 ppm, about 25 ppm to about 300 ppm, about 25 ppm to about 325 ppm, about 25 ppm to about 350 ppm, about 25 ppm to about 375 ppm, about 25 ppm to about 400 ppm, about 50 ppm to about 75 ppm, about 50 ppm to about 100 ppm, about 50 ppm to about 125 ppm, about 50 ppm to about 150 ppm, about 50 ppm to about 175 ppm, about 50 ppm to about 200 ppm, about 50 ppm to about 225 ppm, about 50 ppm to about 250 ppm, about 50 ppm to about 275 ppm, about 50 ppm to about 300 ppm, about 50 ppm to about 325 ppm, about 50 ppm to about 350 ppm, about 50 ppm to about 375 ppm, about 50 ppm to about 400 ppm, about 75 ppm to about 100 ppm, about 75 ppm to about 125 ppm, about 75 ppm to about 150 ppm, about 75 ppm to about 175 ppm, about 75 ppm to about 200 ppm, about 75 ppm to about 225 ppm, about 75 ppm to about 250 ppm, about 75 ppm to about 275 ppm, about 75 ppm to about 300 ppm, about 75 ppm to about 325 ppm, about 75 ppm to about 350 ppm, about 75 ppm to about 375 ppm, about 75 ppm to about 400 ppm, about 100 ppm to about 125 ppm, about 100 ppm to about 150 ppm, about 100 ppm to about 175 ppm, about 100 ppm to about 200 ppm, about 100 ppm to about 225 ppm, about 100 ppm to about 250 ppm, about 100 ppm to about 275 ppm, about 100 ppm to about 300 ppm, about 100 ppm to about 325 ppm, about 100 ppm to about 350 ppm, about 100 ppm to about 375 ppm, about 100 ppm to about 400 ppm, about 150 ppm to about 175 ppm, about 150 ppm to about 200 ppm, about 150 ppm to about 225 ppm, about 150 ppm to about 250 ppm, about 150 ppm to about 275 ppm, about 150 ppm to about 300 ppm, about 150 ppm to about 325 ppm, about 150 ppm to about 350 ppm, about 150 ppm to about 375 ppm, about 150 ppm to about 400 ppm, about 200 ppm to about 225 ppm, about 200 ppm to about 250 ppm, about 200 ppm to about 275 ppm, about 200 ppm to about 300 ppm, about 200 ppm to about 325 ppm, about 200 ppm to about 350 ppm, about 200 ppm to about 375 ppm, about 200 ppm to about 400 ppm, about 200 ppm to about 425 ppm, about 200 ppm to about 450 ppm, about 200 ppm to about 475 ppm, about 200 ppm to about 500 ppm, about 200 ppm to about 525 ppm, about 200 ppm to about 550 ppm, about 200 ppm to about 575 ppm, about 200 ppm to about 600 ppm, about 250 ppm to about 275 ppm, about 250 ppm to about 300 ppm, about 250 ppm to about 325 ppm, about 250 ppm to about 350 ppm, about 250 ppm to about 375 ppm, about 250 ppm to about 400 ppm, about 250 ppm to about 425 ppm, about 250 ppm to about 450 ppm, about 250 ppm to about 475 ppm, about 250 ppm to about 500 ppm, about 250 ppm to about 525 ppm, about 250 ppm to about 550 ppm, about 250 ppm to about 575 ppm, about 250 ppm to about 600 ppm, about 300 ppm to about 325 ppm, about 300 ppm to about 350 ppm, about 300 ppm to about 375 ppm, about 300 ppm to about 400 ppm, about 300 ppm to about 425 ppm, about 300 ppm to about 450 ppm, about 300 ppm to about 475 ppm, about 300 ppm to about 500 ppm, about 300 ppm to about 525 ppm, about 300 ppm to about 550 ppm, about 300 ppm to about 575 ppm, about 300 ppm to about 600 ppm, about 350 ppm to about 375 ppm, about 350 ppm to about 400 ppm, about 375 ppm to about 400 ppm, about 350 ppm to about 425 ppm, about 350 ppm to about 450 ppm, about 350 ppm to about 475 ppm, about 350 ppm to about 500 ppm, about 350 ppm to about 525 ppm, about 350 ppm to about 550 ppm, about 350 ppm to about 575 ppm, about 350 ppm to about 600 ppm, about 400 ppm to about 500 ppm, about 400 ppm to about 600 ppm, about 400 ppm to about 700 ppm, about 400 ppm to about 800 ppm, about 400 ppm to about 900 ppm, about 400 ppm to about 1,000 ppm, about 400 ppm to about 1,100 ppm, about 400 ppm to about 1,200 ppm, about 400 ppm to about 1,300 ppm, about 400 ppm to about 1,400 ppm, about 400 ppm to about 1,500 ppm, about 400 ppm to about 1,600 ppm, about 400 ppm to about 1,700 ppm, about 400 ppm to about 1,800 ppm, about 400 ppm to about 1,900 ppm, about 400 ppm to about 2,000 ppm, about 500 ppm to about 600 ppm, about 500 ppm to about 700 ppm, about 500 ppm to about 800 ppm, about 500 ppm to about 900 ppm, about 500 ppm to about 1,000 ppm, about 500 ppm to about 1,100 ppm, about 500 ppm to about 1,200 ppm, about 500 ppm to about 1,300 ppm, about 500 ppm to about 1,400 ppm, about 500 ppm to about 1,500 ppm, about 500 ppm to about 1,600 ppm, about 500 ppm to about 1,700 ppm, about 500 ppm to about 1,800 ppm, about 500 ppm to about 1,900 ppm, about 500 ppm to about 2,000 ppm, about 600 ppm to about 700 ppm, about 600 ppm to about 800 ppm, about 600 ppm to about 900 ppm, about 600 ppm to about 1,000 ppm, about 600 ppm to about 1,100 ppm, about 600 ppm to about 1,200 ppm, about 600 ppm to about 1,300 ppm, about 600 ppm to about 1,400 ppm, about 600 ppm to about 1,500 ppm, about 600 ppm to about 1,600 ppm, about 600 ppm to about 1,700 ppm, about 600 ppm to about 1,800 ppm, about 600 ppm to about 1,900 ppm, about 600 ppm to about 2,000 ppm, about 700 ppm to about 800 ppm, about 700 ppm to about 900 ppm, about 700 ppm to about 1,000 ppm, about 700 ppm to about 1,100 ppm, about 700 ppm to about 1,200 ppm, about 700 ppm to about 1,300 ppm, about 700 ppm to about 1,400 ppm, about 700 ppm to about 1,500 ppm, about 700 ppm to about 1,600 ppm, about 700 ppm to about 1,700 ppm, about 700 ppm to about 1,800 ppm, about 700 ppm to about 1,900 ppm, about 700 ppm to about 2,000 ppm, about 800 ppm to about 900 ppm, about 800 ppm to about 1,000 ppm, about 800 ppm to about 1,100 ppm, about 800 ppm to about 1,200 ppm, about 800 ppm to about 1,300 ppm, about 800 ppm to about 1,400 ppm, about 800 ppm to about 1,500 ppm, about 800 ppm to about 1,600 ppm, about 800 ppm to about 1,700 ppm, about 800 ppm to about 1,800 ppm, about 800 ppm to about 1,900 ppm, about 800 ppm to about 2,000 ppm, about 900 ppm to about 1,000 ppm, about 900 ppm to about 1,100 ppm, about 900 ppm to about 1,200 ppm, about 900 ppm to about 1,300 ppm, about 900 ppm to about 1,400 ppm, about 900 ppm to about 1,500 ppm, about 900 ppm to about 1,600 ppm, about 900 ppm to about 1,700 ppm, about 900 ppm to about 1,800 ppm, about 900 ppm to about 1,900 ppm, about 900 ppm to about 2,000 ppm, about 1,000 ppm to about 1,100 ppm, about 1,000 ppm to about 1,200 ppm, about 1,000 ppm to about 1,300 ppm, about 1,000 ppm to about 1,400 ppm, about 1,000 ppm to about 1,500 ppm, about 1,000 ppm to about 1,600 ppm, about 1,000 ppm to about 1,700 ppm, about 1,000 ppm to about 1,800 ppm, about 1,000 ppm to about 1,900 ppm, about 1,000 ppm to about 2,000 ppm, about 1,100 ppm to about 1,200 ppm, about 1,100 ppm to about 1,300 ppm, about 1,100 ppm to about 1,400 ppm, about 1,100 ppm to about 1,500 ppm, about 1,100 ppm to about 1,600 ppm, about 1,100 ppm to about 1,700 ppm, about 1,100 ppm to about 1,800 ppm, about 1,100 ppm to about 1,900 ppm, about 1,100 ppm to about 2,000 ppm, about 1,200 ppm to about 1,300 ppm, about 1,200 ppm to about 1,400 ppm, about 1,200 ppm to about 1,500 ppm, about 1,200 ppm to about 1,600 ppm, about 1,200 ppm to about 1,700 ppm, about 1,200 ppm to about 1,800 ppm, about 1,200 ppm to about 1,900 ppm, about 1,200 ppm to about 2,000 ppm, about 1,300 ppm to about 1,400 ppm, about 1,300 ppm to about 1,500 ppm, about 1,300 ppm to about 1,600 ppm, about 1,300 ppm to about 1,700 ppm, about 1,300 ppm to about 1,800 ppm, about 1,300 ppm to about 1,900 ppm, about 1,300 ppm to about 2,000 ppm, about 1,400 ppm to about 1,500 ppm, about 1,400 ppm to about 1,600 ppm, about 1,400 ppm to about 1,700 ppm, about 1,400 ppm to about 1,800 ppm, about 1,400 ppm to about 1,900 ppm, about 1,400 ppm to about 2,000 ppm, about 1,500 ppm to about 1,600 ppm, about 1,500 ppm to about 1,700 ppm, about 1,500 ppm to about 1,800 ppm, about 1,500 ppm to about 1,900 ppm, about 1,500 ppm to about 2,000 ppm, about 1,600 ppm to about 1,700 ppm, about 1,600 ppm to about 1,800 ppm, about 1,600 ppm to about 1,900 ppm, about 1,600 ppm to about 2,000 ppm, about 1,700 ppm to about 1,800 ppm, about 1,700 ppm to about 1,900 ppm, about 1,700 ppm to about 2,000 ppm, about 1,800 ppm to about 1,900 ppm, about 1,800 ppm to about 2,000 ppm, or about 1,900 ppm to about 2,000 ppm.
27. The composition according to any one of embodiments 9, 11, or 13-25, wherein the one or more metallic particles are in an amount of about 0.001%, about 0.005%, about 0.01%, about 0.05%, about 0.10%, about 0.11%, about 0.12%, about 0.13%, about 0.14%, about 0.15%, about 0.16%, about 0.17%, about 0.18%, about 0.19%, about 0.2%, about 0.25%, about 0.3%, about 0.35%, about 0.4%, about 0.45%, or about 0.5% by weight of the composition, or at least 0.001%, at least 0.005%, at least 0.01%, at least 0.05%, at least 0.10%, at least 0.11%, at least 0.12%, at least 0.13%, at least 0.14%, at least 0.15%, at least 0.16%, at least 0.17%, at least 0.18%, at least 0.19%, at least 0.2%, at least 0.25%, at least 0.3%, at least 0.35%, at least 0.4%, at least 0.45%, or at least 0.5% by weight of the composition, or at most 0.001%, at most 0.005%, at most 0.01%, at most 0.05%, at most 0.10%, at most 0.11%, at most 0.12%, at most 0.13%, at most 0.14%, at most 0.15%, at most 0.16%, at most 0.17%, at most 0.18%, at most 0.19%, at most 0.2%, at most 0.25%, at most 0.3%, at most 0.35%, at most 0.4%, at most 0.45%, or at most 0.5% by weight of the composition, or about 0.0010% to about 0.005%, about 0.0010% to about 0.010%, about 0.0010% to about 0.050%, about 0.0010% to about 0.10%, about 0.0010% to about 0.15%, about 0.0010% to about 0.20%, about 0.0010% to about 0.25%, about 0.0010% to about 0.30%, about 0.0010% to about 0.35%, about 0.0010% to about 0.40%, about 0.0010% to about 0.45%, about 0.0010% to about 0.50%, about 0.010% to about 0.050%, about 0.010% to about 0.10%, about 0.010% to about 0.15%, about 0.010% to about 0.20%, about 0.010% to about 0.25%, about 0.010% to about 0.30%, about 0.010% to about 0.35%, about 0.010% to about 0.40%, about 0.010% to about 0.45%, about 0.010% to about 0.50%, about 0.10% to about 0.15%, about 0.10% to about 0.20%, about 0.10% to about 0.25%, about 0.10% to about 0.30%, about 0.10% to about 0.35%, about 0.10% to about 0.40%, about 0.10% to about 0.45%, about 0.10% to about 0.50%, about 0.20% to about 0.25%, about 0.20% to about 0.30%, about 0.20% to about 0.35%, about 0.20% to about 0.40%, about 0.20% to about 0.45%, about 0.20% to about 0.50%, about 0.30% to about 0.35%, about 0.30% to about 0.40%, about 0.30% to about 0.45%, about 0.30% to about 0.50%, about 0.40% to about 0.45%, about 0.40% to about 0.50%, or about 0.40% to about 0.50% by weight of the composition.
28. The composition according to any one of embodiments 9, 11, or 13-27, wherein the one or more metallic particles have a mean diameter of about 10 nm, about 20 nm, about 30 nm, about 40 nm, about 50 nm, about 60 nm, about 70 nm, about 80 nm, about 90 nm, about 100 nm, or at least 10 nm, at least 20 nm, at least 30 nm, at least 40 nm, at least 50 nm, at least 60 nm, at least 70 nm, at least 80 nm, at least 90 nm, at least 10⁰ nm, or at most 10 nm, at most 20 nm, at most 30 nm, at most 40 nm, at most 50 nm, at most 60 nm, at most 70 nm, at most 80 nm, at most 90 nm, at most 10⁰ nm, or about 10 nm to about 20 nm, about 10 nm to about 30 nm, about 10 nm to about 40 nm, about 10 nm to about 50 nm, about 10 nm to about 60 nm, about 10 nm to about 70 nm, about 10 nm to about 80 nm, about 10 nm to about 90 nm, about 10 nm to about 100 nm, about 20 nm to about 30 nm, about 20 nm to about 40 nm, about 20 nm to about 50 nm, about 20 nm to about 60 nm, about 20 nm to about 70 nm, about 20 nm to about 80 nm, about 20 nm to about 90 nm, about 20 nm to about 100 nm, about 30 nm to about 40 nm, about 30 nm to about 50 nm, about 30 nm to about 60 nm, about 30 nm to about 70 nm, about 30 nm to about 80 nm, about 30 nm to about 90 nm, about 30 nm to about 100 nm, about 40 nm to about 50 nm, about 40 nm to about 60 nm, about 40 nm to about 70 nm, about 40 nm to about 80 nm, about 40 nm to about 90 nm, about 40 nm to about 100 nm, about 50 nm to about 60 nm, about 50 nm to about 70 nm, about 50 nm to about 80 nm, about 50 nm to about 90 nm, about 50 nm to about 10⁰ nm, about 60 nm to about 70 nm, about 60 nm to about 80 nm, about 60 nm to about 90 nm, about 60 nm to about 100 nm, about 70 nm to about 80 nm, about 70 nm to about 90 nm, about 70 nm to about 100 nm, about 80 nm to about 90 nm, about 80 nm to about 100 nm, or about 90 nm to about 10⁰ nm.
29. The composition according to any one of embodiments 10 or 12-28, wherein the one or more metal salts include copper chloride, iron chloride, titanium chloride, zinc chloride, silver nitrate, or any combination thereof.
30. The composition according to any one of embodiments 10 or 12-29, wherein the one or more metal salt are in an amount of 0.05 ppm, 0.10 ppm, 0.15 ppm, 0.20 ppm, 0.25 ppm, 0.30 ppm, 0.35 ppm, 0.40 ppm, 0.45 ppm, 0.50 ppm, 0.55 ppm, 0.60 ppm, 0.65 ppm, 0.70 ppm, 0.75 ppm, 0.80 ppm, 0.85 ppm, 0.90 ppm, 0.95 ppm, 1 ppm, 5 ppm, 10 ppm, 15 ppm, 20 ppm, 25 ppm, 30 ppm, 35 ppm, 40 ppm, 45 ppm, 50 ppm, 55 ppm, 60 ppm, 65 ppm, 70 ppm, 75 ppm, 80 ppm, 85 ppm, 90 ppm, 95 ppm, 10⁰ ppm, 125 ppm, 150 ppm, 175 ppm, 200 ppm, 225 ppm, 250 ppm, 275 ppm, 300 ppm, 325 ppm, 350 ppm, 375 ppm, 400 ppm, 425 ppm, 450 ppm, 475 ppm, 500 ppm, 525 ppm, 550 ppm, 575 ppm, 600 ppm, 625 ppm, 650 ppm, 675 ppm, 700 ppm, 725 ppm, 750 ppm, 775 ppm, 800 ppm, 825 ppm, 850 ppm, 875 ppm, 900 ppm, 925 ppm, 950 ppm, 975 ppm, 1,000 ppm, 1,025 ppm, 1,050 ppm, 1075 ppm, 1,100 ppm, 1,125 ppm, 1,150 ppm, 1,175 ppm, 1,200 ppm, 1,225 ppm, 1,250 ppm, 1,275 ppm, 1,300 ppm, 1,325 ppm, 1,350 ppm, 1,375 ppm, 1,400 ppm, 1,425 ppm, 1,450 ppm, 1,475 ppm, 1,500 ppm, 1,600 ppm, 1,700 ppm, 1,800 ppm, 1,900 ppm, or 2,000 ppm or at least 0.05 ppm, at least 0.10 ppm, at least 0.20 ppm, at least 0.30 ppm, at least 0.40 ppm, at least 0.50 ppm, at least 0.60 ppm, at least 0.70 ppm, at least 0.80 ppm, at least 0.90 ppm, at least 1 ppm, at least 10 ppm, at least 20 ppm, at least 30 ppm, at least 40 ppm, at least 50 ppm, at least 60 ppm, at least 70 ppm, at least 80 ppm, at least 90 ppm, at least 100 ppm, at least 125 ppm, at least 150 ppm, at least 175 ppm, at least 200 ppm, at least 225 ppm, at least 250 ppm, at least 275 ppm, at least 300 ppm, at least 325 ppm, at least 350 ppm, at least 375 ppm, at least 400 ppm, at least 425 ppm, at least 450 ppm, at least 475 ppm, at least 500 ppm, at least 525 ppm, at least 550 ppm, at least 575 ppm, at least 600 ppm, at least 625 ppm, at least 650 ppm, at least 675 ppm, at least 700 ppm, at least 725 ppm, at least 750 ppm, at least 775 ppm, at least 800 ppm, at least 825 ppm, at least 850 ppm, at least 875 ppm, at least 900 ppm, at least 925 ppm, at least 950 ppm, at least 975 ppm, at least 1,000 ppm, at least 1,025 ppm, at least 1,050 ppm, at least 1075 ppm, at least 1,100 ppm, at least 1,125 ppm, at least 1,150 ppm, at least 1,175 ppm, at least 1,200 ppm, at least 1,225 ppm, at least 1,250 ppm, at least 1,275 ppm, at least 1,300 ppm, at least 1,325 ppm, at least 1,350 ppm, at least 1,375 ppm, at least 1,400 ppm, at least 1,425 ppm, at least 1,450 ppm, at least 1,475 ppm, at least 1,500 ppm, at least 1,600 ppm, at least 1,700 ppm, at least 1,800 ppm, at least 1,900 ppm, or at least 2,000 ppm or at most 0.05 ppm, at most 0.10 ppm, at most 0.20 ppm, at most 0.30 ppm, at most 0.40 ppm, at most 0.50 ppm, at most 0.60 ppm, at most 0.70 ppm, at most 0.80 ppm, at most 0.90 ppm, at most 1 ppm, at most 10 ppm, at most 20 ppm, at most 30 ppm, at most 40 ppm, at most 50 ppm, at most 60 ppm, at most 70 ppm, at most 80 ppm, at most 90 ppm, at most 100 ppm, at most 125 ppm, at most 150 ppm, at most 175 ppm, at most 200 ppm, at most 225 ppm, at most 250 ppm, at most 275 ppm, at most 300 ppm, at most 325 ppm, at most 350 ppm, at most 375 ppm, at most 400 ppm, at most 425 ppm, at most 450 ppm, at most 475 ppm, at most 500 ppm, at most 525 ppm, at most 550 ppm, at most 575 ppm, at most 600 ppm, at most 625 ppm, at most 650 ppm, at most 675 ppm, at most 700 ppm, at most 725 ppm, at most 750 ppm, at most 775 ppm, at most 800 ppm, at most 825 ppm, at most 850 ppm, at most 875 ppm, at most 900 ppm, at most 925 ppm, at most 950 ppm, at most 975 ppm, at most 1,000 ppm, at most 1,025 ppm, at most 1,050 ppm, at most 1075 ppm, at most 1,100 ppm, at most 1,125 ppm, at most 1,150 ppm, at most 1,175 ppm, at most 1,200 ppm, at most 1,225 ppm, at most 1,250 ppm, at most 1,275 ppm, at most 1,300 ppm, at most 1,325 ppm, at most 1,350 ppm, at most 1,375 ppm, at most 1,400 ppm, at most 1,425 ppm, at most 1,450 ppm, at most 1,475 ppm, at most 1,500 ppm, at most 1,600 ppm, at most 1,700 ppm, at most 1,800 ppm, at most 1,900 ppm, or at most 2,000 ppm or about 0.5 ppm to about 20 ppm, about 0.5 ppm to about 25 ppm, about 0.5 ppm to about 30 ppm, about 0.5 ppm to about 35 ppm, about 0.5 ppm to about 40 ppm, about 0.5 ppm to about 45 ppm, about 0.5 ppm to about 50 ppm, about 0.5 ppm to about 55 ppm, about 0.5 ppm to about 60 ppm, about 0.5 ppm to about 65 ppm, about 0.5 ppm to about 70 ppm, about 0.5 ppm to about 75 ppm, about 0.5 ppm to about 80 ppm, about 0.5 ppm to about 85 ppm, about 0.5 ppm to about 90 ppm, about 0.5 ppm to about 95 ppm, about 0.5 ppm to about 100 ppm, about 0.75 ppm to about 20 ppm, about 0.75 ppm to about 25 ppm, about 0.75 ppm to about 30 ppm, about 0.75 ppm to about 35 ppm, about 0.75 ppm to about 40 ppm, about 0.75 ppm to about 45 ppm, about 0.75 ppm to about 50 ppm, about 0.75 ppm to about 55 ppm, about 0.75 ppm to about 60 ppm, about 0.75 ppm to about 65 ppm, about 0.75 ppm to about 70 ppm, about 0.75 ppm to about 75 ppm, about 0.75 ppm to about 80 ppm, about 0.75 ppm to about 85 ppm, about 0.75 ppm to about 90 ppm, about 0.75 ppm to about 95 ppm, about 0.75 ppm to about 100 ppm, about 1 ppm to about 20 ppm, about 1 ppm to about 25 ppm, about 1 ppm to about 30 ppm, about 1 ppm to about 35 ppm, about 1 ppm to about 40 ppm, about 1 ppm to about 45 ppm, about 1 ppm to about 50 ppm, about 1 ppm to about 55 ppm, about 1 ppm to about 60 ppm, about 1 ppm to about 65 ppm, about 1 ppm to about 70 ppm, about 1 ppm to about 75 ppm, about 1 ppm to about 80 ppm, about 1 ppm to about 85 ppm, about 1 ppm to about 90 ppm, about 1 ppm to about 95 ppm, about 1 ppm to about 100 ppm, about 5 ppm to about 20 ppm, about 5 ppm to about 25 ppm, about 5 ppm to about 30 ppm, about 5 ppm to about 35 ppm, about 5 ppm to about 40 ppm, about 5 ppm to about 45 ppm, about 5 ppm to about 50 ppm, about 5 ppm to about 55 ppm, about 5 ppm to about 60 ppm, about 5 ppm to about 65 ppm, about 5 ppm to about 70 ppm, about 5 ppm to about 75 ppm, about 5 ppm to about 80 ppm, about 5 ppm to about 85 ppm, about 5 ppm to about 90 ppm, about 5 ppm to about 95 ppm, about 5 ppm to about 100 ppm, about 10 ppm to about 20 ppm, about 10 ppm to about 25 ppm, about 10 ppm to about 30 ppm, about 10 ppm to about 35 ppm, about 10 ppm to about 40 ppm, about 10 ppm to about 45 ppm, about 10 ppm to about 50 ppm, about 10 ppm to about 55 ppm, about 10 ppm to about 60 ppm, about 10 ppm to about 65 ppm, about 10 ppm to about 70 ppm, about 10 ppm to about 75 ppm, about 10 ppm to about 80 ppm, about 10 ppm to about 85 ppm, about 10 ppm to about 90 ppm, about 10 ppm to about 95 ppm, or about 10 ppm to about 100 ppm, about 1 ppm to about 25 ppm, about 1 ppm to about 50 ppm, about 1 ppm to about 75 ppm, about 1 ppm to about 100 ppm, about 1 ppm to about 125 ppm, about 1 ppm to about 150 ppm, about 1 ppm to about 175 ppm, about 1 ppm to about 200 ppm, about 1 ppm to about 225 ppm, about 1 ppm to about 250 ppm, about 1 ppm to about 275 ppm, about 1 ppm to about 300 ppm, about 1 ppm to about 325 ppm, about 1 ppm to about 350 ppm, about 1 ppm to about 375 ppm, about 1 ppm to about 400 ppm, about 10 ppm to about 25 ppm, about 10 ppm to about 50 ppm, about 10 ppm to about 75 ppm, about 10 ppm to about 100 ppm, about 10 ppm to about 125 ppm, about 10 ppm to about 150 ppm, about 10 ppm to about 175 ppm, about 10 ppm to about 200 ppm, about 10 ppm to about 225 ppm, about 10 ppm to about 250 ppm, about 10 ppm to about 275 ppm, about 10 ppm to about 300 ppm, about 10 ppm to about 325 ppm, about 10 ppm to about 350 ppm, about 10 ppm to about 375 ppm, about 10 ppm to about 400 ppm, about 25 ppm to about 50 ppm, about 25 ppm to about 75 ppm, about 25 ppm to about 100 ppm, about 25 ppm to about 125 ppm, about 25 ppm to about 150 ppm, about 25 ppm to about 175 ppm, about 25 ppm to about 200 ppm, about 25 ppm to about 225 ppm, about 25 ppm to about 250 ppm, about 25 ppm to about 275 ppm, about 25 ppm to about 300 ppm, about 25 ppm to about 325 ppm, about 25 ppm to about 350 ppm, about 25 ppm to about 375 ppm, about 25 ppm to about 400 ppm, about 50 ppm to about 75 ppm, about 50 ppm to about 100 ppm, about 50 ppm to about 125 ppm, about 50 ppm to about 150 ppm, about 50 ppm to about 175 ppm, about 50 ppm to about 200 ppm, about 50 ppm to about 225 ppm, about 50 ppm to about 250 ppm, about 50 ppm to about 275 ppm, about 50 ppm to about 300 ppm, about 50 ppm to about 325 ppm, about 50 ppm to about 350 ppm, about 50 ppm to about 375 ppm, about 50 ppm to about 400 ppm, about 75 ppm to about 100 ppm, about 75 ppm to about 125 ppm, about 75 ppm to about 150 ppm, about 75 ppm to about 175 ppm, about 75 ppm to about 200 ppm, about 75 ppm to about 225 ppm, about 75 ppm to about 250 ppm, about 75 ppm to about 275 ppm, about 75 ppm to about 300 ppm, about 75 ppm to about 325 ppm, about 75 ppm to about 350 ppm, about 75 ppm to about 375 ppm, about 75 ppm to about 400 ppm, about 100 ppm to about 125 ppm, about 100 ppm to about 150 ppm, about 100 ppm to about 175 ppm, about 100 ppm to about 200 ppm, about 100 ppm to about 225 ppm, about 100 ppm to about 250 ppm, about 100 ppm to about 275 ppm, about 100 ppm to about 300 ppm, about 100 ppm to about 325 ppm, about 100 ppm to about 350 ppm, about 100 ppm to about 375 ppm, about 100 ppm to about 400 ppm, about 150 ppm to about 175 ppm, about 150 ppm to about 200 ppm, about 150 ppm to about 225 ppm, about 150 ppm to about 250 ppm, about 150 ppm to about 275 ppm, about 150 ppm to about 300 ppm, about 150 ppm to about 325 ppm, about 150 ppm to about 350 ppm, about 150 ppm to about 375 ppm, about 150 ppm to about 400 ppm, about 200 ppm to about 225 ppm, about 200 ppm to about 250 ppm, about 200 ppm to about 275 ppm, about 200 ppm to about 300 ppm, about 200 ppm to about 325 ppm, about 200 ppm to about 350 ppm, about 200 ppm to about 375 ppm, about 200 ppm to about 400 ppm, about 200 ppm to about 425 ppm, about 200 ppm to about 450 ppm, about 200 ppm to about 475 ppm, about 200 ppm to about 500 ppm, about 200 ppm to about 525 ppm, about 200 ppm to about 550 ppm, about 200 ppm to about 575 ppm, about 200 ppm to about 600 ppm, about 250 ppm to about 275 ppm, about 250 ppm to about 300 ppm, about 250 ppm to about 325 ppm, about 250 ppm to about 350 ppm, about 250 ppm to about 375 ppm, about 250 ppm to about 400 ppm, about 250 ppm to about 425 ppm, about 250 ppm to about 450 ppm, about 250 ppm to about 475 ppm, about 250 ppm to about 500 ppm, about 250 ppm to about 525 ppm, about 250 ppm to about 550 ppm, about 250 ppm to about 575 ppm, about 250 ppm to about 600 ppm, about 300 ppm to about 325 ppm, about 300 ppm to about 350 ppm, about 300 ppm to about 375 ppm, about 300 ppm to about 400 ppm, about 300 ppm to about 425 ppm, about 300 ppm to about 450 ppm, about 300 ppm to about 475 ppm, about 300 ppm to about 500 ppm, about 300 ppm to about 525 ppm, about 300 ppm to about 550 ppm, about 300 ppm to about 575 ppm, about 300 ppm to about 600 ppm, about 350 ppm to about 375 ppm, about 350 ppm to about 400 ppm, about 375 ppm to about 400 ppm, about 350 ppm to about 425 ppm, about 350 ppm to about 450 ppm, about 350 ppm to about 475 ppm, about 350 ppm to about 500 ppm, about 350 ppm to about 525 ppm, about 350 ppm to about 550 ppm, about 350 ppm to about 575 ppm, about 350 ppm to about 600 ppm, about 400 ppm to about 500 ppm, about 400 ppm to about 600 ppm, about 400 ppm to about 700 ppm, about 400 ppm to about 800 ppm, about 400 ppm to about 900 ppm, about 400 ppm to about 1,000 ppm, about 400 ppm to about 1,100 ppm, about 400 ppm to about 1,200 ppm, about 400 ppm to about 1,300 ppm, about 400 ppm to about 1,400 ppm, about 400 ppm to about 1,500 ppm, about 400 ppm to about 1,600 ppm, about 400 ppm to about 1,700 ppm, about 400 ppm to about 1,800 ppm, about 400 ppm to about 1,900 ppm, about 400 ppm to about 2,000 ppm, about 500 ppm to about 600 ppm, about 500 ppm to about 700 ppm, about 500 ppm to about 800 ppm, about 500 ppm to about 900 ppm, about 500 ppm to about 1,000 ppm, about 500 ppm to about 1,100 ppm, about 500 ppm to about 1,200 ppm, about 500 ppm to about 1,300 ppm, about 500 ppm to about 1,400 ppm, about 500 ppm to about 1,500 ppm, about 500 ppm to about 1,600 ppm, about 500 ppm to about 1,700 ppm, about 500 ppm to about 1,800 ppm, about 500 ppm to about 1,900 ppm, about 500 ppm to about 2,000 ppm, about 600 ppm to about 700 ppm, about 600 ppm to about 800 ppm, about 600 ppm to about 900 ppm, about 600 ppm to about 1,000 ppm, about 600 ppm to about 1,100 ppm, about 600 ppm to about 1,200 ppm, about 600 ppm to about 1,300 ppm, about 600 ppm to about 1,400 ppm, about 600 ppm to about 1,500 ppm, about 600 ppm to about 1,600 ppm, about 600 ppm to about 1,700 ppm, about 600 ppm to about 1,800 ppm, about 600 ppm to about 1,900 ppm, about 600 ppm to about 2,000 ppm, about 700 ppm to about 800 ppm, about 700 ppm to about 900 ppm, about 700 ppm to about 1,000 ppm, about 700 ppm to about 1,100 ppm, about 700 ppm to about 1,200 ppm, about 700 ppm to about 1,300 ppm, about 700 ppm to about 1,400 ppm, about 700 ppm to about 1,500 ppm, about 700 ppm to about 1,600 ppm, about 700 ppm to about 1,700 ppm, about 700 ppm to about 1,800 ppm, about 700 ppm to about 1,900 ppm, about 700 ppm to about 2,000 ppm, about 800 ppm to about 900 ppm, about 800 ppm to about 1,000 ppm, about 800 ppm to about 1,100 ppm, about 800 ppm to about 1,200 ppm, about 800 ppm to about 1,300 ppm, about 800 ppm to about 1,400 ppm, about 800 ppm to about 1,500 ppm, about 800 ppm to about 1,600 ppm, about 800 ppm to about 1,700 ppm, about 800 ppm to about 1,800 ppm, about 800 ppm to about 1,900 ppm, about 800 ppm to about 2,000 ppm, about 900 ppm to about 1,000 ppm, about 900 ppm to about 1,100 ppm, about 900 ppm to about 1,200 ppm, about 900 ppm to about 1,300 ppm, about 900 ppm to about 1,400 ppm, about 900 ppm to about 1,500 ppm, about 900 ppm to about 1,600 ppm, about 900 ppm to about 1,700 ppm, about 900 ppm to about 1,800 ppm, about 900 ppm to about 1,900 ppm, about 900 ppm to about 2,000 ppm, about 1,000 ppm to about 1,100 ppm, about 1,000 ppm to about 1,200 ppm, about 1,000 ppm to about 1,300 ppm, about 1,000 ppm to about 1,400 ppm, about 1,000 ppm to about 1,500 ppm, about 1,000 ppm to about 1,600 ppm, about 1,000 ppm to about 1,700 ppm, about 1,000 ppm to about 1,800 ppm, about 1,000 ppm to about 1,900 ppm, about 1,000 ppm to about 2,000 ppm, about 1,100 ppm to about 1,200 ppm, about 1,100 ppm to about 1,300 ppm, about 1,100 ppm to about 1,400 ppm, about 1,100 ppm to about 1,500 ppm, about 1,100 ppm to about 1,600 ppm, about 1,100 ppm to about 1,700 ppm, about 1,100 ppm to about 1,800 ppm, about 1,100 ppm to about 1,900 ppm, about 1,100 ppm to about 2,000 ppm, about 1,200 ppm to about 1,300 ppm, about 1,200 ppm to about 1,400 ppm, about 1,200 ppm to about 1,500 ppm, about 1,200 ppm to about 1,600 ppm, about 1,200 ppm to about 1,700 ppm, about 1,200 ppm to about 1,800 ppm, about 1,200 ppm to about 1,900 ppm, about 1,200 ppm to about 2,000 ppm, about 1,300 ppm to about 1,400 ppm, about 1,300 ppm to about 1,500 ppm, about 1,300 ppm to about 1,600 ppm, about 1,300 ppm to about 1,700 ppm, about 1,300 ppm to about 1,800 ppm, about 1,300 ppm to about 1,900 ppm, about 1,300 ppm to about 2,000 ppm, about 1,400 ppm to about 1,500 ppm, about 1,400 ppm to about 1,600 ppm, about 1,400 ppm to about 1,700 ppm, about 1,400 ppm to about 1,800 ppm, about 1,400 ppm to about 1,900 ppm, about 1,400 ppm to about 2,000 ppm, about 1,500 ppm to about 1,600 ppm, about 1,500 ppm to about 1,700 ppm, about 1,500 ppm to about 1,800 ppm, about 1,500 ppm to about 1,900 ppm, about 1,500 ppm to about 2,000 ppm, about 1,600 ppm to about 1,700 ppm, about 1,600 ppm to about 1,800 ppm, about 1,600 ppm to about 1,900 ppm, about 1,600 ppm to about 2,000 ppm, about 1,700 ppm to about 1,800 ppm, about 1,700 ppm to about 1,900 ppm, about 1,700 ppm to about 2,000 ppm, about 1,800 ppm to about 1,900 ppm, about 1,800 ppm to about 2,000 ppm, or about 1,900 ppm to about 2,000 ppm.
31. The composition according to any one of embodiments 10 or 12-30, wherein the one or more metal salt are in an amount of about 0.001%, about 0.005%, about 0.01%, about 0.05%, about 0.10%, about 0.11%, about 0.12%, about 0.13%, about 0.14%, about 0.15%, about 0.16%, about 0.17%, about 0.18%, about 0.19%, about 0.2%, about 0.25%, about 0.3%, about 0.35%, about 0.4%, about 0.45%, or about 0.5% by weight of the composition, or at least 0.001%, at least 0.005%, at least 0.01%, at least 0.05%, at least 0.10%, at least 0.11%, at least 0.12%, at least 0.13%, at least 0.14%, at least 0.15%, at least 0.16%, at least 0.17%, at least 0.18%, at least 0.19%, at least 0.2%, at least 0.25%, at least 0.3%, at least 0.35%, at least 0.4%, at least 0.45%, or at least 0.5% by weight of the composition, or at most 0.001%, at most 0.005%, at most 0.01%, at most 0.05%, at most 0.10%, at most 0.11%, at most 0.12%, at most 0.13%, at most 0.14%, at most 0.15%, at most 0.16%, at most 0.17%, at most 0.18%, at most 0.19%, at most 0.2%, at most 0.25%, at most 0.3%, at most 0.35%, at most 0.4%, at most 0.45%, or at most 0.5% by weight of the composition, or about 0.0010% to about 0.005%, about 0.0010% to about 0.010%, about 0.0010% to about 0.050%, about 0.0010% to about 0.10%, about 0.0010% to about 0.15%, about 0.0010% to about 0.20%, about 0.0010% to about 0.25%, about 0.0010% to about 0.30%, about 0.0010% to about 0.35%, about 0.0010% to about 0.40%, about 0.0010% to about 0.45%, about 0.0010% to about 0.50%, about 0.010% to about 0.050%, about 0.010% to about 0.10%, about 0.010% to about 0.15%, about 0.010% to about 0.20%, about 0.010% to about 0.25%, about 0.010% to about 0.30%, about 0.010% to about 0.35%, about 0.010% to about 0.40%, about 0.010% to about 0.45%, about 0.010% to about 0.50%, about 0.10% to about 0.15%, about 0.10% to about 0.20%, about 0.10% to about 0.25%, about 0.10% to about 0.30%, about 0.10% to about 0.35%, about 0.10% to about 0.40%, about 0.10% to about 0.45%, about 0.10% to about 0.50%, about 0.20% to about 0.25%, about 0.20% to about 0.30%, about 0.20% to about 0.35%, about 0.20% to about 0.40%, about 0.20% to about 0.45%, about 0.20% to about 0.50%, about 0.30% to about 0.35%, about 0.30% to about 0.40%, about 0.30% to about 0.45%, about 0.30% to about 0.50%, about 0.40% to about 0.45%, about 0.40% to about 0.50%, or about 0.40% to about 0.50% by weight of the composition.
32. The composition according to any one of embodiments 1-31, further comprising, consisting essentially of, or consisting of one or more carriers.
33. The composition according to embodiment 32, wherein the one or more carrier includes an aqueous carrier, a semi-solid carrier, a solid carrier, or any combination thereof.
34. The composition according to embodiment 32 or 33, wherein the one or more carrier includes water, a vegetable oil, a mineral oil, an ester oil, an ether, an alcohol, a fatty alcohol, an isoparaffin, a hydrocarbon oil, a polyol, a wax, or any combination thereof.
35. The composition according to embodiment 34, wherein the ester oil includes octal palmitate, isopropyl myristate or isopropyl palmitate.
36. The composition according to embodiment 34, wherein the ether includes dicapryl ether or dimethyl isosorbide.
37. The composition according to embodiment 34, wherein the alcohol includes ethanol or isopropanol.
38. The composition according to embodiment 34, wherein the fatty alcohol includes cetyl alcohol, cetearyl alcohol, stearyl alcohol or behenyl alcohol.
39. The composition according to embodiment 34, wherein the isoparaffin includes isooctane, isododecane (IDD) or isohexadecane.
40. The composition according to embodiment 34, wherein the hydrocarbon oil includes mineral oil, petrolatum, isoeicosane or a polyolefin.
41. The composition according to embodiment 34, wherein the polyol includes propylene glycol, glycerin, butylene glycol, pentylene glycol, hexylene glycol, or caprylyl glycol.
42. The composition according to embodiment 34, wherein the wax includes beeswax, carnauba, ozokerite, microcrystalline wax, polyethylene wax, or a botanical wax.
43. The composition according to any one of embodiments 32-42, wherein the one or more carriers are in an amount of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98% or at least 99% by weight of the composition or at most 5%, at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95%, at most 96%, at most 97%, at most 98% or at most 99% by weight of the composition or about 5% to about 25%, about 5% to about 50%, about 5% to about 75%, about 5% to about 90%, about 5% to about 95%, about 5% to about 96%, about 5% to about 97%, about 5% to about 98%, about 5% to about 99%, about 25% to about 50%, about 25% to about 75%, about 25% to about 90%, about 25% to about 95%, about 25% to about 96%, about 25% to about 97%, about 25% to about 98%, about 25% to about 99%, about 50% to about 75%, about 50% to about 90%, about 50% to about 95%, about 50% to about 96%, about 50% to about 97%, about 50% to about 98%, about 50% to about 99%, about 75% to about 80%, about 75% to about 85%, about 75% to about 90%, about 75% to about 95%, about 75% to about 96%, about 75% to about 97%, about 75% to about 98%, about 75% to about 99%, about 80% to about 85%, about 80% to about 90%, about 80% to about 95%, about 80% to about 96%, about 80% to about 97%, about 80% to about 98%, about 80% to about 99%, about 85% to about 90%, about 85% to about 95%, about 85% to about 96%, about 85% to about 97%, about 85% to about 98%, about 85% to about 99%, about 90% to about 95%, about 90% to about 96%, about 90% to about 97%, about 90% to about 98%, about 90% to about 99%, or about 95% to about 99%, by weight of the composition.
44. The composition according to any one of embodiments 1-43, further comprising, consisting essentially of, or consisting of one or more additional ingredients.
45. The composition according to embodiment 44, wherein the one or more additional ingredient includes a preservative, a chelating agent, or any combination thereof.
46. The composition according to any one of embodiments 1-45, having a pH of about 2 to about 5, about 2 to about 5.5, about 2 to about 6, about 2 to about 6.5, about 2 to about 7, about 2 to about 7.5, about 2 to about 8, about 2 to about 8.5, about 2 to about 9, about 2.5 to about 5, about 2.5 to about 5.5, about 2.5 to about 6, about 2.5 to about 6.5, about 2.5 to about 7, about 2.5 to about 7.5, about 2.5 to about 8, about 2.5 to about 8.5, about 2.5 to about 9, about 3 to about 5, about 3 to about 5.5, about 3 to about 6, about 3 to about 6.5, about 3 to about 7, about 3 to about 7.5, about 3 to about 8, about 3 to about 8.5, about 3 to about 9, about 3.5 to about 5, about 3.5 to about 5.5, about 3.5 to about 6, about 3.5 to about 6.5, about 3.5 to about 7, about 3.5 to about 7.5, about 3.5 to about 8, about 3.5 to about 8.5, about 3.5 to about 9, about 4 to about 5, about 4 to about 5.5, about 4 to about 6, about 4 to about 6.5, about 4 to about 7, about 4 to about 7.5, about 4 to about 8, about 4 to about 8.5, about 4 to about 9, about 4.5 to about 5, about 4.5 to about 5.5, about 4.5 to about 6, about 4.5 to about 6.5, about 4.5 to about 7, about 4.5 to about 7.5, about 4.5 to about 8, about 4.5 to about 8.5, about 4.5 to about 9, about 5 to about 5.5, about 5 to about 6, about 5 to about 6.5, about 5 to about 7, about 5 to about 7.5, about 5 to about 8, about 5 to about 8.5, about 5 to about 9, about 5.5 to about 6, about 5.5 to about 6.5, about 5.5 to about 7, about 5.5 to about 7.5, about 5.5 to about 8, about 5.5 to about 8.5, about 5.5 to about 9, about 6 to about 6.5, about 6 to about 7, about 6 to about 7.5, about 6 to about 8, about 6 to about 8.5, about 6 to about 9, about 6.5 to about 7, about 6.5 to about 7.5, about 6.5 to about 8, about 6.5 to about 8.5, about 6.5 to about 9, about 7 to about 7.5, about 7 to about 8, about 7 to about 8.5, about 7 to about 9, about 7.5 to about 8, about 7.5 to about 8.5, about 7.5 to about 9, about 8 to about 8.5, about 8 to about 9, or about 8.5 to about 9.
47. A kit comprising, consisting essentially of, or consisting of a composition as defined in any one of embodiments 1-46.
48. The kit according to embodiment 47, further comprising, consisting essentially of, or consisting of one or more delivery or application systems, and/or instructions, and/or a container.
49. A kit comprising, consisting essentially of, or consisting of a first component including hypochlorous acid or free available chlorine.
50. A kit comprising, consisting essentially of, or consisting of a first component including hypochlorous acid or free available chlorine and a second component including or more quaternary compounds or silicon quaternary compounds.
51. A kit comprising, consisting essentially of, or consisting of a first component including hypochlorous acid or free available chlorine and a second component including one or more guanide-containing compounds.
52. A kit comprising, consisting essentially of, or consisting of a first component including one or more alcohols and a second component including one or more quaternary compounds or silicon quaternary compounds.
53. A kit comprising, consisting essentially of, or consisting of a first component including one or more alcohols and a second component including one or more guanide-containing compounds.
54. A kit comprising, consisting essentially of, or consisting of a first component including hypochlorous acid or free available chlorine and a second component including one or more metallic particles.
55. A kit comprising, consisting essentially of, or consisting of a first component including hypochlorous acid or free available chlorine and a second component including one or more metal salts.
56. A kit comprising, consisting essentially of, or consisting of a first component including hypochlorous acid or free available chlorine, a second component including one or more metallic particles and a third component including one or more metal salts.
57. The kit according to any one of embodiments 50-56, further comprising, consisting essentially of, or consisting of a third component including one or more metallic particles and/or a fourth component including one or more metal salts, and/or a fifth component including one or more cationic surfactants, and/or a sixth component including a rinse solution and/or one or more delivery or application systems, and/or instructions, and/or a container
58. The kit according to embodiments 47-51 and 54-56, wherein the hypochlorous acid or free available chlorine is in an amount of 0.01 ppm, 0.05 ppm, 0.10 ppm, 0.15 ppm, 0.20 ppm, 0.25 ppm, 0.30 ppm, 0.35 ppm, 0.40 ppm, 0.45 ppm, 0.50 ppm, 0.55 ppm, 0.60 ppm, 0.65 ppm, 0.70 ppm, 0.75 ppm, 0.80 ppm, 0.85 ppm, 0.90 ppm, 0.95 ppm, 1 ppm, 5 ppm, 10 ppm, 15 ppm, 20 ppm, 25 ppm, 30 ppm, 35 ppm, 40 ppm, 45 ppm, 50 ppm, 55 ppm, 60 ppm, 65 ppm, 70 ppm, 75 ppm, 80 ppm, 85 ppm, 90 ppm, 95 ppm, 100 ppm, 125 ppm, 150 ppm, 175 ppm, 200 ppm, 225 ppm, 250 ppm, 275 ppm, 300 ppm, 325 ppm, 350 ppm, 375 ppm, 400 ppm, 425 ppm, 450 ppm, 475 ppm, 500 ppm, 525 ppm, 550 ppm, 575 ppm, 600 ppm, 625 ppm, 650 ppm, 675 ppm, 700 ppm, 725 ppm, 750 ppm, 775 ppm, 800 ppm, 825 ppm, 850 ppm, 875 ppm, 900 ppm, 925 ppm, 950 ppm, 975 ppm, 1,000 ppm, 1,025 ppm, 1,050 ppm, 1075 ppm, 1,100 ppm, 1,125 ppm, 1,150 ppm, 1,175 ppm, 1,200 ppm, 1,225 ppm, 1,250 ppm, 1,275 ppm, 1,300 ppm, 1,325 ppm, 1,350 ppm, 1,375 ppm, 1,400 ppm, 1,425 ppm, 1,450 ppm, 1,475 ppm, 1,500 ppm, 1,600 ppm, 1,700 ppm, 1,800 ppm, 1,900 ppm, or 2,000 ppm or at least 0.01 ppm, at least 0.05 ppm, at least 0.10 ppm, at least 0.20 ppm, at least 0.30 ppm, at least 0.40 ppm, at least 0.50 ppm, at least 0.60 ppm, at least 0.70 ppm, at least 0.80 ppm, at least 0.90 ppm, at least 1 ppm, at least 10 ppm, at least 20 ppm, at least 30 ppm, at least 40 ppm, at least 50 ppm, at least 60 ppm, at least 70 ppm, at least 80 ppm, at least 90 ppm, at least 100 ppm, at least 125 ppm, at least 150 ppm, at least 175 ppm, at least 200 ppm, at least 225 ppm, at least 250 ppm, at least 275 ppm, at least 300 ppm, at least 325 ppm, at least 350 ppm, at least 375 ppm, at least 400 ppm, at least 425 ppm, at least 450 ppm, at least 475 ppm, at least 500 ppm, at least 525 ppm, at least 550 ppm, at least 575 ppm, at least 600 ppm, at least 625 ppm, at least 650 ppm, at least 675 ppm, at least 700 ppm, at least 725 ppm, at least 750 ppm, at least 775 ppm, at least 800 ppm, at least 825 ppm, at least 850 ppm, at least 875 ppm, at least 900 ppm, at least 925 ppm, at least 950 ppm, at least 975 ppm, at least 1,000 ppm, at least 1,025 ppm, at least 1,050 ppm, at least 1075 ppm, at least 1,100 ppm, at least 1,125 ppm, at least 1,150 ppm, at least 1,175 ppm, at least 1,200 ppm, at least 1,225 ppm, at least 1,250 ppm, at least 1,275 ppm, at least 1,300 ppm, at least 1,325 ppm, at least 1,350 ppm, at least 1,375 ppm, at least 1,400 ppm, at least 1,425 ppm, at least 1,450 ppm, at least 1,475 ppm, at least 1,500 ppm, at least 1,600 ppm, at least 1,700 ppm, at least 1,800 ppm, at least 1,900 ppm, or at least 2,000 ppm or at most 0.01 ppm, at most 0.05 ppm, at most 0.10 ppm, at most 0.20 ppm, at most 0.30 ppm, at most 0.40 ppm, at most 0.50 ppm, at most 0.60 ppm, at most 0.70 ppm, at most 0.80 ppm, at most 0.90 ppm, at most 1 ppm, at most 10 ppm, at most 20 ppm, at most 30 ppm, at most 40 ppm, at most 50 ppm, at most 60 ppm, at most 70 ppm, at most 80 ppm, at most 90 ppm, at most 100 ppm, at most 125 ppm, at most 150 ppm, at most 175 ppm, at most 200 ppm, at most 225 ppm, at most 250 ppm, at most 275 ppm, at most 300 ppm, at most 325 ppm, at most 350 ppm, at most 375 ppm, at most 400 ppm, at most 425 ppm, at most 450 ppm, at most 475 ppm, at most 500 ppm, at most 525 ppm, at most 550 ppm, at most 575 ppm, at most 600 ppm, at most 625 ppm, at most 650 ppm, at most 675 ppm, at most 700 ppm, at most 725 ppm, at most 750 ppm, at most 775 ppm, at most 800 ppm, at most 825 ppm, at most 850 ppm, at most 875 ppm, at most 900 ppm, at most 925 ppm, at most 950 ppm, at most 975 ppm, at most 1,000 ppm, at most 1,025 ppm, at most 1,050 ppm, at most 1075 ppm, at most 1,100 ppm, at most 1,125 ppm, at most 1,150 ppm, at most 1,175 ppm, at most 1,200 ppm, at most 1,225 ppm, at most 1,250 ppm, at most 1,275 ppm, at most 1,300 ppm, at most 1,325 ppm, at most 1,350 ppm, at most 1,375 ppm, at most 1,400 ppm, at most 1,425 ppm, at most 1,450 ppm, at most 1,475 ppm, at most 1,500 ppm, at most 1,600 ppm, at most 1,700 ppm, at most 1,800 ppm, at most 1,900 ppm, or at most 2,000 ppm or at most 0.05 ppm, about 0.5 ppm to about 55 ppm, about 0.5 ppm to about 60 ppm, about 0.5 ppm to about 65 ppm, about 0.5 ppm to about 70 ppm, about 0.5 ppm to about 75 ppm, about 0.5 ppm to about 80 ppm, about 0.5 ppm to about 85 ppm, about 0.5 ppm to about 90 ppm, about 0.5 ppm to about 95 ppm, about 0.5 ppm to about 100 ppm, about 0.75 ppm to about 20 ppm, about 0.75 ppm to about 25 ppm, about 0.75 ppm to about 30 ppm, about 0.75 ppm to about 35 ppm, about 0.75 ppm to about 40 ppm, about 0.75 ppm to about 45 ppm, about 0.75 ppm to about 50 ppm, about 0.75 ppm to about 55 ppm, about 0.75 ppm to about 60 ppm, about 0.75 ppm to about 65 ppm, about 0.75 ppm to about 70 ppm, about 0.75 ppm to about 75 ppm, about 0.75 ppm to about 80 ppm, about 0.75 ppm to about 85 ppm, about 0.75 ppm to about 90 ppm, about 0.75 ppm to about 95 ppm, about 0.75 ppm to about 100 ppm, about 1 ppm to about 20 ppm, about 1 ppm to about 25 ppm, about 1 ppm to about 30 ppm, about 1 ppm to about 35 ppm, about 1 ppm to about 40 ppm, about 1 ppm to about 45 ppm, about 1 ppm to about 50 ppm, about 1 ppm to about 55 ppm, about 1 ppm to about 60 ppm, about 1 ppm to about 65 ppm, about 1 ppm to about 70 ppm, about 1 ppm to about 75 ppm, about 1 ppm to about 80 ppm, about 1 ppm to about 85 ppm, about 1 ppm to about 90 ppm, about 1 ppm to about 95 ppm, about 1 ppm to about 100 ppm, about 5 ppm to about 20 ppm, about 5 ppm to about 25 ppm, about 5 ppm to about 30 ppm, about 5 ppm to about 35 ppm, about 5 ppm to about 40 ppm, about 5 ppm to about 45 ppm, about 5 ppm to about 50 ppm, about 5 ppm to about 55 ppm, about 5 ppm to about 60 ppm, about 5 ppm to about 65 ppm, about 5 ppm to about 70 ppm, about 5 ppm to about 75 ppm, about 5 ppm to about 80 ppm, about 5 ppm to about 85 ppm, about 5 ppm to about 90 ppm, about 5 ppm to about 95 ppm, about 5 ppm to about 100 ppm, about 10 ppm to about 20 ppm, about 10 ppm to about 25 ppm, about 10 ppm to about 30 ppm, about 10 ppm to about 35 ppm, about 10 ppm to about 40 ppm, about 10 ppm to about 45 ppm, about 10 ppm to about 50 ppm, about 10 ppm to about 55 ppm, about 10 ppm to about 60 ppm, about 10 ppm to about 65 ppm, about 10 ppm to about 70 ppm, about 10 ppm to about 75 ppm, about 10 ppm to about 80 ppm, about 10 ppm to about 85 ppm, about 10 ppm to about 90 ppm, about 10 ppm to about 95 ppm, or about 10 ppm to about 100 ppm, about 1 ppm to about 25 ppm, about 1 ppm to about 50 ppm, about 1 ppm to about 75 ppm, about 1 ppm to about 100 ppm, about 1 ppm to about 125 ppm, about 1 ppm to about 150 ppm, about 1 ppm to about 175 ppm, about 1 ppm to about 200 ppm, about 1 ppm to about 225 ppm, about 1 ppm to about 250 ppm, about 1 ppm to about 275 ppm, about 1 ppm to about 300 ppm, about 1 ppm to about 325 ppm, about 1 ppm to about 350 ppm, about 1 ppm to about 375 ppm, about 1 ppm to about 400 ppm, about 1 ppm to about 425 ppm, about 1 ppm to about 450 ppm, about 1 ppm to about 475 ppm, about 1 ppm to about 500 ppm, about 5 ppm to about 25 ppm, about 5 ppm to about 50 ppm, about 5 ppm to about 75 ppm, about 5 ppm to about 100 ppm, about 5 ppm to about 125 ppm, about 5 ppm to about 150 ppm, about 5 ppm to about 175 ppm, about 5 ppm to about 200 ppm, about 5 ppm to about 225 ppm, about 5 ppm to about 250 ppm, about 5 ppm to about 275 ppm, about 1 ppm to about 300 ppm, about 5 ppm to about 325 ppm, about 5 ppm to about 350 ppm, about 5 ppm to about 375 ppm, about 5 ppm to about 400 ppm, about 5 ppm to about 425 ppm, about 5 ppm to about 450 ppm, about 5 ppm to about 475 ppm, about 5 ppm to about 500 ppm, about 10 ppm to about 25 ppm, about 10 ppm to about 50 ppm, about 10 ppm to about 75 ppm, about 10 ppm to about 100 ppm, about 10 ppm to about 125 ppm, about 10 ppm to about 150 ppm, about 10 ppm to about 175 ppm, about 10 ppm to about 200 ppm, about 10 ppm to about 225 ppm, about 10 ppm to about 250 ppm, about 10 ppm to about 275 ppm, about 10 ppm to about 300 ppm, about 10 ppm to about 325 ppm, about 10 ppm to about 350 ppm, about 10 ppm to about 375 ppm, about 10 ppm to about 400 ppm, about 10 ppm to about 425 ppm, about 10 ppm to about 450 ppm, about 10 ppm to about 475 ppm, about 10 ppm to about 500 ppm, about 25 ppm to about 50 ppm, about 25 ppm to about 75 ppm, about 25 ppm to about 10⁰ ppm, about 25 ppm to about 125 ppm, about 25 ppm to about 150 ppm, about 25 ppm to about 175 ppm, about 25 ppm to about 200 ppm, about 25 ppm to about 225 ppm, about 25 ppm to about 250 ppm, about 25 ppm to about 275 ppm, about 25 ppm to about 300 ppm, about 25 ppm to about 325 ppm, about 25 ppm to about 350 ppm, about 25 ppm to about 375 ppm, about 25 ppm to about 400 ppm, about 25 ppm to about 425 ppm, about 25 ppm to about 450 ppm, about 25 ppm to about 475 ppm, about 25 ppm to about 500 ppm, about 50 ppm to about 75 ppm, about 50 ppm to about 10⁰ ppm, about 50 ppm to about 125 ppm, about 50 ppm to about 150 ppm, about 50 ppm to about 175 ppm, about 50 ppm to about 200 ppm, about 50 ppm to about 225 ppm, about 50 ppm to about 250 ppm, about 50 ppm to about 275 ppm, about 50 ppm to about 300 ppm, about 50 ppm to about 325 ppm, about 50 ppm to about 350 ppm, about 50 ppm to about 375 ppm, about 50 ppm to about 400 ppm, about 50 ppm to about 425 ppm, about 50 ppm to about 450 ppm, about 50 ppm to about 475 ppm, about 50 ppm to about 500 ppm, about 75 ppm to about 100 ppm, about 75 ppm to about 125 ppm, about 75 ppm to about 150 ppm, about 75 ppm to about 175 ppm, about 75 ppm to about 200 ppm, about 75 ppm to about 225 ppm, about 75 ppm to about 250 ppm, about 75 ppm to about 275 ppm, about 75 ppm to about 300 ppm, about 75 ppm to about 325 ppm, about 75 ppm to about 350 ppm, about 75 ppm to about 375 ppm, about 75 ppm to about 400 ppm, about 75 ppm to about 425 ppm, about 75 ppm to about 450 ppm, about 75 ppm to about 475 ppm, about 75 ppm to about 500 ppm, about 100 ppm to about 125 ppm, about 100 ppm to about 150 ppm, about 100 ppm to about 175 ppm, about 100 ppm to about 200 ppm, about 100 ppm to about 225 ppm, about 100 ppm to about 250 ppm, about 100 ppm to about 275 ppm, about 100 ppm to about 300 ppm, about 100 ppm to about 325 ppm, about 100 ppm to about 350 ppm, about 100 ppm to about 375 ppm, about 100 ppm to about 400 ppm, about 150 ppm to about 175 ppm, about 150 ppm to about 200 ppm, about 150 ppm to about 225 ppm, about 150 ppm to about 250 ppm, about 150 ppm to about 275 ppm, about 150 ppm to about 300 ppm, about 150 ppm to about 325 ppm, about 150 ppm to about 350 ppm, about 150 ppm to about 375 ppm, about 150 ppm to about 400 ppm, about 200 ppm to about 225 ppm, about 200 ppm to about 250 ppm, about 200 ppm to about 275 ppm, about 200 ppm to about 300 ppm, about 200 ppm to about 325 ppm, about 200 ppm to about 350 ppm, about 200 ppm to about 375 ppm, about 200 ppm to about 400 ppm, about 200 ppm to about 425 ppm, about 200 ppm to about 450 ppm, about 200 ppm to about 475 ppm, about 200 ppm to about 500 ppm, about 200 ppm to about 525 ppm, about 200 ppm to about 550 ppm, about 200 ppm to about 575 ppm, about 200 ppm to about 600 ppm, about 250 ppm to about 275 ppm, about 250 ppm to about 300 ppm, about 250 ppm to about 325 ppm, about 250 ppm to about 350 ppm, about 250 ppm to about 375 ppm, about 250 ppm to about 400 ppm, about 250 ppm to about 425 ppm, about 250 ppm to about 450 ppm, about 250 ppm to about 475 ppm, about 250 ppm to about 500 ppm, about 250 ppm to about 525 ppm, about 250 ppm to about 550 ppm, about 250 ppm to about 575 ppm, about 250 ppm to about 600 ppm, about 300 ppm to about 325 ppm, about 300 ppm to about 350 ppm, about 300 ppm to about 375 ppm, about 300 ppm to about 400 ppm, about 300 ppm to about 425 ppm, about 300 ppm to about 450 ppm, about 300 ppm to about 475 ppm, about 300 ppm to about 500 ppm, about 300 ppm to about 525 ppm, about 300 ppm to about 550 ppm, about 300 ppm to about 575 ppm, about 300 ppm to about 600 ppm, about 350 ppm to about 375 ppm, about 350 ppm to about 400 ppm, about 375 ppm to about 400 ppm, about 350 ppm to about 425 ppm, about 350 ppm to about 450 ppm, about 350 ppm to about 475 ppm, about 350 ppm to about 500 ppm, about 350 ppm to about 525 ppm, about 350 ppm to about 550 ppm, about 350 ppm to about 575 ppm, about 350 ppm to about 600 ppm, about 400 ppm to about 500 ppm, about 400 ppm to about 600 ppm, about 400 ppm to about 700 ppm, about 400 ppm to about 800 ppm, about 400 ppm to about 900 ppm, about 400 ppm to about 1,000 ppm, about 400 ppm to about 1,100 ppm, about 400 ppm to about 1,200 ppm, about 400 ppm to about 1,300 ppm, about 400 ppm to about 1,400 ppm, about 400 ppm to about 1,500 ppm, about 400 ppm to about 1,600 ppm, about 400 ppm to about 1,700 ppm, about 400 ppm to about 1,800 ppm, about 400 ppm to about 1,900 ppm, about 400 ppm to about 2,000 ppm, about 500 ppm to about 600 ppm, about 500 ppm to about 700 ppm, about 500 ppm to about 800 ppm, about 500 ppm to about 900 ppm, about 500 ppm to about 1,000 ppm, about 500 ppm to about 1,100 ppm, about 500 ppm to about 1,200 ppm, about 500 ppm to about 1,300 ppm, about 500 ppm to about 1,400 ppm, about 500 ppm to about 1,500 ppm, about 500 ppm to about 1,600 ppm, about 500 ppm to about 1,700 ppm, about 500 ppm to about 1,800 ppm, about 500 ppm to about 1,900 ppm, about 500 ppm to about 2,000 ppm, about 600 ppm to about 700 ppm, about 600 ppm to about 800 ppm, about 600 ppm to about 900 ppm, about 600 ppm to about 1,000 ppm, about 600 ppm to about 1,100 ppm, about 600 ppm to about 1,200 ppm, about 600 ppm to about 1,300 ppm, about 600 ppm to about 1,400 ppm, about 600 ppm to about 1,500 ppm, about 600 ppm to about 1,600 ppm, about 600 ppm to about 1,700 ppm, about 600 ppm to about 1,800 ppm, about 600 ppm to about 1,900 ppm, about 600 ppm to about 2,000 ppm, about 700 ppm to about 800 ppm, about 700 ppm to about 900 ppm, about 700 ppm to about 1,000 ppm, about 700 ppm to about 1,100 ppm, about 700 ppm to about 1,200 ppm, about 700 ppm to about 1,300 ppm, about 700 ppm to about 1,400 ppm, about 700 ppm to about 1,500 ppm, about 700 ppm to about 1,600 ppm, about 700 ppm to about 1,700 ppm, about 700 ppm to about 1,800 ppm, about 700 ppm to about 1,900 ppm, about 700 ppm to about 2,000 ppm, about 800 ppm to about 900 ppm, about 800 ppm to about 1,000 ppm, about 800 ppm to about 1,100 ppm, about 800 ppm to about 1,200 ppm, about 800 ppm to about 1,300 ppm, about 800 ppm to about 1,400 ppm, about 800 ppm to about 1,500 ppm, about 800 ppm to about 1,600 ppm, about 800 ppm to about 1,700 ppm, about 800 ppm to about 1,800 ppm, about 800 ppm to about 1,900 ppm, about 800 ppm to about 2,000 ppm, about 900 ppm to about 1,000 ppm, about 900 ppm to about 1,100 ppm, about 900 ppm to about 1,200 ppm, about 900 ppm to about 1,300 ppm, about 900 ppm to about 1,400 ppm, about 900 ppm to about 1,500 ppm, about 900 ppm to about 1,600 ppm, about 900 ppm to about 1,700 ppm, about 900 ppm to about 1,800 ppm, about 900 ppm to about 1,900 ppm, about 900 ppm to about 2,000 ppm, about 1,000 ppm to about 1,100 ppm, about 1,000 ppm to about 1,200 ppm, about 1,000 ppm to about 1,300 ppm, about 1,000 ppm to about 1,400 ppm, about 1,000 ppm to about 1,500 ppm, about 1,000 ppm to about 1,600 ppm, about 1,000 ppm to about 1,700 ppm, about 1,000 ppm to about 1,800 ppm, about 1,000 ppm to about 1,900 ppm, about 1,000 ppm to about 2,000 ppm, about 1,100 ppm to about 1,200 ppm, about 1,100 ppm to about 1,300 ppm, about 1,100 ppm to about 1,400 ppm, about 1,100 ppm to about 1,500 ppm, about 1,100 ppm to about 1,600 ppm, about 1,100 ppm to about 1,700 ppm, about 1,100 ppm to about 1,800 ppm, about 1,100 ppm to about 1,900 ppm, about 1,100 ppm to about 2,000 ppm, about 1,200 ppm to about 1,300 ppm, about 1,200 ppm to about 1,400 ppm, about 1,200 ppm to about 1,500 ppm, about 1,200 ppm to about 1,600 ppm, about 1,200 ppm to about 1,700 ppm, about 1,200 ppm to about 1,800 ppm, about 1,200 ppm to about 1,900 ppm, about 1,200 ppm to about 2,000 ppm, about 1,300 ppm to about 1,400 ppm, about 1,300 ppm to about 1,500 ppm, about 1,300 ppm to about 1,600 ppm, about 1,300 ppm to about 1,700 ppm, about 1,300 ppm to about 1,800 ppm, about 1,300 ppm to about 1,900 ppm, about 1,300 ppm to about 2,000 ppm, about 1,400 ppm to about 1,500 ppm, about 1,400 ppm to about 1,600 ppm, about 1,400 ppm to about 1,700 ppm, about 1,400 ppm to about 1,800 ppm, about 1,400 ppm to about 1,900 ppm, about 1,400 ppm to about 2,000 ppm, about 1,500 ppm to about 1,600 ppm, about 1,500 ppm to about 1,700 ppm, about 1,500 ppm to about 1,800 ppm, about 1,500 ppm to about 1,900 ppm, about 1,500 ppm to about 2,000 ppm, about 1,600 ppm to about 1,700 ppm, about 1,600 ppm to about 1,800 ppm, about 1,600 ppm to about 1,900 ppm, about 1,600 ppm to about 2,000 ppm, about 1,700 ppm to about 1,800 ppm, about 1,700 ppm to about 1,900 ppm, about 1,700 ppm to about 2,000 ppm, about 1,800 ppm to about 1,900 ppm, about 1,800 ppm to about 2,000 ppm, or about 1,900 ppm to about 2,000 ppm.
59. The kit according to any one of embodiments 47-51 and 54-56, wherein the hypochlorous acid or free available chlorine is in an amount of about 0.001%, about 0.005%, about 0.01%, about 0.05%, about 0.10%, about 0.11%, about 0.12%, about 0.13%, about 0.14%, about 0.15%, about 0.16%, about 0.17%, about 0.18%, about 0.19%, about 0.2%, about 0.25%, about 0.3%, about 0.35%, about 0.4%, about 0.45%, or about 0.5% by weight of the composition, or at least 0.001%, at least 0.005%, at least 0.01%, at least 0.05%, at least 0.10%, at least 0.11%, at least 0.12%, at least 0.13%, at least 0.14%, at least 0.15%, at least 0.16%, at least 0.17%, at least 0.18%, at least 0.19%, at least 0.2%, at least 0.25%, at least 0.3%, at least 0.35%, at least 0.4%, at least 0.45%, or at least 0.5% by weight of the composition, or at most 0.001%, at most 0.005%, at most 0.01%, at most 0.05%, at most 0.10%, at most 0.11%, at most 0.12%, at most 0.13%, at most 0.14%, at most 0.15%, at most 0.16%, at most 0.17%, at most 0.18%, at most 0.19%, at most 0.2%, at most 0.25%, at most 0.3%, at most 0.35%, at most 0.4%, at most 0.45%, or at most 0.5% by weight of the composition, or about 0.0010% to about 0.005%, about 0.0010% to about 0.010%, about 0.0010% to about 0.050%, about 0.0010% to about 0.10%, about 0.0010% to about 0.15%, about 0.0010% to about 0.20%, about 0.0010% to about 0.25%, about 0.0010% to about 0.30%, about 0.0010% to about 0.35%, about 0.0010% to about 0.40%, about 0.0010% to about 0.45%, about 0.0010% to about 0.50%, about 0.010% to about 0.050%, about 0.010% to about 0.10%, about 0.010% to about 0.15%, about 0.010% to about 0.20%, about 0.010% to about 0.25%, about 0.010% to about 0.30%, about 0.010% to about 0.35%, about 0.010% to about 0.40%, about 0.010% to about 0.45%, about 0.010% to about 0.50%, about 0.10% to about 0.15%, about 0.10% to about 0.20%, about 0.10% to about 0.25%, about 0.10% to about 0.30%, about 0.10% to about 0.35%, about 0.10% to about 0.40%, about 0.10% to about 0.45%, about 0.10% to about 0.50%, about 0.20% to about 0.25%, about 0.20% to about 0.30%, about 0.20% to about 0.35%, about 0.20% to about 0.40%, about 0.20% to about 0.45%, about 0.20% to about 0.50%, about 0.30% to about 0.35%, about 0.30% to about 0.40%, about 0.30% to about 0.45%, about 0.30% to about 0.50%, about 0.40% to about 0.45%, about 0.40% to about 0.50%, or about 0.40% to about 0.50% by weight of the composition.
60. The kit according to any one of embodiments 47-51, 52 or 56, wherein the one or more quaternary compounds include one or more dialkyl quaternary compounds and/or one or more polyether fatty quaternary compounds and/or wherein the one or more silicon quaternary compounds include one or more silicone dialkyl quaternary compounds and/or one or more silicone polyether fatty quaternary compounds.
61. The kit according to embodiment 60, wherein the one or more dialkyl quaternary compounds include didodecyldimethylammonium chloride and di-n-alkyldimethyl ammonium chloride and/or wherein the one or more silicone dialkyl quaternary compounds include dimethyloctadecyl[3-(trimethoxysilyl)propyl]ammonium chloride and tetradecyldimethyl-(3-trimethoxysilyl)propyl ammonium chloride.
62. The kit according to any one of embodiments 47-51, 52, 56, 60 or 61, wherein the one or more quaternary compounds or silicon quaternary compounds are in an amount of about 0.01%, about 0.05%, about 0.075%, about 0.1%, about 0.2%, about 0.3%, about 0.4%, about 0.5%, about 0.6%, about 0.7%, about 0.8%, about 0.9%, about 1.0%, about 1.5%, about 2.0%, about 2.5%, about 3.0%, about 4.0%, about 5.0%, about 6.0%, about 7.0%, about 7.5%, about 8.0%, about 9.0%, about 10.0%, about 11.0%, about 12.0%, about 13.0%, about 14.0%, about 15.0%, about 16.0%, about 17.0%, 18.0%, about 19.0%, or about 20.0% by weight of the composition, or at least 0.01%, at least 0.05%, at least 0.075%, at least 0.1%, at least 0.25%, at least 0.5%, at least 0.75%, at least 1.0%, at least 1.5%, at least 2.0%, at least 2.5%, at least 3.0%, at least 4.0%, at least 5.0%, at least 6.0%, at least 7.0%, at least 7.5%, at least 8.0%, at least 9.0%, at least 10.0%, at least 11.0%, at least 12.0%, at least 13.0%, at least 14.0%, at least 15.0%, at least 16.0%, at least 17.0%, at least 18.0%, at least 19.0%, or at least 20.0% by weight of the composition by weight of the composition, or at most 0.01%, at most 0.05%, at most 0.075%, at most 0.1%, at most 0.25%, at most 0.5%, at most 0.75%, at most 1.0%, at most 1.5%, at most 2.0%, at most 2.5%, at most 3.0%, at most 4.0%, at most 5.0%, at most 6.0%, at most 7.5%, at most 8.0%, at most 9.0%, at most 10.0%, at most 11.0%, at most 12.0%, at most 13.0%, at most 14.0%, at most 15.0%, at most 16.0%, at most 17.0%, at most 18.0%, at most 19.0%, or at most 20.0% by weight of the composition by weight of the composition, or about 0.1% to about 0.5%, about 0.1% to about 0.75%, about 0.1% to about 1.0%, about 0.1% to about 1.5%, about 0.1% to about 2.0%, about 0.1% to about 2.5%, about 0.2% to about 0.5%, about 0.2% to about 0.75%, about 0.2% to about 1.0%, about 0.2% to about 1.5%, about 0.2% to about 2.0%, about 0.2% to about 2.5%, about 0.5% to about 1.0%, about 0.5% to about 1.5%, about 0.5% to about 2.0%, about 0.5% to about 2.5%, about 0.5% to about 3.0%, about 0.5% to about 4.0%, about 0.5% to about 5.0%, about 1.0% to about 2.0%, about 1.0% to about 3.0%, about 1.0% to about 4.0%, about 1.0% to about 5.0%, about 1.0% to about 6.0%, about 1.0% to about 7.0%, about 1.0% to about 8.0%, about 1.0% to about 9.0%, about 1.0% to about 10%, about 1.0% to about 11%, about 1.0% to about 12%, about 1.0% to about 13%, about 1.0% to about 14%, about 1.0% to about 15%, about 2.0% to about 3.0%, about 2.0% to about 4.0%, about 2.0% to about 5.0%, about 2.0% to about 6.0%, about 2.0% to about 7.0%, about 2.0% to about 8.0%, about 2.0% to about 9.0%, about 2.0% to about 10%, about 2.0% to about 11%, about 2.0% to about 12%, about 2.0% to about 13%, about 2.0% to about 14%, about 2.0% to about 15%, about 3.0% to about 4.0%, about 3.0% to about 5.0%, about 3.0% to about 6.0%, about 3.0% to about 7.0%, about 3.0% to about 8.0%, about 3.0% to about 9.0%, about 3.0% to about 10%, about 3.0% to about 11%, about 3.0% to about 12%, about 3.0% to about 13%, about 3.0% to about 14%, about 3.0% to about 15%, about 4.0% to about 5.0%, about 4.0% to about 6.0%, about 4.0% to about 7.0%, about 4.0% to about 8.0%, about 4.0% to about 9.0%, about 4.0% to about 10%, about 4.0% to about 11%, about 4.0% to about 12%, about 4.0% to about 13%, about 4.0% to about 14%, about 4.0% to about 15%, about 5.0% to about 6.0%, about 5.0% to about 7.0%, about 5.0% to about 8.0%, about 5.0% to about 9.0%, about 5.0% to about 10%, about 5.0% to about 11%, about 5.0% to about 12%, about 5.0% to about 13%, about 5.0% to about 14%, about 5.0% to about 15%, about 6.0% to about 7.0%, about 6.0% to about 8.0%, about 6.0% to about 9.0%, about 6.0% to about 10%, about 6.0% to about 11%, about 6.0% to about 12%, about 6.0% to about 13%, about 6.0% to about 14%, about 6.0% to about 15%, about 7.0% to about 8.0%, about 7.0% to about 9.0%, about 7.0% to about 10%, about 7.0% to about 11%, about 7.0% to about 12%, about 7.0% to about 13%, about 7.0% to about 14%, about 7.0% to about 15%, about 8.0% to about 9.0%, about 8.0% to about 10%, about 8.0% to about 11%, about 8.0% to about 12%, about 8.0% to about 13%, about 8.0% to about 14%, about 8.0% to about 15%, about 9.0% to about 10%, about 9.0% to about 11%, about 9.0% to about 12%, about 9.0% to about 13%, about 9.0% to about 14%, about 9.0% to about 15%, about 10% to about 11%, about 10% to about 12%, about 10% to about 13%, about 10% to about 14%, about 10% to about 15%, about 11% to about 12%, about 11% to about 13%, about 11% to about 14%, about 11% to about 15%, about 12% to about 13%, about 12% to about 14%, about 12% to about 15%, about 13% to about 14%, about 13% to about 15%, or 14% to about 15% by weight of the composition.
63. The kit according to any one of embodiments 47-51, 52, 56, 60 or 61, wherein the one or more quaternary compounds or silicon quaternary compounds is in an amount of 0.05 ppm, 0.10 ppm, 0.15 ppm, 0.20 ppm, 0.25 ppm, 0.30 ppm, 0.35 ppm, 0.40 ppm, 0.45 ppm, 0.50 ppm, 0.55 ppm, 0.60 ppm, 0.65 ppm, 0.70 ppm, 0.75 ppm, 0.80 ppm, 0.85 ppm, 0.90 ppm, 0.95 ppm, 1 ppm, 5 ppm, 10 ppm, 15 ppm, 20 ppm, 25 ppm, 30 ppm, 35 ppm, 40 ppm, 45 ppm, 50 ppm, 55 ppm, 60 ppm, 65 ppm, 70 ppm, 75 ppm, 80 ppm, 85 ppm, 90 ppm, 95 ppm, 100 ppm, 125 ppm, 150 ppm, 175 ppm, 200 ppm, 225 ppm, 250 ppm, 275 ppm, 300 ppm, 325 ppm, 350 ppm, 375 ppm, 400 ppm, 425 ppm, 450 ppm, 475 ppm, 500 ppm, 525 ppm, 550 ppm, 575 ppm, 600 ppm, 625 ppm, 650 ppm, 675 ppm, 700 ppm, 725 ppm, 750 ppm, 775 ppm, 800 ppm, 825 ppm, 850 ppm, 875 ppm, 900 ppm, 925 ppm, 950 ppm, 975 ppm, 1,000 ppm, 1,025 ppm, 1,050 ppm, 1075 ppm, 1,100 ppm, 1,125 ppm, 1,150 ppm, 1,175 ppm, 1,200 ppm, 1,225 ppm, 1,250 ppm, 1,275 ppm, 1,300 ppm, 1,325 ppm, 1,350 ppm, 1,375 ppm, 1,400 ppm, 1,425 ppm, 1,450 ppm, 1,475 ppm, or 1,500 ppm or at least 0.05 ppm, at least 0.10 ppm, at least 0.20 ppm, at least 0.30 ppm, at least 0.40 ppm, at least 0.50 ppm, at least 0.60 ppm, at least 0.70 ppm, at least 0.80 ppm, at least 0.90 ppm, at least 1 ppm, at least 10 ppm, at least 20 ppm, at least 30 ppm, at least 40 ppm, at least 50 ppm, at least 60 ppm, at least 70 ppm, at least 80 ppm, at least 90 ppm, at least 100 ppm, at least 125 ppm, at least 150 ppm, at least 175 ppm, at least 200 ppm, at least 225 ppm, at least 250 ppm, at least 275 ppm, at least 300 ppm, at least 325 ppm, at least 350 ppm, at least 375 ppm, at least 400 ppm, at least 425 ppm, at least 450 ppm, at least 475 ppm, at least 500 ppm, at least 525 ppm, at least 550 ppm, at least 575 ppm, at least 600 ppm, at least 625 ppm, at least 650 ppm, at least 675 ppm, at least 700 ppm, at least 725 ppm, at least 750 ppm, at least 775 ppm, at least 800 ppm, at least 825 ppm, at least 850 ppm, at least 875 ppm, at least 900 ppm, at least 925 ppm, at least 950 ppm, at least 975 ppm, at least 1,000 ppm, at least 1,025 ppm, at least 1,050 ppm, at least 1075 ppm, at least 1,100 ppm, at least 1,125 ppm, at least 1,150 ppm, at least 1,175 ppm, at least 1,200 ppm, at least 1,225 ppm, at least 1,250 ppm, at least 1,275 ppm, at least 1,300 ppm, at least 1,325 ppm, at least 1,350 ppm, at least 1,375 ppm, at least 1,400 ppm, at least 1,425 ppm, at least 1,450 ppm, at least 1,475 ppm, or at least 1,500 ppm or at most 0.05 ppm, at most 0.10 ppm, at most 0.20 ppm, at most 0.30 ppm, at most 0.40 ppm, at most 0.50 ppm, at most 0.60 ppm, at most 0.70 ppm, at most 0.80 ppm, at most 0.90 ppm, at most 1 ppm, at most 10 ppm, at most 20 ppm, at most 30 ppm, at most 40 ppm, at most 50 ppm, at most 60 ppm, at most 70 ppm, at most 80 ppm, at most 90 ppm, at most 100 ppm, at most 125 ppm, at most 150 ppm, at most 175 ppm, at most 200 ppm, at most 225 ppm, at most 250 ppm, at most 275 ppm, at most 300 ppm, at most 325 ppm, at most 350 ppm, at most 375 ppm, at most 400 ppm, at most 425 ppm, at most 450 ppm, at most 475 ppm, at most 500 ppm, at most 525 ppm, at most 550 ppm, at most 575 ppm, at most 600 ppm, at most 625 ppm, at most 650 ppm, at most 675 ppm, at most 700 ppm, at most 725 ppm, at most 750 ppm, at most 775 ppm, at most 800 ppm, at most 825 ppm, at most 850 ppm, at most 875 ppm, at most 900 ppm, at most 925 ppm, at most 950 ppm, at most 975 ppm, at most 1,000 ppm, at most 1,025 ppm, at most 1,050 ppm, at most 1075 ppm, at most 1,100 ppm, at most 1,125 ppm, at most 1,150 ppm, at most 1,175 ppm, at most 1,200 ppm, at most 1,225 ppm, at most 1,250 ppm, at most 1,275 ppm, at most 1,300 ppm, at most 1,325 ppm, at most 1,350 ppm, at most 1,375 ppm, at most 1,400 ppm, at most 1,425 ppm, at most 1,450 ppm, at most 1,475 ppm, or at most 1,500 ppm or about 0.5 ppm to about 20 ppm, about 0.5 ppm to about 25 ppm, about 0.5 ppm to about 30 ppm, about 0.5 ppm to about 35 ppm, about 0.5 ppm to about 40 ppm, about 0.5 ppm to about 45 ppm, about 0.5 ppm to about 50 ppm, about 0.5 ppm to about 55 ppm, about 0.5 ppm to about 60 ppm, about 0.5 ppm to about 65 ppm, about 0.5 ppm to about 70 ppm, about 0.5 ppm to about 75 ppm, about 0.5 ppm to about 80 ppm, about 0.5 ppm to about 85 ppm, about 0.5 ppm to about 90 ppm, about 0.5 ppm to about 95 ppm, about 0.5 ppm to about 100 ppm, about 0.75 ppm to about 20 ppm, about 0.75 ppm to about 25 ppm, about 0.75 ppm to about 30 ppm, about 0.75 ppm to about 35 ppm, about 0.75 ppm to about 40 ppm, about 0.75 ppm to about 45 ppm, about 0.75 ppm to about 50 ppm, about 0.75 ppm to about 55 ppm, about 0.75 ppm to about 60 ppm, about 0.75 ppm to about 65 ppm, about 0.75 ppm to about 70 ppm, about 0.75 ppm to about 75 ppm, about 0.75 ppm to about 80 ppm, about 0.75 ppm to about 85 ppm, about 0.75 ppm to about 90 ppm, about 0.75 ppm to about 95 ppm, about 0.75 ppm to about 100 ppm, about 1 ppm to about 20 ppm, about 1 ppm to about 25 ppm, about 1 ppm to about 30 ppm, about 1 ppm to about 35 ppm, about 1 ppm to about 40 ppm, about 1 ppm to about 45 ppm, about 1 ppm to about 50 ppm, about 1 ppm to about 55 ppm, about 1 ppm to about 60 ppm, about 1 ppm to about 65 ppm, about 1 ppm to about 70 ppm, about 1 ppm to about 75 ppm, about 1 ppm to about 80 ppm, about 1 ppm to about 85 ppm, about 1 ppm to about 90 ppm, about 1 ppm to about 95 ppm, about 1 ppm to about 100 ppm, about 5 ppm to about 20 ppm, about 5 ppm to about 25 ppm, about 5 ppm to about 30 ppm, about 5 ppm to about 35 ppm, about 5 ppm to about 40 ppm, about 5 ppm to about 45 ppm, about 5 ppm to about 50 ppm, about 5 ppm to about 55 ppm, about 5 ppm to about 60 ppm, about 5 ppm to about 65 ppm, about 5 ppm to about 70 ppm, about 5 ppm to about 75 ppm, about 5 ppm to about 80 ppm, about 5 ppm to about 85 ppm, about 5 ppm to about 90 ppm, about 5 ppm to about 95 ppm, about 5 ppm to about 100 ppm, about 10 ppm to about 20 ppm, about 10 ppm to about 25 ppm, about 10 ppm to about 30 ppm, about 10 ppm to about 35 ppm, about 10 ppm to about 40 ppm, about 10 ppm to about 45 ppm, about 10 ppm to about 50 ppm, about 10 ppm to about 55 ppm, about 10 ppm to about 60 ppm, about 10 ppm to about 65 ppm, about 10 ppm to about 70 ppm, about 10 ppm to about 75 ppm, about 10 ppm to about 80 ppm, about 10 ppm to about 85 ppm, about 10 ppm to about 90 ppm, about 10 ppm to about 95 ppm, or about 10 ppm to about 100 ppm or about 1 ppm to about 25 ppm, about 1 ppm to about 50 ppm, about 1 ppm to about 75 ppm, about 1 ppm to about 100 ppm, about 1 ppm to about 125 ppm, about 1 ppm to about 150 ppm, about 1 ppm to about 175 ppm, about 1 ppm to about 200 ppm, about 1 ppm to about 225 ppm, about 1 ppm to about 250 ppm, about 1 ppm to about 275 ppm, about 1 ppm to about 300 ppm, about 1 ppm to about 325 ppm, about 1 ppm to about 350 ppm, about 1 ppm to about 375 ppm, about 1 ppm to about 400 ppm, about 1 ppm to about 425 ppm, about 1 ppm to about 450 ppm, about 1 ppm to about 475 ppm, about 1 ppm to about 500 ppm, about 5 ppm to about 25 ppm, about 5 ppm to about 50 ppm, about 5 ppm to about 75 ppm, about 5 ppm to about 100 ppm, about 5 ppm to about 125 ppm, about 5 ppm to about 150 ppm, about 5 ppm to about 175 ppm, about 5 ppm to about 200 ppm, about 5 ppm to about 225 ppm, about 5 ppm to about 250 ppm, about 5 ppm to about 275 ppm, about 1 ppm to about 300 ppm, about 5 ppm to about 325 ppm, about 5 ppm to about 350 ppm, about 5 ppm to about 375 ppm, about 5 ppm to about 400 ppm, about 5 ppm to about 425 ppm, about 5 ppm to about 450 ppm, about 5 ppm to about 475 ppm, about 5 ppm to about 500 ppm, about 10 ppm to about 25 ppm, about 10 ppm to about 50 ppm, about 10 ppm to about 75 ppm, about 10 ppm to about 100 ppm, about 10 ppm to about 125 ppm, about 10 ppm to about 150 ppm, about 10 ppm to about 175 ppm, about 10 ppm to about 200 ppm, about 10 ppm to about 225 ppm, about 10 ppm to about 250 ppm, about 10 ppm to about 275 ppm, about 10 ppm to about 300 ppm, about 10 ppm to about 325 ppm, about 10 ppm to about 350 ppm, about 10 ppm to about 375 ppm, about 10 ppm to about 400 ppm, about 10 ppm to about 425 ppm, about 10 ppm to about 450 ppm, about 10 ppm to about 475 ppm, about 10 ppm to about 500 ppm, about 25 ppm to about 50 ppm, about 25 ppm to about 75 ppm, about 25 ppm to about 100 ppm, about 25 ppm to about 125 ppm, about 25 ppm to about 150 ppm, about 25 ppm to about 175 ppm, about 25 ppm to about 200 ppm, about 25 ppm to about 225 ppm, about 25 ppm to about 250 ppm, about 25 ppm to about 275 ppm, about 25 ppm to about 300 ppm, about 25 ppm to about 325 ppm, about 25 ppm to about 350 ppm, about 25 ppm to about 375 ppm, about 25 ppm to about 400 ppm, about 25 ppm to about 425 ppm, about 25 ppm to about 450 ppm, about 25 ppm to about 475 ppm, about 25 ppm to about 500 ppm, about 50 ppm to about 75 ppm, about 50 ppm to about 100 ppm, about 50 ppm to about 125 ppm, about 50 ppm to about 150 ppm, about 50 ppm to about 175 ppm, about 50 ppm to about 200 ppm, about 50 ppm to about 225 ppm, about 50 ppm to about 250 ppm, about 50 ppm to about 275 ppm, about 50 ppm to about 300 ppm, about 50 ppm to about 325 ppm, about 50 ppm to about 350 ppm, about 50 ppm to about 375 ppm, about 50 ppm to about 400 ppm, about 50 ppm to about 425 ppm, about 50 ppm to about 450 ppm, about 50 ppm to about 475 ppm, about 50 ppm to about 500 ppm, about 75 ppm to about 100 ppm, about 75 ppm to about 125 ppm, about 75 ppm to about 150 ppm, about 75 ppm to about 175 ppm, about 75 ppm to about 200 ppm, about 75 ppm to about 225 ppm, about 75 ppm to about 250 ppm, about 75 ppm to about 275 ppm, about 75 ppm to about 300 ppm, about 75 ppm to about 325 ppm, about 75 ppm to about 350 ppm, about 75 ppm to about 375 ppm, about 75 ppm to about 400 ppm, about 75 ppm to about 425 ppm, about 75 ppm to about 450 ppm, about 75 ppm to about 475 ppm, about 75 ppm to about 500 ppm, about 100 ppm to about 125 ppm, about 100 ppm to about 150 ppm, about 100 ppm to about 175 ppm, about 100 ppm to about 200 ppm, about 100 ppm to about 225 ppm, about 100 ppm to about 250 ppm, about 100 ppm to about 275 ppm, about 100 ppm to about 300 ppm, about 100 ppm to about 325 ppm, about 100 ppm to about 350 ppm, about 100 ppm to about 375 ppm, about 100 ppm to about 400 ppm, about 150 ppm to about 175 ppm, about 150 ppm to about 200 ppm, about 150 ppm to about 225 ppm, about 150 ppm to about 250 ppm, about 150 ppm to about 275 ppm, about 150 ppm to about 300 ppm, about 150 ppm to about 325 ppm, about 150 ppm to about 350 ppm, about 150 ppm to about 375 ppm, about 150 ppm to about 400 ppm, about 200 ppm to about 225 ppm, about 200 ppm to about 250 ppm, about 200 ppm to about 275 ppm, about 200 ppm to about 300 ppm, about 200 ppm to about 325 ppm, about 200 ppm to about 350 ppm, about 200 ppm to about 375 ppm, about 200 ppm to about 400 ppm, about 200 ppm to about 425 ppm, about 200 ppm to about 450 ppm, about 200 ppm to about 475 ppm, about 200 ppm to about 500 ppm, about 200 ppm to about 525 ppm, about 200 ppm to about 550 ppm, about 200 ppm to about 575 ppm, about 200 ppm to about 600 ppm, about 250 ppm to about 275 ppm, about 250 ppm to about 300 ppm, about 250 ppm to about 325 ppm, about 250 ppm to about 350 ppm, about 250 ppm to about 375 ppm, about 250 ppm to about 400 ppm, about 250 ppm to about 425 ppm, about 250 ppm to about 450 ppm, about 250 ppm to about 475 ppm, about 250 ppm to about 500 ppm, about 250 ppm to about 525 ppm, about 250 ppm to about 550 ppm, about 250 ppm to about 575 ppm, about 250 ppm to about 600 ppm, about 300 ppm to about 325 ppm, about 300 ppm to about 350 ppm, about 300 ppm to about 375 ppm, about 300 ppm to about 400 ppm, about 300 ppm to about 425 ppm, about 300 ppm to about 450 ppm, about 300 ppm to about 475 ppm, about 300 ppm to about 500 ppm, about 350 ppm to about 375 ppm, about 350 ppm to about 400 ppm, about 350 ppm to about 425 ppm, about 350 ppm to about 450 ppm, about 350 ppm to about 475 ppm, about 350 ppm to about 500 ppm, about 400 ppm to about 425 ppm, about 400 ppm to about 450 ppm, about 400 ppm to about 475 ppm, about 400 ppm to about 500 ppm, about 450 ppm to about 475 ppm, or about 450 ppm to about 500 ppm, about 400 ppm to about 600 ppm, about 400 ppm to about 700 ppm, about 400 ppm to about 800 ppm, about 400 ppm to about 900 ppm, about 400 ppm to about 1,000 ppm, about 400 ppm to about 1,100 ppm, about 400 ppm to about 1,200 ppm, about 400 ppm to about 1,300 ppm, about 400 ppm to about 1,400 ppm, about 400 ppm to about 1,500 ppm, about 500 ppm to about 600 ppm, about 500 ppm to about 700 ppm, about 500 ppm to about 800 ppm, about 500 ppm to about 900 ppm, about 500 ppm to about 1,000 ppm, about 500 ppm to about 1,100 ppm, about 500 ppm to about 1,200 ppm, about 500 ppm to about 1,300 ppm, about 500 ppm to about 1,400 ppm, about 500 ppm to about 1,500 ppm, about 600 ppm to about 700 ppm, about 600 ppm to about 800 ppm, about 600 ppm to about 900 ppm, about 600 ppm to about 1,000 ppm, about 600 ppm to about 1,100 ppm, about 600 ppm to about 1,200 ppm, about 600 ppm to about 1,300 ppm, about 600 ppm to about 1,400 ppm, about 600 ppm to about 1,500 ppm, about 700 ppm to about 800 ppm, about 700 ppm to about 900 ppm, about 700 ppm to about 1,000 ppm, about 700 ppm to about 1,100 ppm, about 700 ppm to about 1,200 ppm, about 700 ppm to about 1,300 ppm, about 700 ppm to about 1,400 ppm, about 700 ppm to about 1,500 ppm, about 800 ppm to about 900 ppm, about 800 ppm to about 1,000 ppm, about 800 ppm to about 1,100 ppm, about 800 ppm to about 1,200 ppm, about 800 ppm to about 1,300 ppm, about 800 ppm to about 1,400 ppm, about 800 ppm to about 1,500 ppm, about 900 ppm to about 1,000 ppm, about 900 ppm to about 1,100 ppm, about 900 ppm to about 1,200 ppm, about 900 ppm to about 1,300 ppm, about 900 ppm to about 1,400 ppm, about 900 ppm to about 1,500 ppm, about 1,000 ppm to about 1,100 ppm, about 1,000 ppm to about 1,200 ppm, about 1,000 ppm to about 1,300 ppm, about 1,000 ppm to about 1,400 ppm, about 1,000 ppm to about 1,500 ppm, about 1,100 ppm to about 1,200 ppm, about 1,100 ppm to about 1,300 ppm, about 1,100 ppm to about 1,400 ppm, about 1,100 ppm to about 1,500 ppm, about 1,200 ppm to about 1,300 ppm, about 1,200 ppm to about 1,400 ppm, about 1,200 ppm to about 1,500 ppm, about 1,300 ppm to about 1,400 ppm, about 1,300 ppm to about 1,500 ppm, or about 1,400 ppm to about 1,500 ppm.
64. The kit according to any one of embodiments 47, 48, 51, 53 or 57, wherein the one or more guanide-containing compounds comprise, consist essentially of, or consist of a biguanide, a biguanide-containing compound, a biguanidine, a biguanidine-containing compound, a triguanide, a triguanide-containing compound, or any combination thereof.
65. The kit according to embodiment 64, wherein the one or more biguanide-containing compounds include a polyhexamethylene biguanide (PHMB), a polyaminopropyl biguanide (PAPB), a 1,1′-(1,6-Hexanediyl)bis{2-[N′-(2-ethylhexyl)carbamimidoyl]guanidine} (alexidine), a chlorhexidine, a chlorhexidine gluconate, or any combination thereof.
66. The kit according to any one of embodiments 47, 48, 51, 53, 57, 64 or 65, wherein the one or more guanide-containing compounds are in an amount of about 0.01%, about 0.05%, about 0.075%, about 0.1%, about 0.2%, about 0.3%, about 0.4%, about 0.5%, about 0.6%, about 0.7%, about 0.8%, about 0.9%, about 1.0%, about 1.5%, about 2.0%, about 2.5%, about 3.0%, about 4.0%, about 5.0%, about 6.0%, about 7.0%, about 7.5%, about 8.0%, about 9.0%, about 10.0%. about 11%, about 12%, about 13%, about 14%, about 15%, about 20%, about 25%, or about 30% by weight of the composition, or at least 0.01%, at least 0.05%, at least 0.075%, at least 0.1%, at least 0.25%, at least 0.5%, at least 0.75%, at least 1.0%, at least 1.5%, at least 2.0%, at least 2.5%, at least 3.0%, at least 4.0%, at least 5.0%, at least 6.0%, at least 7.0%, at least 7.5%, at least 8.0%, at least 9.0%, at least 10.0%, at least 11%, at least 12%, at least 13%, at least 14%, at least 15%, at least 20%, at least 25%, or at least 30% by weight of the composition, or at most 0.01%, at most 0.05%, at most 0.075%, at most 0.1%, at most 0.25%, at most 0.5%, at most 0.75%, at most 1.0%, at most 1.5%, at most 2.0%, at most 2.5%, at most 3.0%, at most 4.0%, at most 5.0%, at most 6.0%, at most 7.5%, at most 8.0%, at most 9.0%, at most 10.0%, at most 11%, at most 12%, at most 13%, at most 14%, at most 15%, at most 20%, at most 25%, or at most 30% by weight of the composition, or about 0.1% to about 0.5%, about 0.1% to about 0.75%, about 0.1% to about 1.0%, about 0.1% to about 1.5%, about 0.1% to about 2.0%, about 0.1% to about 2.5%, about 0.1% to about 3.0%, about 0.1% to about 3.5%, about 0.1% to about 4.0%, about 0.1% to about 4.5%, about 0.1% to about 5.0%, about 0.2% to about 0.5%, about 0.2% to about 0.75%, about 0.2% to about 1.0%, about 0.2% to about 1.5%, about 0.2% to about 2.0%, about 0.2% to about 2.5%, about 0.2% to about 3.0%, about 0.2% to about 3.5%, about 0.2% to about 4.0%, about 0.2% to about 4.5%, about 0.2% to about 5.0%, about 0.5% to about 1.0%, about 0.5% to about 1.5%, about 0.5% to about 2.0%, about 0.5% to about 2.5%, about 0.5% to about 3.0%, about 0.5% to about 4.0%, about 0.5% to about 5.0%, about 0.5% to about 6.0%, about 0.5% to about 7.0%, about 0.5% to about 8.0%, about 0.5% to about 9.0%, about 0.5% to about 10.0%, about 1.0% to about 2.5%, about 1.0% to about 3.0%, about 1.0% to about 4.0%, about 1.0% to about 5.0%, about 1.0% to about 6.0%, about 1.0% to about 7.0%, about 1.0% to about 7.5%, about 1.0% to about 8.0%, about 1.0% to about 9.0%, about 1.0% to about 10.0%, about 1.0% to about 15.0%, about 1.0% to about 20.0%, about 1.0% to about 25.0%, about 1.0% to about 30.0%, about 2.0% to about 2.5%, about 2.0% to about 3.0%, about 2.0% to about 4.0%, about 2.0% to about 5.0%, about 2.0% to about 6.0%, about 2.0% to about 7.0%, about 2.0% to about 7.5%, about 2.0% to about 8.0%, about 2.0% to about 9.0%, about 2.0% to about 10.0%, about 2.0% to about 15.0%, about 2.0% to about 20.0%, about 2.0% to about 25.0%, about 2.0% to about 30.0%, about 5.0% to about 6.0%, about 5.0% to about 7.0%, about 5.0% to about 7.5%, about 5.0% to about 8.0%, about 5.0% to about 9.0%, about 5.0% to about 10.0%, about 5.0% to about 11.0%, about 5.0% to about 12.0%, about 5.0% to about 13.0%, about 5.0% to about 14.0%, about 5.0% to about 15.0%, about 5.0% to about 20.0%, about 5.0% to about 25.0%, about 5.0% to about 30.0%, about 10.0% to about 15.0%, about 10.0% to about 20.0%, about 10.0% to about 25.0%, about 10.0% to about 30.0%, about 15.0% to about 20.0%, about 15.0% to about 25.0%, about 15.0% to about 30.0%, about 20.0% to about 25.0%, about 20.0% to about 30.0%, or about 20.0% to about 30.0%, by weight of the composition.
67. The kit according to any one of embodiments 47, 48, 51, 53 or 57, wherein the one or more alcohols include methanol, ethanol, propanol, isopropanol, butanol, pentanol, and/or 1-hexadecanol.
68. The kit according to any one of embodiments 47, 48, 51, 53, 57 or 67, wherein the one or more alcohols are in an amount of about 0.5%, about 1%, about 2%, about 3%, about 4%, about 5%, about 6%, about 7%, about 8%, about 9%, about 10%, about 11%, about 12%, about 13%, about 14%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, or about 99%, by weight of the composition, or at least 0.5%, at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99%, by weight of the composition, or at most 0.5%, at most 1%, at most 2%, at most 3%, at most 4%, at most 5%, at most 6%, at most 7%, at most 8%, at most 9%, at most 10%, at most 11%, at most 12%, at most 13%, at most 14%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95%, at most 96%, at most 97%, at most 98%, or at most 99%, by weight of the composition, about 0.05% to about 0.75%, about 0.05% to about 1.0%, about 0.05% to about 1.5%, about 0.05% to about 2.0%, about 0.05% to about 2.5%, about 0.1% to about 0.5%, about 0.1% to about 0.75%, about 0.1% to about 1.0%, about 0.1% to about 1.5%, about 0.1% to about 2.0%, about 0.1% to about 2.5%, about 0.1% to about 3.0%, about 0.1% to about 4.0%, about 0.1% to about 5.0%, about 0.2% to about 0.5%, about 0.2% to about 0.75%, about 0.2% to about 1.0%, about 0.2% to about 1.5%, about 0.2% to about 2.0%, about 0.2% to about 2.5%, about 0.2% to about 3.0%, about 0.2% to about 4.0%, about 0.2% to about 5.0%, about 0.5% to about 1.0%, about 0.5% to about 1.5%, about 0.5% to about 2.0%, about 0.5% to about 2.5%, about 0.5% to about 3.0%, about 0.5% to about 4.0%, about 0.5% to about 5.0%, about 0.5% to about 6.0%, about 0.5% to about 7.0%, about 0.5% to about 8.0%, about 0.5% to about 9.0%, about 0.5% to about 10.0%, about 1.0% to about 2.5%, about 1.0% to about 3.0%, about 1.0% to about 4.0%, about 1.0% to about 5.0%, about 1.0% to about 6.0%, about 1.0% to about 7.0%, about 1.0% to about 7.5%, about 1.0% to about 8.0%, about 1.0% to about 9.0%, about 1.0% to about 10.0%, about 2.0% to about 2.5%, about 2.0% to about 3.0%, about 2.0% to about 4.0%, about 2.0% to about 5.0%, about 2.0% to about 6.0%, about 2.0% to about 7.0%, about 2.0% to about 7.5%, about 2.0% to about 8.0%, about 2.0% to about 9.0%, about 2.0% to about 10.0%, about 5.0% to about 6.0%, about 5.0% to about 7.0%, about 5.0% to about 7.5%, about 5.0% to about 8.0%, about 5.0% to about 9.0%, about 5.0% to about 10.0%, about 5.0% to about 11.0%, about 5.0% to about 12.0%, about 5.0% to about 13.0%, about 5.0% to about 14.0% or about 5.0% to about 15.0% by weight of the composition, or about 10% to about 20%, about 10% to about 30%, about 10% to about 40%, about 10% to about 50%, about 10% to about 60%, about 10% to about 70%, about 10% to about 80%, about 10% to about 90%, about 10% to about 95%, about 10% to about 97%, about 10% to about 99%, about 20% to about 30%, about 20% to about 40%, about 20% to about 50%, about 20% to about 60%, about 20% to about 70%, about 20% to about 80%, about 20% to about 90%, about 20% to about 95%, about 20% to about 97%, about 20% to about 99%, about 30% to about 40%, about 30% to about 50%, about 30% to about 60%, about 30% to about 70%, about 30% to about 80%, about 30% to about 90%, about 30% to about 95%, about 30% to about 97%, about 30% to about 99%, about 40% to about 50%, about 40% to about 60%, about 40% to about 70%, about 40% to about 80%, about 40% to about 90%, about 40% to about 95%, about 40% to about 97%, about 40% to about 99%, about 50% to about 60%, about 50% to about 70%, about 50% to about 80%, about 50% to about 90%, about 50% to about 95%, about 50% to about 97%, about 50% to about 99%, about 60% to about 70%, about 60% to about 80%, about 60% to about 90%, about 60% to about 95%, about 60% to about 97%, about 60% to about 99%, about 70% to about 80%, about 70% to about 90%, about 70% to about 95%, about 70% to about 97%, about 70% to about 99%, about 80% to about 90%, about 80% to about 95%, about 80% to about 97%, about 80% to about 99%, about 90% to about 95%, about 90% to about 97%, about 90% to about 99%, by weight of the composition.
69. The kit according to any one of embodiments 47, 48, 54, 56, or 57, wherein the one or more metallic particles include a metal acetate particle, a metal chloride particle, a metal nitrate particle, or a metal oxide particle, or any combination thereof.
70. The kit according to any one of embodiments 47, 48, 54, 56, 57 or 69, wherein the one or more metallic particles are in an amount of 0.05 ppm, 0.10 ppm, 0.15 ppm, 0.20 ppm, 0.25 ppm, 0.30 ppm, 0.35 ppm, 0.40 ppm, 0.45 ppm, 0.50 ppm, 0.55 ppm, 0.60 ppm, 0.65 ppm, 0.70 ppm, 0.75 ppm, 0.80 ppm, 0.85 ppm, 0.90 ppm, 0.95 ppm, 1 ppm, 5 ppm, 10 ppm, 15 ppm, 20 ppm, 25 ppm, 30 ppm, 35 ppm, 40 ppm, 45 ppm, 50 ppm, 55 ppm, 60 ppm, 65 ppm, 70 ppm, 75 ppm, 80 ppm, 85 ppm, 90 ppm, 95 ppm, 10⁰ ppm, 125 ppm, 150 ppm, 175 ppm, 200 ppm, 225 ppm, 250 ppm, 275 ppm, 300 ppm, 325 ppm, 350 ppm, 375 ppm, 400 ppm, 425 ppm, 450 ppm, 475 ppm, 500 ppm, 525 ppm, 550 ppm, 575 ppm, 600 ppm, 625 ppm, 650 ppm, 675 ppm, 700 ppm, 725 ppm, 750 ppm, 775 ppm, 800 ppm, 825 ppm, 850 ppm, 875 ppm, 900 ppm, 925 ppm, 950 ppm, 975 ppm, 1,000 ppm, 1,025 ppm, 1,050 ppm, 1075 ppm, 1,100 ppm, 1,125 ppm, 1,150 ppm, 1,175 ppm, 1,200 ppm, 1,225 ppm, 1,250 ppm, 1,275 ppm, 1,300 ppm, 1,325 ppm, 1,350 ppm, 1,375 ppm, 1,400 ppm, 1,425 ppm, 1,450 ppm, 1,475 ppm, 1,500 ppm, 1,600 ppm, 1,700 ppm, 1,800 ppm, 1,900 ppm, or 2,000 ppm or at least 0.05 ppm, at least 0.10 ppm, at least 0.20 ppm, at least 0.30 ppm, at least 0.40 ppm, at least 0.50 ppm, at least 0.60 ppm, at least 0.70 ppm, at least 0.80 ppm, at least 0.90 ppm, at least 1 ppm, at least 10 ppm, at least 20 ppm, at least 30 ppm, at least 40 ppm, at least 50 ppm, at least 60 ppm, at least 70 ppm, at least 80 ppm, at least 90 ppm, at least 100 ppm, at least 125 ppm, at least 150 ppm, at least 175 ppm, at least 200 ppm, at least 225 ppm, at least 250 ppm, at least 275 ppm, at least 300 ppm, at least 325 ppm, at least 350 ppm, at least 375 ppm, at least 400 ppm, at least 425 ppm, at least 450 ppm, at least 475 ppm, at least 500 ppm, at least 525 ppm, at least 550 ppm, at least 575 ppm, at least 600 ppm, at least 625 ppm, at least 650 ppm, at least 675 ppm, at least 700 ppm, at least 725 ppm, at least 750 ppm, at least 775 ppm, at least 800 ppm, at least 825 ppm, at least 850 ppm, at least 875 ppm, at least 900 ppm, at least 925 ppm, at least 950 ppm, at least 975 ppm, at least 1,000 ppm, at least 1,025 ppm, at least 1,050 ppm, at least 1075 ppm, at least 1,100 ppm, at least 1,125 ppm, at least 1,150 ppm, at least 1,175 ppm, at least 1,200 ppm, at least 1,225 ppm, at least 1,250 ppm, at least 1,275 ppm, at least 1,300 ppm, at least 1,325 ppm, at least 1,350 ppm, at least 1,375 ppm, at least 1,400 ppm, at least 1,425 ppm, at least 1,450 ppm, at least 1,475 ppm, at least 1,500 ppm, at least 1,600 ppm, at least 1,700 ppm, at least 1,800 ppm, at least 1,900 ppm, or at least 2,000 ppm or at most 0.05 ppm, at most 0.10 ppm, at most 0.20 ppm, at most 0.30 ppm, at most 0.40 ppm, at most 0.50 ppm, at most 0.60 ppm, at most 0.70 ppm, at most 0.80 ppm, at most 0.90 ppm, at most 1 ppm, at most 10 ppm, at most 20 ppm, at most 30 ppm, at most 40 ppm, at most 50 ppm, at most 60 ppm, at most 70 ppm, at most 80 ppm, at most 90 ppm, at most 100 ppm, at most 125 ppm, at most 150 ppm, at most 175 ppm, at most 200 ppm, at most 225 ppm, at most 250 ppm, at most 275 ppm, at most 300 ppm, at most 325 ppm, at most 350 ppm, at most 375 ppm, at most 400 ppm, at most 425 ppm, at most 450 ppm, at most 475 ppm, at most 500 ppm, at most 525 ppm, at most 550 ppm, at most 575 ppm, at most 600 ppm, at most 625 ppm, at most 650 ppm, at most 675 ppm, at most 700 ppm, at most 725 ppm, at most 750 ppm, at most 775 ppm, at most 800 ppm, at most 825 ppm, at most 850 ppm, at most 875 ppm, at most 900 ppm, at most 925 ppm, at most 950 ppm, at most 975 ppm, at most 1,000 ppm, at most 1,025 ppm, at most 1,050 ppm, at most 1075 ppm, at most 1,100 ppm, at most 1,125 ppm, at most 1,150 ppm, at most 1,175 ppm, at most 1,200 ppm, at most 1,225 ppm, at most 1,250 ppm, at most 1,275 ppm, at most 1,300 ppm, at most 1,325 ppm, at most 1,350 ppm, at most 1,375 ppm, at most 1,400 ppm, at most 1,425 ppm, at most 1,450 ppm, at most 1,475 ppm, at most 1,500 ppm, at most 1,600 ppm, at most 1,700 ppm, at most 1,800 ppm, at most 1,900 ppm, or at most 2,000 ppm or about 0.5 ppm to about 20 ppm, about 0.5 ppm to about 25 ppm, about 0.5 ppm to about 30 ppm, about 0.5 ppm to about 35 ppm, about 0.5 ppm to about 40 ppm, about 0.5 ppm to about 45 ppm, about 0.5 ppm to about 50 ppm, about 0.5 ppm to about 55 ppm, about 0.5 ppm to about 60 ppm, about 0.5 ppm to about 65 ppm, about 0.5 ppm to about 70 ppm, about 0.5 ppm to about 75 ppm, about 0.5 ppm to about 80 ppm, about 0.5 ppm to about 85 ppm, about 0.5 ppm to about 90 ppm, about 0.5 ppm to about 95 ppm, about 0.5 ppm to about 100 ppm, about 0.75 ppm to about 20 ppm, about 0.75 ppm to about 25 ppm, about 0.75 ppm to about 30 ppm, about 0.75 ppm to about 35 ppm, about 0.75 ppm to about 40 ppm, about 0.75 ppm to about 45 ppm, about 0.75 ppm to about 50 ppm, about 0.75 ppm to about 55 ppm, about 0.75 ppm to about 60 ppm, about 0.75 ppm to about 65 ppm, about 0.75 ppm to about 70 ppm, about 0.75 ppm to about 75 ppm, about 0.75 ppm to about 80 ppm, about 0.75 ppm to about 85 ppm, about 0.75 ppm to about 90 ppm, about 0.75 ppm to about 95 ppm, about 0.75 ppm to about 100 ppm, about 1 ppm to about 20 ppm, about 1 ppm to about 25 ppm, about 1 ppm to about 30 ppm, about 1 ppm to about 35 ppm, about 1 ppm to about 40 ppm, about 1 ppm to about 45 ppm, about 1 ppm to about 50 ppm, about 1 ppm to about 55 ppm, about 1 ppm to about 60 ppm, about 1 ppm to about 65 ppm, about 1 ppm to about 70 ppm, about 1 ppm to about 75 ppm, about 1 ppm to about 80 ppm, about 1 ppm to about 85 ppm, about 1 ppm to about 90 ppm, about 1 ppm to about 95 ppm, about 1 ppm to about 100 ppm, about 5 ppm to about 20 ppm, about 5 ppm to about 25 ppm, about 5 ppm to about 30 ppm, about 5 ppm to about 35 ppm, about 5 ppm to about 40 ppm, about 5 ppm to about 45 ppm, about 5 ppm to about 50 ppm, about 5 ppm to about 55 ppm, about 5 ppm to about 60 ppm, about 5 ppm to about 65 ppm, about 5 ppm to about 70 ppm, about 5 ppm to about 75 ppm, about 5 ppm to about 80 ppm, about 5 ppm to about 85 ppm, about 5 ppm to about 90 ppm, about 5 ppm to about 95 ppm, about 5 ppm to about 100 ppm, about 10 ppm to about 20 ppm, about 10 ppm to about 25 ppm, about 10 ppm to about 30 ppm, about 10 ppm to about 35 ppm, about 10 ppm to about 40 ppm, about 10 ppm to about 45 ppm, about 10 ppm to about 50 ppm, about 10 ppm to about 55 ppm, about 10 ppm to about 60 ppm, about 10 ppm to about 65 ppm, about 10 ppm to about 70 ppm, about 10 ppm to about 75 ppm, about 10 ppm to about 80 ppm, about 10 ppm to about 85 ppm, about 10 ppm to about 90 ppm, about 10 ppm to about 95 ppm, or about 10 ppm to about 100 ppm, about 1 ppm to about 25 ppm, about 1 ppm to about 50 ppm, about 1 ppm to about 75 ppm, about 1 ppm to about 100 ppm, about 1 ppm to about 125 ppm, about 1 ppm to about 150 ppm, about 1 ppm to about 175 ppm, about 1 ppm to about 200 ppm, about 1 ppm to about 225 ppm, about 1 ppm to about 250 ppm, about 1 ppm to about 275 ppm, about 1 ppm to about 300 ppm, about 1 ppm to about 325 ppm, about 1 ppm to about 350 ppm, about 1 ppm to about 375 ppm, about 1 ppm to about 400 ppm, about 1 ppm to about 425 ppm, about 1 ppm to about 450 ppm, about 1 ppm to about 475 ppm, about 1 ppm to about 500 ppm, about 5 ppm to about 25 ppm, about 5 ppm to about 50 ppm, about 5 ppm to about 75 ppm, about 5 ppm to about 10⁰ ppm, about 5 ppm to about 125 ppm, about 5 ppm to about 150 ppm, about 5 ppm to about 175 ppm, about 5 ppm to about 200 ppm, about 5 ppm to about 225 ppm, about 5 ppm to about 250 ppm, about 5 ppm to about 275 ppm, about 1 ppm to about 300 ppm, about 5 ppm to about 325 ppm, about 5 ppm to about 350 ppm, about 5 ppm to about 375 ppm, about 5 ppm to about 400 ppm, about 5 ppm to about 425 ppm, about 5 ppm to about 450 ppm, about 5 ppm to about 475 ppm, about 5 ppm to about 500 ppm, about 10 ppm to about 25 ppm, about 10 ppm to about 50 ppm, about 10 ppm to about 75 ppm, about 10 ppm to about 100 ppm, about 10 ppm to about 125 ppm, about 10 ppm to about 150 ppm, about 10 ppm to about 175 ppm, about 10 ppm to about 200 ppm, about 10 ppm to about 225 ppm, about 10 ppm to about 250 ppm, about 10 ppm to about 275 ppm, about 10 ppm to about 300 ppm, about 10 ppm to about 325 ppm, about 10 ppm to about 350 ppm, about 10 ppm to about 375 ppm, about 10 ppm to about 400 ppm, about 10 ppm to about 425 ppm, about 10 ppm to about 450 ppm, about 10 ppm to about 475 ppm, about 10 ppm to about 500 ppm, about 25 ppm to about 50 ppm, about 25 ppm to about 75 ppm, about 25 ppm to about 100 ppm, about 25 ppm to about 125 ppm, about 25 ppm to about 150 ppm, about 25 ppm to about 175 ppm, about 25 ppm to about 200 ppm, about 25 ppm to about 225 ppm, about 25 ppm to about 250 ppm, about 25 ppm to about 275 ppm, about 25 ppm to about 300 ppm, about 25 ppm to about 325 ppm, about 25 ppm to about 350 ppm, about 25 ppm to about 375 ppm, about 25 ppm to about 400 ppm, about 25 ppm to about 425 ppm, about 25 ppm to about 450 ppm, about 25 ppm to about 475 ppm, about 25 ppm to about 500 ppm, about 50 ppm to about 75 ppm, about 50 ppm to about 100 ppm, about 50 ppm to about 125 ppm, about 50 ppm to about 150 ppm, about 50 ppm to about 175 ppm, about 50 ppm to about 200 ppm, about 50 ppm to about 225 ppm, about 50 ppm to about 250 ppm, about 50 ppm to about 275 ppm, about 50 ppm to about 300 ppm, about 50 ppm to about 325 ppm, about 50 ppm to about 350 ppm, about 50 ppm to about 375 ppm, about 50 ppm to about 400 ppm, about 50 ppm to about 425 ppm, about 50 ppm to about 450 ppm, about 50 ppm to about 475 ppm, about 50 ppm to about 500 ppm, about 75 ppm to about 100 ppm, about 75 ppm to about 125 ppm, about 75 ppm to about 150 ppm, about 75 ppm to about 175 ppm, about 75 ppm to about 200 ppm, about 75 ppm to about 225 ppm, about 75 ppm to about 250 ppm, about 75 ppm to about 275 ppm, about 75 ppm to about 300 ppm, about 75 ppm to about 325 ppm, about 75 ppm to about 350 ppm, about 75 ppm to about 375 ppm, about 75 ppm to about 400 ppm, about 75 ppm to about 425 ppm, about 75 ppm to about 450 ppm, about 75 ppm to about 475 ppm, about 75 ppm to about 500 ppm, about 100 ppm to about 125 ppm, about 100 ppm to about 150 ppm, about 100 ppm to about 175 ppm, about 100 ppm to about 200 ppm, about 100 ppm to about 225 ppm, about 100 ppm to about 250 ppm, about 100 ppm to about 275 ppm, about 100 ppm to about 300 ppm, about 100 ppm to about 325 ppm, about 100 ppm to about 350 ppm, about 100 ppm to about 375 ppm, about 100 ppm to about 400 ppm, about 100 ppm to about 425 ppm, about 100 ppm to about 450 ppm, about 100 ppm to about 475 ppm, about 100 ppm to about 500 ppm, about 150 ppm to about 175 ppm, about 150 ppm to about 200 ppm, about 150 ppm to about 225 ppm, about 150 ppm to about 250 ppm, about 150 ppm to about 275 ppm, about 150 ppm to about 300 ppm, about 150 ppm to about 325 ppm, about 150 ppm to about 350 ppm, about 150 ppm to about 375 ppm, about 150 ppm to about 400 ppm, about 150 ppm to about 425 ppm, about 150 ppm to about 450 ppm, about 150 ppm to about 475 ppm, about 150 ppm to about 500 ppm, about 200 ppm to about 225 ppm, about 200 ppm to about 250 ppm, about 200 ppm to about 275 ppm, about 200 ppm to about 300 ppm, about 200 ppm to about 325 ppm, about 200 ppm to about 350 ppm, about 200 ppm to about 375 ppm, about 200 ppm to about 400 ppm, about 200 ppm to about 425 ppm, about 200 ppm to about 450 ppm, about 200 ppm to about 475 ppm, about 200 ppm to about 500 ppm, about 200 ppm to about 525 ppm, about 200 ppm to about 550 ppm, about 200 ppm to about 575 ppm, about 200 ppm to about 600 ppm, about 250 ppm to about 275 ppm, about 250 ppm to about 300 ppm, about 250 ppm to about 325 ppm, about 250 ppm to about 350 ppm, about 250 ppm to about 375 ppm, about 250 ppm to about 400 ppm, about 250 ppm to about 425 ppm, about 250 ppm to about 450 ppm, about 250 ppm to about 475 ppm, about 250 ppm to about 500 ppm, about 250 ppm to about 525 ppm, about 250 ppm to about 550 ppm, about 250 ppm to about 575 ppm, about 250 ppm to about 600 ppm, about 300 ppm to about 325 ppm, about 300 ppm to about 350 ppm, about 300 ppm to about 375 ppm, about 300 ppm to about 400 ppm, about 300 ppm to about 425 ppm, about 300 ppm to about 450 ppm, about 300 ppm to about 475 ppm, about 300 ppm to about 500 ppm, about 300 ppm to about 525 ppm, about 300 ppm to about 550 ppm, about 300 ppm to about 575 ppm, about 300 ppm to about 600 ppm, about 350 ppm to about 375 ppm, about 350 ppm to about 400 ppm, about 375 ppm to about 400 ppm, about 350 ppm to about 425 ppm, about 350 ppm to about 450 ppm, about 350 ppm to about 475 ppm, about 350 ppm to about 500 ppm, about 350 ppm to about 525 ppm, about 350 ppm to about 550 ppm, about 350 ppm to about 575 ppm, about 350 ppm to about 600 ppm, about 400 ppm to about 500 ppm, about 400 ppm to about 600 ppm, about 400 ppm to about 700 ppm, about 400 ppm to about 800 ppm, about 400 ppm to about 900 ppm, about 400 ppm to about 1,000 ppm, about 400 ppm to about 1,100 ppm, about 400 ppm to about 1,200 ppm, about 400 ppm to about 1,300 ppm, about 400 ppm to about 1,400 ppm, about 400 ppm to about 1,500 ppm, about 400 ppm to about 1,600 ppm, about 400 ppm to about 1,700 ppm, about 400 ppm to about 1,800 ppm, about 400 ppm to about 1,900 ppm, about 400 ppm to about 2,000 ppm, about 500 ppm to about 600 ppm, about 500 ppm to about 700 ppm, about 500 ppm to about 800 ppm, about 500 ppm to about 900 ppm, about 500 ppm to about 1,000 ppm, about 500 ppm to about 1,100 ppm, about 500 ppm to about 1,200 ppm, about 500 ppm to about 1,300 ppm, about 500 ppm to about 1,400 ppm, about 500 ppm to about 1,500 ppm, about 500 ppm to about 1,600 ppm, about 500 ppm to about 1,700 ppm, about 500 ppm to about 1,800 ppm, about 500 ppm to about 1,900 ppm, about 500 ppm to about 2,000 ppm, about 600 ppm to about 700 ppm, about 600 ppm to about 800 ppm, about 600 ppm to about 900 ppm, about 600 ppm to about 1,000 ppm, about 600 ppm to about 1,100 ppm, about 600 ppm to about 1,200 ppm, about 600 ppm to about 1,300 ppm, about 600 ppm to about 1,400 ppm, about 600 ppm to about 1,500 ppm, about 600 ppm to about 1,600 ppm, about 600 ppm to about 1,700 ppm, about 600 ppm to about 1,800 ppm, about 600 ppm to about 1,900 ppm, about 600 ppm to about 2,000 ppm, about 700 ppm to about 800 ppm, about 700 ppm to about 900 ppm, about 700 ppm to about 1,000 ppm, about 700 ppm to about 1,100 ppm, about 700 ppm to about 1,200 ppm, about 700 ppm to about 1,300 ppm, about 700 ppm to about 1,400 ppm, about 700 ppm to about 1,500 ppm, about 700 ppm to about 1,600 ppm, about 700 ppm to about 1,700 ppm, about 700 ppm to about 1,800 ppm, about 700 ppm to about 1,900 ppm, about 700 ppm to about 2,000 ppm, about 800 ppm to about 900 ppm, about 800 ppm to about 1,000 ppm, about 800 ppm to about 1,100 ppm, about 800 ppm to about 1,200 ppm, about 800 ppm to about 1,300 ppm, about 800 ppm to about 1,400 ppm, about 800 ppm to about 1,500 ppm, about 800 ppm to about 1,600 ppm, about 800 ppm to about 1,700 ppm, about 800 ppm to about 1,800 ppm, about 800 ppm to about 1,900 ppm, about 800 ppm to about 2,000 ppm, about 900 ppm to about 1,000 ppm, about 900 ppm to about 1,100 ppm, about 900 ppm to about 1,200 ppm, about 900 ppm to about 1,300 ppm, about 900 ppm to about 1,400 ppm, about 900 ppm to about 1,500 ppm, about 900 ppm to about 1,600 ppm, about 900 ppm to about 1,700 ppm, about 900 ppm to about 1,800 ppm, about 900 ppm to about 1,900 ppm, about 900 ppm to about 2,000 ppm, about 1,000 ppm to about 1,100 ppm, about 1,000 ppm to about 1,200 ppm, about 1,000 ppm to about 1,300 ppm, about 1,000 ppm to about 1,400 ppm, about 1,000 ppm to about 1,500 ppm, about 1,000 ppm to about 1,600 ppm, about 1,000 ppm to about 1,700 ppm, about 1,000 ppm to about 1,800 ppm, about 1,000 ppm to about 1,900 ppm, about 1,000 ppm to about 2,000 ppm, about 1,100 ppm to about 1,200 ppm, about 1,100 ppm to about 1,300 ppm, about 1,100 ppm to about 1,400 ppm, about 1,100 ppm to about 1,500 ppm, about 1,100 ppm to about 1,600 ppm, about 1,100 ppm to about 1,700 ppm, about 1,100 ppm to about 1,800 ppm, about 1,100 ppm to about 1,900 ppm, about 1,100 ppm to about 2,000 ppm, about 1,200 ppm to about 1,300 ppm, about 1,200 ppm to about 1,400 ppm, about 1,200 ppm to about 1,500 ppm, about 1,200 ppm to about 1,600 ppm, about 1,200 ppm to about 1,700 ppm, about 1,200 ppm to about 1,800 ppm, about 1,200 ppm to about 1,900 ppm, about 1,200 ppm to about 2,000 ppm, about 1,300 ppm to about 1,400 ppm, about 1,300 ppm to about 1,500 ppm, about 1,300 ppm to about 1,600 ppm, about 1,300 ppm to about 1,700 ppm, about 1,300 ppm to about 1,800 ppm, about 1,300 ppm to about 1,900 ppm, about 1,300 ppm to about 2,000 ppm, about 1,400 ppm to about 1,500 ppm, about 1,400 ppm to about 1,600 ppm, about 1,400 ppm to about 1,700 ppm, about 1,400 ppm to about 1,800 ppm, about 1,400 ppm to about 1,900 ppm, about 1,400 ppm to about 2,000 ppm, about 1,500 ppm to about 1,600 ppm, about 1,500 ppm to about 1,700 ppm, about 1,500 ppm to about 1,800 ppm, about 1,500 ppm to about 1,900 ppm, about 1,500 ppm to about 2,000 ppm, about 1,600 ppm to about 1,700 ppm, about 1,600 ppm to about 1,800 ppm, about 1,600 ppm to about 1,900 ppm, about 1,600 ppm to about 2,000 ppm, about 1,700 ppm to about 1,800 ppm, about 1,700 ppm to about 1,900 ppm, about 1,700 ppm to about 2,000 ppm, about 1,800 ppm to about 1,900 ppm, about 1,800 ppm to about 2,000 ppm, or about 1,900 ppm to about 2,000 ppm.
71. The kit according to any one of embodiments 47, 48, 54, 56, 57 or 69, wherein the one or more metallic particles are in an amount of about 0.001%, about 0.005%, about 0.01%, about 0.05%, about 0.10%, about 0.11%, about 0.12%, about 0.13%, about 0.14%, about 0.15%, about 0.16%, about 0.17%, about 0.18%, about 0.19%, about 0.2%, about 0.25%, about 0.3%, about 0.35%, about 0.4%, about 0.45%, or about 0.5% by weight of the composition, or at least 0.001%, at least 0.005%, at least 0.01%, at least 0.05%, at least 0.10%, at least 0.11%, at least 0.12%, at least 0.13%, at least 0.14%, at least 0.15%, at least 0.16%, at least 0.17%, at least 0.18%, at least 0.19%, at least 0.2%, at least 0.25%, at least 0.3%, at least 0.35%, at least 0.4%, at least 0.45%, or at least 0.5% by weight of the composition, or at most 0.001%, at most 0.005%, at most 0.01%, at most 0.05%, at most 0.10%, at most 0.11%, at most 0.12%, at most 0.13%, at most 0.14%, at most 0.15%, at most 0.16%, at most 0.17%, at most 0.18%, at most 0.19%, at most 0.2%, at most 0.25%, at most 0.3%, at most 0.35%, at most 0.4%, at most 0.45%, or at most 0.5% by weight of the composition, or about 0.0010% to about 0.005%, about 0.0010% to about 0.010%, about 0.0010% to about 0.050%, about 0.0010% to about 0.10%, about 0.0010% to about 0.15%, about 0.0010% to about 0.20%, about 0.0010% to about 0.25%, about 0.0010% to about 0.30%, about 0.0010% to about 0.35%, about 0.0010% to about 0.40%, about 0.0010% to about 0.45%, about 0.0010% to about 0.50%, about 0.010% to about 0.050%, about 0.010% to about 0.10%, about 0.010% to about 0.15%, about 0.010% to about 0.20%, about 0.010% to about 0.25%, about 0.010% to about 0.30%, about 0.010% to about 0.35%, about 0.010% to about 0.40%, about 0.010% to about 0.45%, about 0.010% to about 0.50%, about 0.10% to about 0.15%, about 0.10% to about 0.20%, about 0.10% to about 0.25%, about 0.10% to about 0.30%, about 0.10% to about 0.35%, about 0.10% to about 0.40%, about 0.10% to about 0.45%, about 0.10% to about 0.50%, about 0.20% to about 0.25%, about 0.20% to about 0.30%, about 0.20% to about 0.35%, about 0.20% to about 0.40%, about 0.20% to about 0.45%, about 0.20% to about 0.50%, about 0.30% to about 0.35%, about 0.30% to about 0.40%, about 0.30% to about 0.45%, about 0.30% to about 0.50%, about 0.40% to about 0.45%, about 0.40% to about 0.50%, or about 0.40% to about 0.50% by weight of the composition.
72. The kit according to any one of embodiments 47, 48, 54, 56, 57 or 69-71, wherein the one or more metallic particles have a mean diameter of about 10 nm, about 20 nm, about 30 nm, about 40 nm, about 50 nm, about 60 nm, about 70 nm, about 80 nm, about 90 nm, about 100 nm, or at least 10 nm, at least 20 nm, at least 30 nm, at least 40 nm, at least 50 nm, at least 60 nm, at least 70 nm, at least 80 nm, at least 90 nm, at least 10⁰ nm, or at most 10 nm, at most 20 nm, at most 30 nm, at most 40 nm, at most 50 nm, at most 60 nm, at most 70 nm, at most 80 nm, at most 90 nm, at most 10⁰ nm, or about 10 nm to about 20 nm, about 10 nm to about 30 nm, about 10 nm to about 40 nm, about 10 nm to about 50 nm, about 10 nm to about 60 nm, about 10 nm to about 70 nm, about 10 nm to about 80 nm, about 10 nm to about 90 nm, about 10 nm to about 100 nm, about 20 nm to about 30 nm, about 20 nm to about 40 nm, about 20 nm to about 50 nm, about 20 nm to about 60 nm, about 20 nm to about 70 nm, about 20 nm to about 80 nm, about 20 nm to about 90 nm, about 20 nm to about 100 nm, about 30 nm to about 40 nm, about 30 nm to about 50 nm, about 30 nm to about 60 nm, about 30 nm to about 70 nm, about 30 nm to about 80 nm, about 30 nm to about 90 nm, about 30 nm to about 100 nm, about 40 nm to about 50 nm, about 40 nm to about 60 nm, about 40 nm to about 70 nm, about 40 nm to about 80 nm, about 40 nm to about 90 nm, about 40 nm to about 100 nm, about 50 nm to about 60 nm, about 50 nm to about 70 nm, about 50 nm to about 80 nm, about 50 nm to about 90 nm, about 50 nm to about 100 nm, about 60 nm to about 70 nm, about 60 nm to about 80 nm, about 60 nm to about 90 nm, about 60 nm to about 100 nm, about 70 nm to about 80 nm, about 70 nm to about 90 nm, about 70 nm to about 100 nm, about 80 nm to about 90 nm, about 80 nm to about 100 nm, or about 90 nm to about 100 nm.
73. The kit according to any one of embodiments 47, 48, 56 or 57, wherein the one or more metal salts include copper chloride, iron chloride, titanium chloride, zinc chloride, silver nitrate, or any combination thereof.
74. The kit according to any one of embodiments 47, 48, 56, 57 or 73, wherein the one or more metal salts are in an amount of 0.05 ppm, 0.10 ppm, 0.15 ppm, 0.20 ppm, 0.25 ppm, 0.30 ppm, 0.35 ppm, 0.40 ppm, 0.45 ppm, 0.50 ppm, 0.55 ppm, 0.60 ppm, 0.65 ppm, 0.70 ppm, 0.75 ppm, 0.80 ppm, 0.85 ppm, 0.90 ppm, 0.95 ppm, 1 ppm, 5 ppm, 10 ppm, 15 ppm, 20 ppm, 25 ppm, 30 ppm, 35 ppm, 40 ppm, 45 ppm, 50 ppm, 55 ppm, 60 ppm, 65 ppm, 70 ppm, 75 ppm, 80 ppm, 85 ppm, 90 ppm, 95 ppm, 100 ppm, 125 ppm, 150 ppm, 175 ppm, 200 ppm, 225 ppm, 250 ppm, 275 ppm, 300 ppm, 325 ppm, 350 ppm, 375 ppm, 400 ppm, 425 ppm, 450 ppm, 475 ppm, 500 ppm, 525 ppm, 550 ppm, 575 ppm, 600 ppm, 625 ppm, 650 ppm, 675 ppm, 700 ppm, 725 ppm, 750 ppm, 775 ppm, 800 ppm, 825 ppm, 850 ppm, 875 ppm, 900 ppm, 925 ppm, 950 ppm, 975 ppm, 1,000 ppm, 1,025 ppm, 1,050 ppm, 1075 ppm, 1,100 ppm, 1,125 ppm, 1,150 ppm, 1,175 ppm, 1,200 ppm, 1,225 ppm, 1,250 ppm, 1,275 ppm, 1,300 ppm, 1,325 ppm, 1,350 ppm, 1,375 ppm, 1,400 ppm, 1,425 ppm, 1,450 ppm, 1,475 ppm, 1,500 ppm, 1,600 ppm, 1,700 ppm, 1,800 ppm, 1,900 ppm, or 2,000 ppm or at least 0.05 ppm, at least 0.10 ppm, at least 0.20 ppm, at least 0.30 ppm, at least 0.40 ppm, at least 0.50 ppm, at least 0.60 ppm, at least 0.70 ppm, at least 0.80 ppm, at least 0.90 ppm, at least 1 ppm, at least 10 ppm, at least 20 ppm, at least 30 ppm, at least 40 ppm, at least 50 ppm, at least 60 ppm, at least 70 ppm, at least 80 ppm, at least 90 ppm, at least 100 ppm, at least 125 ppm, at least 150 ppm, at least 175 ppm, at least 200 ppm, at least 225 ppm, at least 250 ppm, at least 275 ppm, at least 300 ppm, at least 325 ppm, at least 350 ppm, at least 375 ppm, at least 400 ppm, at least 425 ppm, at least 450 ppm, at least 475 ppm, at least 500 ppm, at least 525 ppm, at least 550 ppm, at least 575 ppm, at least 600 ppm, at least 625 ppm, at least 650 ppm, at least 675 ppm, at least 700 ppm, at least 725 ppm, at least 750 ppm, at least 775 ppm, at least 800 ppm, at least 825 ppm, at least 850 ppm, at least 875 ppm, at least 900 ppm, at least 925 ppm, at least 950 ppm, at least 975 ppm, at least 1,000 ppm, at least 1,025 ppm, at least 1,050 ppm, at least 1075 ppm, at least 1,100 ppm, at least 1,125 ppm, at least 1,150 ppm, at least 1,175 ppm, at least 1,200 ppm, at least 1,225 ppm, at least 1,250 ppm, at least 1,275 ppm, at least 1,300 ppm, at least 1,325 ppm, at least 1,350 ppm, at least 1,375 ppm, at least 1,400 ppm, at least 1,425 ppm, at least 1,450 ppm, at least 1,475 ppm, at least 1,500 ppm, at least 1,600 ppm, at least 1,700 ppm, at least 1,800 ppm, at least 1,900 ppm, or at least 2,000 ppm or at most 0.05 ppm, at most 0.10 ppm, at most 0.20 ppm, at most 0.30 ppm, at most 0.40 ppm, at most 0.50 ppm, at most 0.60 ppm, at most 0.70 ppm, at most 0.80 ppm, at most 0.90 ppm, at most 1 ppm, at most 10 ppm, at most 20 ppm, at most 30 ppm, at most 40 ppm, at most 50 ppm, at most 60 ppm, at most 70 ppm, at most 80 ppm, at most 90 ppm, at most 100 ppm, at most 125 ppm, at most 150 ppm, at most 175 ppm, at most 200 ppm, at most 225 ppm, at most 250 ppm, at most 275 ppm, at most 300 ppm, at most 325 ppm, at most 350 ppm, at most 375 ppm, at most 400 ppm, at most 425 ppm, at most 450 ppm, at most 475 ppm, at most 500 ppm, at most 525 ppm, at most 550 ppm, at most 575 ppm, at most 600 ppm, at most 625 ppm, at most 650 ppm, at most 675 ppm, at most 700 ppm, at most 725 ppm, at most 750 ppm, at most 775 ppm, at most 800 ppm, at most 825 ppm, at most 850 ppm, at most 875 ppm, at most 900 ppm, at most 925 ppm, at most 950 ppm, at most 975 ppm, at most 1,000 ppm, at most 1,025 ppm, at most 1,050 ppm, at most 1075 ppm, at most 1,100 ppm, at most 1,125 ppm, at most 1,150 ppm, at most 1,175 ppm, at most 1,200 ppm, at most 1,225 ppm, at most 1,250 ppm, at most 1,275 ppm, at most 1,300 ppm, at most 1,325 ppm, at most 1,350 ppm, at most 1,375 ppm, at most 1,400 ppm, at most 1,425 ppm, at most 1,450 ppm, at most 1,475 ppm, at most 1,500 ppm, at most 1,600 ppm, at most 1,700 ppm, at most 1,800 ppm, at most 1,900 ppm, or at most 2,000 ppm or about 0.5 ppm to about 20 ppm, about 0.5 ppm to about 25 ppm, about 0.5 ppm to about 30 ppm, about 0.5 ppm to about 35 ppm, about 0.5 ppm to about 40 ppm, about 0.5 ppm to about 45 ppm, about 0.5 ppm to about 50 ppm, about 0.5 ppm to about 55 ppm, about 0.5 ppm to about 60 ppm, about 0.5 ppm to about 65 ppm, about 0.5 ppm to about 70 ppm, about 0.5 ppm to about 75 ppm, about 0.5 ppm to about 80 ppm, about 0.5 ppm to about 85 ppm, about 0.5 ppm to about 90 ppm, about 0.5 ppm to about 95 ppm, about 0.5 ppm to about 100 ppm, about 0.75 ppm to about 20 ppm, about 0.75 ppm to about 25 ppm, about 0.75 ppm to about 30 ppm, about 0.75 ppm to about 35 ppm, about 0.75 ppm to about 40 ppm, about 0.75 ppm to about 45 ppm, about 0.75 ppm to about 50 ppm, about 0.75 ppm to about 55 ppm, about 0.75 ppm to about 60 ppm, about 0.75 ppm to about 65 ppm, about 0.75 ppm to about 70 ppm, about 0.75 ppm to about 75 ppm, about 0.75 ppm to about 80 ppm, about 0.75 ppm to about 85 ppm, about 0.75 ppm to about 90 ppm, about 0.75 ppm to about 95 ppm, about 0.75 ppm to about 100 ppm, about 1 ppm to about 20 ppm, about 1 ppm to about 25 ppm, about 1 ppm to about 30 ppm, about 1 ppm to about 35 ppm, about 1 ppm to about 40 ppm, about 1 ppm to about 45 ppm, about 1 ppm to about 50 ppm, about 1 ppm to about 55 ppm, about 1 ppm to about 60 ppm, about 1 ppm to about 65 ppm, about 1 ppm to about 70 ppm, about 1 ppm to about 75 ppm, about 1 ppm to about 80 ppm, about 1 ppm to about 85 ppm, about 1 ppm to about 90 ppm, about 1 ppm to about 95 ppm, about 1 ppm to about 100 ppm, about 5 ppm to about 20 ppm, about 5 ppm to about 25 ppm, about 5 ppm to about 30 ppm, about 5 ppm to about 35 ppm, about 5 ppm to about 40 ppm, about 5 ppm to about 45 ppm, about 5 ppm to about 50 ppm, about 5 ppm to about 55 ppm, about 5 ppm to about 60 ppm, about 5 ppm to about 65 ppm, about 5 ppm to about 70 ppm, about 5 ppm to about 75 ppm, about 5 ppm to about 80 ppm, about 5 ppm to about 85 ppm, about 5 ppm to about 90 ppm, about 5 ppm to about 95 ppm, about 5 ppm to about 100 ppm, about 10 ppm to about 20 ppm, about 10 ppm to about 25 ppm, about 10 ppm to about 30 ppm, about 10 ppm to about 35 ppm, about 10 ppm to about 40 ppm, about 10 ppm to about 45 ppm, about 10 ppm to about 50 ppm, about 10 ppm to about 55 ppm, about 10 ppm to about 60 ppm, about 10 ppm to about 65 ppm, about 10 ppm to about 70 ppm, about 10 ppm to about 75 ppm, about 10 ppm to about 80 ppm, about 10 ppm to about 85 ppm, about 10 ppm to about 90 ppm, about 10 ppm to about 95 ppm, or about 10 ppm to about 100 ppm, about 1 ppm to about 25 ppm, about 1 ppm to about 50 ppm, about 1 ppm to about 75 ppm, about 1 ppm to about 100 ppm, about 1 ppm to about 125 ppm, about 1 ppm to about 150 ppm, about 1 ppm to about 175 ppm, about 1 ppm to about 200 ppm, about 1 ppm to about 225 ppm, about 1 ppm to about 250 ppm, about 1 ppm to about 275 ppm, about 1 ppm to about 300 ppm, about 1 ppm to about 325 ppm, about 1 ppm to about 350 ppm, about 1 ppm to about 375 ppm, about 1 ppm to about 400 ppm, about 1 ppm to about 425 ppm, about 1 ppm to about 450 ppm, about 1 ppm to about 475 ppm, about 1 ppm to about 500 ppm, about 5 ppm to about 25 ppm, about 5 ppm to about 50 ppm, about 5 ppm to about 75 ppm, about 5 ppm to about 100 ppm, about 5 ppm to about 125 ppm, about 5 ppm to about 150 ppm, about 5 ppm to about 175 ppm, about 5 ppm to about 200 ppm, about 5 ppm to about 225 ppm, about 5 ppm to about 250 ppm, about 5 ppm to about 275 ppm, about 1 ppm to about 300 ppm, about 5 ppm to about 325 ppm, about 5 ppm to about 350 ppm, about 5 ppm to about 375 ppm, about 5 ppm to about 400 ppm, about 5 ppm to about 425 ppm, about 5 ppm to about 450 ppm, about 5 ppm to about 475 ppm, about 5 ppm to about 500 ppm, about 10 ppm to about 25 ppm, about 10 ppm to about 50 ppm, about 10 ppm to about 75 ppm, about 10 ppm to about 100 ppm, about 10 ppm to about 125 ppm, about 10 ppm to about 150 ppm, about 10 ppm to about 175 ppm, about 10 ppm to about 200 ppm, about 10 ppm to about 225 ppm, about 10 ppm to about 250 ppm, about 10 ppm to about 275 ppm, about 10 ppm to about 300 ppm, about 10 ppm to about 325 ppm, about 10 ppm to about 350 ppm, about 10 ppm to about 375 ppm, about 10 ppm to about 400 ppm, about 10 ppm to about 425 ppm, about 10 ppm to about 450 ppm, about 10 ppm to about 475 ppm, about 10 ppm to about 500 ppm, about 25 ppm to about 50 ppm, about 25 ppm to about 75 ppm, about 25 ppm to about 100 ppm, about 25 ppm to about 125 ppm, about 25 ppm to about 150 ppm, about 25 ppm to about 175 ppm, about 25 ppm to about 200 ppm, about 25 ppm to about 225 ppm, about 25 ppm to about 250 ppm, about 25 ppm to about 275 ppm, about 25 ppm to about 300 ppm, about 25 ppm to about 325 ppm, about 25 ppm to about 350 ppm, about 25 ppm to about 375 ppm, about 25 ppm to about 400 ppm, about 25 ppm to about 425 ppm, about 25 ppm to about 450 ppm, about 25 ppm to about 475 ppm, about 25 ppm to about 500 ppm, about 50 ppm to about 75 ppm, about 50 ppm to about 100 ppm, about 50 ppm to about 125 ppm, about 50 ppm to about 150 ppm, about 50 ppm to about 175 ppm, about 50 ppm to about 200 ppm, about 50 ppm to about 225 ppm, about 50 ppm to about 250 ppm, about 50 ppm to about 275 ppm, about 50 ppm to about 300 ppm, about 50 ppm to about 325 ppm, about 50 ppm to about 350 ppm, about 50 ppm to about 375 ppm, about 50 ppm to about 400 ppm, about 50 ppm to about 425 ppm, about 50 ppm to about 450 ppm, about 50 ppm to about 475 ppm, about 50 ppm to about 500 ppm, about 75 ppm to about 100 ppm, about 75 ppm to about 125 ppm, about 75 ppm to about 150 ppm, about 75 ppm to about 175 ppm, about 75 ppm to about 200 ppm, about 75 ppm to about 225 ppm, about 75 ppm to about 250 ppm, about 75 ppm to about 275 ppm, about 75 ppm to about 300 ppm, about 75 ppm to about 325 ppm, about 75 ppm to about 350 ppm, about 75 ppm to about 375 ppm, about 75 ppm to about 400 ppm, about 75 ppm to about 425 ppm, about 75 ppm to about 450 ppm, about 75 ppm to about 475 ppm, about 75 ppm to about 500 ppm, about 100 ppm to about 125 ppm, about 100 ppm to about 150 ppm, about 100 ppm to about 175 ppm, about 100 ppm to about 200 ppm, about 100 ppm to about 225 ppm, about 100 ppm to about 250 ppm, about 100 ppm to about 275 ppm, about 100 ppm to about 300 ppm, about 100 ppm to about 325 ppm, about 100 ppm to about 350 ppm, about 100 ppm to about 375 ppm, about 100 ppm to about 400 ppm, about 100 ppm to about 425 ppm, about 100 ppm to about 450 ppm, about 100 ppm to about 475 ppm, about 100 ppm to about 500 ppm, about 150 ppm to about 175 ppm, about 150 ppm to about 200 ppm, about 150 ppm to about 225 ppm, about 150 ppm to about 250 ppm, about 150 ppm to about 275 ppm, about 150 ppm to about 300 ppm, about 150 ppm to about 325 ppm, about 150 ppm to about 350 ppm, about 150 ppm to about 375 ppm, about 150 ppm to about 400 ppm, about 150 ppm to about 425 ppm, about 150 ppm to about 450 ppm, about 150 ppm to about 475 ppm, about 150 ppm to about 500 ppm, about 200 ppm to about 225 ppm, about 200 ppm to about 250 ppm, about 200 ppm to about 275 ppm, about 200 ppm to about 300 ppm, about 200 ppm to about 325 ppm, about 200 ppm to about 350 ppm, about 200 ppm to about 375 ppm, about 200 ppm to about 400 ppm, about 200 ppm to about 425 ppm, about 200 ppm to about 450 ppm, about 200 ppm to about 475 ppm, about 200 ppm to about 500 ppm, about 200 ppm to about 525 ppm, about 200 ppm to about 550 ppm, about 200 ppm to about 575 ppm, about 200 ppm to about 600 ppm, about 250 ppm to about 275 ppm, about 250 ppm to about 300 ppm, about 250 ppm to about 325 ppm, about 250 ppm to about 350 ppm, about 250 ppm to about 375 ppm, about 250 ppm to about 400 ppm, about 250 ppm to about 425 ppm, about 250 ppm to about 450 ppm, about 250 ppm to about 475 ppm, about 250 ppm to about 500 ppm, about 250 ppm to about 525 ppm, about 250 ppm to about 550 ppm, about 250 ppm to about 575 ppm, about 250 ppm to about 600 ppm, about 300 ppm to about 325 ppm, about 300 ppm to about 350 ppm, about 300 ppm to about 375 ppm, about 300 ppm to about 400 ppm, about 300 ppm to about 425 ppm, about 300 ppm to about 450 ppm, about 300 ppm to about 475 ppm, about 300 ppm to about 500 ppm, about 300 ppm to about 525 ppm, about 300 ppm to about 550 ppm, about 300 ppm to about 575 ppm, about 300 ppm to about 600 ppm, about 350 ppm to about 375 ppm, about 350 ppm to about 400 ppm, about 375 ppm to about 400 ppm, about 350 ppm to about 425 ppm, about 350 ppm to about 450 ppm, about 350 ppm to about 475 ppm, about 350 ppm to about 500 ppm, about 350 ppm to about 525 ppm, about 350 ppm to about 550 ppm, about 350 ppm to about 575 ppm, about 350 ppm to about 600 ppm, about 400 ppm to about 500 ppm, about 400 ppm to about 600 ppm, about 400 ppm to about 700 ppm, about 400 ppm to about 800 ppm, about 400 ppm to about 900 ppm, about 400 ppm to about 1,000 ppm, about 400 ppm to about 1,100 ppm, about 400 ppm to about 1,200 ppm, about 400 ppm to about 1,300 ppm, about 400 ppm to about 1,400 ppm, about 400 ppm to about 1,500 ppm, about 400 ppm to about 1,600 ppm, about 400 ppm to about 1,700 ppm, about 400 ppm to about 1,800 ppm, about 400 ppm to about 1,900 ppm, about 400 ppm to about 2,000 ppm, about 500 ppm to about 600 ppm, about 500 ppm to about 700 ppm, about 500 ppm to about 800 ppm, about 500 ppm to about 900 ppm, about 500 ppm to about 1,000 ppm, about 500 ppm to about 1,100 ppm, about 500 ppm to about 1,200 ppm, about 500 ppm to about 1,300 ppm, about 500 ppm to about 1,400 ppm, about 500 ppm to about 1,500 ppm, about 500 ppm to about 1,600 ppm, about 500 ppm to about 1,700 ppm, about 500 ppm to about 1,800 ppm, about 500 ppm to about 1,900 ppm, about 500 ppm to about 2,000 ppm, about 600 ppm to about 700 ppm, about 600 ppm to about 800 ppm, about 600 ppm to about 900 ppm, about 600 ppm to about 1,000 ppm, about 600 ppm to about 1,100 ppm, about 600 ppm to about 1,200 ppm, about 600 ppm to about 1,300 ppm, about 600 ppm to about 1,400 ppm, about 600 ppm to about 1,500 ppm, about 600 ppm to about 1,600 ppm, about 600 ppm to about 1,700 ppm, about 600 ppm to about 1,800 ppm, about 600 ppm to about 1,900 ppm, about 600 ppm to about 2,000 ppm, about 700 ppm to about 800 ppm, about 700 ppm to about 900 ppm, about 700 ppm to about 1,000 ppm, about 700 ppm to about 1,100 ppm, about 700 ppm to about 1,200 ppm, about 700 ppm to about 1,300 ppm, about 700 ppm to about 1,400 ppm, about 700 ppm to about 1,500 ppm, about 700 ppm to about 1,600 ppm, about 700 ppm to about 1,700 ppm, about 700 ppm to about 1,800 ppm, about 700 ppm to about 1,900 ppm, about 700 ppm to about 2,000 ppm, about 800 ppm to about 900 ppm, about 800 ppm to about 1,000 ppm, about 800 ppm to about 1,100 ppm, about 800 ppm to about 1,200 ppm, about 800 ppm to about 1,300 ppm, about 800 ppm to about 1,400 ppm, about 800 ppm to about 1,500 ppm, about 800 ppm to about 1,600 ppm, about 800 ppm to about 1,700 ppm, about 800 ppm to about 1,800 ppm, about 800 ppm to about 1,900 ppm, about 800 ppm to about 2,000 ppm, about 900 ppm to about 1,000 ppm, about 900 ppm to about 1,100 ppm, about 900 ppm to about 1,200 ppm, about 900 ppm to about 1,300 ppm, about 900 ppm to about 1,400 ppm, about 900 ppm to about 1,500 ppm, about 900 ppm to about 1,600 ppm, about 900 ppm to about 1,700 ppm, about 900 ppm to about 1,800 ppm, about 900 ppm to about 1,900 ppm, about 900 ppm to about 2,000 ppm, about 1,000 ppm to about 1,100 ppm, about 1,000 ppm to about 1,200 ppm, about 1,000 ppm to about 1,300 ppm, about 1,000 ppm to about 1,400 ppm, about 1,000 ppm to about 1,500 ppm, about 1,000 ppm to about 1,600 ppm, about 1,000 ppm to about 1,700 ppm, about 1,000 ppm to about 1,800 ppm, about 1,000 ppm to about 1,900 ppm, about 1,000 ppm to about 2,000 ppm, about 1,100 ppm to about 1,200 ppm, about 1,100 ppm to about 1,300 ppm, about 1,100 ppm to about 1,400 ppm, about 1,100 ppm to about 1,500 ppm, about 1,100 ppm to about 1,600 ppm, about 1,100 ppm to about 1,700 ppm, about 1,100 ppm to about 1,800 ppm, about 1,100 ppm to about 1,900 ppm, about 1,100 ppm to about 2,000 ppm, about 1,200 ppm to about 1,300 ppm, about 1,200 ppm to about 1,400 ppm, about 1,200 ppm to about 1,500 ppm, about 1,200 ppm to about 1,600 ppm, about 1,200 ppm to about 1,700 ppm, about 1,200 ppm to about 1,800 ppm, about 1,200 ppm to about 1,900 ppm, about 1,200 ppm to about 2,000 ppm, about 1,300 ppm to about 1,400 ppm, about 1,300 ppm to about 1,500 ppm, about 1,300 ppm to about 1,600 ppm, about 1,300 ppm to about 1,700 ppm, about 1,300 ppm to about 1,800 ppm, about 1,300 ppm to about 1,900 ppm, about 1,300 ppm to about 2,000 ppm, about 1,400 ppm to about 1,500 ppm, about 1,400 ppm to about 1,600 ppm, about 1,400 ppm to about 1,700 ppm, about 1,400 ppm to about 1,800 ppm, about 1,400 ppm to about 1,900 ppm, about 1,400 ppm to about 2,000 ppm, about 1,500 ppm to about 1,600 ppm, about 1,500 ppm to about 1,700 ppm, about 1,500 ppm to about 1,800 ppm, about 1,500 ppm to about 1,900 ppm, about 1,500 ppm to about 2,000 ppm, about 1,600 ppm to about 1,700 ppm, about 1,600 ppm to about 1,800 ppm, about 1,600 ppm to about 1,900 ppm, about 1,600 ppm to about 2,000 ppm, about 1,700 ppm to about 1,800 ppm, about 1,700 ppm to about 1,900 ppm, about 1,700 ppm to about 2,000 ppm, about 1,800 ppm to about 1,900 ppm, about 1,800 ppm to about 2,000 ppm, or about 1,900 ppm to about 2,000 ppm.
75. The kit according to any one of embodiments 47, 48, 56, 57 or 73, wherein the one or more metal salts are in an amount of about 0.001%, about 0.005%, about 0.01%, about 0.05%, about 0.10%, about 0.11%, about 0.12%, about 0.13%, about 0.14%, about 0.15%, about 0.16%, about 0.17%, about 0.18%, about 0.19%, about 0.2%, about 0.25%, about 0.3%, about 0.35%, about 0.4%, about 0.45%, or about 0.5% by weight of the composition, or at least 0.001%, at least 0.005%, at least 0.01%, at least 0.05%, at least 0.10%, at least 0.11%, at least 0.12%, at least 0.13%, at least 0.14%, at least 0.15%, at least 0.16%, at least 0.17%, at least 0.18%, at least 0.19%, at least 0.2%, at least 0.25%, at least 0.3%, at least 0.35%, at least 0.4%, at least 0.45%, or at least 0.5% by weight of the composition, or at most 0.001%, at most 0.005%, at most 0.01%, at most 0.05%, at most 0.10%, at most 0.11%, at most 0.12%, at most 0.13%, at most 0.14%, at most 0.15%, at most 0.16%, at most 0.17%, at most 0.18%, at most 0.19%, at most 0.2%, at most 0.25%, at most 0.3%, at most 0.35%, at most 0.4%, at most 0.45%, or at most 0.5% by weight of the composition, or about 0.0010% to about 0.005%, about 0.0010% to about 0.010%, about 0.0010% to about 0.050%, about 0.0010% to about 0.10%, about 0.0010% to about 0.15%, about 0.0010% to about 0.20%, about 0.0010% to about 0.25%, about 0.0010% to about 0.30%, about 0.0010% to about 0.35%, about 0.0010% to about 0.40%, about 0.0010% to about 0.45%, about 0.0010% to about 0.50%, about 0.010% to about 0.050%, about 0.010% to about 0.10%, about 0.010% to about 0.15%, about 0.010% to about 0.20%, about 0.010% to about 0.25%, about 0.010% to about 0.30%, about 0.010% to about 0.35%, about 0.010% to about 0.40%, about 0.010% to about 0.45%, about 0.010% to about 0.50%, about 0.10% to about 0.15%, about 0.10% to about 0.20%, about 0.10% to about 0.25%, about 0.10% to about 0.30%, about 0.10% to about 0.35%, about 0.10% to about 0.40%, about 0.10% to about 0.45%, about 0.10% to about 0.50%, about 0.20% to about 0.25%, about 0.20% to about 0.30%, about 0.20% to about 0.35%, about 0.20% to about 0.40%, about 0.20% to about 0.45%, about 0.20% to about 0.50%, about 0.30% to about 0.35%, about 0.30% to about 0.40%, about 0.30% to about 0.45%, about 0.30% to about 0.50%, about 0.40% to about 0.45%, about 0.40% to about 0.50%, or about 0.40% to about 0.50% by weight of the composition.
76. A method of reducing or inhibiting microbial growth in water or other liquid, the method comprising, consisting essentially of, or consisting of applying a composition as defined in any one of embodiments 1-46 or applying the components of a kit as defined in any one of embodiments 47-75, to the water or the other liquid in a microbiostatic effective amount, wherein application of the composition reduces or inhibits microbial growth.
77. A composition as defined in any one of embodiments 1-46 or applying the components of a kit as defined in any one of embodiments 47-75 for use in reducing or inhibiting microbial growth in water or other liquid.
78. Use of a composition as defined in any one of embodiments 1-46 or applying the components of a kit as defined in any one of embodiments 47-75 to reduce or inhibit microbial growth in water or other liquid.
79. A method of reducing or inhibiting microbial growth in a water reservoir of a medical device, the method comprising, consisting essentially of, or consisting of applying a composition as defined in any one of embodiments 1-46 or applying the components of a kit as defined in any one of embodiments 47-75, to water or other liquid present in the water reservoir of the medical device in a microbiostatic effective amount, wherein application of the composition reduces or inhibits microbial growth.
79. A method of reducing or inhibiting microbial growth in a water reservoir of a medical device, the method comprising, consisting essentially of, or consisting of applying a composition as defined in any one of embodiments 1-46 or applying the components of a kit as defined in any one of embodiments 47-75, to a water reservoir of the medical device, wherein application of the composition reduces or inhibits microbial growth prior and during operation of the medical device.
80. A composition as defined in any one of embodiments 1-46 or applying the components of a kit as defined in any one of embodiments 47-75 for use in reducing or inhibiting microbial growth in a water reservoir of a medical device.
81. Use of a composition as defined in any one of embodiments 1-46 or applying the components of a kit as defined in any one of embodiments 47-75 to reduce or inhibit microbial growth in a water reservoir of a medical device.
82. A method to wash or irrigate a surface area, the method comprising, consisting essentially of, or consisting of applying a composition as defined in any one of embodiments 1-46 or applying the components of a kit as defined in any one of embodiments 47-75 to the surface area, wherein application of the composition washes or irrigates the surface area.
83. A composition as defined in any one of embodiments 1-46 or applying the components of a kit as defined in any one of embodiments 47-75 for use in washing or irrigating a surface area.
84. Use of a composition as defined in any one of embodiments 1-46 or applying the components of a kit as defined in any one of embodiments 47-75 to wash or irrigate a surface area.
85. The method of embodiment 82, or the use of embodiment 83 or 84, wherein the surface area is a porous surface area or a non-porous surface area.
86. The method of embodiment 82 or 85, or the use of any one of embodiments 83-85, wherein the surface area comprises a table top, counter top, floor, wall, ceiling, window, door, door handle, shower, bath, sink, faucet, toilet, toilet seat, drain, equipment, machinery, personal protective gear, personal biohazard gear, a medical device, dental device, pharmaceutical device, veterinary device, mortuary device or human skin.
87. A method to wash or irrigate a wound in an individual, the method comprising, consisting essentially of, or consisting of applying a composition as defined in any one of embodiments 1-46 or applying the components of a kit as defined in any one of embodiments 47-75 to the wound, wherein application of the composition washes or irrigates the wound.
88. A method to treat and/or provide relief from a wound in an individual, the method comprising, consisting essentially of, or consisting of applying a composition as defined in any one of embodiments 1-46 or applying the components of a kit as defined in any one of embodiments 47-75 to the wound, wherein application of the composition treats and/or provides relief the wound.
89. A method to wash or irrigate an infection in an individual, the method comprising, consisting essentially of, or consisting of applying a composition as defined in any one of embodiments 1-46 or applying the components of a kit as defined in any one of embodiments 47-75 to the infection, wherein application of the composition washes or irrigates the wound.
90. A method to treat and/or provide relief from an infection in an individual, the method comprising, consisting essentially of, or consisting of applying a composition as defined in any one of embodiments 1-46 or applying the components of a kit as defined in any one of embodiments 47-75 to the infection, wherein application of the composition treats and/or provides relief from the infection.
91. A method to wash or irrigate an inflammation in an individual, the method comprising, consisting essentially of, or consisting of applying a composition as defined in any one of embodiments 1-46 or applying the components of a kit as defined in any one of embodiments 47-75 to the inflammation, wherein application of the composition washes or irrigates the inflammation.
92. A method to treat and/or provide relief from an inflammation in an individual, the method comprising, consisting essentially of, or consisting of applying a composition as defined in any one of embodiments 1-46 or applying the components of a kit as defined in any one of embodiments 47-75 to the inflammation, wherein application of the composition treats and/or provides relief from the inflammation.
93. A composition as defined in any one of embodiments 1-46 or applying the components of a kit as defined in any one of embodiments 47-75 for use in washing or irrigating a wound in an individual.
94. Use of a composition as defined in any one of embodiments 1-46 or applying the components of a kit as defined in any one of embodiments 47-75 to wash or irrigate a wound in an individual.
95. A composition as defined in any one of embodiments 1-46 or applying the components of a kit as defined in any one of embodiments 47-75 for use in treating and/or providing relief from a wound in an individual.
96. Use of a composition as defined in any one of embodiments 1-46 or applying the components of a kit as defined in any one of embodiments 47-75 to treat and/or provide relief from a wound in an individual.
97. A composition as defined in any one of embodiments 1-46 or applying the components of a kit as defined in any one of embodiments 47-75 for use in washing or irrigating an infection in an individual.
98. Use of a composition as defined in any one of embodiments 1-46 or applying the components of a kit as defined in any one of embodiments 47-75 to wash or irrigate an infection in an individual.
99. A composition as defined in any one of embodiments 1-46 or applying the components of a kit as defined in any one of embodiments 47-75 for use in treating and/or providing relief from an infection in an individual.
100. Use of a composition as defined in any one of embodiments 1-46 or applying the components of a kit as defined in any one of embodiments 47-75 to treat and/or provide relief from an infection in an individual.
101. A composition as defined in any one of embodiments 1-46 or applying the components of a kit as defined in any one of embodiments 47-75 for use in washing or irrigating an inflammation in an individual.
102. Use of a composition as defined in any one of embodiments 1-46 or applying the components of a kit as defined in any one of embodiments 47-75 to wash or irrigate an inflammation in an individual.
103. A composition as defined in any one of embodiments 1-46 or applying the components of a kit as defined in any one of embodiments 47-75 for use in treating and/or providing relief from an inflammation in an individual.
104. Use of a composition as defined in any one of embodiments 1-46 or applying the components of a kit as defined in any one of embodiments 47-75 to treat and/or provide relief from an inflammation in an individual.
105. The method of any one of embodiments 87-92, or the use of any one of embodiments 93-104, wherein application of the composition is applied topically or administered enterally or parenterally.
106. The method of any one of embodiments 97-92, or the use of any one of embodiments 93-104, wherein the composition is applied daily, every other day, every third of day, once a week, multiple times per week, once a month, multiple times per month, once a year or multiple times per year, as desired.
107. The method of any one of embodiments 87-92, or the use of any one of embodiments 93-104, wherein the composition is applied multiple times per day.
108. The method of any one of embodiments 87-92, or the use of any one of embodiments 93-104, wherein the condition is a wound, an ophthalmic condition, a dermatological condition, an inflammation, an ache or a pain.

### EXAMPLES

The following non-limiting examples are provided for illustrative purposes only in order to facilitate a more complete understanding of representative embodiments now contemplated. These examples should not be construed to limit any of the embodiments described in the present specification, including those pertaining to the compositions, methods and uses disclosed herein.

### Example 1

### Compositions

This example illustrates how to formulate a microbiostatic composition disclosed herein. The components listed below were mixed at ambient temperature using a high shear mixer until the mixture was uniform. The pH of the mixture was adjusted as shown. For hypochlorous acid amounts, unless indicated otherwise, the amount is the final concentration after mixing with other components. A microbiostatic composition disclosed herein is produced as follows: 1) for a formulation disclosed in Tables 1-15 comprising hypochlorous acid/FAC, the formulation is diluted, e.g. with water, so that the concentration of hypochlorous acid/FAC is no more than 0.5 ppm, such as, e.g. between 0.01 ppm to 0.3 ppm; 2) for a formulation disclosed in Tables 1-15 not containing hypochlorous acid/FAC, the formulation is diluted, e.g. with water, 10-fold to 100-fold.

TABLE 1
Composition Formulations

| Component | F1 | F2 | F3 | F4 | F5 | F6 |
|---|---|---|---|---|---|---|
| Hypochlorous Acid | 84 | 84 | 84 | 97 | 97 | 97 |
| | ppm | ppm | ppm | ppm | ppm | ppm |
| Polyhexamethylene | — | — | — | 0.25% | 0.5% | 1.0% |
| biguanide | | | | | | |
| Isopropyl Alcohol | — | — | — | — | — | — |
| Glutaraldehyde | 0.1% | 0.25% | 0.5% | — | — | — |
| Water | q.s. | q.s. | q.s. | q.s. | q.s. | q.s. |
| pH | 5.8 | 6.2 | 6.25 | 6.85 | 6.5 | 6.7 |

TABLE 2
Composition Formulations

| Component | F7 | F8 | F9 | F10 | F11 | F12 |
|---|---|---|---|---|---|---|
| Hypochlorous Acid | 97 | 97 | 97 | 97 | 97 | 97 |
| | ppm | ppm | ppm | ppm | ppm | ppm |
| Polyhexamethylene | — | — | — | — | — | — |
| biguanide | | | | | | |
| Isopropyl Alcohol | — | — | — | — | — | — |
| Glutaraldehyde | 0.5% | 0.5% | 0.5% | 0.5% | 0.5% | 0.5% |
| Sodium Lauryl | 2% | 2% | 2% | 2% | 2% | 2% |
| Sulfate | | | | | | |
| Water | q.s. | q.s. | q.s. | q.s. | q.s. | q.s. |
| pH | 3 | 4 | 5 | 3.5 | 4.5 | 5.5 |

TABLE 3
Composition Formulations

| Component | F13 | F14 | F15 | F16 | F17 | F18 |
|---|---|---|---|---|---|---|
| Hypochlorous Acid | 66 | 76 | 80 | 82 | 73 | 75 |
| | ppm | ppm | ppm | ppm | ppm | ppm |
| Polyhexamethylene | — | — | — | — | — | — |
| biguanide | | | | | | |
| Isopropyl Alcohol | — | — | — | — | — | — |
| Glutaraldehyde | 0.5% | 0.5% | 0.5% | 0.25% | 0.25% | 0.25% |
| Water | q.s. | q.s. | q.s. | q.s. | q.s. | q.s. |
| pH | 3 | 4 | 5 | 3.5 | 4.5 | 5.5 |

TABLE 4
Composition Formulations

| Component | F19 | F20 | F21 | F22 | F23 | F24 |
|---|---|---|---|---|---|---|
| Hypochlorous | 22 | 18 | 16 | 11 | — | — |
| Acid | ppm | ppm | ppm | ppm | | |
| Polyhexa- | 1.25% | 1.5% | 1.75% | 2.0% | 2.0% | 3.0% |
| methylene | | | | | | |
| Biguanide | | | | | | |
| Isopropyl Alcohol | — | — | — | — | 70% | 70% |
| Glutaraldehyde | — | — | — | — | — | — |
| Water | q.s. | q.s. | q.s. | q.s. | q.s. | q.s. |
| pH | 5.75 | 6.6 | 6.4 | 6.9 | ND | ND |

ND, Not Determined.

TABLE 5
Composition Formulations

| Component | F25 | F26 | F27 | F28 | F29 | F30 |
|---|---|---|---|---|---|---|
| Hypochlorous | — | — | — | — | — | — |
| Acid | | | | | | |
| Polyhexa- | 4.0% | 6.0% | 8.0% | 10% | 0.25% | 0.5% |
| methylene | | | | | | |
| Biguanide | | | | | | |
| Isopropyl | 70% | 70% | 70% | 70% | 70% | 70% |
| Alcohol | | | | | | |
| Glutaral- | — | — | — | — | — | — |
| dehyde | | | | | | |
| Water | q.s. | q.s. | q.s. | q.s. | q.s. | q.s. |
| pH | ND | 6.3 | 6.4 | 6.4 | ND | ND |

ND, Not Determined.

TABLE 6
Composition Formulations

| Com- | | | | | | |
|---|---|---|---|---|---|---|
| ponent | F31 | F32 | F33 | F34 | F35 | F36 |
| Hypo- | — | — | — | — | — | — |
| chlorous | | | | | | |
| Acid | | | | | | |
| Polyhexa- | 0.75% | 1.0% | 1.25% | 1.5% | 1.75% | 2.0% |
| methylene | | | | | | |
| Biguanide | | | | | | |
| Isopropyl | 70% | 70% | 70% | 70% | 70% | 70% |
| Alcohol | | | | | | |
| Glutaral- | — | — | — | — | — | — |
| dehyde | | | | | | |
| Water | q.s. | q.s. | q.s. | q.s. | q.s. | q.s. |
| pH | ND | ND | ND | ND | ND | ND |

ND, Not Determined.

TABLE 7
Composition Formulations

| Component | F37 | F38 | F39 | F40 | F41 | F42 |
|---|---|---|---|---|---|---|
| Hypochlorous Acid | 96 | 94 | 92 | 90 | 98 | 96 |
| | ppm | ppm | ppm | ppm | ppm | ppm |
| Polyhexamethylene | 4.0% | 6.0% | 8.0% | 10.0% | 2.0% | 4.0% |
| Biguanide | | | | | | |
| Isopropyl Alcohol | — | — | — | — | — | — |
| Glutaraldehyde | — | — | — | — | — | — |
| Water | q.s. | q.s. | q.s. | q.s. | q.s. | q.s. |
| pH | ND | ND | ND | ND | ND | ND |

ND, Not Determined.

TABLE 8
Composition Formulations

| Component | F43 | F44 | F45 | F46 | F47 | F48 |
|---|---|---|---|---|---|---|
| Hypochlorous | 94 | 92 | 90 | 46 | 58 | — |
| Acid | ppm | ppm | ppm | ppm | ppm | |
| Polyhexa- | 6.0% | 8.0% | 10.0% | 0.25% | 0.25% | 0.25% |
| methylene | | | | | | |
| Biguanide | | | | | | |
| Isopropyl | — | — | — | — | — | 40% |
| Alcohol | | | | | | |
| Glutaraldehyde | — | — | — | — | — | — |
| Water | q.s. | q.s. | q.s. | q.s. | q.s. | q.s. |
| pH | ND | ND | ND | 6.8 | 6.5 | 5.4 |

ND, Not Determined.

TABLE 9
Composition Formulations

| Com- | | | | | | |
|---|---|---|---|---|---|---|
| ponent | F49 | F50 | F51 | F52 | F53 | F54 |
| Hypo- | — | — | — | — | 83 | 56 |
| chlorous | | | | | ppm | ppm |
| Acid | | | | | | |
| Polyhexa- | 0.25% | 0.01% | 0.001% | 0.05% | — | 0.01% |
| methylene | | | | | | |
| Biguanide | | | | | | |
| Isopropyl | 20% | 70% | 70% | 70% | — | — |
| Alcohol | | | | | | |
| Glutaral- | — | — | — | — | — | — |
| dehyde | | | | | | |
| Water | q.s. | q.s. | q.s. | q.s. | q.s. | q.s. |
| pH | 5.5 | 5.8 | 5.8 | 6.0 | 6.6 | 7.4 |

TABLE 10
Composition Formulations

| Component | F55 | F56 | F57 | F58 | F59 | F60 |
|---|---|---|---|---|---|---|
| Hypochlorous | — | — | — | — | — | — |
| Acid | | | | | | |
| Polyhexa- | 8.0% | 9.5% | 5.0% | 10.0% | 10.0% | 10.0% |
| methylene | | | | | | |
| Biguanide | | | | | | |
| Isopropyl | 70% | 70% | 70% | 65% | 60% | 50% |
| Alcohol | | | | | | |
| Glutaraldehyde | 2.0% | — | — | — | — | — |
| ortho- | — | 0.5% | — | — | — | — |
| Phthalaldehyde | | | | | | |
| Benzalkonium | — | — | 5.0% | 5.0% | — | — |
| Chloride | | | | | | |
| Water | q.s. | q.s. | q.s. | q.s. | q.s. | q.s. |
| pH | 4.0 | 3.9 | 7.7 | 7.3 | 6.0 | 6.0 |

TABLE 11
Composition Formulations

| Component | F61 | F62 | F63 | F64 | F65 | F66 |
|---|---|---|---|---|---|---|
| Hypochlorous Acid | — | — | — | — | — | — |
| Polyhexamethylene | 10.0% | 10.0% | 10.0% | 10.0% | 1% | 2% |
| Biguanide | | | | | | |
| Isopropyl Alcohol | — | — | 25.0% | 33.33% | 50% | 50% |
| Ethanol | — | 50.0% | 25.0% | 16.67% | — | — |
| Glutaraldehyde | — | — | — | — | — | — |
| Water | q.s. | q.s. | q.s. | q.s. | q.s. | q.s. |
| pH | ND | 6.15 | 6.38 | 6.29 | 6.01 | 6.19 |

ND, Not Determined.

TABLE 12
Composition Formulations

| Component | F67 | F68 | F69 | F70 | F71 | F97 |
|---|---|---|---|---|---|---|
| Hypochlorous | — | — | — | — | — | 530 |
| Acid | | | | | | ppm* |
| Polyhexa- | 4.0% | 6.0% | 8.0% | 10.0% | 10.0% | 500 |
| methylene | | | | | | ppm |
| Biguanide | | | | | | |
| Isopropyl | 50.0% | — | 50.0% | 40% | 30.0% | — |
| Alcohol | | | | | | |
| Ethanol | — | 50.0% | — | — | — | — |
| Cetylpyridinium | — | — | — | — | — | — |
| Chloride | | | | | | |
| Glutaraldehyde | — | — | — | — | — | — |
| Water | q.s. | q.s. | q.s. | q.s. | q.s. | q.s. |
| pH | 6.29 | 6.20 | 6.40 | 6.34 | 6.23 | 2.8 |

*HOCl concentration prior to mixing.

TABLE 13
Formulations for Composition

| Component | F98 | F99 | F111 | F113 | F114 | F136 |
|---|---|---|---|---|---|---|
| Hypochlorous | — | — | — | 107 | 281 | — |
| Acid | | | | ppm* | ppm* | |
| Polyhexa- | 1% | 1% | 0.12% | 1200 | 1200 | 10% |
| methylene | | | | ppm | ppm | |
| Biguanide | | | | | | |
| Isopropyl | 50% | 20% | 15% | — | — | 45% |
| Alcohol | | | | | | |
| Ethanol | — | — | — | — | — | — |
| Cetylpyridinium | — | — | — | — | — | 5.0% |
| Chloride | | | | | | |
| Glutaraldehyde | — | — | — | — | — | — |
| Water | q.s. | q.s. | q.s. | q.s | q.s. | q.s. |
| pH | 6.8 | 6.3 | 7.60 | 5.37 | 3.7 | 8.29 |

*HOCl concentration prior to mixing.

TABLE 14
Composition Formulations

| Component | F137 | F159 | F160 | F161 | F162 | F164 |
|---|---|---|---|---|---|---|
| Hypochlorous Acid | — | — | — | — | — | — |
| Polyhexamethylene | 6.0% | 5.0% | 10% | 10% | — | — |
| Biguanide | | | | | | |
| Isopropyl Alcohol | 50.0% | 5.0% | 5.0% | 5.0% | 5.0% | 50.0% |
| Ethanol | — | — | — | — | — | — |
| Cetylpyridinium Chloride | 4.0% | 5.0% | — | — | 10% | 10% |
| Glutaraldehyde | — | — | — | — | — | — |
| TRITON ® CG-110 | — | 2.0% | 2.0% | — | — | 2.0% |
| Water | q.s. | q.s. | q.s. | q.s. | q.s. | q.s. |
| pH | 7.8 | 7.3 | 7.0 | 7.3 | 6.2 | 5.6 |

TABLE 15
Composition Formulations

| Component | F165 | F166 | F167 |
|---|---|---|---|
| Hypochlorous Acid | — | — | — |
| Polyhexamethylene Biguanide | — | — | 10% |
| Isopropyl Alcohol | 50% | — | — |
| Ethanol | — | — | — |
| Cetylpyridinium Chloride | 6.0% | 10% | — |
| Glutaraldehyde | — | — | — |
| TRITON ® CG-110 | 2% | 2% | 2% |
| Poloxamer-407 | — | 2% | 2% |
| Water | q.s. | q.s. | q.s. |
| pH | 6.1 | ND | ND |
| ND, Not Determined | | | |

### Example 2

### Compositions

This example illustrates how to formulate a microbiostatic composition disclosed herein. The components listed below were mixed at ambient temperature using a high shear mixer until the mixture was uniform. The pH of the mixture was adjusted as shown. A microbiostatic composition disclosed herein is produced as follows: 1) for a formulation disclosed in Tables 16-57 comprising hypochlorous acid/FAC, the formulation is diluted, e.g. with water, so that the concentration of hypochlorous acid/FAC is no more than 0.5 ppm, such as, e.g. between 0.01 ppm to 0.3 ppm; 2) for a formulation disclosed in Tables 1-15 not containing hypochlorous acid/FAC, the formulation is diluted, e.g. with water, 10-fold to 100-fold.

TABLE 16
Composition Formulations

| Component | F72 | F73 | F74 | F75 | F76 | F77 |
|---|---|---|---|---|---|---|
| Hypochlorous | 273 ppm | 273 ppm | 273 ppm | 273 ppm | 273 ppm | 273 ppm |
| Acid/FAC | | | | | | |
| Titanium | — | — | 50 ppm | 100 ppm | — | — |
| dioxide | | | | | | |
| particles | | | | | | |
| Zinc oxide | 50 ppm | 100 ppm | — | — | — | — |
| particles | | | | | | |
| DTSACI^{a} | — | — | — | — | 100 ppm | 200 ppm |
| Water | q.s. | q.s. | q.s. | q.s. | q.s. | q.s. |
| pH | 5.5 | 6.3 | 4.1 | 3.9 | 3.9 | 3.6 |

^{a}DTSACI: Dimethyloctadecyl[3-(trimethoxysilyl)propyl]ammonium chloride in 58% methanol.

TABLE 17
Composition Formulations

| Component | F78 | F79 | F80 | F81 | F82 | F83 |
|---|---|---|---|---|---|---|
| Hypochlorous Acid/FAC | 273 ppm | 273 ppm | 273 ppm | — | — | — |
| Copper oxide particles | 200 ppm | 300 ppm | — | — | — | — |
| DTSACI^{a} | — | — | 300 ppm | 1% | 5% | 1% |
| Isopropanol | — | — | — | 50% | 50% | 98% |
| Methanol | — | — | — | 2% | 6% | 1% |
| Water | q.s. | q.s. | q.s. | q.s. | q.s. | q.s. |
| pH | 4.8 | 5.3 | 3.4 | 5.5 | 5.1 | 6.6 |

^{a}DTSACI: Dimethyloctadecyl[3-(trimethoxysilyl)propyl]ammonium chloride in 58% methanol.

TABLE 18
Composition Formulations

| Component | F84 | F85 | F86 | F87 | F88 | F89 |
|---|---|---|---|---|---|---|
| Hypochlorous | 273 ppm | 273 ppm | 273 ppm | 273 ppm | — | 530 ppm |
| Acid/FAC | | | | | | |
| Titanium | — | — | — | 200 ppm | — | — |
| dioxide | | | | | | |
| particles | | | | | | |
| DTSACI^{a} | — | — | — | — | 5% | _ |
| Copper (I) | — | 300 ppm | — | — | — | — |
| chloride | | | | | | |
| Copper (II) | — | — | 300 ppm | — | — | — |
| chloride | | | | | | |
| Zinc chloride | 300 ppm | — | — | — | — | — |
| Silver nitrate | — | — | — | — | — | 300 ppm |
| Isopropanol | — | — | — | — | 88.4% | — |
| Methanol | — | — | — | — | 6.6% | — |
| Water | q.s. | q.s | q.s | q.s. | q.s. | q.s. |
| pH | 3.9 | 4.4 | 3.9 | 3.6 | 6.4 | 3.7 |

^{a}DTSACI: Dimethyloctadecyl[3-(trimethoxysilyl)propyl]ammonium chloride in 58% methanol.

TABLE 19
Composition Formulations

| Component | F90 | F91 | F92 | F93 | F94 | F95A |
|---|---|---|---|---|---|---|
| Hypochlorous | 530 ppm | 530 ppm | — | 530 ppm | 530 ppm | 530 |
| | | | | | | ppm |
| Acid/FAC | | | | | | |
| Iron oxide particles | — | 300 ppm | — | — | — | — |
| Titanium dioxide | 100 ppm | — | — | — | — | — |
| particles | | | | | | |
| DTSACI^{a} | — | — | — | — | — | 500 |
| | | | | | | ppm |
| Didodecyldimethyl | — | — | 10% | 300 ppm | 300 ppm | — |
| ammonium | | | | | | |
| chloride | | | | | | |
| Silver nitrate | 200 ppm | — | — | — | — | — |
| Isopropanol | — | — | 50% | — | — | — |
| Water | q.s. | q.s | q.s | q.s. | q.s. | q.s. |
| pH | 3.5 | 3.8 | 8.4 | 3.7 | 3.5 | 3.4 |

^{a}DTSACI: Dimethyloctadecyl[3-(trimethoxysilyl)propyl]ammonium chloride in 58% methanol or water.

TABLE 20
Composition Formulations

| Component | F96 | F100 | F101 | F102 | F103 | F104 |
|---|---|---|---|---|---|---|
| Hypochlorous | 107 ppm | 530 ppm | 530 ppm | 530 ppm | — | — |
| Acid/FAC | | | | | | |
| Silver oxide particles | — | — | — | 200 ppm | — | — |
| Zinc oxide particles | — | — | — | 200 ppm | — | — |
| DTSACI^{a} | 100 ppm | — | — | — | — | 5% |
| Didodecyldimethyl | — | — | — | — | 5% | — |
| ammonium | | | | | | |
| chloride | | | | | | |
| Copper (II) chloride | — | 500 ppm | — | — | — | — |
| Potassium chloride | — | — | 4000 ppm | — | — | — |
| Polyhexamethylene | — | — | — | — | 10% | 10% |
| biguanide | | | | | | |
| Isopropanol | — | — | — | — | 45% | 45% |
| Water | q.s. | q.s. | q.s. | q.s. | q.s | q.s |
| pH | 3.7 | 3.4 | 3.7 | 6.4 | 8.3 | 6.8 |

^{a}DTSACI: Dimethyloctadecyl[3-(trimethoxysilyl)propyl]ammonium chloride in 58% methanol.

TABLE 21
Composition Formulations

| Component | F105 | F106 | F107 | F108 | F109 | F110 |
|---|---|---|---|---|---|---|
| Hypochlorous Acid/FAC | — | — | 530 ppm | 530 ppm | 530 ppm | 530 ppm |
| DTSACI^{a} | 4% | — | — | — | — | 1000 ppm |
| Didodecyldimethyl ammonium | — | 4% | — | — | — | — |
| chloride | | | | | | |
| Potassium chloride | — | — | 600 ppm | 600 ppm | 600 ppm | |
| Potassium phosphate | — | — | 6000 ppm | 2000 ppm | 2000 ppm | — |
| Polyhexamethylene biguanide | 6% | 6% | — | — | — | — |
| Isopropanol | 50% | 50% | — | — | — | — |
| Water | q.s. | q.s. | q.s. | q.s. | q.s | q.s |
| pH | 6.6 | 8.5 | 11.9 | 7.7 | 8.9 | 4.4 |

^{a}DTSACI: Dimethyloctadecyl[3-(trimethoxysilyl)propyl]ammonium chloride in 58% methanol.

TABLE 22
Composition Formulations

| Component | F112 | F115 | F116 | F117 | F118 | F119 |
|---|---|---|---|---|---|---|
| Hypochlorous Acid/FAC | — | — | — | 281 ppm | 530 ppm | 1933 ppm |
| Chlorhexidine | 0.06% | 0.12% | — | — | — | — |
| DTSACI^{a} | — | — | — | — | — | 2000 ppm |
| Zinc acetate | — | — | 0.14% | 300 ppm | 600 ppm | — |
| Polyhexamethylene biguanide | 0.06% | — | 0.12% | — | — | — |
| Ethanol | 15% | 15% | 15% | — | — | — |
| Water | q.s | q.s | q.s | q.s | q.s | q.s |
| pH | 7.6 | 7.3 | 6.9 | 5.4 | 5.4 | 3.7 |

^{a}DTSACI: Dimethyloctadecyl[3-(trimethoxysilyl)propyl]ammonium chloride in 58% methanol.

TABLE 23
Composition Formulations

| Component | F120 | F121 | F122 | F123 | F124 | F125 |
|---|---|---|---|---|---|---|
| Hypochlorous Acid/FAC | 105 ppm | 105 ppm | 500 ppm | 281 ppm | 281 ppm | 530 ppm |
| Titanium dioxide particles | — | — | — | — | — | — |
| Copper zinc iron oxide particles | — | — | — | 300 ppm | — | — |
| DTSACI^{a} | — | — | — | — | 300 ppm | 500 ppm |
| Potassium chloride | 600 ppm | 120 ppm | — | — | — | — |
| Potassium phosphate | 2000 ppm | 400 ppm | — | — | — | — |
| Silver nitrate | — | — | — | — | 300 ppm | 500 ppm |
| Zinc Sulfate | — | — | 600 ppm | — | — | — |
| Water | q.s. | q.s | q.s. | q.s | q.s | q.s |
| pH | 7.0 | 7.6 | 3.7 | 5.2 | 3.5 | 3.5 |

^{a}DTSACI: Dimethyloctadecyl[3-(trimethoxysilyl)propyl]ammonium chloride in 58% methanol.

TABLE 24
Composition Formulations

| Component | F126 | F127 | F128 | F129 | F130 | F131 |
|---|---|---|---|---|---|---|
| Hypochlorous | 281 ppm | 530 ppm | 600 ppm | 278 ppm | 1,933 | 600 |
| | | | | | ppm | ppm |
| Acid/FAC | | | | | | |
| Titanium | 100 ppm | 100 ppm | — | — | — | — |
| dioxide | | | | | | |
| particles | | | | | | |
| DTSACI^{a} | 300 ppm | 500 ppm | — | — | — | — |
| Silver nitrate | 200 ppm | 300 ppm | — | — | — | — |
| Poly(acrylic | — | — | 45% | 2% | 2% | 2% |
| acid) | | | | | | |
| Water | q.s. | q.s | q.s. | q.s. | q.s. | q.s. |
| pH | 3.7 | 3.5 | 2.9 | 2.8 | 2.9 | 7.3 |

^{a}DTSACI: Dimethyloctadecyl[3-(trimethoxysilyl)propyl]ammonium chloride in 58% methanol.

TABLE 25
Composition Formulations

| Component | F132 | F133 | F134 | F135 | F138 | F139 |
|---|---|---|---|---|---|---|
| Hypochlorous Acid/FAC | 600 ppm | 600 ppm | 1,933 ppm | 600 ppm | 600 ppm | 600 ppm |
| DTSACI^{a} | — | — | 600 ppm | — | 600 ppm | 1,200 ppm |
| Silver nitrate | 600 ppm | — | — | — | — | — |
| Zinc sulfate | — | 600 ppm | — | — | — | — |
| Polyhexamethylene biguanide | — | — | — | — | 600 ppm | 1,200 ppm |
| Poly(acrylic acid) | 2% | 2% | 2% | — | — | — |
| Cetylpyrdinium chloride | — | — | — | 600 ppm | — | — |
| Water | q.s. | q.s | q.s. | q.s. | q.s. | q.s |
| pH | 2.7 | 2.9 | 2.9 | 4.0 | 3.0 | 3.0 |

^{a}DTSACI: Dimethyloctadecyl[3-(trimethoxysilyl)propyl]ammonium chloride in 58% methanol.

TABLE 26
Composition Formulations

| Component | F140 | F141 | F142 | F143 | F144 | F145 |
|---|---|---|---|---|---|---|
| Hypochlorous Acid/FAC | 600 ppm | 600 ppm | 600 ppm | 500 ppm | 500 ppm | 500 ppm |
| Copper zinc iron oxide particles | — | — | — | 600 ppm | 300 ppm | — |
| DTSACI^{a} | — | 600 ppm | 1,200 ppm | 600 ppm | — | — |
| TTSACI^{b} | — | — | — | — | — | 500 ppm |
| Polyhexamethylene biguanide | — | — | — | — | — | — |
| ortho-phthalaldehyde | 600 ppm | 600 ppm | 1,200 ppm | — | — | — |
| Water | q.s. | q.s. | q.s. | q.s. | q.s. | q.s |
| pH | 2.6 | 3.4 | 3.2 | 5.2 | 5.0 | 3.4 |

^{a}DTSACI: Dimethyloctadecyl[3-(trimethoxysilyl)propyl]ammonium chloride in 58% methanol.
^{b}TTSACI: Tetradecyldimethyl-(3-trimethoxysilyl)propyl ammonium chloride in water.

TABLE 27
Composition Formulations

| Component | F146 | F147 | F148 | F149 | F150 | F151A |
|---|---|---|---|---|---|---|
| Hypochlorous Acid/FAC | 2,200 ppm | 500 ppm | 500 ppm | 500 ppm | 500 ppm | 500 ppm |
| Copper zinc iron oxide particles | 300 ppm | — | — | — | — | 250 ppm |
| DTSACI^{a} | — | 250 ppm | 125 ppm | 50 ppm | 25 ppm | — |
| Water | q.s. | q.s. | q.s. | q.s. | q.s. | q.s |
| pH | 4.6 | 3.6 | 3.7 | 3.7 | 3.6 | 4.7 |

^{a}DTSACI: Dimethyloctadecyl[3-(trimethoxysilyl)propyl]ammonium chloride in 58% methanol.

TABLE 28
Composition Formulations

| Component | F152 | F153 | F154 | F155 | F156 | F157 |
|---|---|---|---|---|---|---|
| Hypochlorous | 500 ppm | 500 ppm | 500 ppm | 500 ppm | 500 ppm | — |
| Acid/FAC | | | | | | |
| Copper zinc iron | 200 ppm | 150 ppm | 100 ppm | 50 ppm | 25 ppm | — |
| oxide particles | | | | | | |
| DTSACI^{a} | — | — | — | — | — | 10% |
| Isopropanol | — | — | — | — | — | 50% |
| Water | q.s. | q.s. | q.s. | q.s. | q.s. | q.s |
| pH | 4.3 | 3.8 | 3.2 | 3.8 | 3.7 | 4.6 |

^{a}DTSACI: Dimethyloctadecyl[3-(trimethoxysilyl)propyl]ammonium chloride in 58% methanol.

TABLE 29
Composition Formulations

| Component | F158 | F163 | F168 | F169 | F170 | F171 |
|---|---|---|---|---|---|---|
| DTSACI^{a} | — | 20% | 10% | — | — | — |
| Didodecyldimethylammonium | 5% | — | — | — | — | — |
| chloride | | | | | | |
| Polyhexamethylene biguanide | 5% | — | — | — | — | — |
| Isopropanol | 5% | — | 50% | — | — | 5% |
| Methanol | — | 7% | — | 50% | — | — |
| TRITON CG-110 | 3% | — | — | — | — | — |
| Poloxamer-407 | — | — | — | — | 2% | — |
| Quaternium-18 | — | — | — | 6% | 10% | 10% |
| Water | q.s. | q.s. | q.s. | q.s. | q.s. | q.s. |
| pH | ND | 6.4 | ND | ND | ND | ND |

^{a}DTSACI: Dimethyloctadecyl[3-(trimethoxysily)propyl]ammonium chloride in 58% methanol.

TABLE 30
Composition Formulations

| Component | F172 | F173 | F177 | F178 | F179 | F180 |
|---|---|---|---|---|---|---|
| Hypochlorous Acid/ | | | | — | — | 540 ppm |
| FAC | | | | | | |
| DTSACI^{a} | 500 ppm | 20% | — | — | — | — |
| Di-n-alkyldimethyl | — | — | 500 ppm | — | — | — |
| ammonium chloride | | | | | | |
| Didodecyldimethyl | — | — | — | 500 ppm | — | — |
| ammonium chloride | | | | | | |
| Methanol | — | 7% | — | — | — | — |
| Copper (II) chloride | — | — | — | — | — | 200 ppm |
| Iron (II) chloride or | — | — | — | — | 200 ppm | — |
| Iron (III) chloride | | | | | | |
| Silver nitrate | — | — | — | — | 500 ppm | — |
| Zinc acetate | — | — | — | — | 500 ppm | — |
| Zinc chloride | — | — | — | — | 200 ppm | |
| Water | q.s. | q.s. | q.s. | q.s. | q.s. | q.s. |
| pH | 5.4 | 6.4 | 7.6 | 6.2 | 6.9 | 2.6 |

^{a}DTSACI: Dimethyloctadecyl[3-(trimethoxysily)propyl]ammonium chloride in 58% methanol.

TABLE 31
Composition Formulations

| Component | F181 | F182 | F183 | F184 | F185 | F186 |
|---|---|---|---|---|---|---|
| Hypochlorous | 540 ppm | 540 ppm | 540 ppm | 540 ppm | 540 ppm | 540 ppm |
| Acid/FAC | | | | | | |
| DTSACI^{a} | — | 500 ppm | 500 ppm | 500 ppm | 500 ppm | 500 ppm |
| Copper (III) | 200 ppm | — | — | — | — | — |
| chloride | | | | | | |
| Iron (II) chloride | 200 ppm | — | — | — | — | — |
| or Iron (III) | | | | | | |
| chloride | | | | | | |
| Zinc chloride | 200 ppm | — | — | — | — | — |
| Water | q.s. | q.s. | q.s. | q.s. | q.s. | q.s. |
| pH | 2.4 | 5.9 | 5.4 | 6.0 | 5.1 | 6.4 |

^{a}DTSACI: Dimethyloctadecyl[3-(trimethoxysily)propyl]ammonium chloride in 58% methanol.

TABLE 32
Composition Formulations

| Component | F187 | F188 | F189 | F190 | F191 | F192 |
|---|---|---|---|---|---|---|
| Hypochlorous | 540 ppm | 350 ppm | 350 ppm | 260 ppm | 260 ppm | 190 ppm |
| Acid/FAC | | | | | | |
| DTSACI^{a} | 300 ppm | 500 ppm | 300 ppm | 500 ppm | 300 ppm | 500 ppm |
| Water | q.s. | q.s. | q.s. | q.s. | q.s. | q.s. |
| pH | 5.5 | 5.7 | 5.5 | 5.2 | 5.4 | 5.4 |

^{a}DTSACI: Dimethyloctadecyl[3-(trimethoxysily)propyl]ammonium chloride in 58% methanol.

TABLE 33
Composition Formulations

| Component | F193 | F194 | F195 | F95B | F95C | F95D |
|---|---|---|---|---|---|---|
| Hypochlorous | 190 ppm | 100 ppm | 100 ppm | 530 ppm | 75 ppm | 75 ppm |
| Acid/FAC | | | | | | |
| DTSACI^{a} | 300 ppm | 500 ppm | 300 ppm | — | 500 ppm | 300 ppm |
| DTSACI^{b} | — | — | — | 530 ppm | — | — |
| Water | q.s. | q.s. | q.s. | q.s. | q.s. | q.s. |

^{a}DTSACI: Dimethyloctadecyl[3-(trimethoxysily)propyl]ammonium chloride in 58% methanol.
^{b}DTSACI: Dimethyloctadecyl[3-(trimethoxysily)propyl]ammonium chloride in water.

TABLE 34
Composition Formulations

| Component | F95E | F95F | F95G | F95H | F95I | F95J |
|---|---|---|---|---|---|---|
| Hypochlorous | 50 ppm | 50 ppm | 40 ppm | 40 ppm | 35 ppm | 35 ppm |
| Acid/FAC | | | | | | |
| DTSACI^{a} | 500 ppm | 300 ppm | 500 ppm | 300 ppm | 500 ppm | 300 ppm |
| Water | q.s. | q.s. | q.s. | q.s. | q.s. | q.s. |
| pH | 5.8 | 5.4 | 5.8 | 5.4 | 5.8 | 5.4 |

^{a}DTSACI: Dimethyloctadecyl[3-(trimethoxysily)propyl]ammonium chloride in 58% methanol.

TABLE 35
Composition Formulations

| Component | F95K | F95L | F95M | F95N | F95O | F95P |
|---|---|---|---|---|---|---|
| Hypochlorous | 25 ppm | 25 ppm | 10 ppm | 10 ppm | 5 ppm | 5 ppm |
| Acid/FAC | | | | | | |
| DTSACI^{a} | 500 ppm | 300 ppm | 500 ppm | 300 ppm | 500 ppm | 300 ppm |
| Water | q.s. | q.s. | q.s. | q.s. | q.s. | q.s. |
| pH | 5.8 | 5.4 | 5.8 | 5.4 | 5.8 | 5.4 |

^{a}DTSACI: Dimethyloctadecyl[3-(trimethoxysily)propyl]ammonium chloride in 58% methanol.

TABLE 36
Composition Formulations

| Component | F151B | F151C | F151D | F151E | F151F | F151G |
|---|---|---|---|---|---|---|
| Hypochlorous | 350 ppm | 260 ppm | 190 ppm | 100 ppm | 75 ppm | 50 ppm |
| Acid/FAC | | | | | | |
| Copper zinc iron | 250 ppm | 250 ppm | 250 ppm | 250 ppm | 250 ppm | 250 ppm |
| oxide particles | | | | | | |
| Water | q.s. | q.s. | q.s. | q.s. | q.s. | q.s. |
| pH | 4.7 | 4.7 | 4.7 | 4.7 | 4.7 | 4.7 |

TABLE 37
Composition Formulations

| Component | F151H | F151I | F151J | F151K | F151L | F180A |
|---|---|---|---|---|---|---|
| Hypochlorous | 40 ppm | 35 ppm | 25 ppm | 10 ppm | 5 ppm | 350 ppm |
| Acid/FAC | | | | | | |
| Copper zinc iron | 250 ppm | 250 ppm | 250 ppm | 250 ppm | 250 ppm | — |
| oxide particles | | | | | | |
| Copper (II) | — | — | — | — | — | 200 ppm |
| chloride | | | | | | |
| Iron (II) chloride | — | — | — | — | — | 200 ppm |
| or Iron (III) | | | | | | |
| chloride | | | | | | |
| Zinc chloride | — | — | — | — | — | 200 ppm |
| Water | q.s. | q.s. | q.s. | q.s. | q.s. | q.s. |
| pH | 4.7 | 4.7 | 4.7 | 4.7 | 4.7 | 2.6 |

TABLE 38
Composition Formulations

| Component | F180B | F180C | F180D | F180E | F180F | F180G |
|---|---|---|---|---|---|---|
| Hypochlorous | 260 ppm | 190 ppm | 100 ppm | 75 ppm | 50 ppm | 40 ppm |
| Acid/FAC | | | | | | |
| Copper (II) | — | — | 200 ppm | 200 ppm | 200 ppm | 200 ppm |
| chloride | | | | | | |
| Iron (II) chloride | 200 ppm | 200 ppm | 200 ppm | 200 ppm | 200 ppm | 200 ppm |
| or Iron (III) | | | | | | |
| chloride | | | | | | |
| Zinc chloride | 200 ppm | 200 ppm | 200 ppm | 200 ppm | 200 ppm | 200 ppm |
| Water | 200 ppm | 200 ppm | q.s. | q.s. | q.s. | q.s. |
| pH | q.s. | q.s. | 2.6 | 2.6 | 2.6 | 2.6 |
| | 2.6 | 2.6 | | | | |

TABLE 39
Composition Formulations

| Component | F180H | F180I | F180J | F180K |
|---|---|---|---|---|
| Hypochlorous Acid/FAC | 35 ppm | 25 ppm | 10 ppm | 5 ppm |
| Copper (II) chloride | 200 ppm | 200 ppm | 200 ppm | 200 ppm |
| Iron (II) chloride or | 200 ppm | 200 ppm | 200 ppm | 200 ppm |
| Iron (III) chloride | | | | |
| Zinc chloride | 200 ppm | 200 ppm | 200 ppm | 200 ppm |
| Water | q.s. | q.s. | q.s. | q.s. |
| pH | 2.6 | 2.6 | 2.6 | 2.6 |

TABLE 40
Composition Formulations

| Component | F461A | F461B | F461C | F461D | F461E | F461F |
|---|---|---|---|---|---|---|
| Hypochlorous Acid/ | 310 ppm | 290 ppm | 260 ppm | 190 ppm | 100 ppm | 75 ppm |
| FAC | | | | | | |
| Copper (II) | 80 ppm | 80 ppm | 80 ppm | 80 ppm | 80 ppm | 80 ppm |
| chloride | | | | | | |
| Zinc chloride | 80 ppm | 80 ppm | 80 ppm | 80 ppm | 80 ppm | 80 ppm |
| Calcium Oxide | 160 ppm | 120 ppm | 80 ppm | 60 ppm | 40 ppm | 20 ppm |
| Water | q.s. | q.s. | q.s. | q.s. | q.s. | q.s. |
| pH | 7.1 | ND | ND | ND | ND | ND |

TABLE 41
Composition Formulations

| Component | F461G | F461H | F461I | F461J | F461K | F461L |
|---|---|---|---|---|---|---|
| Hypochlorous | 50 ppm | 40 ppm | 35 ppm | 25 ppm | 10 ppm | 5 ppm |
| Acid/FAC | | | | | | |
| Copper (II) | 80 ppm | 80 ppm | 80 ppm | 80 ppm | 80 ppm | 80 ppm |
| chloride | | | | | | |
| Zinc chloride | 80 ppm | 80 ppm | 80 ppm | 80 ppm | 80 ppm | 80 ppm |
| Calcium Oxide | 20 ppm | 20 ppm | 10 ppm | 10 ppm | 5 ppm | 5 ppm |
| Water | q.s. | q.s. | q.s. | q.s. | q.s | q.s |
| pH | ND | ND | ND | ND | ND | ND |

TABLE 42
Composition Formulations

| Component | F469A | F469B | F469C | F469D | F469E | F469F |
|---|---|---|---|---|---|---|
| Hypochlorous | 310 ppm | 290 ppm | 260 ppm | 190 ppm | 100 ppm | 75 ppm |
| Acid/FAC | | | | | | |
| Copper (II) | 120 ppm | 120 ppm | 120 ppm | 120 ppm | 120 ppm | 120 ppm |
| chloride | | | | | | |
| Zinc chloride | 80 ppm | 80 ppm | 80 ppm | 80 ppm | 80 ppm | 80 ppm |
| Calcium Oxide | 160 ppm | 120 ppm | 80 ppm | 60 ppm | 40 ppm | 20 ppm |
| Water | q.s. | q.s. | q.s. | q.s. | q.s. | q.s. |
| pH | 5.0 | ND | ND | ND | ND | ND |

TABLE 43
Composition Formulations

| Component | F469G | F469H | F469I | F469J | F469K | F469L |
|---|---|---|---|---|---|---|
| Hypochlorous | 50 ppm | 40 ppm | 35 ppm | 25 ppm | 10 ppm | 5 ppm |
| Acid/FAC | | | | | | |
| Copper (II) | 120 ppm | 120 ppm | 120 ppm | 120 ppm | 120 ppm | 120 ppm |
| chloride | | | | | | |
| Zinc chloride | 80 ppm | 80 ppm | 80 ppm | 80 ppm | 80 ppm | 80 ppm |
| Calcium Oxide | 20 ppm | 20 ppm | 10 ppm | 10 ppm | 5 ppm | 5 ppm |
| Water | q.s. | q.s. | q.s. | q.s. | q.s | q.s |
| pH | ND | ND | ND | ND | ND | ND |

TABLE 44
Composition Formulations

| Component | F471A | F471B | F471C | F4710 | F471E | F471F |
|---|---|---|---|---|---|---|
| Hypochlorous | 310 ppm | 290 ppm | 260 ppm | 190 ppm | 100 ppm | 75 ppm |
| Acid/FAC | | | | | | |
| Copper (II) | 120 ppm | 120 ppm | 120 ppm | 120 ppm | 120 ppm | 120 ppm |
| chloride | | | | | | |
| Zinc chloride | 120 ppm | 120 ppm | 120 ppm | 120 ppm | 120 ppm | 120 ppm |
| Calcium Oxide | 160 ppm | 120 ppm | 80 ppm | 60 ppm | 40 ppm | 20 ppm |
| Water | q.s. | q.s. | q.s. | q.s. | q.s. | q.s. |
| pH | 5.2 | ND | ND | ND | ND | ND |

TABLE 45
Composition Formulations

| Component | F471G | F471H | F471I | F471J | F471K | F471 L |
|---|---|---|---|---|---|---|
| Hypochlorous | 50 ppm | 40 ppm | 35 ppm | 25 ppm | 10 ppm | 5 ppm |
| Acid/FAC | | | | | | |
| Copper (II) | 120 ppm | 120 ppm | 120 ppm | 120 ppm | 120 ppm | 120 ppm |
| chloride | | | | | | |
| Zinc chloride | 120 ppm | 120 ppm | 120 ppm | 120 ppm | 120 ppm | 120 ppm |
| Calcium Oxide | 20 ppm | 20 ppm | 10 ppm | 10 ppm | 5 ppm | 5 ppm |
| Water | q.s. | q.s. | q.s. | q.s. | q.s | q.s |
| pH | ND | ND | ND | ND | ND | ND |

TABLE 46
Composition Formulations

| Component | F486A | F486B | F486C | F4860 | F486E | F486F |
|---|---|---|---|---|---|---|
| Hypochlorous | 310 ppm | 290 ppm | 260 ppm | 190 ppm | 100 ppm | 75 ppm |
| Acid/FAC | | | | | | |
| Copper (II) | 120 ppm | 120 ppm | 120 ppm | 120 ppm | 120 ppm | 120 ppm |
| chloride | | | | | | |
| Zinc chloride | 120 ppm | 120 ppm | 120 ppm | 120 ppm | 120 ppm | 120 ppm |
| Calcium | 80 ppm | 60 ppm | 40 ppm | 20 ppm | 10 ppm | 10 ppm |
| Phosphate | | | | | | |
| Water | q.s. | q.s. | q.s. | q.s. | q.s. | q.s. |
| pH | 5.0 | ND | ND | ND | ND | ND |

TABLE 47
Composition Formulations

| Component | F486G | F486H | F486I | F486J | F486K | F486L |
|---|---|---|---|---|---|---|
| Hypochlorous | 50 ppm | 40 ppm | 35 ppm | 25 ppm | 10 ppm | 5 ppm |
| Acid/FAC | | | | | | |
| Copper (II) | 120 ppm | 120 ppm | 120 ppm | 120 ppm | 120 ppm | 120 ppm |
| chloride | | | | | | |
| Zinc chloride | 120 ppm | 120 ppm | 120 ppm | 120 ppm | 120 ppm | 120 ppm |
| Calcium | 10 ppm | 10 ppm | 5 ppm | 5 ppm | 5 ppm | 5 ppm |
| Phosphate | | | | | | |
| Water | q.s. | q.s. | q.s. | q.s. | q.s | q.s |
| pH | ND | ND | ND | ND | ND | ND |

TABLE 48
Composition Formulations

| Component | F487A | F487B | F487C | F4870 | F487E | F487F |
|---|---|---|---|---|---|---|
| Hypochlorous | 90 ppm | 80 ppm | 70 ppm | 60 ppm | 50 ppm | 40 ppm |
| Acid/FAC | | | | | | |
| Copper (II) | 80 ppm | 80 ppm | 80 ppm | 80 ppm | 80 ppm | 80 ppm |
| chloride | | | | | | |
| Zinc chloride | 80 ppm | 80 ppm | 80 ppm | 80 ppm | 80 ppm | 80 ppm |
| Calcium Oxide | 160 ppm | 120 ppm | 80 ppm | 60 ppm | 40 ppm | 20 ppm |
| Water | q.s. | q.s. | q.s. | q.s. | q.s. | q.s. |
| pH | 5.0 | ND | ND | ND | ND | ND |

TABLE 49
Composition Formulations

| Component | F487G | F487H | F487I | F487J | F487K | F487L |
|---|---|---|---|---|---|---|
| Hypochlorous | 30 ppm | 25 ppm | 20 ppm | 15 ppm | 10 ppm | 5 ppm |
| Acid/FAC | | | | | | |
| Copper (II) | 80 ppm | 80 ppm | 80 ppm | 80 ppm | 80 ppm | 80 ppm |
| chloride | | | | | | |
| Zinc chloride | 80 ppm | 80 ppm | 80 ppm | 80 ppm | 80 ppm | 80 ppm |
| Calcium Oxide | 20 ppm | 20 ppm | 10 ppm | 10 ppm | 5 ppm | 5 ppm |
| Water | q.s. | q.s. | q.s. | q.s. | q.s | q.s |
| pH | ND | ND | ND | ND | ND | ND |

TABLE 50
Composition Formulations

| Component | F489A | F489B | F489C | F4890 | F489E | F489F |
|---|---|---|---|---|---|---|
| Hypochlorous | 90 ppm | 80 ppm | 70 ppm | 60 ppm | 50 ppm | 40 ppm |
| Acid/FAC | | | | | | |
| Copper (II) | 40 ppm | 40 ppm | 40 ppm | 40 ppm | 40 ppm | 40 ppm |
| chloride | | | | | | |
| Zinc chloride | 40 ppm | 40 ppm | 40 ppm | 40 ppm | 40 ppm | 40 ppm |
| Calcium Oxide | 160 ppm | 120 ppm | 80 ppm | 60 ppm | 40 ppm | 20 ppm |
| Water | q.s. | q.s. | q.s. | q.s. | q.s. | q.s. |
| pH | 5.0 | ND | ND | ND | ND | ND |

TABLE 51
Composition Formulations

| Component | F489G | F489H | F489I | F489J | F489K | F489L |
|---|---|---|---|---|---|---|
| Hypochlorous | 30 ppm | 25 ppm | 20 ppm | 15 ppm | 10 ppm | 5 ppm |
| Acid/FAC | | | | | | |
| Copper (II) | 40 ppm | 40 ppm | 40 ppm | 40 ppm | 40 ppm | 40 ppm |
| chloride | | | | | | |
| Zinc chloride | 40 ppm | 40 ppm | 40 ppm | 40 ppm | 40 ppm | 40 ppm |
| Calcium Oxide | 20 ppm | 20 ppm | 10 ppm | 10 ppm | 5 ppm | 5 ppm |
| Water | q.s. | q.s. | q.s. | q.s. | q.s | q.s |
| pH | ND | ND | ND | ND | ND | ND |

TABLE 52
Composition Formulations

| Component | F491A | F491B | F491C | F4910 | F491E | F491F |
|---|---|---|---|---|---|---|
| Hypochlorous | 90 ppm | 80 ppm | 70 ppm | 60 ppm | 50 ppm | 40 ppm |
| Acid/FAC | | | | | | |
| Copper (II) | 20 ppm | 20 ppm | 20 ppm | 20 ppm | 20 ppm | 20 ppm |
| chloride | | | | | | |
| Zinc chloride | 20 ppm | 20 ppm | 20 ppm | 20 ppm | 20 ppm | 20 ppm |
| Calcium Oxide | 160 ppm | 120 ppm | 80 ppm | 60 ppm | 40 ppm | 20 ppm |
| Water | q.s. | q.s. | q.s. | q.s. | q.s. | q.s. |
| pH | 5.0 | ND | ND | ND | ND | ND |

TABLE 53
Composition Formulations

| Component | F491G | F491H | F491I | F491J | F491K | F491 L |
|---|---|---|---|---|---|---|
| Hypochlorous | 30 ppm | 25 ppm | 20 ppm | 15 ppm | 10 ppm | 5 ppm |
| Acid/FAC | | | | | | |
| Copper (II) | 20 ppm | 20 ppm | 20 ppm | 20 ppm | 20 ppm | 20 ppm |
| chloride | | | | | | |
| Zinc chloride | 20 ppm | 20 ppm | 20 ppm | 20 ppm | 20 ppm | 20 ppm |
| Calcium Oxide | 20 ppm | 20 ppm | 10 ppm | 10 ppm | 5 ppm | 5 ppm |
| Water | q.s. | q.s. | q.s. | q.s. | q.s | q.s |
| pH | ND | ND | ND | ND | ND | ND |

TABLE 54
Composition Formulations

| Component | F493A | F493B | F493C | F4930 | F493E | F493F |
|---|---|---|---|---|---|---|
| Hypochlorous | 310 ppm | 290 ppm | 260 ppm | 190 ppm | 100 ppm | 75 ppm |
| Acid/FAC | | | | | | |
| Copper (II) | 80 ppm | 80 ppm | 80 ppm | 80 ppm | 80 ppm | 80 ppm |
| chloride | | | | | | |
| Zinc chloride | 80 ppm | 80 ppm | 80 ppm | 80 ppm | 80 ppm | 80 ppm |
| Calcium | 80 ppm | 60 ppm | 40 ppm | 20 ppm | 10 ppm | 10 ppm |
| Phosphate | | | | | | |
| Water | q.s. | q.s. | q.s. | q.s. | q.s. | q.s. |
| pH | 5.0 | ND | ND | ND | ND | ND |

TABLE 55
Composition Formulations

| Component | F493G | F493H | F493I | F493J | F493K | F493L |
|---|---|---|---|---|---|---|
| Hypochlorous | 50 ppm | 40 ppm | 35 ppm | 25 ppm | 10 ppm | 5 ppm |
| Acid/FAC | | | | | | |
| Copper (II) | 80 ppm | 80 ppm | 80 ppm | 80 ppm | 80 ppm | 80 ppm |
| chloride | | | | | | |
| Zinc chloride | 80 ppm | 80 ppm | 80 ppm | 80 ppm | 80 ppm | 80 ppm |
| Calcium | 10 ppm | 10 ppm | 5 ppm | 5 ppm | 5 ppm | 5 ppm |
| Phosphate | | | | | | |
| Water | q.s. | q.s. | q.s. | q.s. | q.s | q.s |
| pH | ND | ND | ND | ND | ND | ND |

TABLE 56
Composition Formulations

| Component | F494A | F494B | F494C | F4940 | F494E | F494F |
|---|---|---|---|---|---|---|
| Hypochlorous | 310 ppm | 290 ppm | 260 ppm | 190 ppm | 100 ppm | 75 ppm |
| Acid/FAC | | | | | | |
| Copper (II) | 40 ppm | 40 ppm | 40 ppm | 40 ppm | 40 ppm | 40 ppm |
| chloride | | | | | | |
| Zinc chloride | 40 ppm | 40 ppm | 40 ppm | 40 ppm | 40 ppm | 40 ppm |
| Calcium | 80 ppm | 60 ppm | 40 ppm | 20 ppm | 10 ppm | 10 ppm |
| Phosphate | | | | | | |
| Water | q.s. | q.s. | q.s. | q.s. | q.s. | q.s. |
| pH | 5.0 | ND | ND | ND | ND | ND |

TABLE 57
Composition Formulations

| Component | F494G | F494H | F494I | F494J | F494K | F494L |
|---|---|---|---|---|---|---|
| Hypochlorous | 50 ppm | 40 ppm | 35 ppm | 25 ppm | 10 ppm | 5 ppm |
| Acid/FAC | | | | | | |
| Copper (II) | 40 ppm | 40 ppm | 40 ppm | 40 ppm | 40 ppm | 40 ppm |
| chloride | | | | | | |
| Zinc chloride | 40 ppm | 40 ppm | 40 ppm | 40 ppm | 40 ppm | 40 ppm |
| Calcium | 10 ppm | 10 ppm | 5 ppm | 5 ppm | 5 ppm | 5 ppm |
| Phosphate | | | | | | |
| Water | q.s. | q.s. | q.s. | q.s. | q.s | q.s |
| pH | ND | ND | ND | ND | ND | ND |

### Example 3

### Microbiostatic Assay

This example illustrates how to conduct a microbiostatic assay using a composition disclosed herein.

To prepare stock cultures of pathogenic bacteria, 10 mL nutrient broth was inoculated using a stock culture of Staphylococcus aureus (ATCC 29213) and incubated on an orbital shaker for 24±2 hours at approximately 150 rpm at 36±1° C. This culture was used to inoculate Tryptic Soy Agar (TSA) plates. Each plate was inoculated with the culture. Each plate was incubated for up to 48 hours at 30° C. to 35° C. Following incubation, suspensions were prepared by adding growth from TSA plates into a tube containing sterile water. Each tube was then vortexed and an aliquot was serially diluted and plated onto TSA plate for further numeration. The final numeration count uses aliquot volume, count obtained on TSA plate and the total original volume of culture. The remaining amount in the tube was used to inoculate actual test samples.

To assay for microbiostatic activity, an antimicrobial effectiveness test according to the U.S. Pharmacopeia Chapter 51 (USP-51) protocols were performed. Each composition was tested over a seven log dilution series of pathogen bacteria assays by preparing samples containing 200 μL of a pathogenic bacteria suspension, 20 μL of an organic load, and 20,000 μL of a composition disclosed herein. Control samples contain 200 μL of a pathogenic bacteria suspension, 20 μL of an organic load prepared according to USP 51, and 20,000 μL of water. Composition 1 comprised 0.25 ppm hypochlorous acid while Composition 2 comprised 0.5 ppm hypochlorous acid. Samples were continuously incubated for 28 days, with assay aliquots removed on Day 0, 1, 7, 14, and 28. Once removed, each assay aliquot was immediately mixed with a neutralizing agent to inhibit the activity of the microbial agent being tested. The treated assay samples were serially diluted 1 in 10 over a 6-fold range and plated on petri dishes containing TSA growth medium and incubated at 30° C. to 35° C. for up to 48 hours. The presence of viable bacteria was then measured by counting the number of colonies observed on the agar medium for each assay sample. Log reduction is determined by taking the log₁₀ of the number calculated by taking the number of viable pathogenic bacteria colonies present in the assay sample without serum divided by the number of viable pathogenic bacteria colonies present in the assay sample with serum. In this assay, a composition indicative in having microbiostatic activity is one exhibiting a minimum of a 2-log reduction in the amount of pathogenic bacteria present in an assay sample at Day 14 when compared to Day 0. A greater than two-fold log reduction in the number of pathogenic bacteria present in an assay sample was indicative of microbiocidal activity (i.e., the composition was not microbiostatic). The results of this series of experiments are shown in Table 58.

Initial assays demonstrated that compositions comprising at least 0.5 ppm of hypochlorous acid or free available chlorine exhibited on average a 1×10⁷ log reduction, or 7-log reduction, in pathogen bacteria. These results indicate that the minimum microbiocidal concentration of hypochlorous acid or free available chlorine in the disclosed compositions is approximately 0.5 ppm. However, compositions comprising hypochlorous acid or free available chlorine below 0.5 ppm were microbiostatic. For example, as shown in Table 58, Composition 1, which contained 0.25 ppm hypochlorous acid, demonstrated a microbiostatic activity.

TABLE 58
Microbiostatic Assay

| | Control | | Composition 1 | | Composition 2 | |
|---|---|---|---|---|---|---|
| | Log | | Log | | Log | |
| | Re- | Log | Re- | Log | Re- | Log |
| Day | covery | Reduction | covery | Reduction | covery | Reduction |
| 0 | 7.26 | 0 | 7.34 | 0 | 3.91 | 3.35 |
| 1 | 7.08 | 0 | 7.15 | 0 | 0 | 7 |
| 7 | 7 | 0 | 7 | 0 | 0 | 7 |
| 14 | 7.04 | 0 | 4.08 | 2.96 | 0 | 7 |

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular compound, composition, article, apparatus, methodology, protocol, and/or reagent, etc., described herein, unless expressly stated as such. In addition, those of ordinary skill in the art will recognize that certain changes, modifications, permutations, alterations, additions, subtractions and sub-combinations thereof can be made in accordance with the teachings herein without departing from the spirit of the present specification. It is therefore intended that the following appended claims and claims hereafter introduced are interpreted to include all such changes, modifications, permutations, alterations, additions, subtractions and sub-combinations as are within their true spirit and scope.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. For instance, as mass spectrometry instruments can vary slightly in determining the mass of a given analyte, the term “about” in the context of the mass of an ion or the mass/charge ratio of an ion refers to +/−0.50 atomic mass unit. At the very least, and not as an attempt to limit the application of the doctrine of equivalents to the scope of the claims, each numerical indication should at least be construed in light of the number of reported significant digits and by applying ordinary rounding techniques.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (and equivalent open-ended transitional phrases thereof like including, containing and having) encompasses all the expressly recited elements, limitations, steps and/or features alone or in combination with unrecited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amended for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of.” As such embodiments described herein or so claimed with the phrase “comprising” are expressly or inherently unambiguously described, enabled and supported herein for the phrases “consisting essentially of” and “consisting of.”

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Lastly, the terminology used herein is for the purpose of describing particular embodiments only, and is not intended to limit the scope of the present invention, which is defined solely by the claims. Accordingly, the present invention is not limited to that precisely as shown and described.

1-51. (canceled)

52. A composition comprising hypochlorous acid or free available chlorine, the amount of hypochlorous acid or free available chlorine being in an amount that restrains, prohibits, or otherwise prevents microbial population growth, but does not eradicate the microbial population to a degree that achieves disinfection or sterilization.

53. The composition according to claim 52, wherein the hypochlorous acid or free available chlorine is in an amount of about 0.01 ppm to about 5 ppm.

54. The composition according to claim 53, wherein the hypochlorous acid or free available chlorine is in an amount of about 0.025 ppm to about 2 ppm.

55. The composition according to claim 54, wherein the hypochlorous acid or free available chlorine is in an amount of about 0.05 ppm to about 0.5 ppm.

56. The composition of claim 55, wherein the hypochlorous acid or free available chlorine is in an amount of about 0.05 ppm to about 0.4 ppm.

57. The composition of claim 56, wherein the hypochlorous acid or free available chlorine is in an amount of about 0.1 ppm to about 0.4 ppm.

58. The composition according to claim 52, wherein the hypochlorous acid or free available chlorine is in an amount of less than 0.5 ppm.

59. The composition according to claim 58, wherein the hypochlorous acid or free available chlorine is in an amount of at least 0.01 ppm.

60. The composition according to claim 52, wherein the hypochlorous acid or free available chlorine is in an amount of about 0.01 ppm to about 0.1 ppm, about 0.01 ppm to about 0.2 ppm, about 0.01 ppm to about 0.25 ppm, about 0.01 ppm to about 0.3 ppm, about 0.01 ppm to about 0.4 ppm, about 0.01 ppm to about 0.5 ppm, about 0.05 ppm to about 0.1 ppm, about 0.05 ppm to about 0.2 ppm, about 0.05 ppm to about 0.25 ppm, about 0.05 ppm to about 0.3 ppm, about 0.05 ppm to about 0.4 ppm, or about 0.05 ppm to about 0.5 ppm.

61. The composition according to claim 52, further comprising one or more metallic particles and/or one or more metal salts.

62. The composition according to claim 61, wherein the one or more metal salts include a metal chloride, a metal oxide, or any combination thereof.

63. The composition according to claim 62, wherein the one or more metal salts comprise calcium, copper, zinc or any mixture thereof.

64. The composition according to claim 62, wherein the metal chloride includes copper chloride and zinc chloride.

65. The composition according to claim 62, wherein the metal oxide includes calcium oxide.

66. The composition according to claim 52, further comprising a phosphate buffer.

67. The composition according to claim 66, wherein the phosphate buffer is a calcium phosphate or sodium phosphate.

68. The composition according to claim 52, wherein the amount restrains, prohibits, or otherwise prevents microbial population growth for at least 7 days.

69. The composition according to claim 52, having an effective-use stability of about 1 day to about 30 days.

70. The composition according to claim 52, having an shelf-life stability of about 3 months to about 24 months.

71. The composition according to claim 70, having at most 25% degradation of the hypochlorous acid or free available chlorine.
